(19) United States
(12) Patent Application Publication
(10) Pub. No.: US 2019/0119200 A1
(43) Pub. Date: Apr. 25, 2019

(54) TERTIARY AMIDES AND METHOD OF USE

(71) Applicant: AbbVie Inc., North Chicago, IL (US)

(72) Inventors: Lawrence A. Black, Lahaina, HI (US); William H. Bunnelle, Mundelein, IL (US); Da Chen, Shanghai (CN); Bruce Clapham, Lindenhurst, IL (US); David A. Degoey, Salem, IL (US); Xiangjun Deng, Shanghai (CN); Liqiang Fu, Shanghai (CN); Lisa A. Hazelwood, Lincolnshire, IL (US); Linglong Kong, Shanghai (CN); Qingyu Lang, Shanghai (CN); Chih-Hung Lee, Vernon Hills, IL (US); Mingfeng Li, Shanghai (CN); Greta L. Lundgaard, Round Lake, IL (US); Meena V. Patel, Green Oaks, IL (US); Ruihong Tao, Shanghai (CN); Lin Zhang, Shanghai (CN); Qingwei Zhang, Libertyville, IL (US); Qiangang Zheng, Shanghai (CN); Wei Zhu, Shanghai (CN)

(21) Appl. No.: 16/091,919

(22) PCT Filed: Apr. 6, 2017
(86) PCT No.: PCT/US2017/026361

§ 371 Date: Oct. 5, 2018

(30) Foreign Application Priority Data
Apr. 6, 2016 (CN) PCT/CN2016/078531

(51) Int. Cl. C07C 235/34; C07D 213/69; C07D 321/10; C07D 307/86; C07D 317/46; C07D 333/24; C07C 323/52; C07F 9/38; C07C 313/04; C07D 209/20; C07C 311/32; C07D 305/06; C07D 213/71; C07C 311/51; C07D 239/72; C07D 239/52; C07D 257/06; C07D 215/50; C07D 217/26; C07D 498/04; C07D 307/33

(52) U.S. Cl. CPC C07C 235/34; C07C 2601/04; C07D 321/10; C07D 307/86; C07D 317/46; C07D 333/24; C07C 323/52; C07F 9/3808; C07C 313/04; C07D 209/20; C07C 311/32; C07D 305/06; C07D 213/71; C07C 311/51; C07D 239/72; C07D 239/52; C07D 257/06; C07D 215/50; C07D 217/26; C07D 498/04; C07D 307/33; C07B 2200/07; C07C 2602/08; C07C 2601/14; C07C 2601/02; C07D 213/69

(57) ABSTRACT

Compounds of Formula (I) and pharmaceutically acceptable salts thereof, wherein G¹, G², G³, L¹, L², and L³ are as defined in the specification, are useful in treating conditions or disorders prevented by or ameliorated by the modulation of lysophosphatidic acid receptor 1. Methods for making the compounds are described. Also described are pharmaceutical compositions of compounds of formula (I), and methods for using such compounds and compositions.

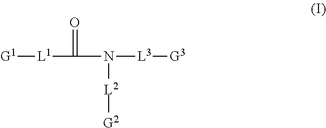

### REFERENCE TO RELATED APPLICATIONS

This application claims the benefit of the filing date of PCT Application No. PCT/CN2016/078531, filed Apr. 6, 2016, the entire content of which is incorporated herein by reference.

### TECHNICAL FIELD

The present disclosure relates to tertiary amide compounds that are modulators of the lysophosphatidic acid receptors (LPARs), useful in treating diseases and conditions mediated and modulated by LPARs. Additionally, the present disclosure relates to compositions containing compounds of the present disclosure and processes for their preparation.

### DESCRIPTION OF RELATED TECHNOLOGY

Lysophosphatidic acid (LPA) mediates a diverse array of physiological processes, including cellular proliferation, migration, adhesion and differentiation. Recently, six subtypes of LPA receptors have been revealed, and it has been demonstrated that their physiological activities occur through LPA receptors (LPARs). These processes are mediated by the interaction of LPA with one of six cognate receptors, termed LPAR1-6. These LPA receptor subtypes are designated as EDG (Endothelial differentiation gene)-2, 4 and 7, respectively, and form part of the EDG receptor family as well as EDG-1, 3, 5, 6 and 8 that are sphingosine-1-phosphate receptors. EDG-2 is also called LPAR1 (Contos, J. J. A. Mol. Pharmacol., 2000: 58(6); 1188-1196.). These LPARs are G protein coupled receptors (GPCRs) that are capable of coupling to both a multitude of G proteins, including G_{i/o}, G_{q} and G_{12/13}, and to arrestin to promote a wide array of downstream intracellular signaling cascades. The activation of these molecular pathways is dependent upon many factors, including receptor expression levels and relative abundance of each effector, cell type, and ligand. While many ligands can evoke equivalent activation of intracellular signals following receptor stimulation, some agonists preferentially signal through a single molecular pathway—a behavior termed biased signaling. Biased agonists are now well-documented for many different GPCRs, and are known to selectively activate one or more effector pathways with greater potency than the others. While biased agonists are well recognized, biased antagonists or biased modulators are a rare and relatively undocumented occurrence. We describe herein the discovery of small molecule modulators of LPAR1 that can preferentially and potently inhibit either the G_{i/o}, and G_{q} pathways or the arrestin pathway, depending upon the compound structure. We also describe compounds that are able to potently inhibit all measured downstream signaling pathways following LPAR1 activation by LPA.

LPA receptors are localized throughout the human body and distributed differently between their subtypes, and it is reflected that the role of each receptor is different dependent upon the tissues and organs where it is located. For example, there is a strong connection between LPA and carcinoma. It is known that LPA enhances the proliferation of the epithelial cells (Sturm, A. et al., Gastroenterology. 1999, 117(2):368-377), and affects proliferation of hepatocytes and stellate cells in liver diseases complicating platelet activation (Ikeda, H. et al. Biochem. Biophys. Res. Commun., 1998, 248, 436) and prostate cancer cells (Guo, C. et al., J. Urol. 2000, 163, 1027; Qi, C. et al., J. Cellular Physiol. 1998 174, 261).

LPA is also connected to the growth of various cells such as smooth muscle cells (Ediger, T. L. et al., Am. J. Physiol. Lung Cell. Mol. Physiol., 2002, 282, L91-8), fibroblasts (Roche, S. et al., Mol. Cell Biol., 1998, 18, 7119), mesangial cells (Inoue, C. N. et al., Clin. Science, 1999, 96, 431), hepatocytes, liver stellate cells (Ikeda, H. et al., Biochem. Biophys. Res. Commun., 1998, 248, 436), vascular smooth muscle cells (Tokumura, A. et al., Am. J. Physiol. Cell Physiol., 1994, 267, C204), vascular endothelial cells (Lee, H. et al., Am. J. Physiol. Cell Physiol., 2000, 278, C612), and adipocytes (Valet, P. et al., J. Clin. Invest., 1998, 101, 1431) as well as different cancer cells. LPA is also known to involve the function of chemotaxis of inflammatory cells and cell growth (Idzko, M. et al., J Immunol. 2004; 172(7):4480-4485). LPA can stimulate cell proliferation and cytokine-secreting activity of immune cells (Goetzl E. J. et al., J. Imunol. 1999, 162, 2049), as well as platelet aggregation activity (Tokumura, A. et al., Biochem. Biophys. Res. Commun., 1981, 99, 391). Based on the LPAR1 knockout mouse, LPAR1 is considered to be related to kidney function.

These results indicate that inhibiting LPAR1 receptor activity is beneficial for the prevention and/or treatment of various diseases such as liver, kidney, skin, heart and lung disease, cancer-associated disease, proliferative disease, inflammation/immune system disease, disease of secretory dysfunction, and fibrosis.

Accordingly, there is a need for novel compounds able to modulate LPAR1 activity. In particular, the present disclosure provides compounds that may act as LPAR1 modulators for the treatment of fibrotic diseases. The present disclosure also provides methods for the preparation of these compounds, pharmaceutical compositions comprising these compounds and methods for the treatment of liver, kidney, skin, heart and lung disease, cancer-associated disease, proliferative disease, inflammation/immune system disease, disease of secretory dysfunction, and fibrosis by administering the compounds of the present disclosure.

### SUMMARY OF THE INVENTION

The present disclosure is directed to tertiary amides having a structure of formula (I)

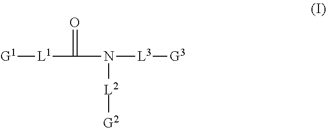

or a pharmaceutically acceptable salt thereof, wherein:

G¹ is selected from

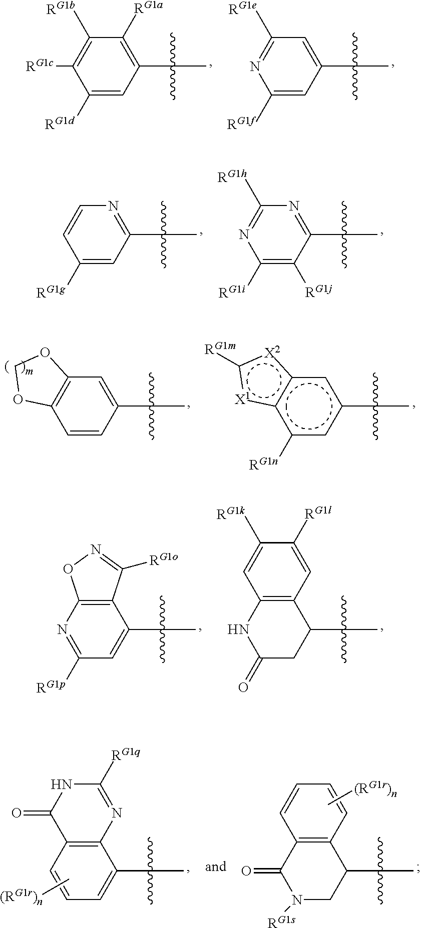

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p}, R^{G1q}, and R^{G1s} are selected from the group consisting of hydrogen and C₁-C₃alkyl; wherein the C₁-C₃alkyl is unsubstituted or optionally substituted with one, two, or three fluorines;

one of X¹ and X² is O and the other is CH;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, and wherein the —CH₂CH₂CH₂— or C₁-C₃alkyl substituent are each independently optionally substituted with one, two, or three fluorines;

G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b},

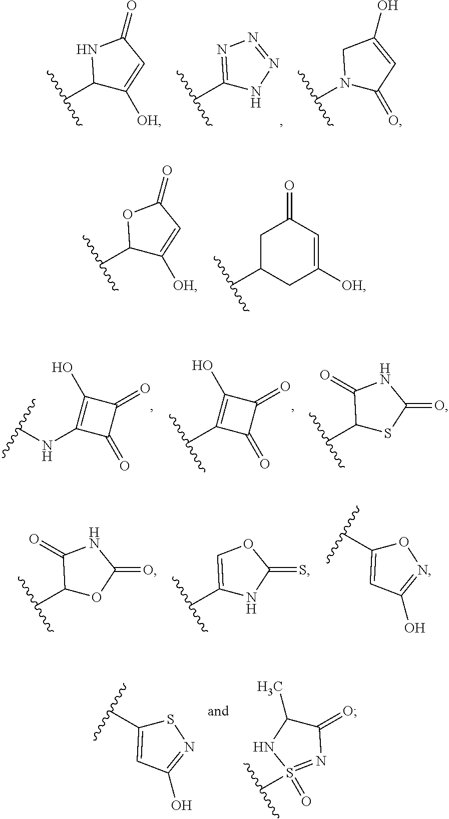

wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein at least one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected, at each occurrence, from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

Another aspect of the present disclosure relates to pharmaceutical compositions comprising compounds of the present disclosure or pharmaceutically acceptable salts thereof, and a pharmaceutical carrier. Such compositions can be administered in accordance with a method of the present disclosure, such as part of a therapeutic regimen for treatment or prevention of conditions and disorders related to lysophosphatidic acid receptor 1 activity. In embodiments, the pharmaceutical compositions may additionally further comprise one or more therapeutically active ingredients suitable for use in combination with the compounds of the present disclosure. For example, in a more particular aspect, the additional therapeutically active ingredient is an agent for the treatment of liver, kidney, skin, heart and lung disease, cancer-associated disease, proliferative disease, inflammation/immune system disease, disease of secretory dysfunction, and fibrosis.

Moreover, the compounds of the present disclosure or pharmaceutically acceptable salts thereof, useful in the pharmaceutical compositions and treatment methods disclosed herein, are pharmaceutically acceptable as prepared and used.

In embodiments, the present disclosure relates to a method of modulating the lysophosphatidic acid receptor 1 activity. The method is useful for treating or preventing conditions and disorders related to fibrotic disease activity in mammals. More particularly, the method is useful for treating or preventing conditions and disorders related to liver, kidney, skin, heart and lung disease, cancer-associated disease, proliferative disease, inflammation/immune system disease, and disease by secretory dysfunction. Accordingly, the compounds and compositions of the present disclosure are useful as a medicament for treating or preventing lysophosphatidic acid receptor 1 modulated disease.

The compounds, compositions comprising the compounds or pharmaceutically acceptable salts thereof, methods for making the compounds, and methods for treating or preventing conditions and disorders by administering the compounds are further described herein.

In embodiments, the compounds of the present disclosure or pharmaceutically acceptable salts thereof are provided for use in the treatment of liver, kidney, skin, heart and lung disease, cancer-associated disease, proliferative disease, inflammation/immune system disease, disease of secretory dysfunction, and fibrosis. In embodiments, the compounds of the present disclosure or pharmaceutically acceptable salts thereof are provided for use in the treatment of liver, kidney, skin, heart and lung disease, cancer-associated disease, proliferative disease, inflammation/immune system disease, disease of secretory dysfunction, and fibrosis by preferentially and potently inhibiting either the G_{i/o} and G_{q} pathways or the arrestin pathway.

The present disclosure also provides pharmaceutical compositions comprising a compound of the present disclosure or pharmaceutically acceptable salts thereof, and a suitable pharmaceutical carrier for use in medicine. In embodiments, the pharmaceutical composition is for use in the treatment of liver, kidney, skin, heart and lung disease, cancer-associated disease, proliferative disease, inflammation/immune system disease, disease of secretory dysfunction, and fibrosis.

These and other objects of the present disclosure are described in the following paragraphs. These objects should not be deemed to narrow the scope of the present disclosure.

### BRIEF DESCRIPTION OF THE FIGURE

FIG. 1 shows a graphical representation of the effect of Example 7 in a mouse unilateral ureteral obstruction (UUO) kidney fibrosis model.

### DETAILED DESCRIPTION OF THE INVENTION

Described herein are compounds of formula (I)

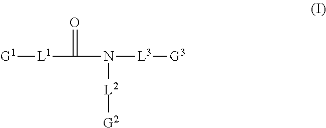

wherein G¹, G², G³, L¹, L² and L³ are defined above in the Summary and below in the Detailed Description. Further, compositions comprising such compounds and methods for treating conditions and disorders using such compounds and compositions are also described.

Compounds included herein may contain one or more variable(s) that occur more than one time in any substituent or in the formulae herein. Definition of a variable on each occurrence is independent of its definition at another occurrence. Further, combinations of substituents are permissible only if such combinations result in stable compounds. Stable compounds are compounds, which can be isolated from a reaction mixture.

### Definition of Terms

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds; reference to “a pharmaceutically acceptable carrier” means a single pharmaceutically acceptable carrier as well as one or more pharmaceutically acceptable carriers, and the like.

As used in the specification and the appended claims, unless specified to the contrary, the following terms have the meaning indicated:

The term “alkenyl” as used herein, means a straight or branched hydrocarbon chain containing from 2 to 10 carbons and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” means a divalent group derived from a straight or branched chain hydrocarbon of from 2 to 10 carbon atoms containing at least one double bond. Representative examples of alkenylene include, but are not limited to, —CH═CH—, —CH═CH₂CH₂—, and —CH═C(CH₃)CH₂—.

The term “alkoxy” as used herein, means a C₁-C₆ alkyl group, as defined herein, appended to the parent molecular moiety through an oxygen atom. Representative, non-limiting examples of alkoxy include methoxy, ethoxy, propoxy, 2-propoxy, butoxy, tert-butoxy, pentyloxy, and hexyloxy.

The term “alkyl” as used herein, means a straight or branched, saturated hydrocarbon chain containing from 1 to 10 carbon atoms. The term “lower alkyl” or “C₁-C₆alkyl” means a straight or branched chain hydrocarbon containing from 1 to 6 carbon atoms. The term “C₁-C₃alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene” or “alkylenyl” denotes a divalent group derived from a straight or branched chain hydrocarbon containing from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkynyl” as used herein, means a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “aryl” as used herein, means phenyl or a bicyclic aryl. The bicyclic aryl is naphthyl, or a phenyl fused to a monocyclic cycloalkyl, or a phenyl fused to a monocyclic cycloalkenyl. Representative examples of the aryl groups include, but are not limited to, dihydroindenyl, indenyl, naphthyl, dihydronaphthalenyl, and tetrahydronaphthalenyl. The bicyclic aryl is attached to the parent molecular moiety through any carbon atom contained within the bicyclic ring system. The aryl groups of the present disclosure can be unsubstituted or substituted.

The term “cycloalkenyl” or “cycloalkene” as used herein, means a monocyclic or a bicyclic hydrocarbon ring system. The monocyclic cycloalkenyl has four-, five-, six-, seven- or eight carbon atoms and zero heteroatoms. The four-membered ring systems have one double bond, the five- or six-membered ring systems have one or two double bonds, and the seven- or eight-membered ring systems have one, two or three double bonds. Representative examples of monocyclic cycloalkenyl groups include, but are not limited to, cyclobutenyl, cyclopentenyl, cyclohexenyl, cycloheptenyl and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group, or a bridged monocyclic ring system in which two non-adjacent carbon atoms of the monocyclic ring are linked by an alkylene bridge containing one, two, three, or four carbon atoms. Representative examples of the bicyclic cycloalkenyl groups include, but are not limited to, 4,5,6,7-tetrahydro-3aH-indene, octahydronaphthalenyl and 1,6-dihydro-pentalene. The monocyclic and bicyclic cycloalkenyl can be attached to the parent molecular moiety through any substitutable atom contained within the ring systems, and can be unsubstituted or substituted.

The term “cycloalkyl” or “cycloalkane” as used herein, means a monocyclic, a bicyclic, a tricyclic, or a spirocyclic cycloalkyl. The monocyclic cycloalkyl is a carbocyclic ring system containing three to eight carbon atoms, zero heteroatoms and zero double bonds. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl ring, or a bridged monocyclic ring system in which two non-adjacent carbon atoms of the monocyclic ring are linked by an alkylene bridge containing one, two, three, or four carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic cycloalkyls are exemplified by a bicyclic cycloalkyl fused to a monocyclic cycloalkyl, or a bicyclic cycloalkyl in which two non-adjacent carbon atoms of the ring systems are linked by an alkylene bridge of 1, 2, 3, or 4 carbon atoms. Representative examples of tricyclic-ring systems include, but are not limited to, tricyclo[3.3.1.0^{3,7}]nonane (octahydro-2,5-methanopentalene or noradamantane), and tricyclo[3.3.1.1^{3,7}]decane (adamantane). The monocyclic, bicyclic, and tricyclic cycloalkyls can be unsubstituted or substituted, and are attached to the parent molecular moiety through any substitutable atom contained within the ring system. Spirocyclic cycloalkyl is exemplified by a monocyclic or a bicyclic cycloalkyl, wherein two of the substituents on the same carbon atom of the ring, together with said carbon atom, form a 4-, 5-, or 6-membered monocyclic cycloalkyl. An example of a spirocyclic cycloalkyl is spiro[2.5]octane. The spirocyclic cycloalkyl groups of the present disclosure can be appended to the parent molecular moiety through any substitutable carbon atom of the groups.

The term “halo” or “halogen” as used herein, means Cl, Br, I, or F.

The term “haloalkyl” as used herein, means an alkyl group, as defined herein, in which one, two, three, four, five, six, seven or eight hydrogen atoms are replaced by halogen. Representative examples of haloalkyl include, but are not limited to, chloromethyl, 2-fluoroethyl, 2,2,2-trifluoroethyl, trifluoromethyl, difluoromethyl, pentafluoroethyl, 2-chloro-3-fluoropentyl, and trifluoropropyl such as 3,3,3-trifluoropropyl.

The term “heteroaryl” as used herein, means a monocyclic heteroaryl or a bicyclic heteroaryl. The monocyclic heteroaryl is a five- or six-membered ring. The five-membered ring contains two double bonds. The five-membered ring may contain one heteroatom selected from O or S; or one, two, three, or four nitrogen atoms and optionally one oxygen or sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but are not limited to, benzofuranyl, benzothienyl, benzoxazolyl, benzimidazolyl, benzoxadiazolyl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, thiazolo[5,4-b]pyridin-2-yl, thiazolo[5,4-d]pyrimidin-2-yl, and 5,6,7,8-tetrahydroquinolin-5-yl. The monocyclic and bicyclic heteroaryl groups of the present disclosure can be substituted or unsubstituted and are connected to the parent molecular moiety through any carbon atom or any nitrogen atom contained within the ring systems.

The term “heterocycle” or “heterocyclic” as used herein, means a monocyclic heterocycle, a bicyclic heterocycle, a tricyclic heterocycle, or a spirocyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), oxa-adamantane (2-oxatricyclo[3.3.1.1^{3,7}]decane), and octahydro-1H-4,7-epiminoisoindole. The spirocyclic heterocycles are exemplified by a monocyclic heterocycle as defined herein wherein one carbon atom of the monocyclic heterocycle is bridged by two ends of an alkylene chain. In the spirocyclic heterocycle, one or more carbon atoms in the bridging alkylene chain may be replaced with a heteroatom. Examples of spirocyclic heterocycles include, but are not limited to, 4,7-diazaspiro[2.5]octane, 2-oxa-6-azaspiro[3.3]heptane, 2,6-diazaspiro[3.3]heptane, 2-oxa-5,8-diazaspiro[3.5]nonane, 2,7-diazaspiro[3.5]nonane, 1,4-dioxa-8-azaspiro[4.5]decane, 1,6-diazaspiro[3.3]heptane, 1-azaspiro[4.4]nonane, 7-azaspiro[3.5]nonane, 1,4-dioxa-7-azaspiro[4.4]nonane, 5,8-diazaspiro[3.5]nonane, 5,8-dioxa-2-azaspiro[3.4]octane, 2-oxa-6-azaspiro[3.4]octane, 6-oxa-1-azaspiro[3.3]heptane, 6-oxa-2-azaspiro[3.4]octane, 6-oxa-2-azaspiro[3.5]nonane, and 7-oxa-2-azaspiro[3.5]nonane. The monocyclic, bicyclic, tricyclic, and spirocyclic heterocycles are connected to the parent molecular moiety through any carbon atom or any nitrogen atom contained within the rings, and can be unsubstituted or substituted.

The term “heteroatom” as used herein, means a nitrogen, oxygen, phosphorous or sulfur atom.

The term “hydroxyl” or “hydroxy” as used herein, means an —OH group.

The term “oxo” as used herein means (═O).

In some instances, the number of carbon atoms in a hydrocarbyl substituent (e.g., alkyl, alkenyl, alkynyl, cycloalkyl or cycloalkenyl) is indicated by the prefix “Cₓ-C_{y}”, wherein x is the minimum and y is the maximum number of carbon atoms in the substituent. Thus, for example, “C₁-C₆alkyl” refers to an alkyl substituent containing from 1 to 6 carbon atoms. Illustrating further, C₃-C₆cycloalkyl means a saturated hydrocarbyl ring containing from 3 to 6 carbon ring atoms.

As used herein, the term “radiolabel” refers to a compound of the present disclosure in which at least one of the atoms is a radioactive atom or radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F, ³⁵S, ¹²³I, and ¹²⁵I.

If a moiety is described as “substituted”, a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

The terms “treat”, “treating”, and “treatment” refer to a method of alleviating or abrogating a disease and/or its attendant symptoms.

The terms “prevent”, “preventing”, and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

The phrase “therapeutically effective amount” means an amount of a compound, or a pharmaceutically acceptable salt thereof, sufficient to prevent the development of or to alleviate to some extent one or more of the symptoms of the condition or disorder being treated when administered alone or in conjunction with another therapeutic agent or treatment in a particular subject or subject population. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

The term “subject” is defined herein to refer to animals such as mammals, including, but not limited to, primates (e.g., humans), cows, sheep, goats, pigs, horses, dogs, cats, rabbits, rats, mice and the like. In preferred embodiments, the subject is a human.

The term ‘one or more’ refers to one to four. In one embodiment it refers to one or three. In another embodiment it refers to one to three. In a further embodiment it refers to one to two. In yet other embodiment it refers to two. In yet other further embodiment it refers to one.

### Compounds

Compounds of the present disclosure can have the formula (I) as described in the Summary.

Particular values of variable groups in compounds of formula (I) are as follows. Such values can be used where appropriate with any of the other values, definitions, claims or embodiments defined hereinbefore or hereinafter.

In one embodiment, G¹ is selected from

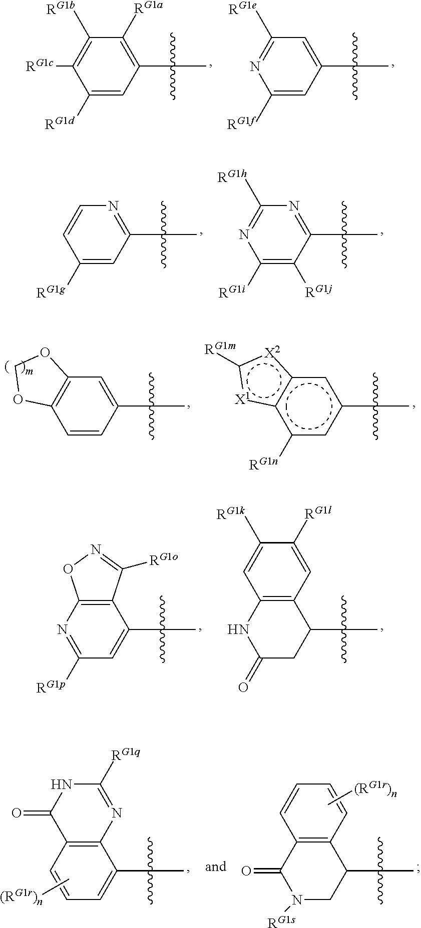

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines; and

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines.

In one embodiment, G¹ is

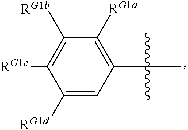

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines; and

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen.

In one embodiment, G¹ is

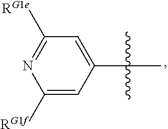

wherein

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines; and

R^{x} is C₁-C₃alkyl; wherein the C₁-C₃alkyl is unsubstituted or optionally substituted with one, two or three fluorines.

In one embodiment, G¹ is

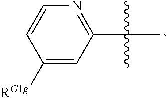

wherein

R^{G1g} is selected from the group consisting of halogen and C₁-C₃alkoxy; wherein the C₁-C₃alkoxy is unsubstituted or optionally substituted with one, two, or three fluorines.

In one embodiment, G¹ is

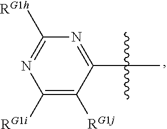

wherein

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; and

R^{G1j} is selected from the group consisting of hydrogen or halogen.

In one embodiment, G¹ is

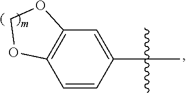

wherein is one, two or three.

In one embodiment, G¹ is

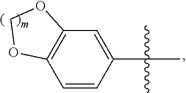

wherein m is one.

In one embodiment, G¹ is

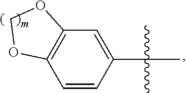

wherein m is two.

In one embodiment, G¹ is

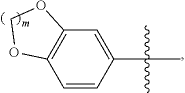

wherein m is three.

In one embodiment, G¹ is

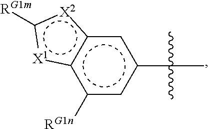

wherein

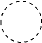

means the ring in which it is drawn is aromatic.

R^{G1m} is selected from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines; and

one of X¹ and X² is O and the other is CH.

In one embodiment, G¹ is

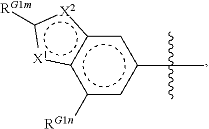

wherein

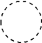

means the ring in which it is drawn is aromatic.

R^{G1m} is selected from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines; and

X¹ is O and X² is CH.

In one embodiment, G¹ is

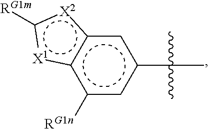

wherein

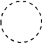

means the ring in which it is drawn is aromatic.

R^{G1m} is selected from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines; and

X¹ is CH and X² is O. In one embodiment, G¹ is

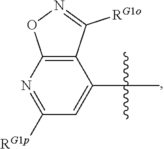

wherein

R^{G1o} and R^{G1p} are independently selected from the group consisting of hydrogen and C₁-C₃alkyl; wherein the C₁-C₃alkyl is unsubstituted or optionally substituted with one, two, or three fluorines.

In one embodiment, G¹ is

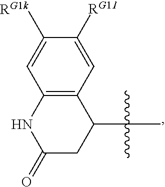

wherein

R^{G1k} and R^{G1l} are independently selected from the group consisting of hydrogen and fluorine. In one embodiment, G¹ is

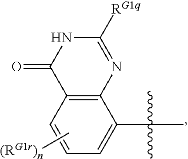

wherein

R^{G1q} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is one, two or three.

In one embodiment, G¹ is

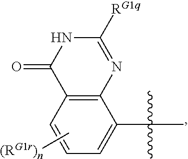

wherein

R^{G1q} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is one.

In one embodiment, G¹ is

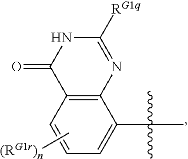

wherein

R^{G1q} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C1-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is two.

In one embodiment, G¹ is

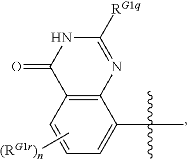

wherein

R^{G1q} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is three.

In one embodiment, G¹ is

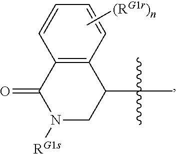

wherein

R^{G1s} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is one, two or three.

In one embodiment, G¹ is

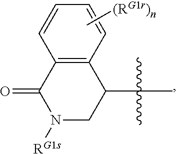

wherein

R^{G1s} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is one.

In one embodiment, G¹ is

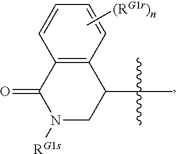

wherein

R^{G1s} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is two.

In one embodiment, G¹ is

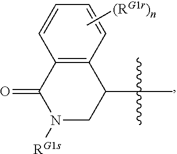

wherein

R^{G1s} is selected from the group consisting of hydrogen and C₁-C₃alkyl; wherein the C₁-C₃alkyl is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines; and

n is three.

In one embodiment, L¹ is a bond or C(R¹R²); wherein

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines.

In one embodiment, L¹ is a bond.

In one embodiment, L¹ is C(R¹R²); wherein

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines.

In one embodiment, L¹ is C(R¹R²); wherein

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines.

In one embodiment, G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines.

In one embodiment, G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines.

In one embodiment, G² is 2-furanyl or 2-thiophenyl; wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines.

In one embodiment, L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G².

In one embodiment, L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines.

In one embodiment, L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G².

In one embodiment, L² is —CH₂CH₂CH₂—, wherein the —CH₂CH₂CH₂— is unsubstituted or optionally substituted with C₁-C₃alkyl, and wherein the central carbon of —CH₂CH₂CH₂— can optionally be attached with a methylene bridge to G².

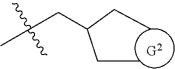 [SMILES: *CC(CC)CC]

In one embodiment, G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b},

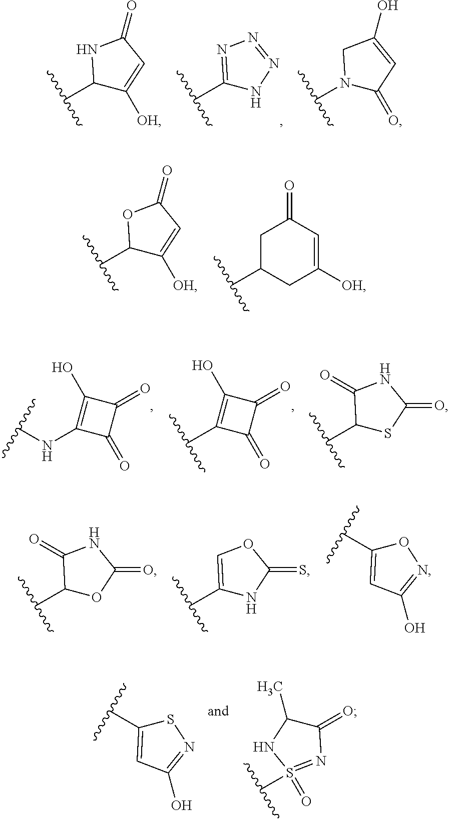

wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl; and

R^{k}, at each occurrence, is independently selected from the group consisting of C₁-C₆alkyl or C₁-C₆haloalkyl.

In one embodiment, G³ is —CO₂H.

In one embodiment, G³ is selected from the group consisting of —P(O)(OH)₂, —P(O)(OH)(OC₁-C₆alkyl), and —P(O)(CH₃)(OH).

In one embodiment, G³ is selected from the group consisting of —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b}; wherein

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl; and

R^{k}, at each occurrence, is independently selected from the group consisting of C₁-C₆alkyl or C₁-C₆haloalkyl.

In one embodiment, G³ is selected from the group consisting of —C(O)NHSO₂R^{G3a}, —C(O)NHSO₂NHR^{G3a}, —OC(O)NHSO₂R^{G3a}; wherein

R^{G3a} is C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl; and

R^{k}, at each occurrence, is independently selected from the group consisting of C₁-C₆alkyl or C₁-C₆haloalkyl.

In one embodiment, G³ is selected from the group consisting of —B(OH)₂, —SO₃H, —CH(OH)CF₃, —C(O)NH(OH), and —C(O)NH(CN).

In one embodiment, G³ is selected from the group consisting of

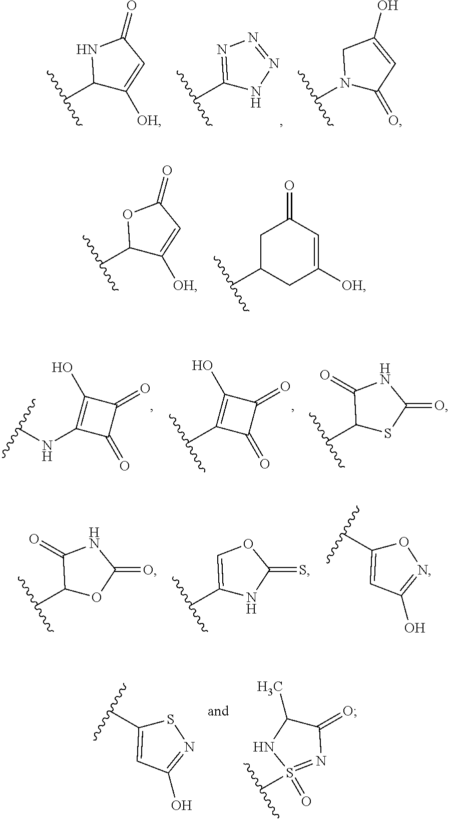

In one embodiment, G³ is

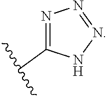 [SMILES: *c1nnn[nH]1]

In one embodiment, L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;
R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;
R^{j}, at each occurrence, is independently selected from the group consisting of hydrogen, C₁-C₆alkyl, or C₁-C₆haloalkyl;
R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected, at each occurrence, from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, L³ is —(CH₂)₂₋₅—.

In one embodiment, L³ is —(CH₂)₂—.

In one embodiment, L³ is —(CH₂)₃—.

In one embodiment, L³ is —(CH₂)₄—.

In one embodiment, L³ is —(CH₂)₅—.

In one embodiment, L³ is —(CH₂)₁₋₄—(CR³R⁴)—, wherein

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;
R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;
R^{j}, at each occurrence, is independently selected from the group consisting of hydrogen, C₁-C₆alkyl, or C₁-C₆haloalkyl; and
R^{k}, at each occurrence, is independently selected from the group consisting of C₁-C₆alkyl or C₁-C₆haloalkyl.

In one embodiment, L³ is —(CH₂)—(CR³R⁴)—, wherein

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen, and

G^{B} is aryl or heteroaryl, each of which is independently unsubstituted or substituted with 1, 2, or 3 independently selected R^{u} groups;
R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;
R^{j}, at each occurrence, is independently selected from the group consisting of hydrogen, C₁-C₆alkyl, or C₁-C₆haloalkyl; and
R^{k}, at each occurrence, is independently selected from the group consisting of C₁-C₆alkyl or C₁-C₆haloalkyl.

In one embodiment, L³ is —(CH₂)₂—(CR³R⁴)—, wherein

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen, and

G^{B} is aryl or heteroaryl, each of which is independently unsubstituted or substituted with 1, 2, or 3 independently selected R^{u} groups;
R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;
R^{j}, at each occurrence, is independently selected from the group consisting of hydrogen, C₁-C₆alkyl, or C₁-C₆haloalkyl; and
R^{k}, at each occurrence, is independently selected from the group consisting of C₁-C₆alkyl or C₁-C₆haloalkyl.

In one embodiment, L³ is —(CH₂)₃—(CR³R⁴)—, wherein

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen, and

G^{B} is aryl or heteroaryl, each of which is independently unsubstituted or substituted with 1, 2, or 3 independently selected R^{u} groups;
R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;
R^{j}, at each occurrence, is independently selected from the group consisting of hydrogen, C₁-C₆alkyl, or C₁-C₆haloalkyl; and
R^{k}, at each occurrence, is independently selected from the group consisting of C₁-C₆alkyl or C₁-C₆haloalkyl.

In one embodiment, L³ is —(CH₂)₄—(CR³R⁴)—, wherein

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen, and

G^{B} is aryl or heteroaryl, each of which is independently unsubstituted or substituted with 1, 2, or 3 independently selected R^{u} groups;
R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;
R^{j}, at each occurrence, is independently selected from the group consisting of hydrogen, C₁-C₆alkyl, or C₁-C₆haloalkyl; and
R^{k}, at each occurrence, is independently selected from the group consisting of C₁-C₆alkyl or C₁-C₆haloalkyl.

In one embodiment, L³ is —(CH₂)₁₋₄—(CR³R⁴)—, wherein

R³ and R⁴ and the carbon to which they are attached form a C₃-C₆cycloalkylene.

In one embodiment, L³ is —(CH₂)—(CR³R⁴)—, wherein

R³ and R⁴ and the carbon to which they are attached form a C₃-C₆cycloalkylene.

In one embodiment, L³ is —(CH₂)₂—(CR³R⁴)—, wherein

R³ and R⁴ and the carbon to which they are attached form a C₃-C₆cycloalkylene.

In one embodiment, L³ is —(CH₂)₃—(CR³R⁴)—, wherein

R³ and R⁴ and the carbon to which they are attached form a C₃-C₆cycloalkylene.

In one embodiment, L³ is —(CH₂)₄—(CR³R⁴)—, wherein

R³ and R⁴ and the carbon to which they are attached form a C₃-C₆cycloalkylene.

In one embodiment, L³ is —(CH₂)—(CR⁵R⁶)₁₋₃—(CH₂)—, wherein

R⁵ is independently selected, at each occurrence, from the group consisting of hydrogen and C₁-C₆alkyl; and

one R⁶ is hydroxy, and any additional R⁶ is independently selected, at each occurrence, from the group consisting of hydrogen and C₁-C₆alkyl.

In one embodiment, L³ is —(CH₂)—(CR⁵R⁶)—(CH₂)—, wherein

R⁵ is independently selected from the group consisting of hydrogen and C₁-C₆alkyl; and

R⁶ is hydroxy.

In one embodiment, L³ is —(CH₂)—(CR⁵R⁶)₂—(CH₂)—, wherein

R⁵ is independently selected, at each occurrence, from the group consisting of hydrogen and C₁-C₆alkyl; and

one R⁶ is hydroxy, and any additional R⁶ is independently selected, at each occurrence, from the group consisting of hydrogen and C₁-C₆alkyl.

In one embodiment, L³ and G³ together are

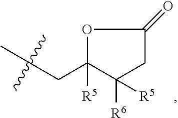

wherein

R⁵ is independently selected, at each occurrence, from the group consisting of hydrogen and C₁-C₆alkyl; and

one R⁶ is selected from the group consisting of hydrogen and C₁-C₆alkyl.

In one embodiment, L³ is —(CR⁷R⁸)₁₋₄—(CH₂)—, wherein

R⁷ and R⁸ are independently selected, at each occurrence, from the group consisting of hydrogen and C₁-C₆alkyl.

In one embodiment, L³ is —(CR⁷R⁸)—(CH₂)—, wherein

R⁷ and R⁸ are independently selected from the group consisting of hydrogen and C₁-C₆alkyl.

In one embodiment, L³ is —(CR⁷R⁸)₂—(CH₂)—, wherein

R⁷ and R⁸ are independently selected, at each occurrence, from the group consisting of hydrogen and C₁-C₆alkyl.

In one embodiment, L³ is —(CR⁷R⁸)₃—(CH₂)—, wherein

R⁷ and R⁸ are independently selected, at each occurrence, from the group consisting of hydrogen and C₁-C₆alkyl.

In one embodiment, L³ is —(CR⁷R⁸)₄—(CH₂)—, wherein

R⁷ and R⁸ are independently selected, at each occurrence, from the group consisting of hydrogen and C₁-C₆alkyl.

In one embodiment, L³ is —CH₂CH₂—X³—(CR⁹R¹⁰)₁₋₂—, wherein

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl; and

X³ is O, S, or S(O)₁₋₂.

In one embodiment, L³ is —CH₂CH₂—X³—(CR⁹R¹⁰)—, wherein

R⁹ and R¹⁰ are independently selected from the group consisting of hydrogen and C₁-C₆alkyl; and

X³ is O.

In one embodiment, L³ is —CH₂CH₂—X³—(CR⁹R¹⁰)—, wherein

R⁹ and R¹⁰ are independently selected from the group consisting of hydrogen and C₁-C₆alkyl; and

X³ is S.

In one embodiment, L³ is —CH₂CH₂—X³—(CR⁹R¹⁰)—, wherein

R⁹ and R¹⁰ are independently selected from the group consisting of hydrogen and C₁-C₆alkyl; and

X³ is S(O)₁₋₂.

In one embodiment, L³ is —CH₂CH₂—X³—(CR⁹R¹⁰)₂—, wherein

R⁹ and R¹⁰ are independently selected, at each occurrence, from the group consisting of hydrogen and C₁-C₆alkyl; and

X³ is O.

In one embodiment, L³ is —CH₂CH₂—X³—(CR⁹R¹⁰)₂—, wherein

R⁹ and R¹⁰ are independently selected, at each occurrence, from the group consisting of hydrogen and C₁-C₆alkyl; and

X³ is S.

In one embodiment, L³ is —CH₂CH₂—X³—(CR⁹R¹⁰)₂—, wherein

R⁹ and R¹⁰ are independently selected, at each occurrence, from the group consisting of hydrogen and C₁-C₆alkyl; and

X³ is S(O)₁₋₂.

In one embodiment, L³ is —CH₂CH₂—X³—(CR⁹R¹⁰)—, wherein

R⁹ and R¹⁰ and the carbon to which they are attached form a C₃-C₆cycloalkylene; and

X³ is O.

In one embodiment, L³ is —CH₂CH₂—X³—(CR⁹R¹⁰)—, wherein

R⁹ and R¹⁰ and the carbon to which they are attached form a C₃-C₆cycloalkylene; and

X³ is S.

In one embodiment, L³ is —CH₂CH₂—X³—(CR⁹R¹⁰)—, wherein

R⁹ and R¹⁰ and the carbon to which they are attached form a C₃-C₆cycloalkylene; and

X³ is S(O)₁₋₂.

In one embodiment, L³ is —CH₂CH₂—X³—(CR⁹R¹⁰)₂—, wherein

one R⁹ and R¹⁰ and the carbon to which they are attached form a C₃-C₆cycloalkylene;

the other R⁹ and R¹⁰ are independently selected from the group consisting of hydrogen and C₁-C₆alkyl; and

X³ is O.

In one embodiment, L³ is —CH₂CH₂—X³—(CR⁹R¹⁰)₂—, wherein

one R⁹ and R¹⁰ and the carbon to which they are attached form a C₃-C₆cycloalkylene;

the other R⁹ and R¹⁰ are independently selected from the group consisting of hydrogen and C₁-C₆alkyl; and

X³ is S.

In one embodiment, L³ is —CH₂CH₂—X³—(CR⁹R¹⁰)₂—, wherein

one R⁹ and R¹⁰ and the carbon to which they are attached form a C₃-C₆cycloalkylene;

the other R⁹ and R¹⁰ are independently selected from the group consisting of hydrogen and C₁-C₆alkyl; and

X³ is S(O)₁₋₂.

In one embodiment, L³ is —(CH₂)₁₋₂CH═CH—(CH₂)₁₋₂—.

In one embodiment, L³ is —(CH₂)CH═CH—(CH₂)—.

In one embodiment, L³ is —(CH₂)CH═CH—(CH₂)₂—.

In one embodiment, L³ is —(CH₂)₂CH═CH—(CH₂)—.

In one embodiment, L³ is —(CH₂)₂CH═CH—(CH₂)₂—.

In one embodiment, L³ is —CH₂C(O)NH(CR¹¹R¹²)—; wherein

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring; wherein

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl; and

R^{k}, at each occurrence, is independently selected from the group consisting of C₁-C₆alkyl or C₁-C₆haloalkyl.

In one embodiment, L³ is —CH₂C(O)NH(CR¹¹R¹²)—; wherein R¹¹ and R¹² are independently selected from the group consisting of hydrogen, C₁-C₆alkyl, and —(C₁-C₆alkylenyl)-G^{B}; wherein

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl; and

R^{k}, at each occurrence, is independently selected from the group consisting of C₁-C₆alkyl or C₁-C₆haloalkyl.

In one embodiment, L³ is —CH₂C(O)NH(CR¹¹R¹²)—; wherein

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is

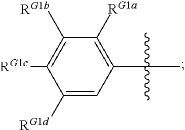

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b},

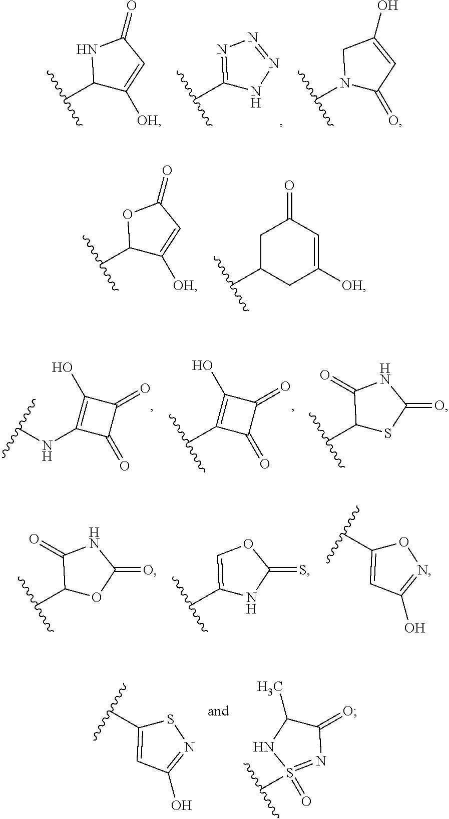

wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I), G¹ is selected from

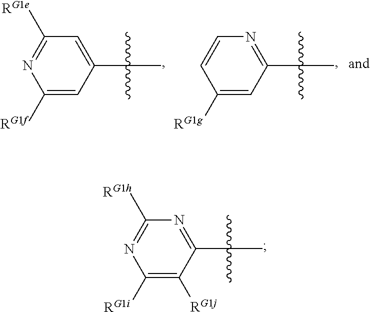

wherein

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b},

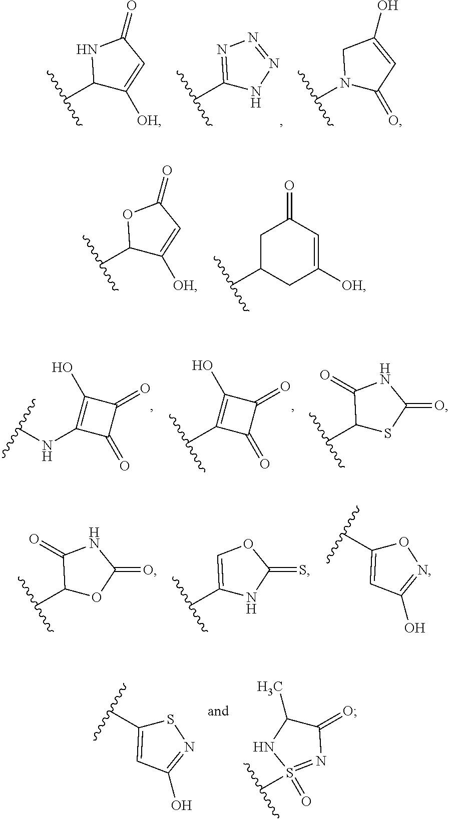

wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

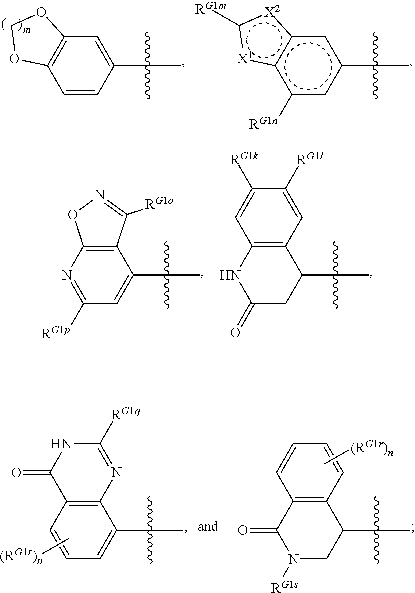

wherein

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b},

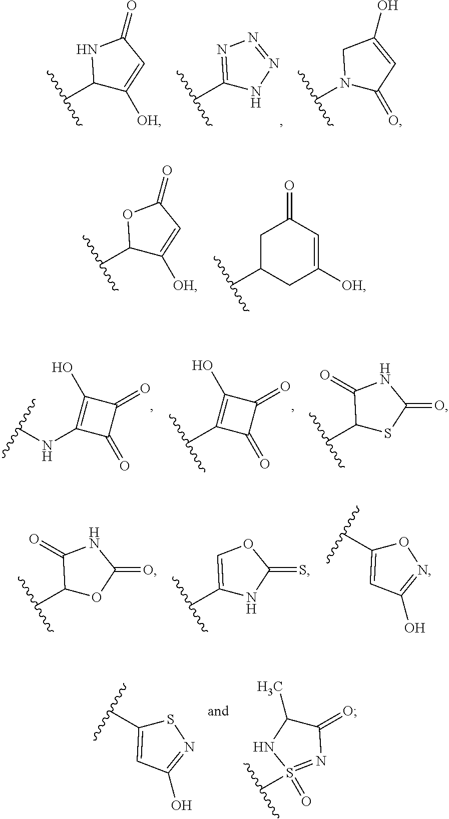

wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

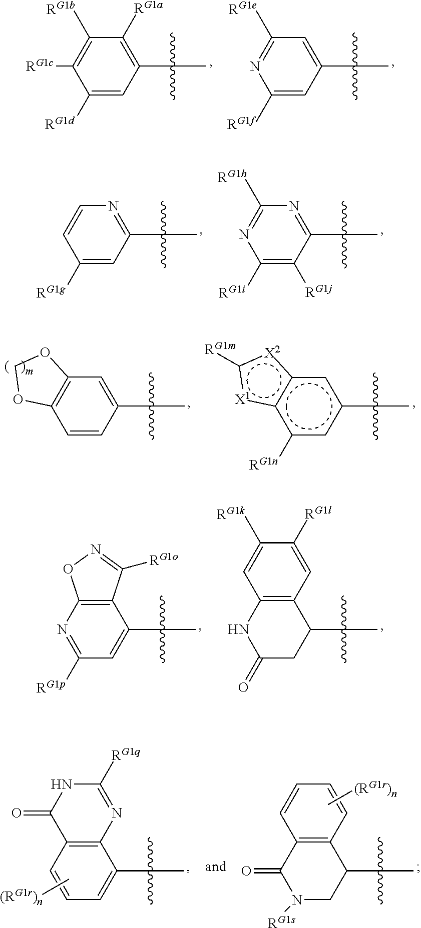

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b},

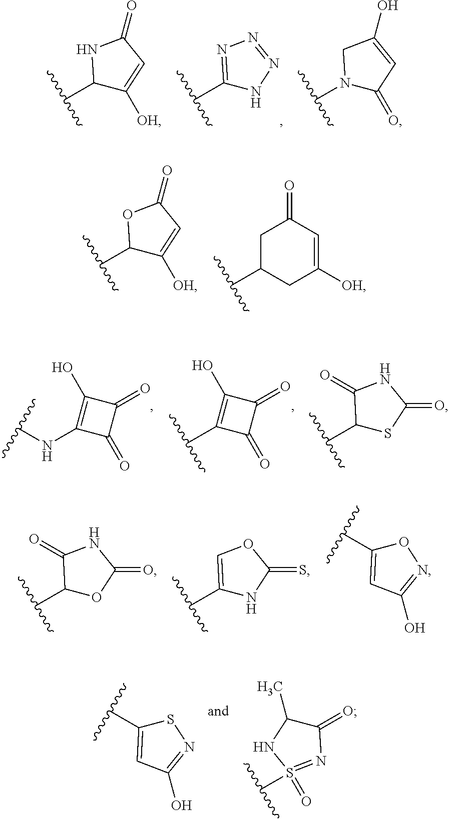

wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

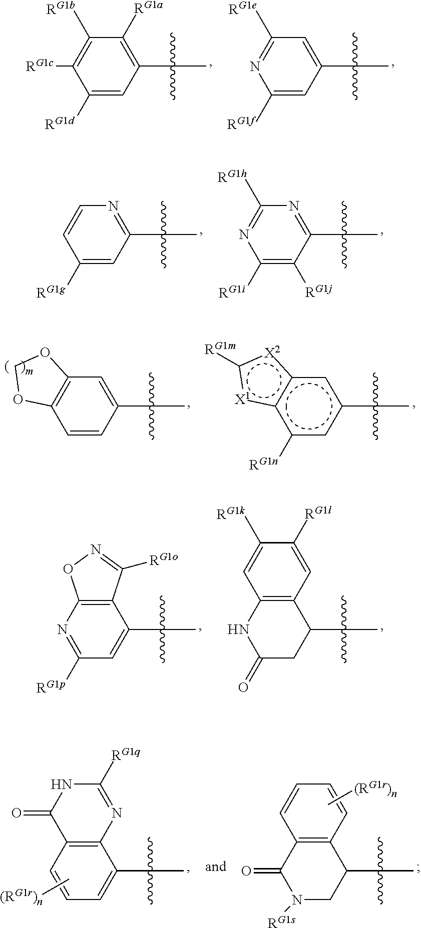

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of 2-furanyl and 2-thiophenyl; wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b},

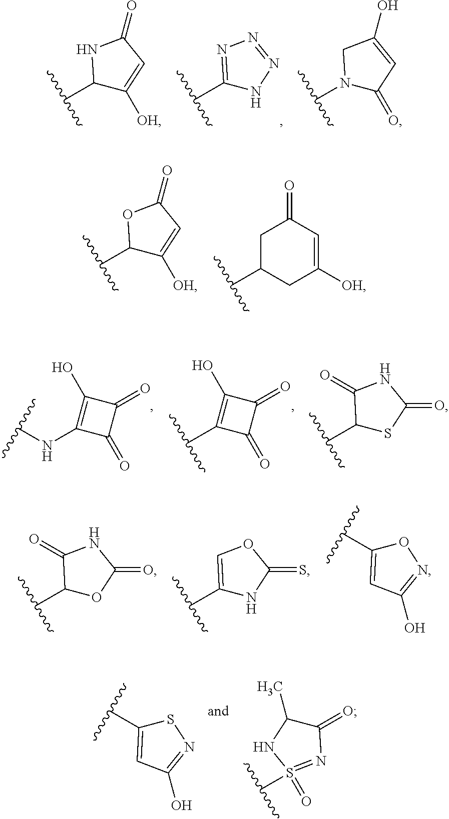

wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

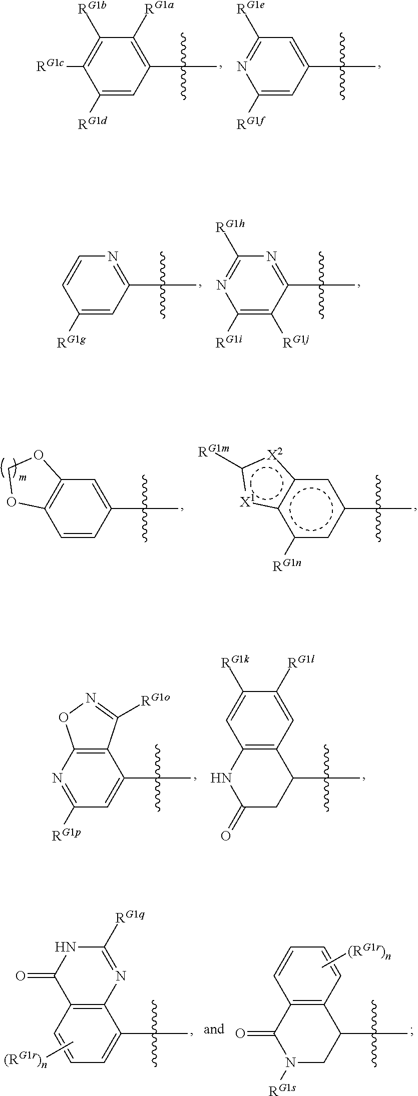

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

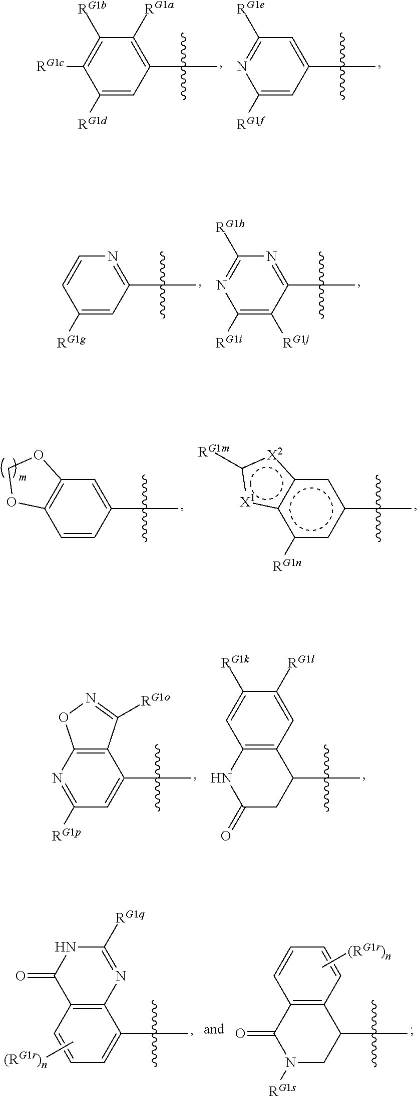

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C1-C₃alkoxy is unsubstituted or optionally substituted with one, two, or three fluorines;

R^{G1r} is independently selected at each occurrence from the group consisting of hydrogen, C1-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C1-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —P(O)(OH)₂, —P(O)(OH)(OC₁-C₆alkyl), and —P(O)(CH₃)(OH);

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

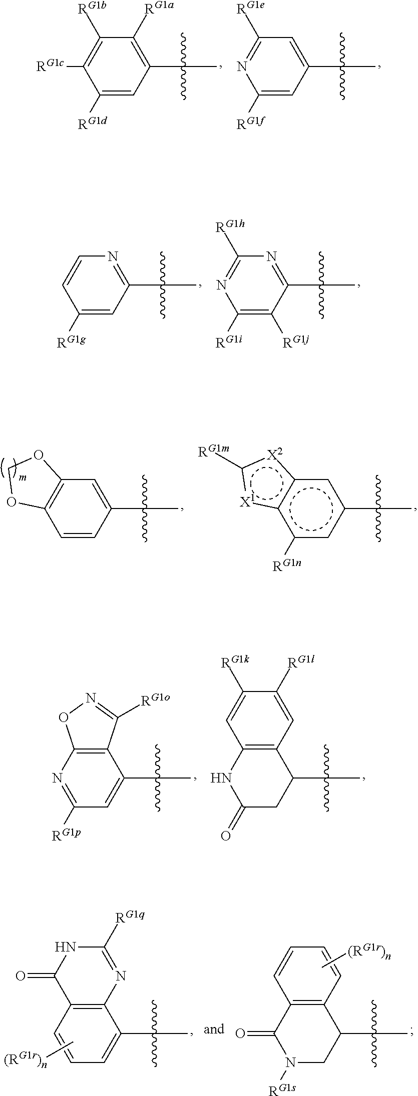

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —B(OH)₂, —SO₃H, —CH(OH)CF₃, —C(O)NH(OH), and —C(O)NH(CN);

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

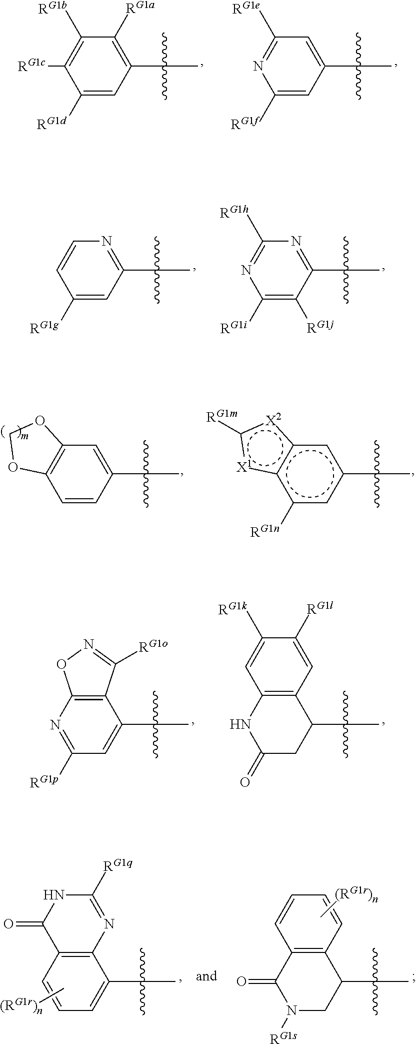

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C1-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, and —C(O)NHS(O)(R^{G3a})═NR^{G3b}; wherein,

R^{G3a} is C₁-C₆alkyl, C₁-C₆haloalkyl, or G^{A};

R^{G3b} is hydrogen, C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

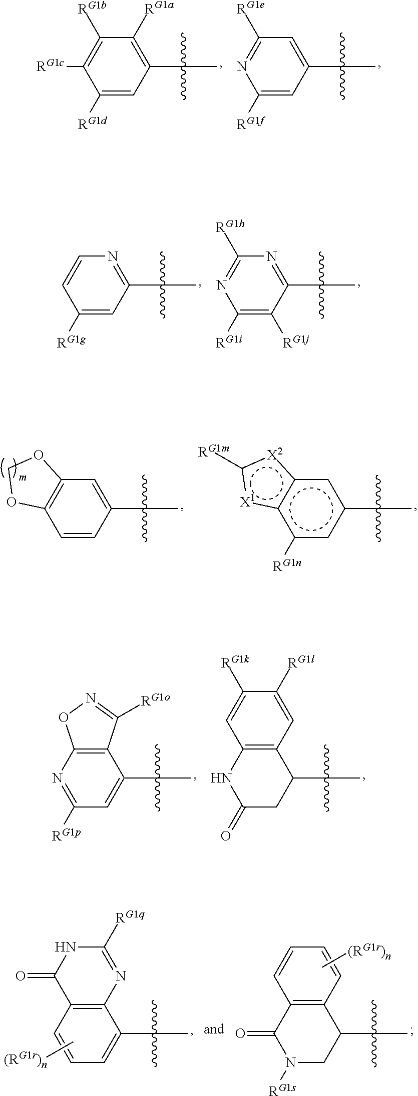

wherein

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C1-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of

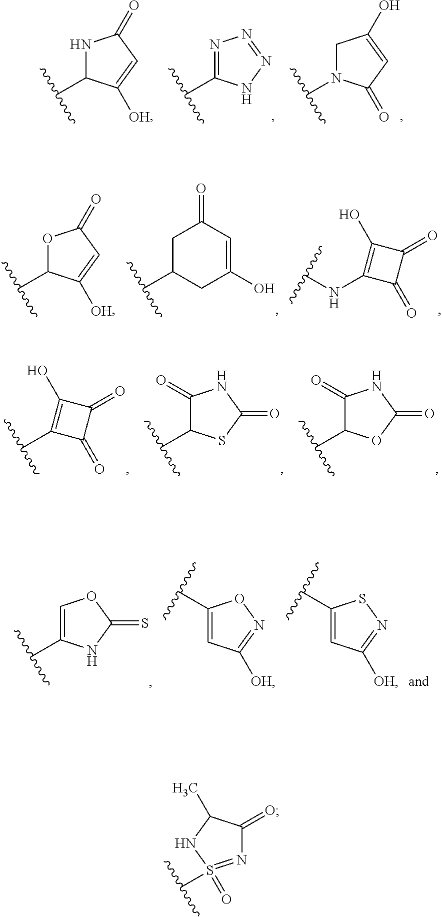

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

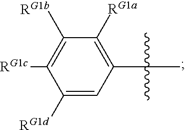

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is —CO₂H;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

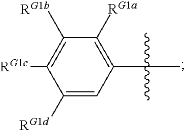

R^{G1a} is selected from the group consisting of hydrogen and fluorine;

R^{G1b} and R^{G1d} are independently selected from the group consisting of hydrogen, and C₁-C₃alkoxy;

R^{G1c} is selected from the group consisting of hydrogen, C₁-C₃alkoxy, C₁-C₃alkyl, C₃-C₅cycloalkyl, and halogen; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² and the carbon atom to which they are attached form a C₃-C₅cycloalkylene;

G² is phenyl; wherein the phenyl is unsubstituted or optionally substituted with 1, 2 or 3 substituents independently selected from C1-C₃alkoxy, C₁-C₃alkyl, or halogen;

L² is —CH₂CH₂CH₂—;

G³ is —CO₂H;

L³ is selected from the group consisting of —(CH₂)₄₋₅—, —(CH₂)₃₋₄—(CR³R⁴)—, —CH₂CH₂—X³—(CR⁹R¹⁰)₁₂—, and —CH₂C(O)NH(CR¹¹R¹²)—;

R³ and R⁴ are selected from the group consisting of hydrogen, C₁-C₃alkyl, and hydroxy, wherein one of R³ and R⁴ is other than hydrogen; or

R⁹ and R¹⁰ are independently selected, at each occurrence, from the group consisting of hydrogen and C₁-C₆alkyl;

X³ is O; and

R¹¹ and R¹² are independently selected from the group consisting of hydrogen and C₁-C₃alkyl; or

R¹¹ and R¹² and the carbon to which they are attached form a C₃-C₆cycloalkylene, wherein the C₃-C₆cycloalkylene is optionally fused to a phenyl ring.

In one embodiment, in a compound or pharmaceutically acceptable salt of formula (I),

G¹ is selected from

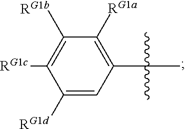

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is selected from the group consisting of 2-furanyl and 2-thiophenyl; wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is —CO₂H;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

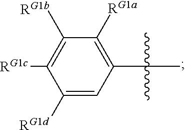

wherein

R^{G1a} is selected from the group consisting of hydrogen and fluorine;

R^{G1b} and R^{G1d} are C₁-C₃alkoxy;

R^{G1c} is selected from the group consisting of C₁-C₃alkoxy and halogen;

wherein at least one of R^{G1b}, R^{G1c}, and R^{G1d} is other than hydrogen;

L¹ is a bond;

G² is selected from the group consisting of 2-furanyl and 2-thiophenyl; wherein the 2-furanyl and 2-thiophenyl are unsubstituted or optionally substituted with 1 C₁-C₃alkyl;

L² is —CH₂CH₂CH₂—;

G³ is —CO₂H; and

L³ is —(CH₂)₄₋₅—.

In one embodiment, in a compound or pharmaceutically acceptable salt of formula (I),

G¹ is selected from

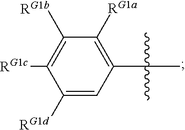

wherein

R^{G1a} is selected from the group consisting of hydrogen and fluorine;

R^{G1b} and R^{G1d} are C₁-C₃alkoxy;

R^{G1c} is selected from the group consisting of C₁-C₃alkoxy and halogen;

wherein at least one of R^{G1b}, R^{G1c}, and R^{G1d} is other than hydrogen;

L¹ is a bond;

G² is selected from the group consisting of 2-furanyl and 2-thiophenyl; wherein the 2-furanyl and 2-thiophenyl are unsubstituted or optionally substituted with 1 C₁-C₃alkyl;

L² is —CH₂CH₂CH₂—;

L³-G³ is

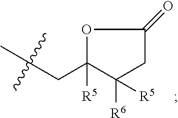

R⁵ is independently selected, at each occurrence, from the group consisting of hydrogen and C₁-C₆alkyl; and

one R⁶ is selected from the group consisting of hydrogen and C₁-C₆alkyl.

In one embodiment, in a compound or pharmaceutically acceptable salt of formula (I),

G¹ is selected from

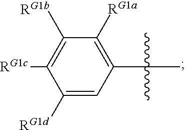

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —P(O)(OH)₂, —P(O)(OH)(OC₁-C₆alkyl), and —P(O)(CH₃)(OH);

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

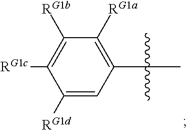

wherein

R^{G1a} is hydrogen;

R^{G1b} and R^{G1c} are each C₁-C₃alkoxy;

R^{G1c} is selected from the group consisting of hydrogen and C₁-C₃alkyl;

wherein at least one of R^{G1b}, R^{G1c}, and R^{G1d} is other than hydrogen;

L¹ is a bond;

G² is phenyl;

L² is —CH₂CH₂CH₂—;

G³ is —P(O)(OH)₂;

L³ is selected from the group consisting of —(CH₂)₄₋₅—, —(CH₂)₃₋₄—(CR³R⁴)—, and —CH₂CH₂—X³—(CR⁹R¹⁰)₂—;

R³ and R⁴ are selected from the group consisting of hydrogen and C₁-C₃alkyl, wherein one of R³ and R⁴ is other than hydrogen;

R⁹ and R¹⁰ are each hydrogen; and

X³ is O.

In one embodiment, in a compound or pharmaceutically acceptable salt of formula (I),

G¹ is selected from

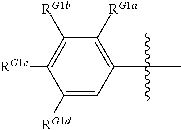

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —C(O)NHSO₂R^{G3a}, —C(O)NHSO₂NHR^{G3a}, and —OC(O)NHSO₂R^{G3a};

R^{G3a} is C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

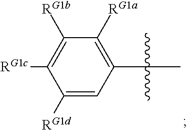

wherein
R^{G1a} is hydrogen;
R^{G1b} and R^{G1d} are each C₁-C₃alkoxy;
R^{G1c} is C₁-C₃alkyl;

L¹ is a bond;

G² is phenyl;

L² is —CH₂CH₂CH₂—;

G³ is —OC(O)NHSO₂R^{G3a};
R^{G3a} is C₁-C₆alkyl; and

L³ is —(CH₂)₂₋₃—.

In one embodiment, in a compound or pharmaceutically acceptable salt of formula (I),

G¹ is selected from

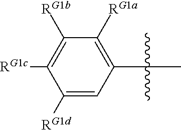

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of

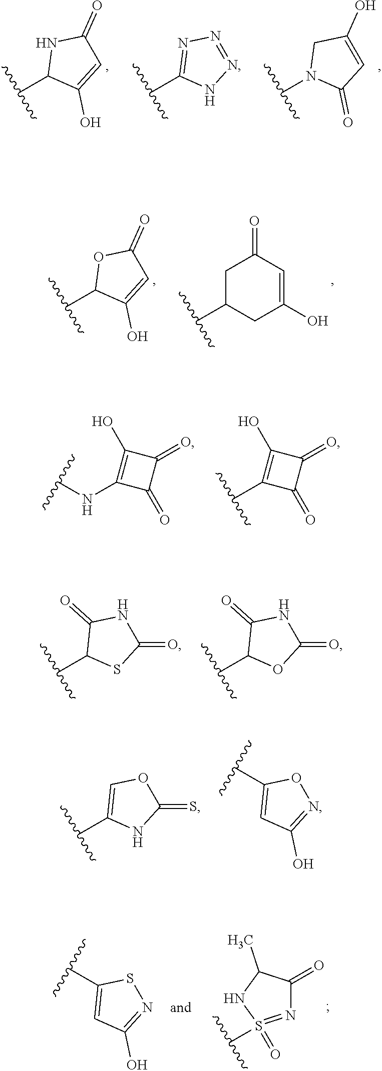

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from

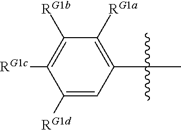

R^{G1a} is selected from the group consisting of hydrogen and fluorine;

R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;

R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

wherein at least one of R^{G1b}, R^{G1c}, and R^{G1d} is other than hydrogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is

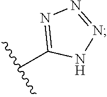 [SMILES: *c1nnn[nH]1]

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from the group consisting of

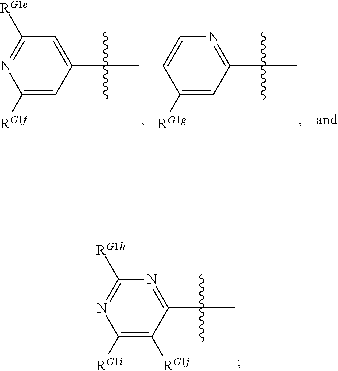

wherein

R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;

R^{x} is C₁-C₃alkyl; wherein the C₁-C₃alkyl is unsubstituted or optionally substituted with one, two or three fluorines;

R^{G1g} is selected from the group consisting of halogen and C₁-C₃alkoxy; wherein the C₁-C₃alkoxy is unsubstituted or optionally substituted with one, two, or three fluorines;

R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;

R^{G1j} is selected from the group consisting of hydrogen or halogen;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H, —P(O)(OH)₂, —P(O)(OH)(OC₁-C₆alkyl), —P(O)(CH₃)(OH), —C(O)NHSO₂R^{G3a}, —C(O)NHSO₂NHR^{G3a}, —NHSO₂NHC(O)R^{G3a}, and —OC(O)NHSO₂R^{G3a};

R^{G3a} is C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected, at each occurrence, from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from the group consisting of

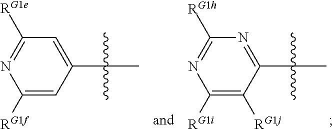

wherein

R^{G1e} and R^{G1f} are each C₁-C₃alkoxy;

R^{G1h} and R^{G1i} are each C₁-C₃alkoxy;

R^{G1j} is hydrogen;

L¹ is a bond;

G² is phenyl;

L² is —CH₂CH₂CH₂—;

G³ is —CO₂H; and

L³ is —(CH₂)₄₋₅—.

In one embodiment, in a compound or pharmaceutically acceptable salt of formula (I),

G¹ is selected from the group consisting of

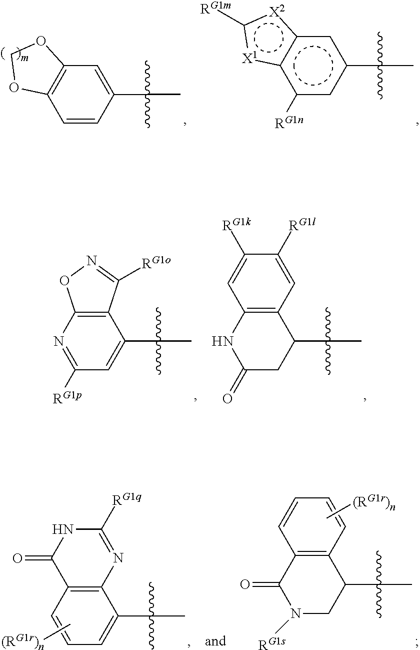

wherein

R^{G1k} and R^{G1l} are independently selected from the group consisting of hydrogen and fluorine;

R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;

R^{G1n} is selected from the group consisting of halogen and C₁-C₃alkoxy; wherein the C₁-C₃alkoxy is unsubstituted or optionally substituted with one, two, or three fluorines;

R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;

one of X¹ and X² is O and the other is CH;

m is one, two or three;

n is one, two or three;

L¹ is a bond or C(R¹R²);

R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or

R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;

G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines;

L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;

G³ is selected from the group consisting of —CO₂H, —P(O)(OH)₂, —P(O)(OH)(OC₁-C₆alkyl), —P(O)(CH₃)(OH), —C(O)NHSO₂R^{G3a}, —C(O)NHSO₂NHR^{G3a}, —NHSO₂NHC(O)R^{G3a}, and —OC(O)NHSO₂R^{G3a};

R^{G3a} is C₁-C₆alkyl, C₁-C₆haloalkyl, or G^{A};

G^{A} is cycloalkyl, cycloalkenyl, aryl, or heteroaryl, each of which is independently unsubstituted or substituted with 1, 2, or 3 independently selected R^{u} groups; wherein

R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R^{j})₂;

R^{j}, at each occurrence, is independently selected from the group consisting of hydrogen, C₁-C₆alkyl, or C₁-C₆haloalkyl;

R^{k}, at each occurrence, is independently selected from the group consisting of C₁-C₆alkyl or C₁-C₆haloalkyl;

L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;

R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or

R³ and R⁴ and the carbon to which they are attached form a C₃-C₆cycloalkylene;

G^{B} is aryl or heteroaryl, each of which is independently unsubstituted or substituted with 1, 2, or 3 independently selected R^{u} groups;

R⁵ is independently selected, at each occurrence, from the group consisting of hydrogen and C₁-C₆alkyl;

one R⁶ is hydroxy, and any additional R⁶ is independently selected, at each occurrence, from the group consisting of hydrogen and C₁-C₆alkyl;

R⁷ and R⁸ are independently selected, at each occurrence, from the group consisting of hydrogen and C₁-C₆alkyl;

R⁹ and R¹⁰ are independently selected, at each occurrence, from the group consisting of hydrogen and C₁-C₆alkyl; or

one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;

X³ is O, S, or S(O)₁₋₂; and

R¹¹ and R¹² are independently selected from the group consisting of hydrogen, C₁-C₆alkyl, and —(C₁-C₆alkylenyl)-G^{B}; or

R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

In one embodiment, in a compound or pharmaceutically acceptable salt of formula (I),

G¹ is selected from the group consisting of

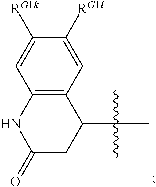

wherein

R^{G1k} and R^{G1l} are independently selected from the group consisting of hydrogen and fluorine;

L¹ is a bond;

G² is phenyl;

L² is —CH₂CH₂CH₂—;

G³ is —CO₂H; and

L³ is —(CH₂)₄₋₅—.

Specific embodiments contemplated as part of the present disclosure also include, but are not limited to, compounds or pharmaceutically acceptable salts of formula (I), as defined, for example:
2-{[N-(3,5-dimethoxy-4-methylbenzoyl)-N-(3-phenylpropyl)glycyl]amino}-2,3-dihydro-1H-indene-2-carboxylic acid;
1-{[N-(3,5-dimethoxy-4-methylbenzoyl)-N-(3-phenylpropyl)glycyl]amino}cyclohexane-1-carboxylic acid;
1-{[N-(3,5-dimethoxy-4-methylbenzoyl)-N-(3-phenylpropyl)glycyl]amino}cyclopropane-1-carboxylic acid;
5-[(3,5-dimethoxy-4-methylbenzoyl)(3-phenylpropyl)amino]pentanoic acid;
6-[(3,5-dimethoxy-4-methylbenzoyl)(3-phenylpropyl)amino]hexanoic acid;
{2-[(3,5-dimethoxy-4-methylbenzoyl)(3-phenylpropyl)amino]ethoxy}acetic acid;
5-[(3,5-dimethoxybenzoyl)(3-phenylpropyl)amino]pentanoic acid;
5-{[1-(4-methoxyphenyl)cyclopropane-1-carbonyl](3-phenylpropyl)amino}pentanoic acid;
5-{[(3,5-dimethoxyphenyl)acetyl](3-phenylpropyl)amino}pentanoic acid;
5-[(3-phenylpropyl)(3,4,5-trimethoxybenzoyl)amino]pentanoic acid;
5-{(3,5-dimethoxy-4-methylbenzoyl)[3-(3-methylphenyl)propyl]amino}pentanoic acid;
5-[(3,5-dichlorobenzoyl)(3-phenylpropyl)amino]pentanoic acid;
5-[(3,5-difluoro-4-methoxybenzoyl)(3-phenylpropyl)amino]pentanoic acid;
5-[(2-ethoxypyridine-4-carbonyl)(3-phenylpropyl)amino]pentanoic acid;
{2-[(3,5-dimethoxybenzoyl)(3-phenylpropyl)amino]ethoxy}acetic acid;
5-[(3,5-dichloro-4-methylbenzoyl)(3-phenylpropyl)amino]pentanoic acid;
5-[(4-chloro-3-methoxybenzoyl)(3-phenylpropyl)amino]pentanoic acid;
5-[(3,5-diethoxybenzoyl)(3-phenylpropyl)amino]pentanoic acid;
5-[(3,5-dimethoxy-2-nitrobenzoyl)(3-phenylpropyl)amino]pentanoic acid;
5-[(4-bromo-3,5-dimethoxybenzoyl)(3-phenylpropyl)amino]pentanoic acid;
5-[(3-phenylpropyl)(3,4,5-triethoxybenzoyl)amino]pentanoic acid;
5-[(3-methoxy-4-nitrobenzoyl)(3-phenylpropyl)amino]pentanoic acid;
5-[(3,4-dihydro-2H-1,5-benzodioxepine-7-carbonyl)(3-phenylpropyl)amino]pentanoic acid;
5-[(7-methoxy-1-benzofuran-5-carbonyl)(3-phenylpropyl)amino]pentanoic acid;
5-[(3-phenylpropyl){1-[4-(trifluoromethoxy)phenyl]cyclopropane-1-carbonyl}amino]pentanoic acid;
5-{(3-phenylpropyl)[3-(trifluoromethoxy)benzoyl]amino}pentanoic acid;
5-{[1-(2H-1,3-benzodioxol-5-yl)cyclopropane-1-carbonyl](3-phenylpropyl)amino}pentanoic acid;
5-[(3-methoxy-4-methylbenzoyl)(3-phenylpropyl)amino]pentanoic acid;
5-{[3-methoxy-5-(trifluoromethoxy)benzoyl](3-phenylpropyl)amino}pentanoic acid;
5-[(2,4-difluoro-3,5-dimethoxybenzoyl)(3-phenylpropyl)amino]pentanoic acid;
5-[(4-methoxy-2-methyl-1-benzofuran-6-carbonyl)(3-phenylpropyl)amino]pentanoic acid;
5-{(3,5-dimethoxy-4-methylbenzoyl)[3-(2-fluorophenyl)propyl]amino}pentanoic acid;
5-{(3,5-dimethoxy-4-methylbenzoyl)[3-(3-fluorophenyl)propyl]amino}pentanoic acid;
5-{(3,5-dimethoxy-4-methylbenzoyl)[3-(4-fluorophenyl)propyl]amino}pentanoic acid;
5-[(4-cyclopropyl-3,5-dimethoxybenzoyl)(3-phenylpropyl)amino]pentanoic acid;
5-{[3-(4-chlorophenyl)propyl](3,5-dimethoxy-4-methylbenzoyl)amino}pentanoic acid;
5-{[3,5-dimethoxy-4-(trifluoromethyl)benzoyl](3-phenylpropyl)amino}pentanoic acid;
5-{[3-(3-chlorophenyl)propyl](3,5-dimethoxy-4-methylbenzoyl)amino}pentanoic acid;
5-[(2-chloro-3,5-dimethoxybenzoyl)(3-phenylpropyl)amino]pentanoic acid;
5-[(3,5-dimethoxy-4-methylbenzoyl){3-[3-(trifluoromethyl)phenyl]propyl}amino]pentanoic acid;
5-{(3,5-dimethoxy-4-methylbenzoyl)[3-(5-methylthiophen-2-yl)propyl]amino}pentanoic acid;
5-{(3,5-dimethoxy-4-methylbenzoyl)[3-(4-methylphenyl)propyl]amino}pentanoic acid;
5-{(3,5-dimethoxy-4-methylbenzoyl)[3-(4-methoxyphenyl)propyl]amino}pentanoic acid;
({2-[(3,5-dimethoxy-4-methylbenzoyl)(3-phenylpropyl)amino]ethyl}sulfanyl)acetic acid;
N-(3,5-dimethoxy-4-methylbenzoyl)-N-(3-phenylpropyl)glycyl-2-methylalanine;
N-(3,5-dimethoxy-4-methylbenzoyl)-N-(3-phenylpropyl)glycylphenylalanine;
N-(3,5-dimethoxy-4-methylbenzoyl)-N-(3-phenylpropyl)glycyl-3-thiophen-2-ylalanine;
5-[(2,6-dimethoxypyridine-4-carbonyl)(3-phenylpropyl)amino]pentanoic acid;
5-{[3-(2,4-dichlorophenyl)propyl](3,5-dimethoxy-4-methylbenzoyl)amino}pentanoic acid;
({2-[(3,5-dimethoxy-4-methylbenzoyl)(3-phenylpropyl)amino]ethoxy}methyl)phosphonic acid;
N-(3,5-dimethoxy-4-methylbenzoyl)-N-(3-phenylpropyl)glycylglycine;
2-benzyl-5-[(3,5-dimethoxy-4-methylbenzoyl)(3-phenylpropyl)amino]pentanoic acid;
{2-[(3,5-dimethoxy-4-methylbenzoyl)(3-phenylpropyl)amino]ethanesulfinyl}acetic acid;
5-[(3,5-dimethoxy-4-methylbenzoyl)(3-phenylpropyl)amino]-2-methylpentanoic acid;
2-{2-[(3,5-dimethoxy-4-methylbenzoyl)(3-phenylpropyl)amino]ethoxy}-2-methylpropanoic acid;
3-{2-[(3,5-dimethoxy-4-methylbenzoyl)(3-phenylpropyl)amino]ethoxy}propanoic acid;
5-[(3,5-dimethoxy-4-methylbenzoyl)(3-phenylpropyl)amino]-2,2-dimethylpentanoic acid;
5-[(2-fluoro-3,5-dimethoxybenzoyl)(3-phenylpropyl)amino]pentanoic acid;
1-({2-[(3,5-dimethoxy-4-methylbenzoyl)(3-phenylpropyl)amino]ethoxy}methyl)cyclopropane-1-carboxylic acid;
3-({2-[(3,5-dimethoxy-4-methylbenzoyl)(3-phenylpropyl)amino]ethyl}sulfanyl)propanoic acid;
1-[({2-[(3,5-dimethoxy-4-methylbenzoyl)(3-phenylpropyl)amino]ethyl}sulfanyl)methyl]cyclopropane-1-carboxylic acid;
3-({2-[(3,5-dimethoxy-4-methylbenzoyl)(3-phenylpropyl)amino]ethyl}sulfanyl)butanoic acid;
5-{[1-(5-methoxypyridin-2-yl)cyclopropane-1-carbonyl](3-phenylpropyl)amino}pentanoic acid;
5-{(3-phenylpropyl)[1-(pyridin-4-yl)cyclopropane-1-carbonyl]amino}pentanoic acid;
5-[(6-methoxy-1H-indole-3-carbonyl)(3-phenylpropyl)amino]pentanoic acid;
5-{[(2R)-2-methoxy-2-(4-methoxyphenyl)acetyl](3-phenylpropyl)amino}pentanoic acid;
N-(3,5-dimethoxy-4-methylbenzoyl)-N-(3-phenylpropyl)-beta-alanine;
3,5-dimethoxy-4-methyl-N-{3-[(methylsulfamoyl)amino]-3-oxopropyl}-N-(3-phenylpropyl)benzamide;
4-[(3,5-dimethoxy-4-methylbenzoyl)(3-phenylpropyl)amino]butanoic acid;
3,5-dimethoxy-4-methyl-N-{5-[(methylsulfamoyl)amino]-5-oxopentyl}-N-(3-phenylpropyl)benzamide;
{4-[(3,5-dimethoxy-4-methylbenzoyl)(3-phenylpropyl)amino]butyl}phosphonic acid;
{5-[(3,5-dimethoxy-4-methylbenzoyl)(3-phenylpropyl)amino]pentan-2-yl}phosphonic acid;
1-(4-methoxyphenyl)-N-{5-[(methylsulfamoyl)amino]-5-oxopentyl}-N-(3-phenylpropyl)cyclopropane-1-carboxamide;
3,5-dimethoxy-N-{5-[(methylsulfamoyl)amino]-5-oxopentyl}-N-(3-phenylpropyl)benzamide;
{4-[(3,5-dimethoxybenzoyl)(3-phenylpropyl)amino]butyl}phosphonic acid;
ethyl hydrogen {4-[(3,5-dimethoxybenzoyl)(3-phenylpropyl)amino]butyl}phosphonate;
(−)-(2R)-5-[(3,5-dimethoxy-4-methylbenzoyl)(3-phenylpropyl)amino]-2-methylpentanoic acid;
(+)-(2S)-5-[(3,5-dimethoxy-4-methylbenzoyl)(3-phenylpropyl)amino]-2-methylpentanoic acid;
5-[(3-fluoro-4-methoxybenzoyl)(3-phenylpropyl)amino]pentanoic acid;
5-[(2H-1,3-benzodioxole-5-carbonyl)(3-phenylpropyl)amino]pentanoic acid;
5-[(4-fluoro-3-methoxybenzoyl)(3-phenylpropyl)amino]pentanoic acid;
5-{[1-(3-methoxyphenyl)cyclopropane-1-carbonyl](3-phenylpropyl)amino}pentanoic acid;
5-[(3,4-dimethoxybenzoyl)(3-phenylpropyl)amino]pentanoic acid;
5-[(4-methoxybenzoyl)(3-phenylpropyl)amino]pentanoic acid;
5-{[2-(4-methoxyphenyl)-2-methylpropanoyl](3-phenylpropyl)amino}pentanoic acid;
5-{[1-(4-methoxyphenyl)cyclobutane-1-carbonyl](3-phenylpropyl)amino}pentanoic acid;
(2-{[1-(4-methoxyphenyl)cyclopropane-1-carbonyl](3-phenylpropyl)amino}ethoxy)acetic acid;
5-{[4-(2-hydroxyethoxy)-3,5-dimethoxybenzoyl](3-phenylpropyl)amino}pentanoic acid;
5-{[3-(4-methoxyphenyl)oxetane-3-carbonyl](3-phenylpropyl)amino}pentanoic acid;
5-{(3,5-dimethoxybenzoyl)[3-(3-fluorophenyl)propyl]amino}pentanoic acid;
5-{[3-(3-chlorophenyl)propyl](3,5-dimethoxybenzoyl)amino}pentanoic acid;
5-{[3-(3-fluorophenyl)propyl][1-(4-methoxyphenyl)cyclopropane-1-carbonyl]amino}pentanoic acid;
2-{3-[(3,5-dimethoxy-4-methylbenzoyl)(3-phenylpropyl)amino]propyl}hexanoic acid;
2-{3-[(3,5-dimethoxy-4-methylbenzoyl)(3-phenylpropyl)amino]propyl}-2-methylhexanoic acid;
5-[(3,5-dimethoxybenzoyl)(3-phenylpropyl)amino]-2-methylpentanoic acid;
5-[(4-fluoro-3,5-dimethoxybenzoyl)(3-phenylpropyl)amino]pentanoic acid;
({2-[(4-fluoro-3,5-dimethoxybenzoyl)(3-phenylpropyl)amino]ethyl}sulfanyl)acetic acid;
({2-[(3,5-dimethoxybenzoyl)(3-phenylpropyl)amino]ethyl}sulfanyl)acetic acid;
5-{[3-(3-chlorophenyl)propyl][1-(4-methoxyphenyl)cyclopropane-1-carbonyl]amino}pentanoic acid;
5-{[1-(2-fluoro-4-methoxyphenyl)cyclopropane-1-carbonyl](3-phenylpropyl)amino}pentanoic acid;
1-{[N-(3,5-dimethoxy-4-methylbenzoyl)-N-(3-phenylpropyl)glycyl]amino}-4-ethylcyclohexane-1-carboxylic acid;
5-{[1-(3-fluoro-4-methoxyphenyl)cyclopropane-1-carbonyl](3-phenylpropyl)amino}pentanoic acid;
5-{[1-(4-methoxyphenyl)cyclopentane-1-carbonyl](3-phenylpropyl)amino}pentanoic acid;
5-{[1-(3-chloro-4-methoxyphenyl)cyclopropane-1-carbonyl](3-phenylpropyl)amino}pentanoic acid;
5-[(3,5-dimethoxybenzoyl)(3-phenylpropyl)amino]-2,2-dimethylpentanoic acid;
(2-{[3-(3-chlorophenyl)propyl](3,5-dimethoxy-4-methylbenzoyl)amino}ethoxy)acetic acid;
(2-{(3,5-dimethoxy-4-methylbenzoyl)[3-(2-fluorophenyl)propyl]amino}ethoxy)acetic acid;
N-{5-[(methanesulfonyl)amino]-5-oxopentyl}-3,5-dimethoxy-4-methyl-N-(3-phenylpropyl)benzamide;
[(2-{[3-(2-chlorophenyl)propyl](3,5-dimethoxy-4-methylbenzoyl)amino}ethyl)sulfanyl]acetic acid;
[(2-{[3-(2,6-difluorophenyl)propyl](3,5-dimethoxy-4-methylbenzoyl)amino}ethyl)sulfanyl]acetic acid;
[(2-{[3-(2,4-difluorophenyl)propyl](3,5-dimethoxy-4-methylbenzoyl)amino}ethyl)sulfanyl]acetic acid;
[(2-{[1-(4-methoxyphenyl)cyclopropane-1-carbonyl](3-phenylpropyl)amino}ethyl)sulfanyl]acetic acid;
[(2-{(3,5-dimethoxy-4-methylbenzoyl)[3-(2-fluorophenyl)propyl]amino}ethyl)sulfanyl]acetic acid;
[(2-{(3,5-dimethoxy-4-methylbenzoyl)[3-(3-fluorophenyl)propyl]amino}ethyl)sulfanyl]acetic acid;
[(2-{(3,5-dimethoxy-4-methylbenzoyl)[3-(4-fluorophenyl)propyl]amino}ethyl)sulfanyl]acetic acid;
N-{5-[(cyclopropanesulfonyl)amino]-5-oxopentyl}-3,5-dimethoxy-4-methyl-N-(3-phenylpropyl)benzamide;
(4R)-5-[(3,5-dimethoxy-4-methylbenzoyl)(3-phenylpropyl)amino]-4-hydroxypentanoic acid;
(3E)-5-[(3,5-dimethoxy-4-methylbenzoyl)(3-phenylpropyl)amino]pent-3-enoic acid;
[(2-{[3-(3-chlorophenyl)propyl](3,5-dimethoxy-4-methylbenzoyl)amino}ethyl)sulfanyl]acetic acid;
N-{5-[(ethanesulfonyl)amino]-5-oxopentyl}-3,5-dimethoxy-4-methyl-N-(3-phenylpropyl)benzamide;
3,5-dimethoxy-4-methyl-N-{5-oxo-5-[(propane-2-sulfonyl)amino]pentyl}-N-(3-phenylpropyl)benzamide;
[(2-{[3-(3,5-difluorophenyl)propyl](3,5-dimethoxy-4-methylbenzoyl)amino}ethyl)sulfanyl]acetic acid;
5-{[3-(3,5-difluorophenyl)propyl](3,5-dimethoxy-4-methylbenzoyl)amino}pentanoic acid;
(2-{[3-(3,5-dichlorophenyl)propyl](3,5-dimethoxy-4-methylbenzoyl)amino}ethoxy)acetic acid;
(2-{[3-(2,6-difluorophenyl)propyl](3,5-dimethoxy-4-methylbenzoyl)amino}ethoxy)acetic acid;
(2-{(3,5-dimethoxy-4-methylbenzoyl)[3-(3-fluorophenyl)propyl]amino}ethoxy)acetic acid;
(2-{[3-(2,4-difluorophenyl)propyl](3,5-dimethoxy-4-methylbenzoyl)amino}ethoxy)acetic acid;
(2-{[3-(2-chlorophenyl)propyl](3,5-dimethoxy-4-methylbenzoyl)amino}ethoxy)acetic acid;
[(2-{(3,5-dimethoxy-4-methylbenzoyl)[3-(5-methylfuran-2-yl)propyl]amino}ethyl)sulfanyl]acetic acid;
5-{(3,5-dimethoxybenzoyl)[3-(5-methylfuran-2-yl)propyl]amino}pentanoic acid;
5-{(3,5-dimethoxybenzoyl)[3-(2-fluorophenyl)propyl]amino}pentanoic acid;
5-{[3-(2-chlorophenyl)propyl](3,5-dimethoxybenzoyl)amino}pentanoic acid;
5-{[3-(3,5-difluorophenyl)propyl](3,5-dimethoxybenzoyl)amino}pentanoic acid;
5-{[3-(2,6-difluorophenyl)propyl](3,5-dimethoxybenzoyl)amino}pentanoic acid;
5-{(3,5-dimethoxy-4-methylbenzoyl)[3-(5-methylfuran-2-yl)propyl]amino}pentanoic acid;
(2-{(3,5-dimethoxy-4-methylbenzoyl)[3-(4-fluorophenyl)propyl]amino}ethoxy)acetic acid;
N-{5-[(4-fluorobenzene-1-sulfonyl)amino]-5-oxopentyl}-3,5-dimethoxy-4-methyl-N-(3-phenylpropyl)benzamide;
3,5-dimethoxy-4-methyl-N-{5-oxo-5-[(pyridine-2-sulfonyl)amino]pentyl}-N-(3-phenylpropyl)benzamide;
3,5-dimethoxy-4-methyl-N-{5-oxo-5-[(pyridine-3-sulfonyl)amino]pentyl}-N-(3-phenylpropyl)benzamide;
3,5-dimethoxy-4-methyl-N-{5-oxo-5-[(pyridine-4-sulfonyl)amino]pentyl}-N-(3-phenylpropyl)benzamide;
N-{5-[(benzenesulfonyl)amino]-5-oxopentyl}-3,5-dimethoxy-4-methyl-N-(3-phenylpropyl)benzamide;
3-[(3,5-dimethoxy-4-methylbenzoyl)(3-phenylpropyl)amino]propyl (4-fluorobenzene-1-sulfonyl)carbamate;
5-{[3-(2,4-difluorophenyl)propyl](3,5-dimethoxy-4-methylbenzoyl)amino}pentanoic acid;
5-{[3-(2,6-difluorophenyl)propyl](3,5-dimethoxy-4-methylbenzoyl)amino}pentanoic acid;
5-{(3,5-dimethoxybenzoyl)[3-(4-fluorophenyl)propyl]amino}pentanoic acid;
5-{[2-ethoxy-1-(4-methoxyphenyl)cyclopropane-1-carbonyl](3-phenylpropyl)amino}pentanoic acid;
3,5-dimethoxy-4-methyl-N-{5-oxo-5-[(trifluoromethanesulfonyl)amino]pentyl}-N-(3-phenylpropyl)benzamide;
5-[(3,5-dimethoxy-4-methylbenzoyl)(3-phenylpropyl)amino]-2-hydroxypentanoic acid;
5-{[1-(4-methoxyphenyl)-3-oxocyclobutane-1-carbonyl](3-phenylpropyl)amino}pentanoic acid;
5-{(3,5-dimethoxy-4-methylbenzoyl) [(2R)-4-phenylbutan-2-yl]amino}pentanoic acid;
5-{(3,5-dimethoxy-4-methylbenzoyl) [(2S)-4-phenylbutan-2-yl]amino}pentanoic acid;
5-[(3,5-dimethoxy-4-methylbenzoyl)(3-phenylpropyl)amino]-2-hydroxy-2-methylpentanoic acid;
N-[2-({1-[(methanesulfonyl)amino]-2-methyl-1-oxopropan-2-yl}oxy)ethyl]-3,5-dimethoxy-4-methyl-N-(3-phenylpropyl)benzamide;
N-(2-{2-[(methanesulfonyl)amino]-2-oxoethoxy}ethyl)-3,5-dimethoxy-4-methyl-N-(3-phenylpropyl)benzamide;
5-{[cis-3-methoxy-1-(4-methoxyphenyl)cyclobutane-1-carbonyl](3-phenylpropyl)amino}pentanoic acid;
5-{[trans-3-methoxy-1-(4-methoxyphenyl)cyclobutane-1-carbonyl](3-phenylpropyl)amino}pentanoic acid;
5-[(2-methyl-4-oxo-3,4-dihydroquinazoline-8-carbonyl)(3-phenylpropyl)amino]pentanoic acid;
N-{5-[(methanesulfonyl)amino]-4-methyl-5-oxopentyl}-3,5-dimethoxy-4-methyl-N-(3-phenylpropyl)benzamide;
N-{5-[(methanesulfonyl)amino]-4,4-dimethyl-5-oxopentyl}-3,5-dimethoxy-4-methyl-N-(3-phenylpropyl)benzamide;
5-[(2,6-dimethoxypyrimidine-4-carbonyl)(3-phenylpropyl)amino]pentanoic acid;
5-[(3,5-dimethoxy-4-methylbenzoyl)(3-phenylpropyl)amino]hexanoic acid;
5-[(4-cyclopropyl-3,5-dimethoxybenzoyl)(3-phenylpropyl)amino]-2-hydroxypentanoic acid;
3,5-dimethoxy-4-methyl-N-(3-phenylpropyl)-N-[4-(1H-tetrazol-5-yl)butyl]benzamide;
5-[(5-chloro-2-methylpyrimidine-4-carbonyl)(3-phenylpropyl)amino]pentanoic acid;
5-[(6-fluoro-2-oxo-1,2,3,4-tetrahydroquinoline-4-carbonyl)(3-phenylpropyl)amino]pentanoic acid;
5-[(2-methyl-1-oxo-1,2-dihydroisoquinoline-4-carbonyl)(3-phenylpropyl)amino]pentanoic acid;
5-[(7-fluoro-2-oxo-1,2,3,4-tetrahydroquinoline-4-carbonyl)(3-phenylpropyl)amino]pentanoic acid;
5-[(2-acetamidopyridine-4-carbonyl)(3-phenylpropyl)amino]pentanoic acid;
5-[(3,6-dimethyl[1,2]oxazolo[5,4-b]pyridine-4-carbonyl)(3-phenylpropyl)amino]pentanoic acid;
5-{(3,5-dimethoxy-4-methylbenzoyl)[3-(furan-2-yl)propyl]amino}pentanoic acid;
5-{(3,5-dimethoxy-4-methylbenzoyl)[3-(5-methylfuran-2-yl)propyl]amino}-2-hydroxy-2-methylpentanoic acid;
5-{(2,4-difluoro-3,5-dimethoxybenzoyl)[3-(5-methylfuran-2-yl)propyl]amino}pentanoic acid;
5-{(3,5-diethoxybenzoyl)[3-(5-methylfuran-2-yl)propyl]amino}pentanoic acid;
5-{[3,5-dimethoxy-4-(trifluoromethyl)benzoyl][3-(5-methylfuran-2-yl)propyl]amino}pentanoic acid;
5-{(4-cyclopropyl-3,5-dimethoxybenzoyl)[3-(5-methylfuran-2-yl)propyl]amino}pentanoic acid;
5-{(3,5-dimethoxy-4-methylbenzoyl)[3-(5-methylfuran-2-yl)propyl]amino}-2-methylpentanoic acid;
5-{(3,5-dimethoxy-4-methylbenzoyl)[3-(5-methylfuran-2-yl)propyl]amino}-2,2-dimethylpentanoic acid;
3,5-dimethoxy-4-methyl-N-{[(2R)-5-oxooxolan-2-yl]methyl}-N-(3-phenylpropyl)benzamide;
5-[(3,5-dimethoxy-4-methylbenzoyl)(3-phenylbutyl)amino]pentanoic acid;
2-[(3,5-dimethoxy-4-methylbenzoyl)(3-phenylpropyl)amino]ethyl (4-fluorobenzene-1-sulfonyl)carbamate;
2-[(3,5-dimethoxy-4-methylbenzoyl)(3-phenylpropyl)amino]ethyl (methanesulfonyl)carbamate;
5-{[3-(5-chlorofuran-2-yl)propyl](3,5-dimethoxy-4-methylbenzoyl)amino}pentanoic acid; and
3-[(3,5-dimethoxy-4-methylbenzoyl)(3-phenylpropyl)amino]propyl (methanesulfonyl)carbamate.

Compound names are assigned by using Name 2015 naming algorithm by Advanced Chemical Development or Struct=Name naming algorithm as part of CHEMDRAW® ULTRA v. 12.0.2.1076.

Compounds of the present disclosure may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The present disclosure contemplates various stereoisomers and mixtures thereof and these are specifically included within the scope of this disclosure. Stereoisomers include enantiomers and diastereomers, and mixtures of enantiomers or diastereomers. Individual stereoisomers of compounds of the present disclosure may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Compounds of the present disclosure may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the present disclosure may be prepared synthetically from commercially available starting materials using selective organic transformations, or prepared in single isomeric form by purification of mixtures of the cis and trans isomers. Such methods are well-known to those of ordinary skill in the art, and may include separation of isomers by recrystallization or chromatography.

It should be understood that the compounds of the present disclosure may possess tautomeric forms, as well as geometric isomers, and that these also constitute an aspect of the present disclosure.

It should be understood that the compounds of the present disclosure that have a carboxylic acid which is substituted with a hydroxy group on the γ-carbon can dehydrate to form a butyrolactone. Under certain biological conditions, the process can be reversed.

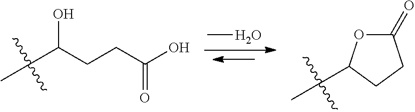

The present disclosure includes all pharmaceutically acceptable isotopically-labelled compounds of formula (I) wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the accompanying Examples using an appropriate isotopically-labeled reagents in place of the non-labeled reagent previously employed.

Thus, the formula drawings within this specification can represent only one of the possible tautomeric, geometric, or stereoisomeric forms. It is to be understood that the present disclosure encompasses any tautomeric, geometric, or stereoisomeric form, and mixtures thereof, and is not to be limited merely to any one tautomeric, geometric, or stereoisomeric form utilized within the formula drawings.

Present compounds may be used in the form of pharmaceutically acceptable salts. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts have been described in S. M. Berge et al. J. Pharmaceutical Sciences, 1977, 66: 1-19.

Compounds of the present disclosure may contain either a basic or an acidic functionality, or both, and can be converted to a pharmaceutically acceptable salt, when desired, by using a suitable acid or base. The salts may be prepared in situ during the final isolation and purification of the compounds of the present disclosure.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts may be prepared in situ during the final isolation and purification of compounds of this disclosure by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminium salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

The term “pharmaceutically acceptable prodrug” or “prodrug” as used herein, represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use.

The present disclosure contemplates compounds formed by synthetic means or formed by in vivo biotransformation of a prodrug.

Compounds described herein can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the present disclosure.

### General Synthesis

The compounds of the present disclosure can be better understood in connection with the following synthetic schemes and methods which illustrate a means by which the compounds can be prepared.

The compounds of this disclosure can be prepared by a variety of synthetic procedures. Representative procedures are shown in, but are not limited to, Schemes 1-20. In Schemes 1-20, the variables L¹, L², L³, G¹, G², G^{B}, R¹, R², R⁹, R¹⁰, R^{G3a} are as described in the Summary.

### Abbreviations

Ac for acetyl; Boc₂O for di-tert-butyl dicarbonate; Bu for butyl; Et for ethyl; CDI for carbonyldiimidazole; DBU for 1,8-diazabicyclo[5.4.0]undec-7-ene; HMPA for hexamethylphosphoramide; PyAOP for ((3H-[1,2,3]triazolo[4,5-b]pyridin-3-yl)oxy)tri(pyrrolidin-1-yl)phosphonium hexafluorophosphate(V); TBAF for tetrabutylammonium fluoride; TBS for tert-butyl(dimethyl)silyl; Tf₂O for trifluoromethanesulfonic anhydride; and TMS for trimethylsilyl.

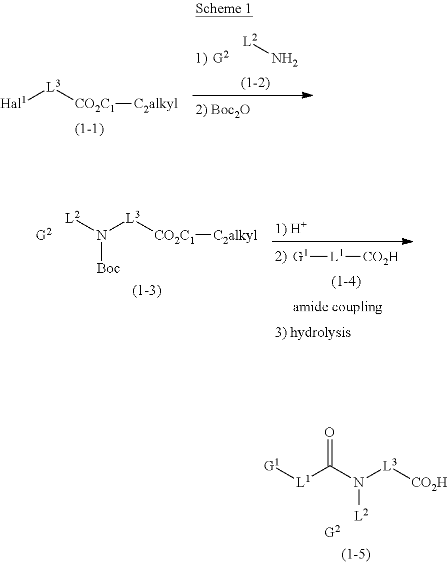

As shown in Scheme 1, compounds of formula (1-5) can be prepared from compounds of formula (1-1). Compounds of formula (1-1); wherein Hal¹ is chlorine, bromine or iodine; can be reacted with an amine of formula (1-2) heated in a solvent such as acetonitrile for 0.25-24 hours. Then the intermediate amine can be treated with di-tert-butyl dicarbonate in the presence of a tertiary amine base at ambient temperature for 0.25 to 24 hours to give compounds of formula (1-3) that can be chromatographically purified. Compounds of formula (1-3) can then be reacted in a three-step process to give compounds of formula (1-5). First, the tert-butoxycarbonyl protecting group can be removed by acidic treatment such as trifluoroacetic acid in methylene chloride or hydrochloric acid in dioxane. The exposed amine can then be coupled with a carboxylic acid of formula (1-4) using a reagent such as (1-[bis(dimethylamino)methylene]-1H-1,2,3-triazolo[4,5-b]pyridinium 3-oxid hexafluorophosphate (HATU) in the presence of a tertiary amine base in a solvent such as N,N-dimethylformamide at ambient temperature over 1 to 24 hours. Alternatively, carboxylic acids of formula (1-4) can be converted to the corresponding acid chlorides by reaction with thionyl chloride, PCl₃, PCl₅, cyanuric chloride, or oxalyl chloride. The reactions with thionyl chloride and oxalyl chloride can be catalyzed with N,N-dimethylformamide at ambient temperature in a solvent such as dichloromethane. The resultant acid chlorides can then reacted with the amines derived from carbamates of formula (1-3) optionally in the presence of a base such as a tertiary amine base such as but not limited to triethylamine or diisopropylethylamine or an aromatic base such as pyridine, at room temperature in a solvent such as dichloromethane to give the corresponding amides. Lastly, the ester can be hydrolyzed to the corresponding carboxylic acid using a base such as aqueous lithium hydroxide in a solvent such as tetrahydrofuran at ambient temperature to give compounds of formula (1-5). Compounds of formula (1-5) are representative of compounds of formula (I).

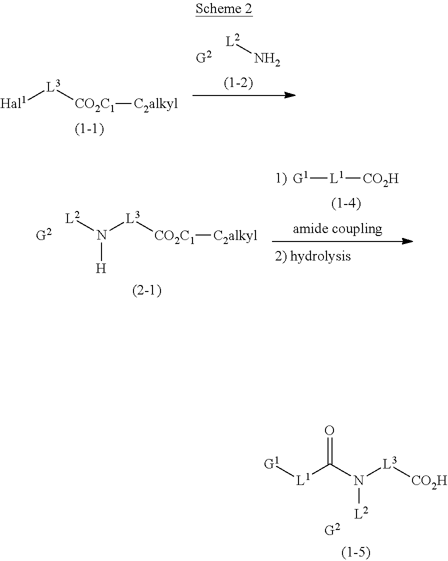

As shown in Scheme 2, compounds of formula (1-5) can also be prepared from compounds of formula (1-1) without the protection/deprotection sequence described in Scheme 1. Compounds of formula (1-1); wherein Hal¹ is chlorine, bromine or iodine; can be reacted with an amine of formula (1-2) heated in a solvent such as acetonitrile for 0.25-24 hours to give compounds of formula (2-1). Compounds of formula (2-1) can then be reacted in a two-step process to give compounds of formula (1-5). First, the amine of formula (2-1) can be coupled with a carboxylic acid of formula (1-4) using a reagent such as (1-[bis(dimethylamino)methylene]-1H-1,2,3-triazolo[4,5-b]pyridinium 3-oxide hexafluorophosphate (HATU) in the presence of a tertiary amine base in a solvent such as N,N-dimethylformamide at ambient temperature over 1 to 24 hours. Alternatively, carboxylic acids of formula (1-4) can be converted to the corresponding acid chlorides by reaction with thionyl chloride, PCl₃, PCl₅, cyanuric chloride, or oxalyl chloride. The reactions with thionyl chloride and oxalyl chloride can be catalyzed with N,N-dimethylformamide at ambient temperature in a solvent such as dichloromethane. The resultant acid chlorides can then reacted with the amines of formula (2-1) optionally in the presence of a base such as a tertiary amine base such as but not limited to triethylamine or diisopropylethylamine or an aromatic base such as pyridine, at room temperature in a solvent such as dichloromethane to give the corresponding amides. Then, the ester can be hydrolyzed to the corresponding carboxylic acid using a base such as aqueous lithium hydroxide in a solvent such as tetrahydrofuran at ambient temperature to give compounds of formula (1-5). Compounds of formula (1-5) are representative of compounds of formula (I).

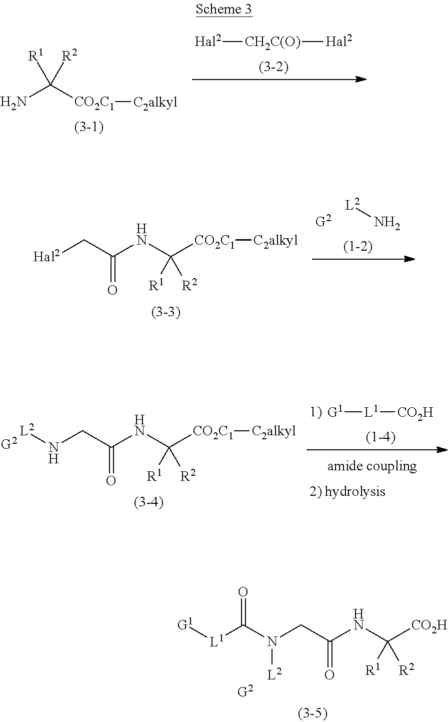

As shown in Scheme 3, compounds of formula (3-5) can be prepared from compounds of formula (3-1). Compounds of formula (3-1) can be reacted with an acid chloride of formula (3-2); wherein Hal² is chlorine or bromine; at ambient temperature in a solvent such as dichloromethane for 0.25-6 hours to give compounds of formula (3-3). Compounds of formula (3-3) can then be reacted with amines of formula (1-2) in the presence of a base such as potassium carbonate in a solvent such as acetonitrile at ambient temperature over 1-6 hours to give compounds of formula (3-4). The amine of formula (3-4) can be coupled with a carboxylic acid of formula (1-4) using a reagent such as (1-[bis(dimethylamino)methylene]-1H-1,2,3-triazolo[4,5-b]pyridinium 3-oxide hexafluorophosphate (HATU) in the presence of a tertiary amine base in a solvent such as N,N-dimethylformamide at ambient temperature over 1 to 24 hours. Alternatively, carboxylic acids of formula (1-4) can be converted to the corresponding acid chlorides as described in Schemes 1 and 2, and then the resultant acid chloride can be reacted with amines of formula (3-4) to give the corresponding amides. Then, the ester can be hydrolyzed to the corresponding carboxylic acid using a base such as aqueous lithium hydroxide in a solvent such as tetrahydrofuran at ambient temperature to give compounds of formula (3-5). Compounds of formula (3-5) are representative of compounds of formula (I).

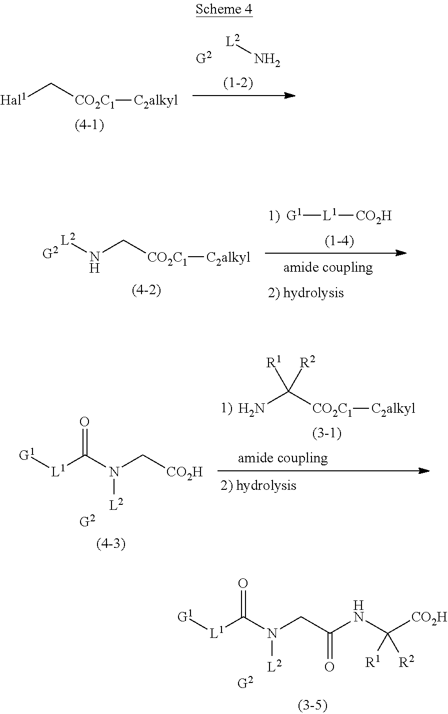

As shown in Scheme 4, compounds of formula (3-5) can also be prepared from compounds of formula (4-1). Compounds of formula (4-1), wherein Hal¹ is chlorine, bromine or iodine; can be reacted with an amines of formula (1-2) heated in a solvent such as acetonitrile in the presence of a base such as potassium carbonate for 2-24 hours to give compounds of formula (4-2). The amines of formula (4-2) can be coupled with a carboxylic acids of formula (1-4) using a reagent such as (1-[bis(dimethylamino)methylene]-1H-1,2,3-triazolo[4,5-b]pyridinium 3-oxid hexafluorophosphate (HATU) in the presence of a tertiary amine base in a solvent such as N,N-dimethylformamide at ambient temperature over 1 to 24 hours. Alternatively, carboxylic acids of formula (1-4) can be converted to the corresponding acid chlorides as described in Schemes 1 and 2, and then the resultant acid chloride can be reacted with amines of formula (4-2) to give the corresponding amides. Then, the ester can be hydrolyzed to the corresponding carboxylic acid using a base such as aqueous lithium hydroxide in a solvent such as tetrahydrofuran at ambient temperature or heated in a solvent such as dioxane to give compounds of formula (4-3). Compounds of formula (4-3) can be coupled with compounds of formula (3-1) using a reagent such as (1-[bis(dimethylamino)methylene]-1H-1,2,3-triazolo[4,5-b]pyridinium 3-oxid hexafluorophosphate (HATU) in the presence of a tertiary amine base in a solvent such as N,N-dimethylformamide at ambient temperature over 1 to 24 hours. Alternatively, carboxylic acids of formula (4-3) can be converted to the corresponding acid chlorides as described in Schemes 1 and 2, and then the resultant acid chloride can be reacted with amines of formula (3-1) to give the corresponding amides. Then, the ester can be hydrolyzed to the corresponding carboxylic acid using a base such as aqueous lithium hydroxide in a solvent such as tetrahydrofuran at ambient temperature or heated in a solvent such as dioxane to give compounds of formula (3-5). Compounds of formula (3-5) are representative of compounds of formula (I).

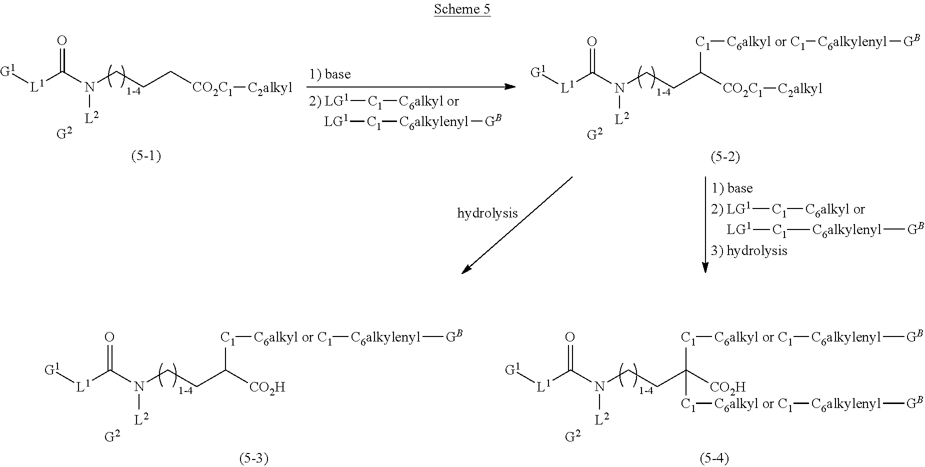

As shown in Scheme 5, compounds of formula (5-3) and formula (5-4) can be prepared from compounds of formula (5-1). Compounds of formula (5-1) can be prepared as described in Schemes 1 or 2. Compounds of formula (5-1) can be reacted with a base such as lithium diisopropylamide or lithium bis(trimethylsilyl)amide at −78° C. in a solvent such as tetrahydrofuran and then treated with LG¹-C₁-C₆alkyl or LG¹-C₁-C₆alkylenyl-G^{B}; wherein LG¹ is a leaving group such as chlorine, bromine, iodine or a sulfonate; with warming to ambient temperature to give compounds of formula (5-2). Compounds of formula (5-2) can be hydrolyzed to the corresponding carboxylic acid of formula (5-3) using a base such as aqueous lithium hydroxide in a solvent such as tetrahydrofuran at ambient temperature or heated in a solvent such as dioxane. Additionally, compounds of formula (5-2) can be deprotonated and alkylated with LG¹-C₁-C₆alkyl or LG¹-C₁-C₆alkylenyl-G^{B} as previously described to give an alpha,alpha-dialkylated ester. The ester can be hydrolyzed using the conditions previously described to give compounds of formula (5-4). Compounds of formula (5-3) and formula (5-4) are representative of compounds of formula (I).

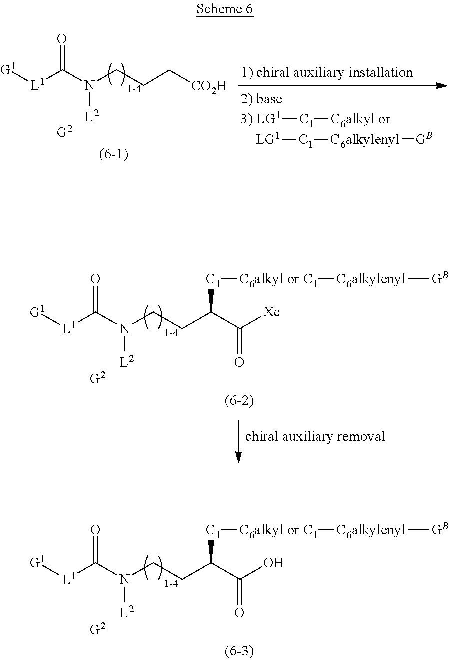

Compounds of formula (6-1) can be converted to chiral compounds of formula (6-3) using a sequence similar to that described in Scheme 5. Compounds of formula (6-1) can be reacted with a chiral auxiliary (Xc) such as the Evans' oxazolidinones. To this end, compounds of formula (6-1) can be converted to an anhydride by treatment with pivaloyl chloride in the presence of triethylamine. The anhydride can be reacted with the lithium salt of a chiral oxazolidinones. This intermediate imide can then be alkylated as described in Scheme 5 to give chiral compounds of formula (6-2). Removal of the chiral auxiliary gives compounds of formula (6-3). In the case of the oxazolidinones chiral auxiliaries, this can be accomplished with lithium hydroperoxide in a mixture of water and tetrahydrofuran. Compounds of formula (6-3) are representative of compounds of formula (I).

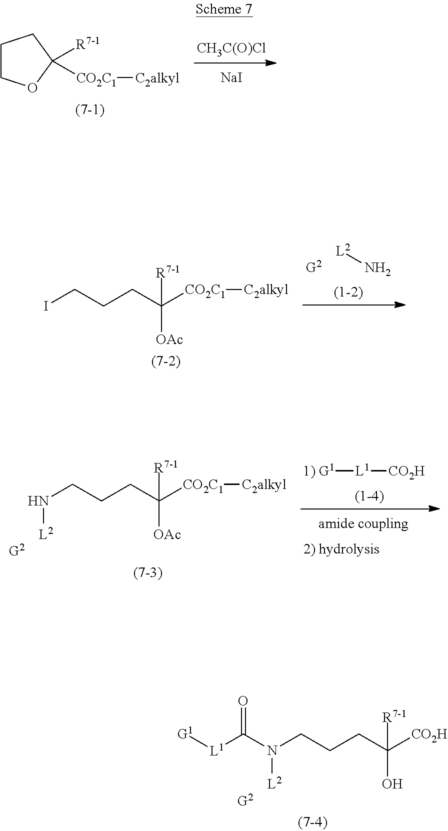

As shown in Scheme 7, compounds of formula (7-1) can be converted to compounds of formula (7-4). Accordingly, compounds of formula (7-1), wherein R⁷ 1 is either hydrogen or an alkyl group, can be reacted with acetyl chloride and sodium iodide at ambient temperature in a solvent such as acetonitrile over 6 to 30 hours to give a compound of formula (7-2). Compounds of formula (7-2) can be reacted with amines of formula (1-2) in optionally heated acetonitrile to give compounds of formula (7-3). Compounds of formula (7-3) can then be coupled with carboxylic acids of formula (1-4) or the corresponding acid chlorides and hydrolyzed as described in Schemes 1 and 2 to give compounds of formula (7-4). Compounds of formula (7-4) are representative of compounds of formula (I).

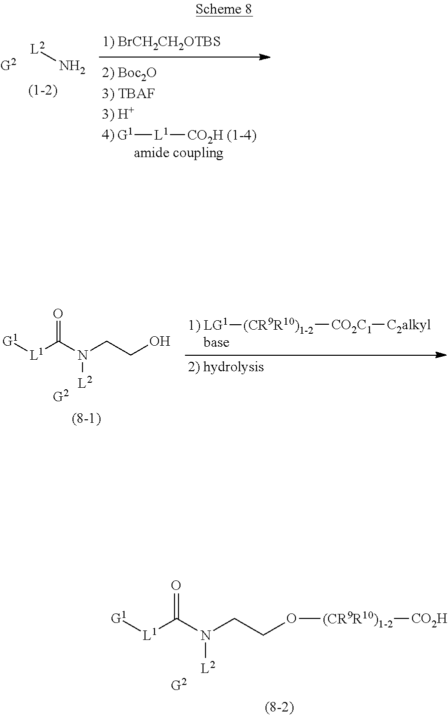

As shown in Scheme 8, compounds of formula (1-2) can be transformed to compounds of formula (8-2). Amines of formula (1-2) can be alkylated with (2-bromoethoxy)(tert-butyl)dimethylsilane in optionally heated acetonitrile. The amine thus obtained can be protected by treatment with di-tert-butyl dicarbonate. Then the silyl ether can be cleaved by treatment with tetrabutylammonium fluoride in a solvent such as tetrahydrofuran to reveal the primary hydroxyl group. The tert-butoxycarbonyl protecting group can be removed by acidic treatment with trifluoroacetic acid in dichloromethane or hydrochloric acid in dioxane to expose the amine. The amine can then be coupled with carboxylic acids of formula (1-4) using the amide coupling conditions described in Schemes 1 and 2 or the alternative acid chloride methodology also described in Schemes 1 and 2 to give compounds of formula (8-1). Compounds of formula (8-1) can treated with a base such as potassium tert-butoxide in an optionally heated solvent such as tetrahydrofuran and then alkylated with LG¹-(CR⁹R¹⁰)₁₋₂—CO₂C₁-C₂alkyl; wherein LG¹ is a leaving group such as chlorine, bromine, iodine or a sulfonate. The intermediate ester can be hydrolyzed using the conditions described in Scheme 4 to give compounds of formula (8-2). Ethers of formula (8-2) are representative of compounds of formula (I).

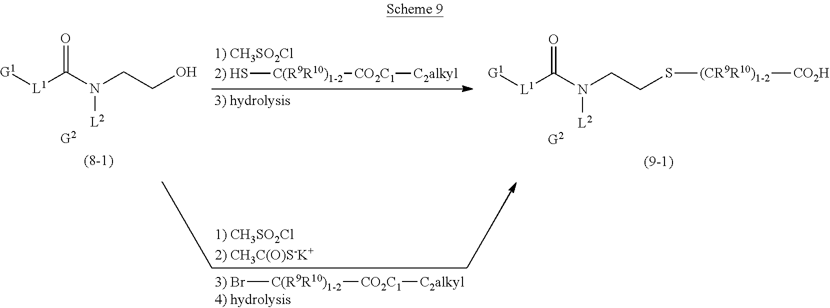

As shown in Scheme 9, compounds of formula (8-1) can be converted to thioethers of formula (9-1) by two different sequences. In the first option, compounds of formula (8-1) can first be reacted with methanesulfonyl chloride in the presence of a tertiary amine base in dichloromethane. The intermediate sulfonate can then be displaced by reaction with HS—C(R⁹R¹⁰)₁₋₂—CO₂C₁-C₂alkyl in a mixture of a solvent such as dimethyl sulfoxide and acetone in the presence of a base such as potassium carbonate. Lastly, the ester can be hydrolyzed to the corresponding carboxylic acid using a base such as aqueous lithium hydroxide in a solvent such as tetrahydrofuran at ambient temperature or heated in a solvent such as dioxane to give compounds of formula (9-1).

Alternatively, compounds of formula (8-1) can be reacted with methanesulfonyl chloride in the presence of a tertiary amine base in dichloromethane. The intermediate sulfonate can then be displaced by reaction with potassium ethanethioate in optionally heated N,N-dimethylformamide. Then the intermediate ethanethioate can be reacted with Br—C(R⁹R¹⁰)₁₋₂—CO₂C₁-C₂alkyl in the presence of a base such as potassium hydroxide in a solvent such as optionally heated methanol. Lastly, the ester thus formed can be hydrolyzed to the corresponding carboxylic acid using a base such as aqueous lithium hydroxide in a solvent such as tetrahydrofuran at ambient temperature or heated in a solvent such as dioxane to give compounds of formula (9-1). Compounds of formula (9-1) are representative of compounds of formula (I).

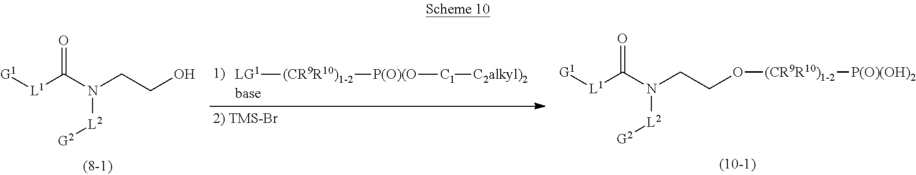

As shown in Scheme 10, phosphonic acids of formula (10-1) can be prepared from the primary alcohols of formula (8-1). Compounds of formula (8-1) can be alkylated with LG¹-C(R⁹R¹⁰)₁₋₂—P(O)(O—C₁-C₂alkyl)₂; wherein LG¹ is a leaving group such as chlorine, bromine, iodine or a sulfonate; in a solvent such as tetrahydrofuran and the presence of a base such as sodium hydride. Conversion of the intermediate phosphonate to the corresponding phosphonic acids of formula (10-1) is achieved by treatment with bromotrimethylsilane in a solvent such as dichloromethane. Compounds of formula (10-1) are representative of compounds of formula (I).

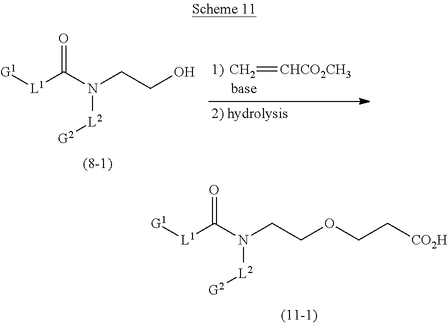

As shown in Scheme 11, carboxylic acids of formula (11-1) can be prepared from the primary alcohols of formula (8-1). Compounds of formula (8-1) can be reacted methyl acrylate in the presence of a base such as sodium hydride in a solvent such as tetrahydrofuran. The Michael reaction adducts can then be hydrolyzed using a base such as aqueous lithium hydroxide in a solvent such as tetrahydrofuran at ambient temperature or heated in a solvent such as dioxane to give compounds of formula (11-1). Compounds of formula (11-1) are representative of compounds of formula (I).

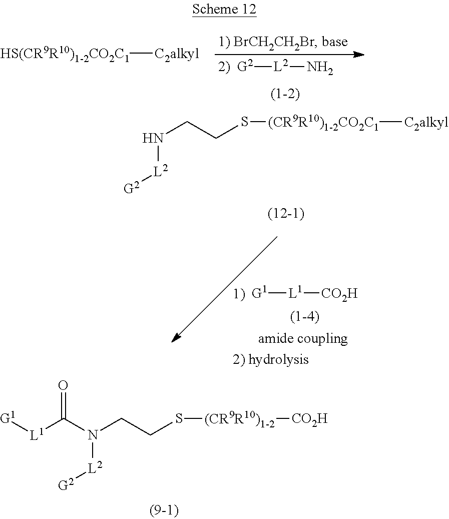

As shown in Scheme 12, mercapto esters, HS—C(R⁹R¹⁰)₁₋₂—CO₂C₁-C₂alkyl, can be converted to compounds of formula (9-1) using an alternative sequence. Accordingly, mercapto esters, HS—C(R⁹R¹⁰)₁₋₂—CO₂C₁-C₂alkyl, can be alkylated with 1,2-dibromoethane in the presence of a base such as potassium carbonate in a heated solvent such as acetonitrile. The intermediate bromide can then be reacted with amines of formula (1-2) in heated acetonitrile to give compounds of formula (12-1). Compounds of formula (12-1) can be coupled with compounds of formula (1-4) using a reagent such as (1-[bis(dimethylamino)methylene]-1H-1,2,3-triazolo[4,5-b]pyridinium 3-oxid hexafluorophosphate (HATU) in the presence of a tertiary amine base in a solvent such as N,N-dimethylformamide at ambient temperature over 1 to 24 hours. Alternatively, carboxylic acids of formula (1-4) can be converted to the corresponding acid chlorides as described in Schemes 1 and 2, and then the resultant acid chloride can be reacted with amines of formula (12-1) to give the corresponding amides. Then, the ester can be hydrolyzed to the corresponding carboxylic acid using a base such as aqueous lithium hydroxide in a solvent such as tetrahydrofuran at ambient temperature or heated in a solvent such as dioxane to give compounds of formula (9-1). Compounds of formula (9-1) are representative of compounds of formula (I).

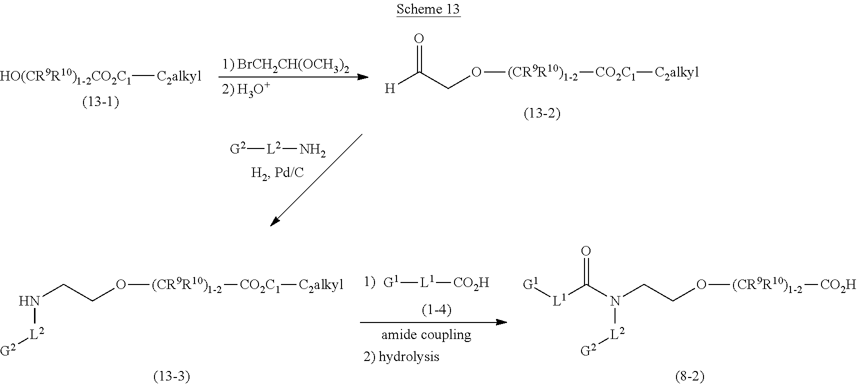

As shown in Scheme 13, compounds of formula (13-1) can be transformed to compounds of formula (8-2) in the illustrated alternative sequence. Accordingly, compounds of formula (13-1) can be alkylated with 2-bromo-1,1-dimethoxyethane in the presence of sodium hydride in a solvent such as N,N-dimethylformamide. The resultant acetals can be transformed to the corresponding aldehydes, compounds of formula (13-2), by treatment with aqueous acid. Aldehydes of formula (13-2) can be reductively aminated with G²-L²-NH₂ under a hydrogen atmosphere in the presence of palladium on carbon in a solvent such 1,2-dichloroethane to give compounds of formula (13-3). Compounds of formula (13-3) can be coupled with compounds of formula (1-4) using a reagent such as (1-[bis(dimethylamino)methylene]-1H-1,2,3-triazolo[4,5-b]pyridinium 3-oxid hexafluorophosphate (HATU) in the presence of a tertiary amine base in a solvent such as N,N-dimethylformamide at ambient temperature over 1 to 24 hours. Alternatively, carboxylic acids of formula (1-4) can be converted to the corresponding acid chlorides as described in Schemes 1 and 2, and then the resultant acid chloride can be reacted with amines of formula (13-3) to give the corresponding amides. Then, the ester can be hydrolyzed to the corresponding carboxylic acid using a base such as aqueous lithium hydroxide in a solvent such as tetrahydrofuran at ambient temperature or heated in a solvent such as dioxane to give compounds of formula (8-2). Compounds of formula (8-2) are representative of compounds of formula (I).

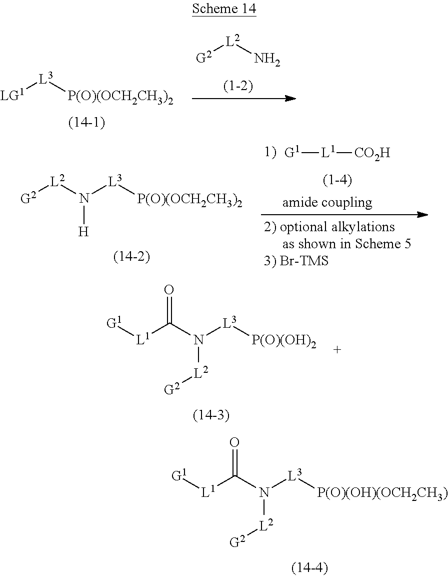

As shown in Scheme 14, compounds of formula (14-3) and formula (14-4) can be prepared from compounds of formula (14-1) in a sequence similar to that described for the corresponding carboxylic acids in Scheme 2. Compounds of formula (14-1); wherein LG¹ is chlorine, bromine, iodine or a sulfonate; can be reacted with an amine of formula (1-2) heated in a solvent such as acetonitrile or tetrahydrofuran in the presence of a tertiary amine for 0.25-24 hours to give compounds of formula (14-2). Compounds of formula (14-2) can then be reacted in a three-step process to give compounds of formula (14-3) and formula (14-4). First, the amines of formula (14-2) can be coupled with a carboxylic acids of formula (1-4) using a reagent such as (1-[bis(dimethylamino)methylene]-1H-1,2,3-triazolo[4,5-b]pyridinium 3-oxid hexafluorophosphate (HATU) in the presence of a tertiary amine base in a solvent such as N,N-dimethylformamide at ambient temperature over 1 to 24 hours. Alternatively, carboxylic acids of formula (1-4) can be converted to the corresponding acid chlorides as described in Schemes 1 and 2, and then the resultant acid chloride can be reacted with amines of formula (14-2) to give the corresponding amides. The intermediate phosphanates can then optionally be alkylated on the alpha carbon using the conditions shown in Scheme 5. Then, the phosphonate can be converted to a mixture of the corresponding phosphonic acid of formula (14-3) and monophosphonate of formula (14-4) using bromotrimethylsilane in a solvent such as dichloromethane at ambient. Compounds of formula (14-3) and formula (14-4) are representative of compounds of formula (I).

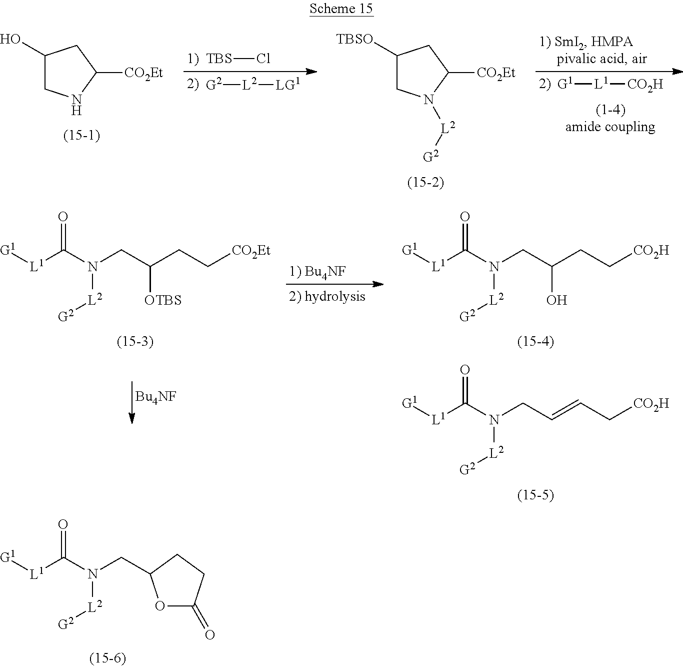

As shown in Scheme 15, compounds of formula (15-1) can be converted to compounds of formula (15-4), formula (15-5) and formula (15-6). Ethyl 4-hydroxyprolinate can be reacted first with tert-butyldimethylsilyl chloride in the presence of imidazole in dichloromethane, and then the intermediate silyl ether can be reacted with G²-L²-LG¹; wherein LG¹ is chlorine, bromine, iodine or a sulfonate; in heated acetonitrile to give compounds of formula (15-2). Compounds of formula (15-2) can be reacted with samarium iodide in hexamethylphosphoramide and tetrahydrofuran in the presence of air and pivalic acid. Subsequent amide bond formation by coupling with a carboxylic acids of formula (1-4) using a reagent such as (1-[bis(dimethylamino)methylene]-1H-1,2,3-triazolo[4,5-b]pyridinium 3-oxid hexafluorophosphate (HATU) in the presence of a tertiary amine base in a solvent such as N,N-dimethylformamide at ambient temperature over 1 to 24 hours gives compounds of formula (15-3). Alternatively, carboxylic acids of formula (1-4) can be converted to the corresponding acid chlorides as described in Schemes 1 and 2, and then the resultant acid chloride can be reacted with amines derived from compounds of formula (15-2) to give the corresponding amides. Compounds of formula (15-3) can be treated with tetra-n-butylammonium fluoride in tetrahydrofuran for about 1 hour to remove the silyl protecting group. Subsequent ester hydrolysis using a base such as aqueous lithium hydroxide in a solvent such as tetrahydrofuran at ambient temperature or heated in a solvent such as dioxane to gives a separable mixture of compounds of formula (15-4) and formula (15-5). Compounds of formula (15-3) can be reacted with tetra-n-butylammonium fluoride overnight in tetrahydrofuran to give compounds of formula (15-6). Compounds of formula (15-4), formula (15-5) and formula (15-6) are representative of compounds of formula (I).

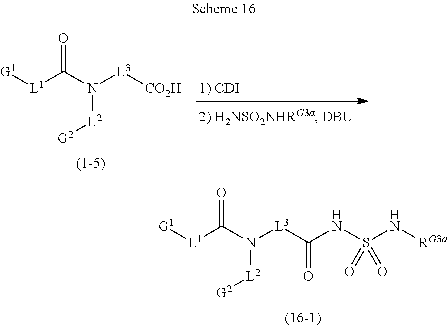

As is shown in Scheme 16, compounds of formula (1-5) can be transformed to compounds of formula (16-1). Compounds of formula (1-5) can be treated with carbonyldiimidazole in heated tetrahydrofuran. Then, an N-substituted sulfuric diamide, H₂NSO₂NHR^{G3a}, can be added in the presence of a base such as 1,8-diazabicyclo[5.4.0]undec-7-ene at or near ambient temperature to give compounds of formula (16-1). Compounds of formula (16-1) are representative of compounds of formula (I).

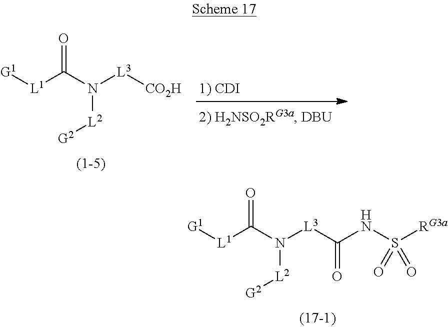

As is shown in Scheme 17, compounds of formula (1-5) can be transformed to compounds of formula (17-1). Compounds of formula (1-5) can be treated with carbonyldiimidazole in heated isopropyl acetate. Then, a sulfonamide, H₂NSO₂R^{G3a}, can be added in the presence of a base such as 1,8-diazabicyclo[5.4.0]undec-7-ene with continued heating to give compounds of formula (17-1). Compounds of formula (17-1) are representative of compounds of formula (I).

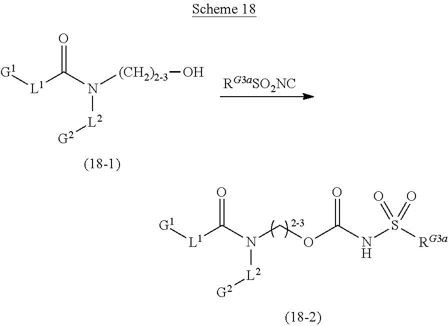

As is shown in Scheme 18, compounds of formula (18-1) can be transformed to compounds of formula (18-2). Compounds of formula (18-1) can be treated with a sulfonyl isocyanate, CNSO₂R^{G3a}, at or near ambient temperature in a solvent such as tert-butyl methyl ether to give compound of formula (18-2). Compounds of formula (18-2) are representative of compounds of formula (I).

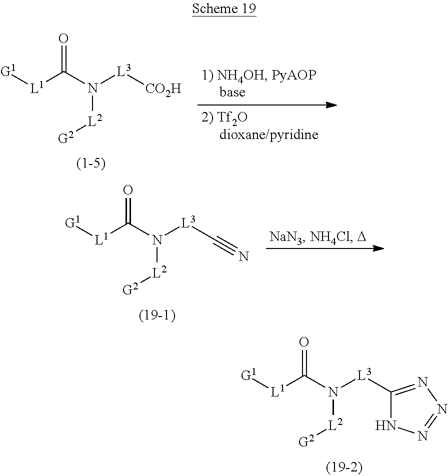

As is shown in Scheme 19, compounds of formula (1-5) can be transformed to compounds of formula (19-2). Compounds of formula (1-5) can be converted to the corresponding nitrile in a two-step process. Compounds of formula (1-5) can be reacted with ammonium hydroxide and ((3H-[1,2,3]triazolo[4,5-b]pyridin-3-yl)oxy)tri(pyrrolidin-1-yl)phosphonium hexafluorophosphate(V) (PyAOP) in the presence of a tertiary amine base in a solvent such as tetrahydrofuran to give the corresponding amide. The amide can then be dehydrated in the second step by treatment with trifluoroacetic anhydride in a solvent such as a mixture of dioxane and pyridine to give compounds of formula (19-1). Compounds of formula (19-1) can then be reacted with sodium azide in the presence of ammonium chloride in heated N,N-dimethylformamide to give compounds of formula (19-2). Compounds of formula (19-2) are representative of compounds of formula (I).

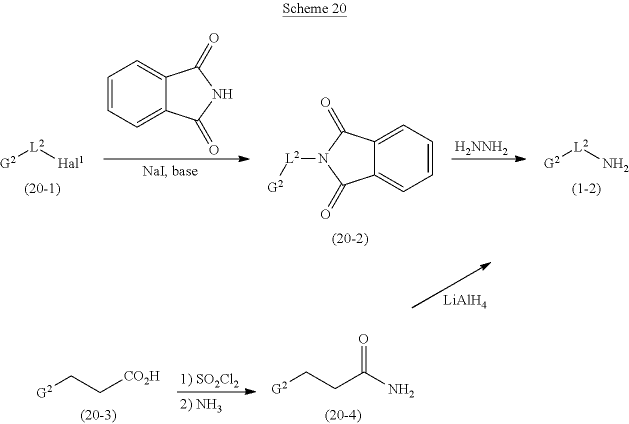

As shown in Scheme 20, amines of formula (1-2) can be prepared from compounds of formula (20-1). Compounds of formula (20-1); wherein Hal¹ is chlorine, bromine, or iodine; can be reacted with isoindoline-1,3-dione in the presence of sodium iodide and a base such as potassium carbonate in a solvent such as optionally heated acetonitrile to give compounds of formula (20-2). Compounds of formula (20-2) can be reacted with hydrazine or hydrazine hydrate in an optionally heated solvent such as methanol to give compounds of formula (1-2).

Alternatively, compounds of formula (20-3) can be reacted first with thionyl chloride, and then the intermediate acid chloride in optionally cooled tetrahydrofuran can be reacted with ammonia to give compounds of formula (20-4). Compounds of formula (20-4) can be reduced with a reductant such as lithium aluminum hydride in optionally heated tetrahydrofuran to give compounds of formula (1-2).

Compounds of formula (1-2) can be used as described in Schemes 1, 2, 3, 4, 7, 8, 13 and 14.

The compounds and intermediates of the present disclosure may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

Many of the compounds of the present disclosure have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Unless otherwise specified, solvents, temperatures and other reaction conditions can be readily selected by one of ordinary skill in the art. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the present disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the present disclosure can be accomplished by methods analogous to those described in the synthetic schemes described hereinabove and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the present disclosure is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the present disclosure is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as illustrated in the Examples section are illustrative and are not to be read as limiting the scope of the present disclosure as it is defined in the appended claims. All alternatives, modifications, and equivalents of the synthetic methods and specific examples are included within the scope of the claims.

### Pharmaceutical Compositions

This disclosure also provides for pharmaceutical compositions comprising a therapeutically effective amount of a compound of formula (I), or a pharmaceutically acceptable salt thereof together with a pharmaceutically acceptable carrier, diluent, or excipient thereof. The phrase “pharmaceutical composition” refers to a composition suitable for administration in medical or veterinary use.

The pharmaceutical compositions that comprise a compound of formula (I), alone or in combination with a second therapeutic agent, may be administered to the subjects orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which may serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate), and suitable mixtures thereof. Proper fluidity may be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations may be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In certain embodiments, solid dosage forms may contain from 1% to 95% (w/w) of a compound of formula (I). In certain embodiments, the compound of formula (I) may be present in the solid dosage form in a range of from 5% to 70% (w/w). In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

The pharmaceutical composition may be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form may be a capsule, tablet, cachet, or lozenge itself, or it may be the appropriate number of any of these in packaged form. The quantity of active component in a unit dose preparation may be varied or adjusted from 0.1 mg to 1000 mg, from 1 mg to 100 mg, or from 1% to 95% (w/w) of a unit dose, according to the particular application and the potency of the active component. The composition may, if desired, also contain other compatible therapeutic agents.

The dose to be administered to a subject may be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician may evaluate factors such as the circulating plasma levels of the compound, compound toxicities, and/or the progression of the disease, etc.

For administration, compounds may be administered at a rate determined by factors that may include, but are not limited to, the LD₅₀ of the compound, the pharmacokinetic profile of the compound, contraindicated drugs, and the side-effects of the compound at various concentrations, as applied to the mass and overall health of the subject. Administration may be accomplished via single or divided doses.

The compounds utilized in the pharmaceutical method of the present disclosure may be administered, for example, at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Treatment may be initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds may also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols, and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which may be prepared by mixing the compounds with suitable non-irritating carriers or carriers such as cocoa butter, polyethylene glycol, or a suppository wax which are solid at room temperature but liquid at body temperature and therefore melt in the rectum or vaginal cavity and release the active compound.

Compounds may also be administered in the form of liposomes. Liposomes generally may be derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes may be used. The present compositions in liposome form may contain, in addition to a compound of the present disclosure, stabilizers, preservatives, excipients, and the like. Examples of lipids include, but are not limited to, natural and synthetic phospholipids, and phosphatidyl cholines (lecithins), used separately or together.

Methods to form liposomes have been described, see example, Prescott, Ed., Methods in Cell Biology, Volume XIV, Academic Press, New York, N.Y. (1976), p. 33 et seq.

Dosage forms for topical administration of a compound described herein include powders, sprays, ointments, and inhalants. The active compound may be mixed under sterile conditions with a pharmaceutically acceptable carrier and any needed preservatives, buffers or propellants which may be required. Ophthalmic formulations, eye ointments, powders and solutions are also contemplated as being within the scope of this disclosure.

### Methods of Use

The compounds and compositions using any amount and any route of administration may be administered to a subject for the treatment or prevention of liver, kidney, skin, heart and lung disease, cancer-associated disease, proliferative disease, inflammation/immune system disease, disease by secretory dysfunction and fibrosis.

The term “administering” refers to the method of contacting a compound with a subject. Thus, the compounds may be administered by injection, that is, intravenously, intramuscularly, intracutaneously, subcutaneously, intraduodenally, parentally, or intraperitoneally. Also, the compounds described herein may be administered by inhalation, for example, intranasally. Additionally, the compounds may be administered transdermally, topically, via implantation, transdermally, topically, and via implantation. In certain embodiments, the compounds and compositions thereof may be delivered orally. The compounds may also be delivered rectally, bucally, intravaginally, ocularly, or by insufflation. Lysophosphatidic acid receptor 1 (LPAR1) modulated disorders and conditions may be treated prophylactically, acutely, and chronically using compounds and compositions thereof, depending on the nature of the disorder or condition. Typically, the host or subject in each of these methods is human, although other mammals may also benefit from the administration of compounds and compositions thereof as set forth hereinabove.

Compounds of the present disclosure are useful as modulators of LPAR1. Thus, the compounds and compositions are particularly useful for treating or lessening the severity or progression of a disease, disorder, or a condition where the lysophosphatidic acid receptor 1 is involved. Accordingly, the present disclosure provides a method for treating of liver, kidney, skin, heart and lung disease, cancer-associated disease, proliferative disease, inflammation/immune system disease, disease by secretory dysfunction or fibrosis in a subject, wherein the method comprises the step of administering to said subject a therapeutically effective amount of a compound of formula (I) or a preferred embodiment thereof as set forth above, with or without a pharmaceutically acceptable carrier.

A compound according to formula (I) or a pharmaceutically acceptable salt thereof for use in medicine.

A compound according to formula (I) or a pharmaceutically acceptable salt thereof for use in the treatment of liver, kidney, skin, heart and lung disease, cancer-associated disease, proliferative disease, inflammation/immune system disease, disease by secretory dysfunction or fibrosis.

The use of a compound according to formula (I) or a pharmaceutically acceptable salt thereof in the preparation of a medicament.

The use of a compound according to formula (I) in the preparation of a medicament for use in the treatment of liver, kidney, skin, heart and lung disease, cancer-associated disease, proliferative disease, inflammation/immune system disease, disease by secretory dysfunction or fibrosis.

The present compounds may be co-administered to a subject. The term “co-administered” means the administration of two or more different therapeutic agents that are administered to a subject by combination in the same pharmaceutical composition or separate pharmaceutical compositions. Thus co-administration involves administration at the same time of a single pharmaceutical composition comprising two or more therapeutic agents or administration of two or more different compositions to the same subject at the same or different times.

In some embodiments, the methods comprise combination therapy, wherein the compound(s) and/or salt(s) of the present disclosure is/are co-administered with a second (or even a third, fourth, etc.) compound, such as, for example, another therapeutic agent used to treat liver, kidney, skin, heart and lung disease, cancer-associated disease, proliferative disease, inflammation/immune system disease, disease of secretory dysfunction, and fibrosis. The compound(s) and/or salt(s) of this present disclosure can also be co-administered with therapeutic agents other than therapeutic agents used to treat liver, kidney, skin, heart and lung disease, cancer-associated disease, proliferative disease, inflammation/immune system disease, disease of secretory dysfunction, and fibrosis. In these co-administration embodiments, the compound(s) and/or salt(s) of the present disclosure and the second, (third, fourth, fifth, etc.) therapeutic agent(s) may be administered, for example, in a substantially simultaneous manner (e.g., or within about five minutes of each other), in a sequential manner, or both. It is contemplated that such combination therapies may include administering one therapeutic agent multiple times between the administrations of the other. The time period between the administration of each agent may range from a few seconds (or less) to several hours or days, and will depend on, for example, the properties of each composition and active ingredient (e.g., potency, solubility, bioavailability, half-life, and kinetic profile), as well as the condition of the patient. The compound(s) and/or salt(s) of this disclosure and the second, (third, fourth, fifth, etc.) therapeutic agent may also be administered in a single formulation.

In certain embodiments, the method comprises co-administering to the subject the compound(s) and/or salt(s) of the present disclosure with one or more compounds selected from the group consisting of corticosteroids, immunosuppresant, analgesics, anti-cancer agent, anti-inflammatories, chemokine receptor antagonists, bronchodilators, leukotriene receptor antagonists, leukotriene formation inhibitors, monoacylglycerol kinase inhibitors, phospholipase A1 inhibitors, phospholipase A2 inhibitors, and lysophospholipase D (lysoPLD) inhibitors, autotaxin inhibitors, decongestants, antihistamines, mucolytics, anticholinergics, antitussives, expectorants, and β-2 agonists.

This disclosure also is directed to kits that comprise one or more compounds and/or salts of the present disclosure, and, optionally, one or more additional therapeutic agents.

This disclosure also is directed to methods of use of the compounds, salts, compositions, and/or kits of the present disclosure to, for example, modulate the lysophosphatidic acid receptor 1, and treat a disease treatable by modulating the lysophosphatidic acid receptor 1 (including liver, kidney, skin, heart and lung disease, cancer-associated disease, proliferative disease, inflammation/immune system disease, disease by secretory dysfunction or fibrosis).

This disclosure also is directed to a use of one or more compounds and/or salts of the present disclosure in the preparation of a medicament. The medicament optionally can comprise one or more additional therapeutic agents. In some embodiments, the medicament is useful for treating liver, kidney, skin, heart and lung disease, cancer-associated disease, proliferative disease, inflammation/immune system disease, disease by secretory dysfunction or fibrosis.

This disclosure also is directed to a use of one or more compounds and/or salts of the present disclosure in the manufacture of a medicament for the treatment of liver, kidney, skin, heart and lung disease, cancer-associated disease, proliferative disease, inflammation/immune system disease, disease by secretory dysfunction, pain or fibrosis. The medicament optionally can comprise one or more additional therapeutic agents.

Further benefits of Applicants' disclosure will be apparent to one skilled in the art from reading this patent application.

The following Examples may be used for illustrative purposes and should not be deemed to narrow the scope of the present disclosure.

### EXAMPLES

### General

Chemicals were purchased from commercial suppliers. ¹H NMR spectra were recorded on a Bruker AVANCE™ III 400 instrument. LC-MS measurements were run on Agilent 1260 HPLC/6120B MS System using the method described below. Final products were purified by preparative HPLC: Waters 2489 UV/Vis Detector and Waters 2545 Binary Gradient Pump; RT=retention time in minutes; using the methods described below.

LC-MS Methods:

LC-MS analyses were performed with the following methods.

Mobile phase, solution A: water (0.1% CF₃CO₂H); solution B: CH₃CN
Gradient: 5% B increase to 95% B within 1.5 minutes, 95% B for 2.3 minutes, back to 5% B within
0.1 minute and run for another 0.6 minute.
Flow Rate: 1.2 mL/minute.

### Column: Phenomenex® Kinetex® C18, 2.6 μm, 3.0×30 mm. 100 Å

### Column Temperature: 40° C.

Agilent 1100/1200 HPLC system running Xcalibur 2.0.7, Open-Access 1.4, and custom login software. The mass spectrometer was operated under positive APCI or ESI ionization conditions dependent on the system used. The HPLC system comprised an Agilent Binary pump, degasser, column compartment, autosampler and diode-array detector, with a Polymer Labs ELS-2100 evaporative light-scattering detector. The column used was a Phenomenex® Kinetex® C8, 2.6 μm 100 Å (2.1 mm×30 mm), at a temperature of 65° C.

“TFA method”: A gradient of 5-100% acetonitrile (A) and 0.1% trifluoroacetic acid in water (B) was used, at a flow rate of 1.5 mL/minute (0-0.05 minutes 5% A, 0.05-1.2 minutes 5-100% A, 1.2-1.4 minutes 100% A, 1.4-1.5 minutes 100-5% A. 0.25 minutes post-run delay).

“Ammonium acetate method”: A gradient of 5-100% acetonitrile (A) and 10 mM ammonium acetate in water (B) was used, at a flow rate of 1.5 mL/minute (0-0.05 minutes 5% A, 0.05-1.2 minutes 5-100% A, 1.2-1.4 minutes 100% A, 1.4-1.5 minutes 100-5% A. 0.25 minutes post-run delay).

“TFA long with integration method”: A gradient of 5-100% acetonitrile (A) and 0.1% trifluoroacetic acid in water (B) was used, at a flow rate of 1.5 mL/minute (0-0.1 minute 5% A, 0.1-5.2 minutes 5-100% A, 5.2-5.7 minutes 100% A, 5.7-6.0 minutes 100-5% A. 0.25 minutes post-run delay).

Analytical Chiral HPLC Method

Analytical supercritical fluid chromatography (SFC) was performed on an Agilent 1260 Infinity system utilizing an Agilent 1100 HPLC stack running under Agilent OpenLab software control. The SFC system included a 6-way column switcher, CO₂ pump, modifier pump, oven, UV detector, and backpressure regulator. The analytical method set the oven temperature to 35° C., the outlet pressure to 150 bar, and UV detection to 220 nm and 254 nm. The mobile phase was comprised of supercritical CO₂ supplied by a beverage-grade CO₂ cylinder modified with isopropanol. The mobile phase was held isocratically at 15% isopropanol in CO₂ over 5 minutes at a flow rate of 3 mL/minute. The instrument was fitted with a Chiralpak® AD-H column with dimensions of 4.6 mm i.d.×150 mm length with 5 μm particles.

Preparative HPLC Method:

HPLC purifications were completed using the following methods.

Mobile phase, solution A: water (0.1% CF₃CO₂H or 0.02% NH₄OH); solution B: CH₃CN
Gradient: 30% B increase to 95% B within 10.5 minutes, 95% B for 3 minutes, back to 30% B within
0.1 minute and run for another 1.5 minutes.
Flow Rate: 15 mL/minute.

### Column: Waters® Xbridge®, Prep C18, 5.0 μm OBD, 19×150 mm

### Column Temperature: 23° C.

APS Waters Prep-HPLC Purification, small scale (10 mg-300 mg):
Samples were purified by preparative HPLC on a Phenomenex® Luna® C8(2) 5 μm 100 Å AXIA™ column (30 mm×75 mm).

“TFA method”: A gradient of acetonitrile (A) and 0.1% trifluoroacetic acid in water (B) was used, at a flow rate of 50 mL/minute (0-1.0 minute 5% A, 1.0-8.5 minutes linear gradient 5-100% A, 8.5-11.5 minutes 100% A, 11.5-12.0 minutes linear gradient 95-5% A).

“Ammonium acetate method”: A gradient of acetonitrile (A) and 10 mM ammonium acetate in water (B) was used, at a flow rate of 50 mL/minute (0-1.0 minute 5% A, 1.0-8.5 minutes linear gradient 5-100% A, 8.5-11.5 minutes 100% A, 11.5-12.0 minutes linear gradient 95-5% A).

Samples were injected in 1.5 mL dimethyl sulfoxide:methanol (1:1). A custom purification system was used, consisting of the following modules: Waters LC4000 preparative pump; Waters 996 diode-array detector; Waters 717+ autosampler; Waters SAT/IN module, Alltech Varex III evaporative light-scattering detector; Gilson 506C interface box; and two Gilson FC204 fraction collectors. The system was controlled using Waters Millennium32 software, automated using an in-house developed Visual Basic application for fraction collector control and fraction tracking. Fractions were collected based upon UV signal threshold and selected fractions subsequently analyzed by flow injection analysis mass spectrometry using positive APCI ionization on a Finnigan Navigator using 70:30 CH₃OH:10 mM NH₄OH(aqueous) at a flow rate of 0.8 mL/minute. Loop-injection mass spectra were acquired using a Finnigan Navigator running Navigator 1.8 software and a Gilson 215 liquid handler for fraction injection controlled by an in-house developed Visual Basic application.

Abbreviations: APCI for atmospheric pressure chemical ionization; atm for atmospheres; DCI for desorption chemical ionization; DMSO for dimethyl sulfoxide; ESI for electrospray ionization; HATU for N-[(dimethylamino)-1H-1,2,3-triazolo-[4,5-b]pyridin-1-ylmethylene]-N-methylmethanaminium hexafluorophosphate N-oxide or (1-[bis(dimethylamino)methylene]-1H-1,2,3-triazolo[4,5-b]pyridinium 3-oxid hexafluorophosphate; HMPA for hexamethylphosphoramide; HPLC for high performance liquid chromatography; LC-MS for liquid chromatography-mass spectrometry; NMR for nuclear magnetic resonance; Pd₂(dba)₃ for tris(dibenzylideneacetone)dipalladium(0); RT for retention time; and TLC for thin layer chromatography

### Example 1

### 2-{[N-(3,5-dimethoxy-4-methylbenzoyl)-N-(3-phenylpropyl)glycyl]amino}-2,3-dihydro-1H-indene-2-carboxylic Acid

### Step 1: ethyl 2-(2-chloroacetamido)-2,3-dihydro-1H-indene-2-carboxylate

To a solution of ethyl 2-amino-2,3-dihydro-1H-indene-2-carboxylate (500 mg, 2.44 mmol) and diisopropylethylamine (0.851 mL, 4.87 mmol) in CH₂Cl₂ (20 mL) was added a solution of 2-chloroacetyl chloride (275 mg, 2.44 mmol) in CH₂Cl₂ (5 mL) dropwise at 0° C. Then the resulting mixture was stirred at room temperature for 30 minutes. A saturated aqueous ammonium chloride solution was added to the reaction mixture followed by extraction with CH₂Cl₂ (10 mL×2). The combined organic fractions were washed with brine, dried over anhydrous Na₂SO₄, filtered and concentrated to give a residue that was purified by flash column chromatography on silica gel eluted with ethyl acetate in hexanes (0-50%) to give the titled compound. LC-MS (ESI+) m/z 282.2 (M+H)⁺, RT=1.751 minutes.

### Step 2: ethyl 2-{[N-(3-phenylpropyl)glycyl]amino}-2,3-dihydro-1H-indene-2-carboxylate

A mixture of ethyl 2-(2-chloroacetamido)-2,3-dihydro-1H-indene-2-carboxylate (70.0 mg, 0.248 mmol, Step 1), 3-phenylpropan-1-amine (33.6 mg, 0.248 mmol) and potassium carbonate (34.3 mg, 0.248 mmol) in CH₃CN (1.5 mL) was stirred at room temperature for 3 hours. Then the mixture was filtered, and the solids were washed with CH₃CN. The filtrate was used directly in the next step without further purification.

### Step 3: ethyl 2-{[N-(3,5-dimethoxy-4-methylbenzoyl)-N-(3-phenylpropyl)glycyl]amino}-2,3-dihydro-1H-indene-2-carboxylate

To a solution of 3,5-dimethoxy-4-methylbenzoic acid (48.7 mg, 0.248 mmol) and 1-[bis(dimethylamino)methylene]-1H-1,2,3-triazolo[4,5-b]pyridinium 3-oxid hexafluorophosphate (99.0 mg, 0.260 mmol, HATU) in N,N-dimethylformamide (2 mL) was added triethylamine (0.036 mL, 0.260 mmol). The resulting solution was stirred at room temperature for 5 minutes. Then a solution of ethyl 2-{[N-(3-phenylpropyl)glycyl]amino}-2,3-dihydro-1H-indene-2-carboxylate in CH₃CN (2 mL) from previous step was added in one portion. The solution was stirred at room temperature overnight. The solution was diluted with water and extracted with ethyl acetate (10 mL×2). The combined organic layers were washed with brine three times, dried over anhydrous Na₂SO₄, filtered and concentrated to give a residue that was purified by flash column chromatography on silica gel eluted with ethyl acetate in hexanes (0-100%) to give the titled compound. LC-MS (ESI+) m/z 559.4 (M+H)⁺, RT=1.982 minutes.

### Step 4: 2-{[N-(3,5-dimethoxy-4-methylbenzoyl)-N-(3-phenylpropyl)glycyl]amino}-2,3-dihydro-1H-indene-2-carboxylic Acid

Ethyl 2-{[N-(3,5-dimethoxy-4-methylbenzoyl)-N-(3-phenylpropyl)glycyl]amino}-2,3-dihydro-1H-indene-2-carboxylate (39.0 mg, 0.070 mmol, Step 3) was dissolved in tetrahydrofuran (2 mL) and methanol (0.2 mL) followed by the addition of 1 N lithium hydroxide (1.0 mL). The reaction was stirred at room temperature for 3 hours. The mixture was concentrated, and the residue was treated with water (5 mL) followed by the addition of 1 N hydrochloric acid to adjust the pH to 5. Then the aqueous mixture was extracted with ethyl acetate (5 mL×2). The combined organic fractions were washed with brine and dried over anhydrous Na₂SO₄. The mixture was concentrated, and the residue was purified by was purified by preparative HPLC to provide the titled compound. ¹H NMR (400 MHz, DMSO-d₆) δ ppm 12.50 (s, 1H), 8.53 (s, 1H), 7.43-6.86 (m, 9H), 6.55 (d, J=54.4 Hz, 2H), 4.03 (s, 1H), 3.82 (s, 1H), 3.72 (d, J=9.0 Hz, 6H), 3.46 (s, 2H), 3.36 (d, J=7.6 Hz, 1H), 3.24-3.04 (m, 3H), 2.57 (t, J=7.8 Hz, 1H), 2.37 (t, J=7.6 Hz, 1H), 1.97 (d, J=18.9 Hz, 3H), 1.81 (dd, J=18.3, 10.7 Hz, 2H), T=25° C.; LC-MS (ESI+) m/z 531.4 (M+H)⁺, RT=1.982 minutes.

### Example 2

### 1-{[N-(3,5-dimethoxy-4-methylbenzoyl)-N-(3-phenylpropyl)glycyl]amino}cyclohexane-1-carboxylic Acid

### Step 1: methyl N-(3-phenylpropyl)glycinate

A mixture of methyl 2-chloroacetate (400 mg, 3.69 mmol), 3-phenylpropan-1-amine (498 mg, 3.69 mmol) and potassium carbonate (611 mg, 4.42 mmol) in acetonitrile (12 mL) was stirred at 50° C. overnight. LC-MS showed 69% conversion to the titled compound. The mixture was cooled and filtered. The filtrate was used directly in the next step without further purification. LC-MS (ESI+) m/z 208.2 (M+H)⁺, RT=1.380 minutes.

### Step 2: methyl N-(3,5-dimethoxy-4-methylbenzoyl)-N-(3-phenylpropyl)glycinate

To a solution of 3, 5-dimethoxy-4-methylbenzoic acid (724 mg, 3.69 mmol) and 1-[bis(dimethylamino)methylene]-1H-1,2,3-triazolo[4,5-b]pyridinium 3-oxid hexafluorophosphate (1473 mg, 3.87 mmol, HATU) in N,N-dimethylformamide (20 mL) was added triethylamine (0.540 mL, 3.87 mmol). The resulting solution was stirred at room temperature for 5 minutes. Then the methyl N-(3-phenylpropyl)glycinate filtrate from the previous step was added in one portion. The solution was stirred at room temperature for 1 hour, then diluted with water and extracted with ethyl acetate (15 mL×2). The combined organic layers were washed with brine, dried over anhydrous Na₂SO₄, filtered and concentrated to give a residue that was purified by flash column chromatography on silica gel eluted with hexanes and ethyl acetate (0-60%) to give the titled compound. LC-MS (ESI+) m/z 386.2 (M+H)⁺, RT=2.036 minutes.

### Step 3: N-(3,5-dimethoxy-4-methylbenzoyl)-N-(3-phenylpropyl)glycine

To a solution of methyl N-(3,5-dimethoxy-4-methylbenzoyl)-N-(3-phenylpropyl)glycinate (640 mg, 1.660 mmol) in dioxane (4 mL) was added 1 N lithium hydroxide solution (4.98 mL, 9.96 mmol). The mixture was heated to 50° C. for 1.5 hours. Then the mixture was cooled down to room temperature and acidified with 1 N hydrochloric acid to adjust the pH to 2-3. The mixture was extracted with ethyl acetate (10 mL×3). The combined organic layers were washed with brine twice, dried over anhydrous Na₂SO₄, filtered and concentrated to give the titled compound. ¹H NMR (400 MHz, DMSO-d₆) δ ppm 12.77 (s, 1H), 7.26 (dt, J=13.7, 7.3 Hz, 2H), 7.20-7.08 (m, 2H), 7.05 (d, J=7.4 Hz, 1H), 6.51 (d, J=3.0 Hz, 2H), 4.07 (s, 1H), 3.91 (s, 1H), 3.74 (d, J=7.5 Hz, 6H), 3.43 (t, J=7.7 Hz, 1H), 3.23 (t, J=7.8 Hz, 1H), 2.61 (t, J=7.9 Hz, 1H), 2.42 (t, J=7.7 Hz, 1H), 1.98 (d, J=10.2 Hz, 3H), 1.91-1.77 (m, 2H), T=60° C.; LC-MS (ESI+) m/z 372.2 (M+H)⁺, RT=1.897 minutes.

### Step 4: methyl 1-{[N-(3,5-dimethoxy-4-methylbenzoyl)-N-(3-phenylpropyl)glycyl]amino}cyclohexane-1-carboxylate

To a solution of N-(3,5-dimethoxy-4-methylbenzoyl)-N-(3-phenylpropyl)glycine (70 mg, 0.188 mmol) and 1-[bis(dimethylamino)methylene]-1H-1,2,3-triazolo[4,5-b]pyridinium 3-oxid hexafluorophosphate (79 mg, 0.207 mmol, HATU) in N,N-dimethylformamide (2 mL) was added triethylamine (28.9 μL, 0.207 mmol). The resulting solution was stirred at room temperature for 5 minutes. Then methyl 1-aminocyclohexanecarboxylate (32.6 mg, 0.207 mmol) was added in one portion. The solution was stirred at room temperature for 1 hour. The mixture was diluted with water and extracted with ethyl acetate (10 mL×3). The combined organic layers were washed with brine twice, dried over anhydrous Na₂SO₄, filtered and concentrated to give a residue, which was purified by flash column chromatography on silica gel eluted with hexanes and ethyl acetate (0-50%) to give the titled compound. LC-MS (ESI+) m/z 511.4 (M+H)⁺, RT=2.087 minutes.

### Step 5: 1-{[N-(3,5-dimethoxy-4-methylbenzoyl)-N-(3-phenylpropyl)glycyl]amino}cyclohexane-1-carboxylic acid

To a solution of methyl 1-{[N-(3,5-dimethoxy-4-methylbenzoyl)-N-(3-phenylpropyl)glycyl]amino}cyclohexane-1-carboxylate (69.0 mg, 0.135 mmol) in tetrahydrofuran (1.5 mL) was added 2 N lithium hydroxide (0.405 mL). The solution was heated to 50° C. for 2 hours. The solution was acidified by addition of 1 N hydrochloric acid to adjust the pH to 3, and the aqueous phase was then extracted with ethyl acetate (5 mL×2). The combined organic layers were washed with brine, dried, and concentrated. The residue was purified by was purified by preparative HPLC to provide the titled compound. ¹H NMR (400 MHz, DMSO-d₆) δ ppm 7.73 (s, 1H), 7.20 (d, J=28.0 Hz, 3H), 7.13 (d, J=7.5 Hz, 2H), 6.62 (s, 2H), 3.94 (d, J=19.3 Hz, 2H), 3.74 (s, 6H), 3.34 (s, 2H), 2.53 (d, J=28.4 Hz, 2H), 1.99 (s, 3H), 1.94 (d, J=13.5 Hz, 2H), 1.85 (d, J=11.9 Hz, 2H), 1.76-1.61 (m, 2H), 1.48 (d, J=11.5 Hz, 3H), 1.37 (s, 2H), 1.23 (s, 1H), T=60° C.; LC-MS (ESI+) m/z 497.4 (M+H)⁺, RT=1.955 minutes.

### Example 3

### 1-{[N-(3,5-dimethoxy-4-methylbenzoyl)-N-(3-phenylpropyl)glycyl]amino}cyclopropane-1-carboxylic Acid

The titled compound was prepared according to the procedure used for the preparation of Example 2, substituting 1-aminocyclopropanecarboxylic acid for methyl 1-aminocyclohexanecarboxylate, to provide the titled compound. ¹H NMR (400 MHz, DMSO-d₆) δ ppm 12.17 (s, 1H), 8.36 (s, 1H), 7.23 (s, 3H), 7.15 (d, J=5.5 Hz, 2H), 6.61 (s, 2H), 3.90 (d, J=18.9 Hz, 2H), 3.74 (s, 6H), 3.34 (s, 2H), 2.66-2.49 (m, 2H), 1.99 (s, 3H), 1.95-1.72 (m, 2H), 1.32 (q, J=4.4 Hz, 2H), 1.06-0.72 (m, 2H), T=60° C.; LC-MS (ESI+) m/z 455.2 (M+H)⁺, RT=1.824 minutes.

### Example 4

### 5-[(3,5-dimethoxy-4-methylbenzoyl)(3-phenylpropyl)amino]pentanoic Acid

### Step 1: methyl 5-[(tert-butoxycarbonyl)(3-phenylpropyl)amino]pentanoate

A solution of methyl 5-bromopentanoate (25.0 g, 128 mmol) in acetonitrile (200 mL) was heated to reflux, and then a solution of 3-phenylpropan-1-amine (19.1 g, 141 mmol) in acetonitrile (5 mL) was added dropwise to the mixture. The mixture was heated to reflux for 0.5 hour and then cooled to 0° C. Di-tert-butyl dicarbonate (30.8 g, 141 mmol) was added to the mixture, and triethylamine (13.0 g, 128 mmol) was subsequently added. The mixture was stirred at room temperature for 30 minutes and then concentrated to dryness. The residue was diluted with water (100 mL) and extracted with ethyl acetate (300 mL×2). The combined organic layers were dried over Na₂SO₄, filtered and concentrated. The crude residue was purified by flash chromatography eluted with hexane and ethyl acetate (0-10%) to give the titled compound. LC-MS (ESI+) m/z 250 (M−100+H)⁺, RT=2.19 minutes.

### Step 2: methyl 5-[(3-phenylpropyl)amino]pentanoate

To a solution of methyl 5-[(tert-butoxycarbonyl)(3-phenylpropyl)amino]pentanoate (9.60 g, 27.5 mmol) in dichloromethane (100 mL) was added trifluoroacetic acid (10.6 mL, 137 mmol) at room temperature. After 2 hours, the mixture was concentrated to dryness to give the titled compound as a trifluoroacetic acid salt which was used in the next step without additional purification. LC-MS (ESI+) m/z 250 (M+H)⁺, RT=1.50 minutes.

### Step 3: methyl 5-[(3,5-dimethoxy-4-methylbenzoyl)(3-phenylpropyl)amino]pentanoate

To a solution of 3,5-dimethoxy-4-methylbenzoic acid (5.40 g, 27.5 mmol) in N,N-dimethylformamide (60 mL) was added 1-[bis(dimethylamino)methylene]-1H-1,2,3-triazolo[4,5-b]pyridinium 3-oxid hexafluorophosphate (10.5 g, 27.5 mmol, HATU), and the mixture was stirred at room temperature for 15 minutes. Then a mixture of methyl 5-[(3-phenylpropyl)amino]pentanoate (trifluoroacetic acid salt) (9.52 g, 27.5 mmol) and diisopropylethylamine (14.4 mL, 83.0 mmol) in N,N-dimethylformamide (5 mL) were added to the mixture. The mixture was stirred at room temperature for 2 hours. Then water was added to the mixture, and the mixture was extracted with ethyl acetate (30 mL×3). The combined organic layers were washed with brine, dried over Na₂SO₄, filtered and concentrated. The residue was purified by flash chromatography eluting with hexane and ethyl acetate (0-40%) to give the titled compound. ¹H NMR (400 MHz, CDCl₃) δ ppm 7.26-6.96 (m, 5H), 6.49 (s, 2H), 3.81 (s, 6H), 3.66 (s, 3H), 3.38 (brs, 4H), 2.58 (brs, 2H), 2.29 (brs, 2H), 2.10 (s, 3H), 1.94 (brs, 2H), 1.61 (brs, 4H); LC-MS (ESI+) m/z 428 (M+H)⁺, RT=2.11 minutes.

### Step 4: 5-[(3,5-dimethoxy-4-methylbenzoyl)(3-phenylpropyl)amino]pentanoic Acid

To a solution of methyl 5-[(3,5-dimethoxy-4-methylbenzoyl)(3-phenylpropyl)amino]pentanoate (1.03 g, 2.41 mmol) in tetrahydrofuran (10 mL) was added 1 N lithium hydroxide solution (11.7 mL). The mixture was stirred at room temperature overnight. Then the solution was concentrated, and the residue was washed with diethyl ether twice. The aqueous layer was acidified with 1 N hydrochloric acid solution to pH=3, and extracted with ethyl acetate (30.0 mL×3). The combined organic layers were dried over Na₂SO₄, filtered and concentrated to give a residue that was purified by preparative HPLC to give the titled compound. ¹H NMR (400 MHz, DMSO-d₆) δ ppm 7.18 (dt, J=34.8, 7.6 Hz, 5H), 6.49 (s, 2H), 3.76 (s, 6H), 3.29 (s, 4H), 2.50 (s, 2H), 2.17 (s, 2H), 2.00 (s, 3H), 1.95-1.76 (m, 2H), 1.54 (d, J=8.7 Hz, 2H), 1.44 (s, 2H), T=60° C.; LC-MS (ESI+) m/z 414.2 (M+H)⁺, RT=1.943 minutes.

### Example 5

### 6-[(3,5-dimethoxy-4-methylbenzoyl)(3-phenylpropyl)amino]hexanoic Acid

### Step 1: methyl 6-((3-phenylpropyl)amino)hexanoate

A mixture of methyl 6-bromohexanoate (250 mg, 1.20 mmol), 3-phenylpropan-1-amine (162 mg, 1.20 mmol) and potassium carbonate (198 mg, 1.44 mmol) in CH₃CN (6 mL) was stirred at reflux for 1.5 hours. Then the mixture was cooled down to room temperature and filtered. The filtrate was used for the next step without further purification. LC-MS (ESI+) m/z 264.2 (M+H)⁺, RT=1.513 minutes.

### Step 2: methyl 6-[(3,5-dimethoxy-4-methylbenzoyl)(3-phenylpropyl)amino]hexanoate

To a solution of 3,5-dimethoxy-4-methylbenzoic acid (235 mg, 1.20 mmol) and 1-[bis(dimethylamino)methylene]-1H-1,2,3-triazolo[4,5-b]pyridinium 3-oxid hexafluorophosphate (477 mg, 1.26 mmol, HATU) in N,N-dimethylformamide (8 mL) was added triethylamine (0.175 mL, 1.26 mmol). The resulting solution was stirred at room temperature for 10 minutes. Then the filtrate from the previous step was added in one portion. The mixture was stirred at room temperature overnight. The solution was diluted with water (30 mL) and extracted with ethyl acetate (20 mL×3). The combined organic layers were washed with brine three times, dried over anhydrous Na₂SO₄, filtered and concentrated to give a residue that was purified by flash column chromatography on silica gel eluted with ethyl acetate in hexanes (0-50%) to provide the titled compound. LC-MS (ESI+) m/z 442.2 (M+H)⁺, RT=2.147 minutes.

### Step 3: 6-[(3,5-dimethoxy-4-methylbenzoyl)(3-phenylpropyl)amino]hexanoic Acid

To a solution of methyl 6-[(3,5-dimethoxy-4-methylbenzoyl)(3-phenylpropyl)amino]hexanoate (256 mg, 0.580 mmol) in tetrahydrofuran (4 mL) was added aqueous lithium hydroxide solution (1.0 N, 3.48 mL). The mixture was stirred at room temperature for 2 hours. The mixture was acidified with 1 N hydrochloric acid solution to adjust pH=3. Then the mixture was extracted with ethyl acetate twice, and the combined organic layers were concentrated to give a residue that was purified by preparative HPLC and lyophilized to give the titled compound. ¹H NMR (400 MHz, DMSO-d₆) δ ppm 7.18 (dt, J=35.1, 7.5 Hz, 5H), 6.49 (s, 2H), 3.75 (s, 6H), 3.25 (s, 4H), 2.50 (s, 2H), 2.12 (d, J=7.6 Hz, 2H), 2.00 (s, 3H), 1.85 (t, J=7.6 Hz, 2H), 1.59-1.32 (m, 4H), 1.22 (d, J=16.3 Hz, 2H), T=60° C.; LC-MS (ESI+) m/z 428.2 (M+H)⁺, RT=1.981 minutes.

### Example 6

### {2-[(3,5-dimethoxy-4-methylbenzoyl)(3-phenylpropyl)amino]ethoxy}acetic Acid

### Step 1: tert-butyl (2-{[tert-butyl(dimethyl)silyl]oxy}ethyl)(3-phenylpropyl)carbamate

To a solution of 3-phenylpropan-1-amine (27.1 g, 201 mmol) in acetonitrile (300 mL) was added a solution of (2-bromoethoxy)(tert-butyl)dimethylsilane (40.0 g, 167 mmol) in acetonitrile (20 mL) dropwise at 60° C. The mixture was refluxed overnight. Then the mixture was cooled to room temperature, and di-tert-butyl dicarbonate (31.1 mL, 134 mmol) was added to the mixture followed by stirring at room temperature for 1 hour. The mixture was concentrated, and the residue was partitioned between ethyl acetate and water. The aqueous phase was further extracted with ethyl acetate (50 mL×3). The combined organic layers were dried over Na₂SO₄, filtered and concentrated to give the crude titled compound which was used directly in the next step. LC-MS (ESI+) m/z 294.2 (M-100+H)⁺, RT=2.56 minutes.

### Step 2: tert-butyl (2-hydroxyethyl)(3-phenylpropyl)carbamate

To a solution of crude tert-butyl (2-{[tert-butyl(dimethyl)silyl]oxy}ethyl)(3-phenylpropyl)carbamate (65.7 g, 167 mmol) in tetrahydrofuran (200 mL) was added tetrabutylammonium fluoride (43.7 g, 167 mmol) at room temperature. The mixture was stirred at room temperature for 3 hours. The solution was concentrated, and the residue was diluted with ethyl acetate and water. The mixture was extracted with ethyl acetate, and the organic layer was dried over Na₂SO₄, filtered and concentrated in vacuo. The crude residue was purified by flash column chromatography on silica gel eluted with hexanes and ethyl acetate (0-50%) to give the titled compound. ¹H NMR (400 MHz, CDCl₃) δ ppm 7.35-7.24 (m, 2H), 7.24-7.13 (m, 3H), 3.73 (t, J=5.3 Hz, 2H), 3.37 (t, J=5.3 Hz, 2H), 3.26 (t, J=7.5 Hz, 2H), 2.69-2.53 (m, 2H), 2.36 (s, 1H), 1.86 (tt, J=9.4, 6.7 Hz, 2H), 1.45 (s, 9H); LC-MS (ESI+) m/z 180.1 (M−100+H)⁺, RT=2.00 minutes.

### Step 3: 2-[(3-phenylpropyl)amino]ethan-1-ol

To a solution of tert-butyl (2-hydroxyethyl)(3-phenylpropyl)carbamate (1.50 g, 5.37 mmol) in 1,4-dioxane (3 mL) was added a solution of hydrochloric acid in 1,4-dioxane (4 N, 20 mL). The mixture was stirred at room temperature overnight. Then the mixture was concentrated in vacuo to give the titled compound as a hydrochloride salt which directly in the next step. LC-MS (ESI+) m/z 180 (M+H)⁺, RT=0.187 minutes.

### Step 4: N-(2-hydroxyethyl)-3,5-dimethoxy-4-methyl-N-(3-phenylpropyl)benzamide

To a solution of 3, 5-dimethoxy-4-methylbenzoic acid (219 mg, 1.12 mmol) and 1-[bis(dimethylamino)methylene]-1H-1,2,3-triazolo[4,5-b]pyridinium 3-oxid hexafluorophosphate (445 mg, 1.17 mmol, HATU) in N,N-dimethylformamide (6 mL) was added triethylamine (0.163 mL, 1.17 mmol). The resulting solution was stirred at room temperature for 10 minutes. Then 2-[(3-phenylpropyl)amino]ethan-1-ol (200 mg, 1.12 mmol) from the previous step in CH₃CN (10 mL) was treated with triethylamine to adjust pH to 8-9. Then the mixture was added to the N,N-dimethylformamide solution in one portion. The resulting mixture was stirred at room temperature for 2 hours. The mixture was diluted with water (20 mL) and extracted with ethyl acetate (20 mL×2). The combined organic layers were washed with brine 3 times, dried over anhydrous Na₂SO₄, filtered and concentrated to give a residue that was purified by flash column chromatography on silica gel eluted with ethyl acetate in hexanes (20-100%) to give the titled compound. ¹H NMR (400 MHz, DMSO-d₆) δ ppm 7.22 (d, J=7.4 Hz, 2H), 7.14 (dd, J=10.8, 3.9 Hz, 3H), 6.56 (s, 2H), 4.60 (s, 1H), 3.76 (s, 6H), 3.56 (d, J=16.4 Hz, 2H), 3.37 (s, 4H), 2.51 (d, J=9.0 Hz, 2H), 2.00 (s, 3H), 1.93-1.82 (m, 2H), T=60° C.; LC-MS (ESI+) m/z 358.2 (M+H)⁺, RT=1.886 minutes.

### Step 5: methyl {2-[(3,5-dimethoxy-4-methylbenzoyl)(3-phenylpropyl)amino]ethoxy}acetate

To a solution of N-(2-hydroxyethyl)-3,5-dimethoxy-4-methyl-N-(3-phenylpropyl)benzamide (2.50 g, 6.99 mmol) and methyl 2-chloroacetate (2.15 mL, 24.5 mmol) in tetrahydrofuran (60 mL) was added potassium 2-methylpropan-2-olate (2.75 g, 24.5 mmol) in one portion. Then the mixture was heated to reflux for 1 hour. Additional methyl 2-chloroacetate (2.15 mL, 24.5 mmol) and potassium 2-methylpropan-2-olate (2.75 g, 24.5 mmol) were added sequentially after the reaction was cooled down, and the resulting mixture was heated to reflux for additional 5 hours. Then the mixture was cooled down and quenched with saturated ammonium chloride solution (50 mL) and diluted with water (30 mL). This mixture was extracted with ethyl acetate (80 mL×3). The combined organic layers were washed with brine once, dried over anhydrous Na₂SO₄, filtered and concentrated in vacuo to give a residue that was purified by flash column chromatography on silica gel eluted with ethyl acetate in hexanes (0-50%) to give the titled compound. LC-MS (ESI+) m/z 430.2 (M+H)⁺, RT=2.041 minutes.

### Step 6: {2-[(3,5-dimethoxy-4-methylbenzoyl)(3-phenylpropyl)amino]ethoxy}acetic Acid

To a solution of methyl {2-[(3,5-dimethoxy-4-methylbenzoyl)(3-phenylpropyl)amino]ethoxy}acetate (1.50 g, 3.49 mmol) in tetrahydrofuran (30 mL) was added aqueous lithium hydroxide (1 N, 21.0 mL). It was heated at room temperature for 1 hour, and then the mixture was acidified with 1 N hydrochloric acid to pH=3 while chilled in an ice-water bath. Then the mixture was extracted with ethyl acetate twice. The combined organic layers were washed with brine and dried over anhydrous Na₂SO₄, filtered and concentrated in vacuo to give a residue that was diluted with CH₃CN and purified by preparative HPLC to give the titled compound. ¹H NMR (400 MHz, DMSO-d₆) δ ppm 7.30-7.18 (m, 2H), 7.14 (d, J=5.5 Hz, 3H), 6.57 (s, 2H), 3.76 (s, 6H), 3.74 (s, 2H), 3.58 (d, J=6.3 Hz, 2H), 3.46 (s, 2H), 3.38 (s, 2H), 2.51 (d, J=13.6 Hz, 2H), 2.00 (s, 3H), 1.87 (p, J=7.1 Hz, 2H), T=60° C.; LC-MS (ESI+) m/z 416.2 (M+H)⁺, RT=1.897 minutes.

### Example 7

### 5-[(3,5-dimethoxybenzoyl)(3-phenylpropyl)amino]pentanoic Acid

Example 7 was prepared according to the procedure used for the preparation of Example 4, substituting 3,5-dimethoxybenzoic acid for 3,5-dimethoxy-4-methylbenzoic acid, to provide the titled compound. ¹H NMR (400 MHz, DMSO-d₆) δ ppm 7.23 (d, J=31.8 Hz, 4H), 7.05 (d, J=5.7 Hz, 1H), 6.51 (s, 1H), 6.40 (s, 2H), 3.75 (s, 6H), 3.14 (s, 3H), 2.62 (s, 1H), 2.23 (s, 1H), 2.05 (s, 1H), 1.83 (d, J=36.0 Hz, 3H), 1.53 (m, 3H), 1.26 (d, J=14.8 Hz, 2H); LC-MS (ESI+) m/z 400.2 (M+H)⁺, RT=1.716 minutes.

### Example 8

### 5-{[1-(4-methoxyphenyl)cyclopropane-1-carbonyl](3-phenylpropyl)amino}pentanoic Acid

Example 8 was prepared according to the procedure used for the preparation of Example 4, substituting 1-(3,5-dimethoxy-4-methylphenyl)cyclopropanecarboxylic acid for 3,5-dimethoxy-4-methylbenzoic acid, to provide the titled compound. ¹H NMR (400 MHz, DMSO-d₆) δ ppm 7.26 (t, J=7.6 Hz, 2H), 7.19 (d, J=7.8 Hz, 2H), 7.10 (d, J=7.9 Hz, 1H), 7.01 (dd, J=14.0, 7.9 Hz, 2H), 6.86 (t, J=6.4 Hz, 2H), 3.72 (s, 4H), 2.27 (d, J=8.1 Hz, 1H), 2.21 (s, 1H), 2.01 (s, 1H), 1.73 (s, 1H), 1.42 (s, 3H), 1.35 (s, 1H), 1.18 (s, 2H), 1.10 (d, J=23.5 Hz, 3H), 0.93 (d, J=5.2 Hz, 1H); LC-MS (ESI+) m/z 410.5 (M+H)⁺, RT=1.918 minutes.

### Example 9

### 5-{[(3,5-dimethoxyphenyl)acetyl](3-phenylpropyl)amino}pentanoic Acid

Example 9 was prepared according to the procedure used for the preparation of Example 4, substituting 2-(3,5-dimethoxy-4-methylphenyl)acetic acid for 3,5-dimethoxy-4-methylbenzoic acid, to provide the titled compound. ¹H NMR (400 MHz, DMSO-d₆) δ ppm 7.25 (td, J=7.6, 2.8 Hz, 2H), 7.21-7.10 (m, 3H), 6.97 (t, J=9.0 Hz, 1H), 6.53-6.39 (m, 2H), 3.71 (d, J=7.9 Hz, 5H), 3.42 (d, J=18.1 Hz, 2H), 3.23 (q, J=7.1, 5.2 Hz, 4H), 2.13 (dt, J=6.8, 3.5 Hz, 2H), 1.79-1.67 (m, 2H), 1.51-1.36 (m, 4H); LC-MS (ESI+) m/z 414.5 (M+H)⁺, RT=1.680 minutes.

### Example 10

### 5-[(3-phenylpropyl)(3,4,5-trimethoxybenzoyl)amino]pentanoic Acid

Example 10 was prepared according to the procedure used for the preparation of Example 4, substituting 3,4,5-trimethoxybenzoic acid for 3,5-dimethoxy-4-methylbenzoic acid, to provide the titled compound. ¹H NMR (400 MHz, DMSO-d₆) δ ppm 7.22 (d, J=7.4 Hz, 2H), 7.14 (t, J=7.1 Hz, 3H), 6.56 (s, 2H), 3.76 (s, 6H), 3.69 (s, 3H), 3.51-3.45 (m, 4H), 2.53 (brs, 2H), 2.16 (brs, 2H), 1.96 1.77 (m, 2H), 1.53 (d, J=7.5 Hz, 2H), 1.44 (br s, 2H); LC-MS (ESI+) m/z 430 (M+H)⁺, RT=1.80 minutes.

### Example 11

### 5-{(3,5-dimethoxy-4-methylbenzoyl)[3-(3-methylphenyl)propyl]amino}pentanoic Acid

### Step 1: methyl 5-{[3-(3-methylphenyl)propyl]amino}pentanoate

A mixture of 3-(m-tolyl)propan-1-amine (200 mg, 1.34 mmol), methyl 5-bromopentanoate (261 mg, 1.34 mmol) and potassium carbonate (222 mg, 1.61 mmol) in CH₃CN (6 mL) was stirred at reflux for 3 hours. The mixture was cooled down and filtered. The filtrate was used directly in the next step without further purification. LC-MS (ESI+) m/z 264.2 (M+H)⁺, RT=1.571 minutes.

### Step 2: methyl 5-{(3,5-dimethoxy-4-methylbenzoyl)[3-(3-methylphenyl)propyl]amino}pentanoate

To a solution of 3,5-dimethoxy-4-methylbenzoic acid (263 mg, 1.34 mmol) and 1-[bis(dimethylamino)methylene]-1H-1,2,3-triazolo[4,5-b]pyridinium 3-oxid hexafluorophosphate (535 mg, 1.41 mmol, HATU) in N,N-dimethylformamide (8 mL) was added triethylamine (0.196 mL, 1.41 mmol). The resulting solution was stirred at room temperature for 10 minutes. Then the filtrate from the previous step was added in one portion. The solution was stirred at room temperature overnight. The solution was diluted with water (20 mL) and extracted with ethyl acetate (10 mL×3). The combined organic layers were washed with brine three times, dried over anhydrous Na₂SO₄, filtered and concentrated to give a residue that was purified by flash column chromatography on silica gel eluted with ethyl acetate in hexanes (0-50%) to give the titled compound. LC-MS (ESI+) m/z 442.2 (M+H)⁺, RT=2.163 minutes.

### Step 3: 5-{(3,5-dimethoxy-4-methylbenzoyl)[3-(3-methylphenyl)propyl]amino}pentanoic Acid

To a solution of methyl 5-{(3,5-dimethoxy-4-methylbenzoyl)[3-(3-methylphenyl)propyl]amino}pentanoate (174 mg, 0.39 mmol) in tetrahydrofuran (4 mL) was added aqueous lithium hydroxide solution (1.0 N, 2.364 mL). The mixture was stirred at room temperature for 2 hours. The mixture was then acidified with 1 N hydrochloric acid to adjust pH=3. Then the mixture was extracted with ethyl acetate twice, and the combined organic layers were concentrated to give a residue that was purified by preparative HPLC and lyophilized to give the titled compound. ¹H NMR (400 MHz, DMSO-d₆) δ ppm 7.10 (t, J=7.3 Hz, 1H), 6.93 (t, J=9.8 Hz, 3H), 6.49 (s, 2H), 3.76 (s, 6H), 3.28 (s, 4H), 2.48 (s, 2H), 2.24 (s, 3H), 2.17 (d, J=6.5 Hz, 2H), 2.00 (s, 3H), 1.91-1.73 (m, 2H), 1.54 (q, J=7.4 Hz, 2H), 1.44 (s, 2H), T=60° C.; LC-MS (ESI+) m/z 428.2 (M+H)⁺, RT=2.001 minutes.

### Example 12

### 5-[(3,5-dichlorobenzoyl)(3-phenylpropyl)amino]pentanoic Acid

Example 12 was prepared according to the procedure used for the preparation of Example 4, substituting 3,5-dichlorobenzoic acid for 3,5-dimethoxy-4-methylbenzoic acid, to provide the titled compound. ¹H NMR (400 MHz, DMSO-d₆) δ ppm 12.00 (s, 1H), 7.73-7.56 (m, 1H), 7.34 (dd, J=18.6, 1.9 Hz, 2H), 7.26 (q, J=7.3 Hz, 2H), 7.14 (dt, J=28.8, 7.3 Hz, 2H), 7.00 (d, J=7.3 Hz, 1H), 3.40 (d, J=7.1 Hz, 2H), 3.10 (d, J=7.6 Hz, 1H), 3.04 (t, J=7.8 Hz, 1H), 2.62 (t, J=7.9 Hz, 1H), 2.39 (t, J=7.3 Hz, 1H), 2.24 (t, J=6.7 Hz, 1H), 2.12-1.99 (m, 1H), 1.87 (t, J=7.9 Hz, 1H), 1.75 (t, J=7.8 Hz, 1H), 1.51 (d, J=15.7 Hz, 2H), 1.43 (s, 1H), 1.27 (t, J=7.6 Hz, 1H), T=25° C.; LC-MS (ESI+) m/z 408.2 (M+H)⁺, RT=2.008 minutes.

### Example 13

### 5-[(3,5-difluoro-4-methoxybenzoyl)(3-phenylpropyl)amino]pentanoic Acid

Example 13 was prepared according to the procedure used for the preparation of Example 4, substituting 3,5-difluoro-4-methoxybenzoic acid for 3,5-dimethoxy-4-methylbenzoic acid, to provide the titled compound. ¹H NMR (400 MHz, DMSO-d₆) δ ppm 11.79 (s, 1H), 7.35-7.00 (m, 7H), 3.95 (d, J=1.1 Hz, 3H), 3.15 (s, 4H), 2.51 (d, J=7.7 Hz, 2H), 2.16 (s, 2H), 1.83 (s, 2H), 1.52 (s, 2H), 1.44 (s, 2H), T=60° C.; LC-MS (ESI+) m/z 406.2 (M+H)⁺, RT=1.902 minutes.

### Example 14

### 5-[(2-ethoxypyridine-4-carbonyl)(3-phenylpropyl)amino]pentanoic Acid

Example 14 was prepared according to the procedure used for the preparation of Example 4, substituting 2-ethoxyisonicotinic acid for 3,5-dimethoxy-4-methylbenzoic acid, to provide the titled compound. ¹H NMR (400 MHz, CDCl₃) δ ppm 8.23 (dd, J=7.6 Hz, 1H), 7.33 (t, J=7.5 Hz, 1H), 7.25 (td, J=7.4, 6.7, 2.2 Hz, 1H), 7.20 (d, J=6.9 Hz, 1H), 7.05-6.99 (m, 1H), 6.83-6.77 (m, 1H), 6.74 (s, 1H), 6.67 (s, 1H), 4.41 (dq, J=9.9, 7.0 Hz, 2H), 3.53 (p, J=6.9, 5.8 Hz, 2H), 3.17 (t, J=7.9 Hz, 2H), 2.72 (t, J=7.8 Hz, 1H), 2.55-2.38 (m, 3H), 2.26 (t, J=6.8 Hz, 2H), 2.10-1.94 (m, 2H), 1.86 (p, J=7.5 Hz, 2H), 1.71 (p, J=3.4 Hz, 5H); LC-MS (ESI+) m/z 385.5(M+H)⁺, RT=1.759 minutes.

### Example 15

### {2-[(3,5-dimethoxybenzoyl)(3-phenylpropyl)amino]ethoxy}acetic Acid

Example 15 was prepared according to the procedure used for the preparation of Example 6, substituting 3,5-dimethoxybenzoic acid for 3,5-dimethoxy-4-methylbenzoic acid, to provide the titled compound. ¹H NMR (400 MHz, DMSO-d₆) δ ppm 7.35-7.12 (m, 4H), 7.02 (d, J=7.3 Hz, 1H), 6.48 (d, J=1.8 Hz, 2H), 6.41 (s, 1H), 3.74 (s, 1H), 3.72 (s, 6H), 3.64 (s, 1H), 3.60 (s, 1H), 3.57-3.53 (m, 1H), 3.47 (d, J=9.4 Hz, 2H), 3.32 (d, J=6.3 Hz, 1H), 3.24-3.16 (m, 1H), 2.60 (t, J=7.8 Hz, 1H), 2.38 (d, J=6.5 Hz, 1H), 1.90-1.82 (m, 1H), 1.82-1.71 (m, 1H), T=25° C.; LC-MS (ESI+) m/z 402.2 (M+H)⁺, RT=1.813 minutes.

### Example 16

### 5-[(3,5-dichloro-4-methylbenzoyl)(3-phenylpropyl)amino]pentanoic Acid

Example 16 was prepared according to the procedure used for the preparation of Example 4, substituting 3,5-dichloro-4-methylbenzoic acid for 3,5-dimethoxy-4-methylbenzoic acid, to provide the titled compound. ¹H NMR (400 MHz, DMSO-d₆) δ ppm 7.37 (s, 1H), 7.30-7.23 (m, 3H), 7.12 (dt, J=7.0 Hz, 2H), 6.98 (d, J=7.2 Hz, 1H), 3.38 (t, J=7.1 Hz, 2H), 3.14 (t, J=7.6 Hz, 1H), 3.05 (t, J=7.8 Hz, 1H), 2.61 (t, J=7.8 Hz, 1H), 2.41-2.37 (m, 4H), 2.24 (t, J=6.8 Hz, 1H), 2.08-2.05 (m, 1H), 1.90-1.85 (m, 1H), 1.78-1.73 (m, 1H), 1.55-1.54 (m, 3H), 1.28-1.26 (m, 1H); LC-MS (ESI+) m/z 422 (M+H)⁺, RT=2.06 minutes.

### Example 17

### 5-[(4-chloro-3-methoxybenzoyl)(3-phenylpropyl)amino]pentanoic Acid

Example 17 was prepared according to the procedure used for the preparation of Example 4, substituting 4-chloro-3-methoxybenzoic acid for 3,5-dimethoxy-4-methylbenzoic acid, to provide the titled compound. ¹H NMR (400 MHz, DMSO-d₆) δ ppm 11.96 (s, 1H), 7.47-7.32 (m, 1H), 7.25 (s, 2H), 7.16 (s, 2H), 7.00 (d, J=9.7 Hz, 2H), 6.83 (d, J=22.3 Hz, 1H), 3.83 (d, J=13.4 Hz, 3H), 3.39 (s, 2H), 3.12 (s, 2H), 2.67-2.57 (m, 1H), 2.39 (s, 1H), 2.25 (s, 1H), 2.06 (s, 1H), 1.88 (d, J=8.0 Hz, 1H), 1.77 (s, 1H), 1.53 (s, 2H), 1.49 (s, 1H), 1.27 (s, 1H), T=25° C.; LC-MS (ESI+) m/z 404.2 (M+H)⁺, RT=1.918 minutes.

### Example 18

### 5-[(3,5-diethoxybenzoyl)(3-phenylpropyl)amino]pentanoic Acid

Example 18 was prepared according to the procedure used for the preparation of Example 4, substituting 3,5-diethoxybenzoic acid for 3,5-dimethoxy-4-methylbenzoic acid, to provide the titled compound. ¹H NMR (400 MHz, DMSO-d₆) δ ppm 11.99 (s, 1H), 7.38-7.05 (m, 4H), 7.03 (s, 1H), 6.46 (s, 1H), 6.34 (s, 2H), 3.98 (d, J=7.3 Hz, 4H), 3.37 (s, 2H), 3.13 (s, 2H), 2.60 (s, 1H), 2.37 (s, 1H), 2.24 (s, 1H), 2.06 (s, 1H), 1.85 (s, 1H), 1.76 (s, 1H), 1.51 (d, J=9.5 Hz, 2H), 1.48-1.39 (m, 1H), 1.29 (t, J=7.0 Hz, 6H), 1.23 (s, 1H), T=25° C.; LC-MS (ESI+) m/z 428.2 (M+H)⁺, RT=1.978 minutes.

### Example 19

### 5-[(3,5-dimethoxy-2-nitrobenzoyl)(3-phenylpropyl)amino]pentanoic Acid

Example 19 was prepared according to the procedure used for the preparation of Example 4, substituting 3,5-dimethoxy-2-nitrobenzoic acid for 3,5-dimethoxy-4-methylbenzoic acid, to provide the titled compound. ¹H NMR (400 MHz, DMSO-d₆) δ ppm 12.06 (s, 1H), 7.32-7.25 (m, 1H), 7.25-7.19 (m, 1H), 7.19-7.09 (m, 2H), 7.05-7.00 (m, 1H), 6.80 (dd, J=28.1, 2.5 Hz, 1H), 6.48 (dd, J=24.8, 2.4 Hz, 1H), 3.90 (d, J=2.4 Hz, 3H), 3.85 (d, J=12.5 Hz, 3H), 3.34 (s, 2H), 3.10 (dd, J=14.3, 7.3 Hz, 2H), 2.62-2.53 (m, 1H), 2.43 (d, J=7.3 Hz, 1H), 2.25-2.19 (m, 1H), 2.10 (t, J=7.2 Hz, 1H), 1.82 (q, J=7.7 Hz, 2H), 1.56-1.45 (m, 3H), 1.33 (p, J=7.4 Hz, 1H), T=25° C.; LC-MS (ESI+) m/z 445.2 (M+H)⁺, RT=1.861 minutes.

### Example 20

### 5-[(4-bromo-3,5-dimethoxybenzoyl)(3-phenylpropyl)amino]pentanoic Acid

Example 20 was prepared according to the procedure used for the preparation of Example 4, substituting 4-bromo-3,5-dimethoxybenzoic acid for 3,5-dimethoxy-4-methylbenzoic acid, to provide the titled compound. ¹H NMR (400 MHz, DMSO-d₆) δ ppm 12.01 (s, 1H), 7.27 (d, J=10.7 Hz, 2H), 7.13 (dt, J=16.3, 7.5 Hz, 2H), 7.00 (d, J=7.2 Hz, 1H), 6.59 (d, J=9.1 Hz, 2H), 3.81 (d, J=13.1 Hz, 6H), 3.39 (s, 2H), 3.12 (d, J=7.8 Hz, 2H), 2.63 (d, J=9.1 Hz, 1H), 2.40 (s, 1H), 2.25 (s, 1H), 2.07 (d, J=9.3 Hz, 1H), 1.89 (s, 1H), 1.84-1.70 (m, 1H), 1.54 (s, 3H), 1.29 (s, 1H); LC-MS (ESI+) m/z 478 (M+H)⁺, RT=1.91 minutes.

### Example 21

### 5-[(3-phenylpropyl)(3,4,5-triethoxybenzoyl)amino]pentanoic Acid

Example 21 was prepared according to the procedure used for the preparation of Example 4, substituting 3,4,5-triethoxybenzoic acid for 3,5-dimethoxy-4-methylbenzoic acid, to provide the titled compound. ¹H NMR (400 MHz, DMSO-d₆) δ ppm 7.23 (t, J=7.3 Hz, 2H), 7.14 (t, J=7.2 Hz, 3H), 6.52 (s, 2H), 4.04-3.96 (m, 6H), 3.28 (s, 4H), 2.52 (d, J=5.4 Hz, 2H), 2.16 (s, 2H), 1.89-1.79 (m, 2H), 1.52 (d, J=7.7 Hz, 2H), 1.48-1.38 (m, 2H), 1.30 (t, J=6.9 Hz, 6H), 1.24 (t, J=7.0 Hz, 3H), T=60° C.; LC-MS (ESI+) m/z 472.2 (M+H)⁺, RT=1.981 minutes.

### Example 22

### 5-[(3-methoxy-4-nitrobenzoyl)(3-phenylpropyl)amino]pentanoic Acid

Example 22 was prepared according to the procedure used for the preparation of Example 4, substituting 3-methoxy-4-nitrobenzoic acid for 3,5-dimethoxy-4-methylbenzoic acid, to provide the titled compound. ¹H NMR (400 MHz, DMSO-d₆) δ ppm 12.07 (s, 1H), 7.86 (dd, J=37.2, 8.2 Hz, 1H), 7.31-7.23 (m, 2H), 7.21-7.12 (m, 2H), 7.10-6.89 (m, 3H), 3.90 (d, J=21.2 Hz, 3H), 3.42 (q, J=7.6 Hz, 2H), 3.09 (dt, J=22.8, 7.7 Hz, 2H), 2.63 (t, J=7.8 Hz, 1H), 2.41 (d, J=7.3 Hz, 1H), 2.26 (t, J=6.8 Hz, 1H), 2.07 (d, J=4.6 Hz, 1H), 1.97-1.85 (m, 1H), 1.77 (t, J=7.9 Hz, 1H), 1.63-1.52 (m, 2H), 1.47 (q, J=7.4, 7.0 Hz, 1H), 1.29 (q, J=7.5 Hz, 1H), T=25° C.; LC-MS (ESI+) m/z 415.2 (M+H)⁺, RT=1.853 minutes.

### Example 23

### 5-[(3,4-dihydro-2H-1,5-benzodioxepine-7-carbonyl)(3-phenylpropyl)amino]pentanoic Acid

Example 23 was prepared according to the procedure used for the preparation of Example 4, substituting 3,4-dihydro-2H-benzo[b][1,4]dioxepine-7-carboxylic acid for 3,5-dimethoxy-4-methylbenzoic acid, to provide the titled compound. ¹H NMR (400 MHz, DMSO-d₆) δ ppm 7.24 (dd, J=8.4, 6.4 Hz, 2H), 7.14 (t, J=7.4 Hz, 3H), 6.96-6.90 (m, 1H), 6.86 (s, 1H), 6.86-6.83 (m, 1H), 4.15 (dt, J=7.7, 5.5 Hz, 4H), 3.28 (s, 4H), 2.57-2.49 (m, 2H), 2.19-2.06 (m, 4H), 1.81 (p, J=7.9 Hz, 2H), 1.51 (t, J=7.6 Hz, 2H), 1.42 (s, 2H), T=60° C.; LC-MS (ESI+) m/z 412.2 (M+H)⁺, RT=1.849 minutes.

### Example 24

### 5-[(7-methoxy-1-benzofuran-5-carbonyl)(3-phenylpropyl)amino]pentanoic Acid

Example 24 was prepared according to the procedure used for the preparation of Example 4, substituting 7-methoxybenzofuran-5-carboxylic acid for 3,5-dimethoxy-4-methylbenzoic acid, to provide the titled compound. ¹H NMR (400 MHz, DMSO-d₆) 6 ppm 7.97 (d, J=2.1 Hz, 1H), 7.18 (s, 2H), 7.14 (s, 1H), 7.12 (d, J=12.8 Hz, 3H), 6.93 (d, J=2.2 Hz, 1H), 6.80 (d, J=1.3 Hz, 1H), 3.93 (s, 3H), 3.31 (s, 6H), 2.16 (s, 2H), 1.92-1.80 (m, 2H), 1.59-1.50 (m, 2H), 1.44 (s, 2H), T=60° C.; LC-MS (ESI+) m/z 410.2 (M+H)⁺, RT=1.874 minutes.

### Example 25

### 5-[(3-phenylpropyl){1-[4-(trifluoromethoxy)phenyl]cyclopropane-1-carbonyl}amino]pentanoic Acid

Example 25 was prepared according to the procedure used for the preparation of Example 4, substituting 1-(4-(trifluoromethoxy)phenyl)cyclopropanecarboxylic acid for 3,5-dimethoxy-4-methylbenzoic acid, to provide the titled compound. ¹H NMR (400 MHz, DMSO-d₆) δ ppm 7.40-7.08 (m, 8H), 7.04-6.92 (m, 1H), 3.19 (dt, J=15.4, 6.7 Hz, 4H), 2.49 (d, J=1.9 Hz, 2H), 2.27 (t, J=7.6 Hz, 1H), 2.20 (t, J=6.7 Hz, 1H), 1.96 (t, J=7.3 Hz, 1H), 1.85-1.63 (m, 1H), 1.39 (q, J=7.8, 6.8 Hz, 3H), 1.24 (d, J=4.4 Hz, 1H), 1.22-1.12 (m, 3H), 1.02 (dd, J=11.2, 6.8 Hz, 2H); LC-MS (ESI+) m/z 464 (M+H)⁺, RT=2.06 minutes.

### Example 26

### 5-{(3-phenylpropyl)[3-(trifluoromethoxy)benzoyl]amino}pentanoic Acid

Example 26 was prepared according to the procedure used for the preparation of Example 4, substituting 3-(trifluoromethoxy)benzoic acid for 3,5-dimethoxy-4-methylbenzoic acid, to provide the titled compound. ¹H NMR (400 MHz, DMSO-d₆) δ ppm 12.00 (s, 1H), 7.51 (dd, J=24.8, 7.9 Hz, 1H), 7.45-7.31 (m, 2H), 7.27 (d, J=12.3 Hz, 3H), 7.20-7.05 (m, 2H), 7.00 (d, J=7.4 Hz, 1H), 3.41 (d, J=6.9 Hz, 3H), 3.19-2.99 (m, 2H), 2.61 (d, J=8.1 Hz, 1H), 2.35 (t, J=7.4 Hz, 1H), 2.24 (d, J=6.6 Hz, 1H), 2.03 (t, J=7.3 Hz, 1H), 1.88 (s, 1H), 1.75 (s, 1H), 1.48 (d, J=43.7 Hz, 4H), 1.31-1.15 (m, 1H); LC-MS (ESI+) m/z 424 (M+H)⁺, RT=1.98 minutes.

### Example 27

### 5-{[1-(2H-1,3-benzodioxol-5-yl)cyclopropane-1-carbonyl](3-phenylpropyl)amino}pentanoic Acid

Example 27 was prepared according to the procedure used for the preparation of Example 4, substituting 1-(benzo[d][1,3]dioxol-5-yl)cyclopropanecarboxylic acid for 3,5-dimethoxy-4-methylbenzoic acid, to provide the titled compound. ¹H NMR (400 MHz, DMSO-d₆) δ ppm 11.98 (s, 1H), 7.34-7.23 (m, 2H), 7.17 (td, J=8.2, 7.6, 3.9 Hz, 2H), 7.12-7.00 (m, 1H), 6.90-6.47 (m, 3H), 5.96 (s, 2H), 3.19 (p, J=7.1, 5.9 Hz, 4H), 2.49 (d, J=1.9 Hz, 2H), 2.30 (t, J=7.6 Hz, 1H), 2.19 (s, 1H), 2.04 (d, J=14.7 Hz, 1H), 1.71 (p, J=7.8 Hz, 1H), 1.47-1.31 (m, 3H), 1.21 (q, J=7.2 Hz, 1H), 1.17 1.01 (m, 4H), 0.92 (q, J=4.6 Hz, 1H); LC-MS (ESI+) m/z 424 (M+H)⁺, RT=1.91 minutes.

### Example 28

### 5-[(3-methoxy-4-methylbenzoyl)(3-phenylpropyl)amino]pentanoic Acid

The titled compound was prepared according to the procedure used for the preparation of Example 4, substituting 3-methoxy-4-methylbenzoic acid for 3,5-dimethoxy-4-methylbenzoic acid. ¹H NMR (400 MHz, DMSO-d₆) δ ppm 12.00 (s, 1H), 7.14 (t, J=45.3 Hz, 6H), 6.76 (m, 2H), 3.76 (s, 3H), 3.38 (s, 2H), 3.15 (s, 2H), 2.61 (s, 1H), 2.37 (s, 1H), 2.24 (s, 1H), 2.14 (s, 3H), 2.06 (s, 1H), 1.82 (d, J=31.2 Hz, 2H), 1.52 (s, 3H), 1.26 (s, 1H); LC-MS (ESI+) m/z 398 (M+H)⁺, RT=1.82 minutes.

### Example 29

### 5-{[3-methoxy-5-(trifluoromethoxy)benzoyl](3-phenylpropyl)amino}pentanoic Acid

Example 29 was prepared according to the procedure used for the preparation of Example 4, substituting 3-methoxy-5-(trifluoromethoxy)benzoic acid for 3,5-dimethoxy-4-methylbenzoic acid, to provide the titled compound. ¹H NMR (400 MHz, DMSO-d₆) δ ppm 12.01 (s, 1H), 7.37-7.20 (m, 2H), 7.20-7.07 (m, 2H), 7.05-6.91 (m, 2H), 6.84 (t, J=15.3 Hz, 2H), 3.79 (d, J=8.0 Hz, 3H), 3.38 (s, 2H), 3.09 (q, J=9.2, 8.1 Hz, 2H), 2.66-2.57 (m, 1H), 2.37 (t, J=7.4 Hz, 1H), 2.25 (d, J=7.5 Hz, 1H), 2.03 (d, J=7.5 Hz, 1H), 1.88 (d, J=8.2 Hz, 1H), 1.75 (s, 1H), 1.51 (d, J=15.2 Hz, 2H), 1.44 (s, 1H), 1.25 (s, 1H), T=25° C.; LC-MS (ESI+) m/z 454.2 (M+H)⁺, RT=2.002 minutes.

### Example 30

### 5-[(2,4-difluoro-3,5-dimethoxybenzoyl)(3-phenylpropyl)amino]pentanoic Acid

Example 30 was prepared according to the procedure used for the preparation of Example 4, substituting 2,4-difluoro-3,5-dimethoxybenzoic acid for 3,5-dimethoxy-4-methylbenzoic acid, to provide the titled compound. ¹H NMR (400 MHz, DMSO-d₆) δ ppm 11.72 (s, 1H), 7.33-7.05 (m, 4H), 6.99 (d, J=7.3 Hz, 1H), 6.74 (ddd, J=21.4, 8.6, 5.5 Hz, 1H), 3.91 (d, J=21.2 Hz, 3H), 3.81 (d, J=13.3 Hz, 3H), 3.49-3.38 (m, 2H), 3.18-3.02 (m, 7H), 2.63 (t, J=7.8 Hz, 1H), 2.42 (t, J=7.3 Hz, 1H), 2.25 (t, J=6.9 Hz, 1H), 2.11-2.02 (m, 1H), 1.89 (p, J=7.7 Hz, 1H), 1.75 (p, J=7.3 Hz, 1H), 1.57 (dq, J=15.4, 7.5 Hz, 2H), 1.45 (p, J=7.4 Hz, 1H), 1.31 (p, J=7.4 Hz, 1H); LC-MS (ESI+) m/z 436.4 (M+H)⁺, RT=1.899 minutes.

### Example 31

### 5-[(4-methoxy-2-methyl-1-benzofuran-6-carbonyl)(3-phenylpropyl)amino]pentanoic Acid

Example 31 was prepared according to the procedure used for the preparation of Example 4, substituting 4-methoxy-2-methylbenzofuran-6-carboxylic acid for 3,5-dimethoxy-4-methylbenzoic acid, to provide the titled compound. ¹H NMR (400 MHz, DMSO-d₆) δ ppm 7.45-6.90 (m, 6H), 6.62 (d, J=15.3 Hz, 2H), 3.87 (s, 4H), 3.42 (s, 2H), 3.20 (s, 2H), 2.45 (s, 4H), 2.27 (s, 1H), 1.84 (s, 2H), 1.55 (s, 3H), 1.38-1.16 (m, 1H); LC-MS (ESI+) m/z 424.4 (M+H)⁺, RT=1.955 minutes.

### Example 32

### 5-{(3,5-dimethoxy-4-methylbenzoyl)[3-(2-fluorophenyl)propyl]amino}pentanoic Acid

Example 32 was prepared according to the procedure used for the preparation of Example 11, substituting 3-(2-fluorophenyl)propan-1-amine for 3-(o-tolyl)propan-1-amine, to provide the titled compound. ¹H NMR (400 MHz, DMSO-d₆) δ ppm 7.33-7.10 (m, 2H), 7.05 (t, J=8.9 Hz, 2H), 6.48 (s, 2H), 3.75 (s, 6H), 3.29 (s, 4H), 2.55 (s, 2H), 2.17 (s, 2H), 2.00 (s, 3H), 1.84 (s, 2H), 1.56 (d, J=9.3 Hz, 2H), 1.44 (s, 2H), T=60° C.; LC-MS (ESI+) m/z 432.2 (M+H)⁺, RT=1.945 minutes.

### Example 33

### 5-{(3,5-dimethoxy-4-methylbenzoyl)[3-(3-fluorophenyl)propyl]amino}pentanoic Acid

Example 33 was prepared according to the procedure used for the preparation of Example 11, substituting 3-(3-fluorophenyl)propan-1-amine for 3-(o-tolyl)propan-1-amine, to provide the titled compound. ¹H NMR (400 MHz, DMSO-d₆) δ ppm 7.25 (d, J=7.3 Hz, 1H), 7.08-6.84 (m, 3H), 6.49 (s, 2H), 3.76 (s, 6H), 3.29 (s, 4H), 2.54 (s, 2H), 2.16 (d, J=7.1 Hz, 2H), 2.00 (s, 3H), 1.85 (s, 2H), 1.55 (s, 2H), 1.44 (s, 2H), T=60° C.; LC-MS (ESI+) m/z 432.2 (M+H)⁺, RT=1.940 minutes.

### Example 34

### 5-{(3,5-dimethoxy-4-methylbenzoyl)[3-(4-fluorophenyl)propyl]amino}pentanoic Acid

Example 34 was prepared according to the procedure used for the preparation of Example 11, substituting 3-(4-fluorophenyl)propan-1-amine for 3-(o-tolyl)propan-1-amine, to provide the titled compound. ¹H NMR (400 MHz, DMSO-d₆) δ ppm 12.03 (s, 1H), 7.27 (s, 1H), 6.99 (d, J=35.2 Hz, 3H), 6.45 (s, 2H), 3.74 (d, J=4.8 Hz, 6H), 3.37 (s, 2H), 3.13 (s, 2H), 2.61 (s, 1H), 2.38 (s, 1H), 2.25 (s, 1H), 2.07 (d, J=9.0 Hz, 1H), 1.98 (s, 3H), 1.81 (d, J=33.9 Hz, 2H), 1.53 (s, 3H), 1.29 (s, 1H), T=25° C.; LC-MS (ESI+) m/z 432.2 (M+H)⁺, RT=1.943 minutes.

### Example 35

### 5-[(4-cyclopropyl-3,5-dimethoxybenzoyl)(3-phenylpropyl)amino]pentanoic Acid

### Step 1: methyl 5-[(4-cyclopropyl-3,5-dimethoxybenzoyl)(3-phenylpropyl)amino]pentanoate

To a mixture of methyl 5-[(4-bromo-3,5-dimethoxybenzoyl)(3-phenylpropyl)amino]pentanoate (47.0 mg, 0.095 mmol) (prepared according to the procedure of Example 20), cyclopropylboronic acid (12.3 mg, 0.143 mmol) and tricyclohexylphosphine (5.35 mg, 0.019 mmol), potassium phosphate tribasic (60.8 mg, 0.286 mmol) in the co-solvent of toluene (1.0 mL) and water (0.15 mL) was added palladium(II) acetate (2.14 mg, 9.55 μmol). Then the mixture was bubbled with N₂ for 2-3 minutes and heated to 110° C. for 2 hours. Then the mixture was cooled down and extracted with ethyl acetate 3 times. The combined organic layers were washed brine once, dried over anhydrous Na₂SO₄, filtered and concentrated to give a residue that was purified by flash column chromatography on silica gel eluted with ethyl acetate in hexanes (50%-100%) to give the titled compound. LC-MS (ESI+) m/z 454.2 (M+H)⁺, RT=2.157 minutes.

### Step 2: 5-[(4-cyclopropyl-3,5-dimethoxybenzoyl)(3-phenylpropyl)amino]pentanoic Acid

To a solution of methyl 5-[(4-cyclopropyl-3,5-dimethoxybenzoyl)(3-phenylpropyl)amino]pentanoate (43.1 mg, 0.095 mmol) in tetrahydrofuran (2 mL) was added aqueous lithium hydroxide solution. (1.0 N, 0.57 mL). The mixture was stirred at room temperature overnight. The mixture was acidified with 1 N hydrochloric acid to adjust pH=3. Then the mixture was extracted with ethyl acetate twice, and the combined organic layers were concentrated to give a residue that was purified by preparative HPLC and lyophilized to give the titled compound. ¹H NMR (400 MHz, DMSO-d₆) δ ppm 12.01 (s, 1H), 7.21 (d, J=33.0 Hz, 4H), 7.03 (s, 1H), 6.45 (s, 2H), 3.70 (s, 6H), 3.37 (s, 2H), 3.15 (s, 2H), 2.61 (s, 1H), 2.40 (s, 1H), 2.24 (s, 1H), 2.06 (s, 1H), 1.84 (tt, J=8.8, 5.6 Hz, 3H), 1.51 (s, 3H), 1.29 (s, 1H), 0.94 (m, 2H), 0.74 (m, 2H), T=25° C.; LC-MS (ESI+) m/z 440.2 (M+H)⁺, RT=1.994 minutes.

### Example 36

### 5-{[3-(4-chlorophenyl)propyl](3,5-dimethoxy-4-methylbenzoyl)amino}pentanoic Acid

Example 36 was prepared according to the procedure used for the preparation of Example 11, substituting 3-(4-chlorophenyl)propan-1-amine for 3-(o-tolyl)propan-1-amine, to provide the titled compound. ¹H NMR (400 MHz, DMSO-d₆) δ ppm 11.93 (s, 1H), 7.30 (d, J=16.9 Hz, 2H), 7.19 (s, 1H), 7.03 (s, 1H), 6.56-6.37 (m, 2H), 3.74 (d, J=6.4 Hz, 6H), 3.37 (s, 2H), 3.13 (s, 2H), 2.61 (s, 1H), 2.39 (s, 1H), 2.25 (s, 1H), 2.07 (d, J=8.7 Hz, 1H), 1.98 (s, 3H), 1.84 (dd, J=30.7, 17.8 Hz, 2H), 1.52 (s, 3H), 1.28 (s, 1H), T=25° C.; LC-MS (ESI+) m/z 448.2 (M+H)⁺, RT=2.001 minutes.

### Example 37

### 5-{[3,5-dimethoxy-4-(trifluoromethyl)benzoyl](3-phenylpropyl)amino}pentanoic Acid

### Step 1: methyl 4-bromo-3,5-dimethoxybenzoate

To a mixture of 4-bromo-3,5-dimethoxybenzoic acid (900 mg, 3.45 mmol) in N,N-dimethylformamide (10 mL) was added potassium carbonate (953 mg, 6.89 mmol) and then methyl iodide (0.259 mL, 4.14 mmol). The mixture was stirred at room temperature for 1 hour. Then water (30 mL) was added to the mixture, and the mixture was extracted with ethyl acetate (40 mL). The organic layer was dried over Na₂SO₄, filtered and concentrated to give the titled compound. ¹H NMR (400 MHz, CDCl₃) δ ppm 7.25 (s, 2H), 3.96 (s, 6H), 3.94 (s, 3H); LC-MS (ESI+) m/z 275.0, 277.0 (M+H)⁺, RT=1.878 minutes.

### Step 2: methyl 3,5-dimethoxy-4-(trifluoromethyl)benzoate

To a mixture of methyl 4-bromo-3,5-dimethoxybenzoate (100 mg, 0.364 mmol), copper(I) iodide (13.9 mg, 0.073 mmol) and cesium trifluoroacetate (107 mg, 0.436 mmol) in dry N,N-dimethylformamide (0.5 mL) was added methyl trifluoroacetate (186 mg, 1.45 mmol). Then the mixture was purged with N₂ for 2-3 minutes and heated to 160° C. overnight. Then additional copper(I) iodide (13.9 mg, 0.073 mmol), cesium trifluoroacetate (107 mg, 0.436 mmol) and 1,10-phenanthroline (13.1 mg, 0.073 mmol) were added to the mixture. The mixture was purged with N₂ for 2-3 minutes and heated to 160° C. again. Methyl trifluoroacetate (186 mg, 1.45 mmol) was added dropwise over 10 minutes, and additional methyl trifluoroacetate (186 mg, 1.45 mmol) was added 30 minutes later. The mixture was heated for 3 hours. Then the mixture was cooled down to room temperature and tert-butyl methyl ether (4 mL) was added. The mixture was filtered, and the solids were washed with tert-butyl methyl ether. The filtrate was concentrated in vacuo to give a residue that was purified by flash column chromatography on silica gel eluted with ethyl acetate in hexanes (0-15%) to give the titled compound. ¹H NMR (400 MHz, DMSO-d₆) δ ppm 7.28 (d, J=0.9 Hz, 2H), 3.90 (s, 6H), 3.89 (s, 3H); LC-MS (ESI+) m/z 265.0 (M+H)⁺, RT=1.953 minutes.

### Step 3: 3,5-dimethoxy-4-(trifluoromethyl)benzoic Acid

To a solution of methyl 3,5-dimethoxy-4-(trifluoromethyl)benzoate (69.0 mg, 0.261 mmol) in dioxane (2 mL) was added aqueous lithium hydroxide solution. (1.0 N, 1.57 mL). The mixture was stirred at room temperature overnight. The mixture was acidified with 1 N hydrochloric acid to adjust pH=2. Then the mixture was extracted with ethyl acetate twice, and the combined organic layers were concentrated to give the titled compound. LC-MS (ESI+) m/z 251.0 (M+H)⁺, RT=1.758 minutes.

### Step 4: 5-{[3,5-dimethoxy-4-(trifluoromethyl)benzoyl](3-phenylpropyl)amino}pentanoic Acid

The titled compound was prepared according to the procedure used for the preparation of Example 4, substituting 3,5-dimethoxy-4-(trifluoromethyl)benzoic acid for 3,5-dimethoxy-4-methylbenzoic acid, to provide the titled compound. ¹H NMR (400 MHz, DMSO-d₆) δ ppm 12.04 (s, 1H), 7.43-6.93 (m, 5H), 6.65 (d, J=18.1 Hz, 2H), 3.81 (d, J=18.9 Hz, 6H), 3.40 (q, J=7.7, 7.3 Hz, 2H), 3.10 (dt, J=15.8, 7.4 Hz, 2H), 2.63 (t, J=8.0 Hz, 1H), 2.42 (t, J=7.3 Hz, 1H), 2.26 (t, J=6.8 Hz, 1H), 2.08 (t, J=7.2 Hz, 1H), 1.84 (dt, J=41.0, 7.2 Hz, 2H), 1.67-1.41 (m, 3H), 1.39-1.22 (m, 1H), T=25° C.; LC-MS (ESI+) m/z 468.2 (M+H)⁺, RT=1.954 minutes.

### Example 38

### 5-{[3-(3-chlorophenyl)propyl](3,5-dimethoxy-4-methylbenzoyl)amino}pentanoic Acid

Example 38 was prepared according to the procedure used for the preparation of Example 11, substituting 3-(3-chlorophenyl)propan-1-amine for 3-(o-tolyl)propan-1-amine, to provide the titled compound. ¹H NMR (400 MHz, DMSO-d₆) δ ppm 11.81 (s, 1H), 7.35-7.15 (m, 3H), 7.10 (s, 1H), 6.49 (s, 2H), 3.76 (s, 6H), 3.29 (s, 4H), 2.53 (s, 2H), 2.17 (s, 2H), 2.00 (s, 3H), 1.87 (d, J=22.2 Hz, 2H), 1.55 (s, 2H), 1.44 (s, 2H), T=60° C.; LC-MS (ESI+) m/z 448.2 (M+H)⁺, RT=1.998 minutes.

### Example 39

### 5-[(2-chloro-3,5-dimethoxybenzoyl)(3-phenylpropyl)amino]pentanoic Acid

### Step 1: 3,5-dimethoxy-2-nitrobenzoic Acid

To 3,5-dimethoxybenzoic acid (4.01 g, 22 mmol) in acetic anhydride (25 mL, 264 mmol) was added nitric acid (3.95 mL, 95 mmol) slowly at 0° C. The mixture was stirred at 0° C. for 1 hour, and then warmed to room temperature for 1 hour. The mixture was poured into 150 g of ice water. The solid was collected by filtration, and washed with water (3×5 mL). The solid was dried under vacuum to give the titled compound, 4.65 g (93% yield). ¹H NMR (400 MHz, DMSO-d₆) δ ppm 7.05 (d, J=2 Hz, 1H), 6.97 (d, J=2 Hz, 1H), 3.91 (s, 3H), 3.88 (s, 3H); MS (ESI−) m/z 226 (M−H)⁻.

### Step 2: methyl 3,5-dimethoxy-2-nitrobenzoate

3,5-Dimethoxy-2-nitrobenzoic acid (1 g, 4.40 mmol, Step 1) was dissolved in CH₃OH (10 mL) and cooled to 0° C. Concentrated sulfuric acid (1.98 g, 20.19 mmol) was added slowly, and then the reaction mixture was stirred at 70° C. for 6 hours. The mixture was poured into ice water. The solid was collected by filtration and dried to give the titled compound, 1 g (94% yield). ¹H NMR (400 MHz, DMSO-d₆) δ ppm 7.07 (d, J=2.5 Hz, 1H), 6.97 (d, J=2.5 Hz, 1H), 3.91 (s, 3H), 3.89 (s, 3H), 3.81 (s, 3H).

### Step 3: methyl 2-amino-3,5-dimethoxybenzoate

A solution of methyl 3,5-dimethoxy-2-nitrobenzoate (700 mg, 2.90 mmol, Step 2) in CH₃OH (5 mL) and tetrahydrofuran (5 mL) was stirred for 12 hours under H₂ (15 psi) in the presence of 10% Pd/charcoal (535 mg). The catalyst was removed by filtration through Celite®, and the filtrate was concentrated in vacuo to give the titled compound (600 mg, 98% yield). LC-MS (ESI+) m/z 212 (M+H)⁺.

### Step 4: methyl 2-chloro-3,5-dimethoxybenzoate

A mixture of methyl 2-amino-3,5-dimethoxybenzoate (220 mg, 1.042 mmol, Step 3) and 2 N hydrochloric acid (0.1 mL) was stirred at 0° C. for 3 minutes. Then sodium nitrite (79 mg, 1.146 mmol) in water (1 mL) was added dropwise, and the mixture was stirred for 30 minutes at 0° C. Copper(I) chloride (516 mg, 5.21 mmol) in 2 N HCl (1 mL) was added at room temperature, and the mixture was stirred at room temperature overnight. A saturated aqueous ammonium chloride solution was added to the reaction mixture followed by extraction with CH₂Cl₂ (10 mL×2). The combined organic fractions were dried over anhydrous Na₂SO₄, filtered and concentrated. The residue was purified by flash column chromatography on silica gel eluted with ethyl acetate in hexanes (0-50%) to give 70 mg (29% yield) of the titled compound. LC-MS (ESI+) m/z 231 (M+H)⁺.

### Step 5: 2-chloro-3,5-dimethoxybenzoic Acid

A mixture of methyl 2-chloro-3,5-dimethoxybenzoate (45 mg, 0.195 mmol, Step 4) in tetrahydrofuran (2 mL) and lithium hydroxide (14.02 mg, 0.585 mmol) in water (1 mL) was stirred at room temperature for 2 hours. Then 1 N HCl was added dropwise to pH˜6. The mixture was extracted with ethyl acetate. The organic phase was dried over anhydrous Na₂SO₄, filtered and concentrated to give 40 mg (95% yield) of the titled compound. LC-MS (ESI+) m/z 217 (M+H)⁺.

### Step 6: 5-[(2-chloro-3,5-dimethoxybenzoyl)(3-phenylpropyl)amino]pentanoic Acid

5-[(2-Chloro-3,5-dimethoxybenzoyl)(3-phenylpropyl)amino]pentanoic acid was prepared according to the procedure used for the preparation of Example 4, substituting 2-chloro-3,5-dimethoxybenzoic acid (Step 5) for 3,5-dimethoxy-4-methylbenzoic acid, to provide the titled compound. ¹H NMR (400 MHz, DMSO-d₆) δ ppm 7.35-7.23 (m, 2H), 7.22-7.08 (m, 2H), 7.00 (dd, J=6.8, 1.7 Hz, 1H), 6.69 (dd, J=21.7, 2.7 Hz, 1H), 6.44 (dd, J=10.5, 2.7 Hz, 1H), 3.86 (d, J=3.2 Hz, 3H), 3.78 (d, J=6.9 Hz, 3H), 3.21 (td, J=13.4, 6.6 Hz, 1H), 3.15-3.01 (m, 1H), 2.97 (dd, J=12.7, 7.4 Hz, 1H), 2.65 (s, 1H), 2.39 (p, J=7.1 Hz, 1H), 2.26 (t, J=6.9 Hz, 1H), 2.07 (t, J=7.3 Hz, 1H), 1.86 (d, J=24.8 Hz, 1H), 1.63-1.51 (m, 2H), 1.49-1.19 (m, 2H); LC-MS (ESI+) m/z 434.2 (M+H)⁺, RT=1.874 minutes.

### Example 40

### 5-[(3,5-dimethoxy-4-methylbenzoyl){3-[3-(trifluoromethyl)phenyl]propyl}amino]pentanoic Acid

Example 40 was prepared according to the procedure used for the preparation of Example 11, substituting 3-(3-(trifluoromethyl)phenyl)propan-1-amine for 3-(o-tolyl)propan-1-amine, to provide the titled compound. ¹H NMR (400 MHz, DMSO-d₆) δ ppm 7.79-7.23 (m, 4H), 6.49 (d, J=13.8 Hz, 2H), 3.75 (d, J=10.7 Hz, 9H), 3.41 (s, 2H), 2.75 (s, 1H), 2.27 (s, 1H), 2.10 (s, 1H), 1.99 (s, 3H), 1.88 (d, J=33.6 Hz, 2H), 1.55 (s, 3H), 1.31 (s, 1H), T=60° C.; LC-MS (ESI+) m/z 481.2 (M+H)⁺, RT=2.024 minutes.

### Example 41

### 5-{(3,5-dimethoxy-4-methylbenzoyl)[3-(5-methylthiophen-2-yl)propyl]amino}pentanoic Acid

Example 41 was prepared according to the procedure used for the preparation of Example 11, substituting 3-(5-methylthiophen-2-yl)propan-1-amine for 3-(o-tolyl)propan-1-amine, to provide the titled compound. ¹H NMR (400 MHz, DMSO-d₆) δ ppm 6.51 (s, 4H), 3.77 (s, 6H), 3.65 (m, 2H), 3.55 (m, 3H), 3.18 (s, 3H), 2.77 (s, 1H), 2.42-2.18 (m, 4H), 2.10 (s, 1H), 2.01 (s, 3H), 1.85 (d, J=39.4 Hz, 2H), 1.55 (s, 3H), 1.31 (s, 1H); LC-MS (ESI+) m/z 434.2 (M+H)⁺, RT=1.978 minutes.

### Example 42

### 5-{(3,5-dimethoxy-4-methylbenzoyl)[3-(4-methylphenyl)propyl]amino}pentanoic Acid

Example 42 was prepared according to the procedure used for the preparation of Example 11, substituting 3-(p-tolyl)propan-1-amine for 3-(o-tolyl)propan-1-amine, to provide the titled compound. ¹H NMR (400 MHz, DMSO-d₆) δ ppm 7.11 (s, 2H), 6.98 (s, 2H), 6.49 (s, 2H), 3.76 (s, 6H), 3.39 (s, 2H), 3.15 (s, 2H), 2.58 (s, 1H), 2.36 (s, 1H), 2.32-2.18 (m, 4H), 2.10 (s, 1H), 2.01 (s, 3H), 1.82 (d, J=29.4 Hz, 2H), 1.54 (s, 3H), 1.30 (s, 1H); LC-MS (ESI+) m/z 428.2 (M+H)⁺, RT=1.978 minutes.

### Example 43

### 5-{(3,5-dimethoxy-4-methylbenzoyl)[3-(4-methoxyphenyl)propyl]amino}pentanoic Acid

Example 43 was prepared according to the procedure used for the preparation of Example 11, substituting 3-(4-methoxyphenyl)propan-1-amine for 3-(o-tolyl)propan-1-amine, to provide the titled compound. ¹H NMR (400 MHz, DMSO-d₆) δ ppm 11.47 (s, 1H), 7.17 (s, 1H), 6.94 (s, 1H), 6.86 (s, 1H), 6.74 (s, 1H), 6.49 (d, 2H), 3.76 (s, 6H), 3.71 (s, 3H), 3.39 (m, 2H), 3.15 (m, 2H), 2.35 (s, 1H), 2.27 (s, 1H), 2.10 (s, 1H), 2.00 (s, 3H), 1.85 (s, 1H), 1.77 (s, 1H), 1.54 (s, 3H), 1.31 (s, 1H); LC-MS (ESI+) m/z 444.2 (M+H)⁺, RT=1.917 minutes.

### Example 44

### ({2-[(3,5-dimethoxy-4-methylbenzoyl)(3-phenylpropyl)amino]ethyl}sulfanyl)acetic Acid

### Step 1: N-(2-chloroethyl)-3,5-dimethoxy-4-methyl-N-(3-phenylpropyl)benzamide

To a solution of N-(2-hydroxyethyl)-3,5-dimethoxy-4-methyl-N-(3-phenylpropyl)benzamide (100 mg, 0.280 mmol, Example 6—Step 4) in CH₂Cl₂ (3 mL) was added diisopropylethylamine (0.147 mL, 0.839 mmol). The solution was cooled down to 0° C., and methanesulfonyl chloride (0.044 mL, 0.560 mmol) in CH₂Cl₂ (0.5 mL) was added dropwise. After addition, the solution was stirred at room temperature for 1 hour. The solution was concentrated, and the residue was purified by flash column chromatography on silica gel eluted with ethyl acetate in hexanes (0-60%) to give the titled compound. LC-MS (ESI+) m/z 376.2 (M+H)⁺, RT=2.136 minutes.

### Step 2: ethyl ({2-[(3,5-dimethoxy-4-methylbenzoyl)(3-phenylpropyl)amino]ethyl}sulfanyl)acetate

To a mixture of N-(2-chloroethyl)-3,5-dimethoxy-4-methyl-N-(3-phenylpropyl)benzamide (210 mg, 0.559 mmol) in dimethyl sulfoxide (3 mL) and acetone (3 mL) was added ethyl 2-mercaptoacetate (33.6 mg, 0.279 mmol) and K₂CO₃ (77.0 mg, 0.559 mmol) at room temperature. The mixture was stirred at room temperature overnight. Then water (20 mL) was added, and the mixture was extracted with ethyl acetate (20 mL). The organic layer was dried over Na₂SO₄, filtered and concentrated. The residue was purified by flash chromatography on silica eluted with hexane and ethyl acetate (0-35%) to give the titled compound. LC-MS (ESI+) m/z 460 (M+H)⁺, RT=2.14 minutes.

### Step 3: ({2-[(3,5-dimethoxy-4-methylbenzoyl)(3-phenylpropyl)amino]ethyl}sulfanyl)acetic Acid

To a mixture of ethyl ({2-[(3,5-dimethoxy-4-methylbenzoyl)(3-phenylpropyl)amino]ethyl}sulfanyl)acetate (111 mg, 0.241 mmol) in tetrahydrofuran (2 mL) was added aqueous lithium hydroxide solution. (1.0 N, 1.45 mL). The mixture was stirred at room temperature for 1 hour. The mixture was acidified with 1 N hydrochloric acid to adjust pH=2-3. Then the mixture was extracted with ethyl acetate twice, and the combined organic layers were concentrated to give a residue that was purified by preparative HPLC and lyophilized to give the titled compound. ¹H NMR (400 MHz, DMSO-d₆) δ ppm 7.42-6.95 (m, 5H), 6.54 (s, 2H), 3.76 (s, 6H), 3.60 (m, 2H), 3.35 (m, 2H), 3.21 (s, 1H), 3.05 (s, 1H), 2.80 (d, 2H), 2.63 (s, 1H), 2.42 (s, 1H), 2.01 (s, 3H), 1.84 (s, 2H); LC-MS (ESI+) m/z 432.2 (M+H)⁺, RT=1.945 minutes.

### Example 45

### N-(3,5-dimethoxy-4-methylbenzoyl)-N-(3-phenylpropyl)glycyl-2-methylalanine

Example 45 was prepared according to the procedure used for the preparation of Example 2, substituting methyl 2-amino-2-methylpropanoate for methyl 1-aminocyclohexanecarboxylate, to provide the titled compound. ¹H NMR (400 MHz, DMSO-d₆) δ ppm 7.94 (brs, 1H), 7.33-7.00 (m, 5H), 6.61 (brs, 2H), 3.92 (brs, 2H), 3.75 (s, 6H), 3.32 (brs, 2H), 2.54 (brs, 2H), 2.00 (s, 3H), 1.85 (brs, 2H), 1.36 (s, 6H); LC-MS (ESI+) m/z 457 (M+H)⁺, RT=1.85 minutes.

### Example 46

### N-(3,5-dimethoxy-4-methylbenzoyl)-N-(3-phenylpropyl)glycylphenylalanine

Example 46 was prepared according to the procedure used for the preparation of Example 2, substituting methyl 2-amino-3-phenylpropanoate for methyl 1-aminocyclohexanecarboxylate, to provide the titled compound. ¹H NMR (400 MHz, DMSO-d₆) δ ppm 8.09 (brs, 1H), 7.37-6.95 (m, 10H), 6.57 (s, 2H), 4.53 (td, J=8.5, 5.2 Hz, 1H), 3.89 (brs, 2H), 3.71 (s, 6H), 3.18 (brs, 2H), 3.07 (dd, J=13.9, 5.2 Hz, 2H), 2.91-2.89 (m, 2H), 1.99 (s, 3H), 1.77 (s, 2H); LC-MS (ESI+) m/z 519 (M+H)⁺, RT=1.97 minutes.

### Example 47

### N-(3,5-dimethoxy-4-methylbenzoyl)-N-(3-phenylpropyl)glycyl-3-thiophen-2-ylalanine

Example 47 was prepared according to the procedure used for the preparation of Example 2, substituting methyl 2-amino-3-(thiophen-2-yl)propanoate for methyl 1-aminocyclohexanecarboxylate, to provide the titled compound. ¹H NMR (400 MHz, DMSO-d₆) δ ppm 12.42 (brs, 1H), 8.62 (d, J=8.2 Hz, 1H), 7.44-7.32 (m, 1H), 7.29-7.11 (m, 4H), 7.03-6.92 (m, 3H), 6.63 (s, 1H), 6.54 (s, 1H), 5.44 (d, J=7.9 Hz, 1H), 4.05 (d, J=11.9 Hz, 1H), 3.84 (s, 1H), 3.74 (s, 3H), 3.66 (s, 3H), 3.22-3.17 (m, 1H), 2.80-2.73 (m, 2H), 2.60-2.53 (m, 1H), 2.42-2.36 (m, 2H), 1.99 (s, 1H), 1.96 (s, 2H), 1.86-1.80 (m, 2H); LC-MS (ESI+) m/z 525 (M+H)⁺, RT=1.93 minutes.

### Example 48

5-[(2,6-dimethoxypyridine-4-carbonyl)(3-phenylpropyl)amino]pentanoic Acid

Example 48 was prepared according to the procedure used for the preparation of Example 4, substituting 2,6-dimethoxyisonicotinic acid for 3,5-dimethoxy-4-methylbenzoic acid, to provide the titled compound. ¹H NMR (400 MHz, DMSO-d₆) δ ppm 7.32-7.06 (m, 4H), 7.05-6.98 (m, 1H), 6.24 (s, 1H), 6.18 (s, 1H), 3.85 (d, J=2.1 Hz, 6H), 3.37 (q, J=7.1 Hz, 2H), 3.07 (dt, J=19.2, 7.6 Hz, 2H), 2.60 (t, J=7.8 Hz, 1H), 2.39 (t, J=7.4 Hz, 1H), 2.23 (t, J=6.8 Hz, 1H), 2.07 (d, J=6.1 Hz, 1H), 1.89-1.81 (m, 1H), 1.56-1.39 (m, 3H), 1.27 (p, J=7.4 Hz, 1H); LC-MS (ESI+) m/z 401 (M+H)⁺, RT=1.87 minutes.

### Example 49

### 5-{[3-(2,4-dichlorophenyl)propyl](3,5-dimethoxy-4-methylbenzoyl)amino}pentanoic Acid

Example 49 was prepared according to the procedure used for the preparation of Example 11, substituting 3-(2,4-dichlorophenyl)propan-1-amine for 3-(o-tolyl)propan-1-amine, to provide the titled compound. ¹H NMR (400 MHz, DMSO-d₆) δ ppm 11.83 (s, 1H), 7.46 (s, 1H), 7.27 (s, 2H), 6.47 (s, 2H), 3.75 (s, 6H), 3.30 (s, 2H), 3.14 (s, 2H), 2.67-2.52 (m, 2H), 2.17 (s, 2H), 2.00 (s, 3H), 1.81 (s, 2H), 1.56 (s, 2H), 1.45 (s, 2H), T=60 OC; LC-MS (ESI+) m/z 482.2, 484.2 (M+H)⁺, RT=2.074 minutes.

### Example 50

### ({2-[(3,5-dimethoxy-4-methylbenzoyl)(3-phenylpropyl)amino]ethoxy}methyl)phosphonic Acid

### Step 1: diethyl ({2-[(3,5-dimethoxy-4-methylbenzoyl)(3-phenylpropyl)amino]ethoxy}methyl)phosphonate

To a solution of N-(2-hydroxyethyl)-3,5-dimethoxy-4-methyl-N-(3-phenylpropyl)benzamide (40 mg, 0.112 mmol, Example 6—Step 4) in tetrahydrofuran (2 mL) was added NaH (13.43 mg, 0.336 mmol), and the mixture was stirred at room temperature for 1 hour. (Diethoxyphosphoryl)methyl 4-methylbenzenesulfonate (108 mg, 0.336 mmol) was added, and stirring was continued overnight. The reaction was quenched with water (15 mL), and the mixture was extracted with ethyl acetate (10 mL×3). The organic phase was concentrated. Flash chromatography on silica gel (0-100% ethyl acetate in hexanes) gave the titled compound (40 mg, 0.059 mmol, 52.8% yield) that was used directly in the next step. MS (APCI⁺) m/z 508.2 (M+H)⁺.

### Step 2: ({2-[(3,5-dimethoxy-4-methylbenzoyl)(3-phenylpropyl)amino]ethoxy}methyl)phosphonic Acid

To a solution of diethyl ({2-[(3,5-dimethoxy-4-methylbenzoyl)(3-phenylpropyl)amino]ethoxy}methyl)phosphonate (40 mg, 0.079 mmol, Step 1) in dichloromethane (5 mL) was added bromotrimethylsilane (12.07 mg, 0.079 mmol), and the mixture stirred at room temperature overnight. The reaction was quenched with methanol, and the mixture was then stirred for 2 hours and concentrated. The residue was purified by preparative HPLC to give the titled compound (22.7 mg, 0.049 mmol, 61.9% yield). ¹H NMR (400 MHz, DMSO-d₆) δ ppm 7.33-7.01 (m, 5H), 6.54 (d, J=22.4 Hz, 2H), 3.83-3.68 (m, 6H), 3.71-3.30 (m, 7H), 3.22 (s, 1H), 2.61 (s, 1H), 2.38 (s, 1H), 1.98 (s, 3H), 1.87 (s, 2H); MS (APCI⁺) m/z 452.2 (M+H)⁺.

### Example 51

### N-(3,5-dimethoxy-4-methylbenzoyl)-N-(3-phenylpropyl)glycylglycine

Example 51 was prepared according to the procedure used for the preparation of Example 2, substituting methyl 2-aminoacetate for methyl 1-aminocyclohexanecarboxylate, to provide the titled compound. ¹H NMR (400 MHz, DMSO-d₆) δ ppm 8.11 (s, 1H), 7.43-6.91 (m, 5H), 6.60 (s, 2H), 3.92 (s, 2H), 3.79 (d, J=5.8 Hz, 2H), 3.74 (s, 6H), 3.14 (s, 4H), 2.00 (s, 3H), 1.86 (s, 2H); LC-MS (ESI+) m/z 429.2 (M+H)⁺, RT=1.536 minutes.

### Example 52

### 2-benzyl-5-[(3,5-dimethoxy-4-methylbenzoyl)(3-phenylpropyl)amino]pentanoic Acid

### Step 1: methyl 2-benzyl-5-[(3,5-dimethoxy-4-methylbenzoyl)(3-phenylpropyl)amino]pentanoate

To a solution of methyl 5-[(3,5-dimethoxy-4-methylbenzoyl)(3-phenylpropyl)amino]pentanoate (200 mg, 0.468 mmol, Example 4—Step 3) in tetrahydrofuran (10 mL) was added a solution of lithium diisopropylamide (55.1 mg, 0.515 mmol) in tetrahydrofuran at −78° C. The mixture was stirred at −78° C. for 1 hour. Then a solution of benzyl bromide (0.056 mL, 0.468 mmol) in tetrahydrofuran (2 mL) was added to the mixture, and the mixture was stirred at −78° C. to room temperature overnight. Saturated NH₄Cl (5 mL) was added to the mixture, and the mixture was diluted with water (20 mL) and ethyl acetate (20 mL). The mixture was extracted with ethyl acetate (20 mL). The organic layer was dried over Na₂SO₄, filtered and concentrated. The residue was purified by preparative TLC to give the titled compound. LC-MS (ESI+) m/z 518 (M+H)⁺, RT=2.26 minutes.

### Step 2: 2-benzyl-5-[(3,5-dimethoxy-4-methylbenzoyl)(3-phenylpropyl)amino]pentanoic Acid

To a solution of methyl 2-benzyl-5-[(3,5-dimethoxy-4-methylbenzoyl)(3-phenylpropyl)amino]pentanoate (30 mg, 0.058 mmol) in tetrahydrofuran (2 mL) was added aqueous lithium hydroxide solution (1.0 N, 1.0 mL). The mixture was stirred at room temperature for 2 hours. The mixture was acidified with 1 N hydrochloric acid to adjust pH=3. The mixture was then extracted with ethyl acetate twice, and the combined organic layers were concentrated to give a residue that was purified by preparative HPLC and lyophilized to give the titled compound. ¹H NMR (400 MHz, DMSO-d₆) δ ppm 7.19 (dq, J=8.6, 8.0 Hz, 10H), 6.48 (s, 2H), 3.75 (s, 6H), 3.42-3.12 (m, 4H), 2.81 (t, J=10.8 Hz, 1H), 2.65 (d, J=13.8 Hz, 1H), 2.53 (s, 3H), 2.00 (s, 3H), 1.82 (d, J=10.1 Hz, 2H), 1.68-1.22 (m, 4H); LC-MS (ESI+) m/z 504 (M+H)⁺, RT=2.11 minutes.

### Example 53

### {2-[(3,5-dimethoxy-4-methylbenzoyl)(3-phenylpropyl)amino]ethanesulfinyl}acetic Acid

({2-[(3,5-Dimethoxy-4-methylbenzoyl)(3-phenylpropyl)amino]ethyl}sulfanyl)acetic acid (20 mg, 0.046 mmol, Example 44) was dissolved in acetic acid (1 mL). The mixture was cooled to 5° C., and hydrogen peroxide (1.576 mg, 0.046 mmol) was added. Then the mixture was stirred at room temperature for 3 hours. The reaction mixture was diluted with water and extracted with ethyl acetate (2×20 mL). The combined organic layers were washed with brine (3×20 mL), dried over anhydrous Na₂SO₄, filtered and concentrated in vacuo to give a residue that was purified by preparative HPLC and lyophilized to give the titled compound. ¹H NMR (400 MHz, DMSO-d₆) δ ppm 7.17-7.03 (m, 5H), 6.53 (s, 2H), 3.93 (s, 2H), 3.74 (s, 6H), 3.64 (m, 2H), 3.21 (m, 2H), 3.11 (m, 1H), 2.63 (s, 1H), 2.45-2.38 (m, 2H), 1.98 (s, 3H), 1.85 (s, 2H); LC-MS (ESI+) m/z 448.2 (M+H)⁺, RT=1.795 minutes.

### Example 54

### 5-[(3,5-dimethoxy-4-methylbenzoyl)(3-phenylpropyl)amino]-2-methylpentanoic Acid

### Step 1: methyl 5-[(3,5-dimethoxy-4-methylbenzoyl)(3-phenylpropyl)amino]-2-methylpentanoate

To a solution of diisopropylamine (1.467 mL, 10.29 mmol) in tetrahydrofuran (10 mL) was added n-butyllithium (10.29 mmol, 6.4 mL, 1.6 M in hexane) at 0° C. under a N₂ atmosphere. The mixture was stirred at 0° C. for 0.5 hour, and then cooled to −78° C. Then a solution of methyl 5-[(3,5-dimethoxy-4-methylbenzoyl)(3-phenylpropyl)amino]pentanoate (2 g, 4.68 mmol, Example 4—Step 3) in tetrahydrofuran (10 mL) was added to the mixture dropwise, and the mixture was stirred at −78° C. for 10 minutes. Subsequently, methyl iodide (0.878 mL, 14.03 mmol) was added to the mixture, and the resultant mixture was stirred at −78° C. to room temperature for 2 hours. Then saturated NH₄Cl was added to the mixture, and the resultant mixture was extracted with ethyl acetate (20 mL). The organic layer was dried over Na₂SO₄, filtered and concentrated to dryness. The residue was purified by flash chromatography on silica gel eluted with hexane and ethyl acetate to give the titled compound (1.74 g, 3.94 mmol, 84% yield) that was used directly in the next step.

### Step 2: 5-[(3,5-dimethoxy-4-methylbenzoyl)(3-phenylpropyl)amino]-2-methylpentanoic Acid

To a solution of methyl 5-[(3,5-dimethoxy-4-methylbenzoyl)(3-phenylpropyl)amino]-2-methylpentanoate (800 mg, 1.812 mmol, Step 1) in tetrahydrofuran (6 mL) was added LiOH (8.15 mL, 8.15 mmol), and methanol (1 mL) was added to mix the layers. The reaction mixture was stirred at room temperature. Progress of the reaction was monitored by LC-MS (“TFA method”). After 3.5 hours, LC-MS indicated complete reaction. The reaction mixture was concentrated to remove volatiles. The residue was diluted with water, and this basic layer was acidified to pH˜1 with 1 N HCl. The aqueous mixture was then extracted with ethyl acetate. The organic layer was washed with brine, dried (MgSO₄) and concentrated. The residue was purified by flash chromatography on silica gel eluted with 0-100% ethyl acetate/heptanes gave the titled compound (0.75 g, 97%). ¹H NMR (400 MHz, DMSO-d₆) δ ppm 11.85 (brs, 1H), 7.18 (dt, J=34.8, 7.4 Hz, 5H), 6.49 (s, 2H), 3.75 (s, 6H), 3.14 (brs, 4H), 2.52-2.51 (m, 1H), 2.27 (s, 1H), 2.00 (s, 3H), 1.84 (t, J=8.0 Hz, 2H), 1.52 (s, 3H), 1.24 (s, 1H), 1.02 (d, J=7.0 Hz, 3H); LC-MS (ESI+) m/z 428 (M+H)⁺, RT=1.99 minutes.

### Example 55

### 2-{2-[(3,5-dimethoxy-4-methylbenzoyl)(3-phenylpropyl)amino]ethoxy}-2-methylpropanoic Acid

### Step 1: methyl 2-(2,2-dimethoxyethoxy)-2-methylpropanoate

To a mixture of sodium hydride (2.71 g, 67.7 mmol) in N,N-dimethylformamide (16 mL) was added methyl 2-hydroxy-2-methylpropanoate (4.00 g, 33.9 mmol) slowly over 5 minutes at room temperature. After addition, the mixture was stirred for 20 minutes. Then 2-bromo-1,1-dimethoxyethane (28.6 g, 169 mmol) was added dropwise to the mixture cooled in an ice-water bath. The reaction mixture was stirred at room temperature overnight. Then the mixture was poured into a vigorously stirred mixture of saturated aqueous ammonium chloride (50 mL) and ethyl acetate/hexanes(1:3, 50 mL). Then the organic layer was separated, washed with water and brine once each, dried over anhydrous Na₂SO₄, filtered and concentrated under vacuum at 70° C. to give a residue that was purified by flash column chromatography on silica gel eluted with ethyl acetate in hexanes (0-100%) to give the titled compound. ¹H NMR (400 MHz, DMSO-d₆) δ ppm 4.52 (t, J=5.2 Hz, 1H), 3.74 (s, 3H), 3.46 (d, J=5.2 Hz, 2H), 3.40 (s, 6H), 1.44 (s, 6H).

### Step 2: methyl 2-methyl-2-(2-oxoethoxy)propanoate

To a solution of methyl 2-(2,2-dimethoxyethoxy)-2-methylpropanoate (545 mg, 2.64 mmol) in CHCl₃ (5 mL) was added water (0.238 mL, 13.2 mmol) and trifluoroacetic acid (1.02 mL, 13.2 mmol), sequentially. The resulting mixture was stirred at room temperature overnight. Then the mixture was washed with water (5 mL×2) and saturated aqueous sodium bicarbonate solution (5 mL). The combined aqueous solution was extracted with dichloromethane (10 mL×2). The combined organic layers were washed with brine once, dried over anhydrous Na₂SO₄, filtered and concentrated in vacuo at 35° C. to give the titled compound. ¹H NMR (400 MHz, DMSO-d₆) δ ppm 9.75 (t, J=1.2 Hz, 1H), 4.05 (d, J=1.2 Hz, 2H), 3.74 (s, 3H), 1.49 (s, 6H).

### Step 3: methyl 2-methyl-2-{2-[(3-phenylpropyl)amino]ethoxy}propanoate

A solution of methyl 2-methyl-2-(2-oxoethoxy)propanoate (200 mg, 1.25 mmol) and 3-phenylpropan-1-amine (169 mg, 1.25 mmol) in 1,2-dichloroethane (6 mL) was stirred at room temperature for 3 hours. Then 10% Pd/C (26.6 mg, 0.250 mmol) was added, and the mixture was stirred at room temperature for 1.5 hours under an atmosphere of hydrogen (balloon). Then additional Pd/C (26.6 mg, 0.250 mmol) was added, and the mixture was stirred for an additional 2 hours under a hydrogen balloon. The mixture was filtered, and the solids were washed with methanol. The filtrate was concentrated to provide the titled compound that was used directly in the next step without further purification. LC-MS (ESI+) m/z 280.2 (M+H)⁺, RT=1.554 minutes.

### Step 4: methyl 2-{2-[(3,5-dimethoxy-4-methylbenzoyl)(3-phenylpropyl)amino]ethoxy}-2-methylpropanoate

To a solution of 3,5-dimethoxy-4-methylbenzoic acid (257 mg, 1.31 mmol) and 1-[bis(dimethylamino)methylene]-1H-1,2,3-triazolo[4,5-b]pyridinium 3-oxid hexafluorophosphate (523 mg, 1.38 mmol, HATU) in N,N-dimethylformamide (5 mL) was added triethylamine (0.192 mL, 1.38 mmol). The resulting solution was stirred at room temperature for 10 minutes. Then the mixture of methyl 2-methyl-2-{2-[(3-phenylpropyl)amino]ethoxy}propanoate from the previous step was added to the mixture in one portion. The mixture was stirred at room temperature for approximately 60 hours. The reaction mixture was diluted with water (30 mL) and extracted with ethyl acetate (20 mL×3). The combined organic layers were washed with brine three times, dried over anhydrous Na₂SO₄, filtered and concentrated to give a residue that was purified by flash column chromatography on silica gel eluted with ethyl acetate in hexanes (0-40%) to give the titled compound. LC-MS (ESI+) m/z 458.2 (M+H)⁺, RT=2.139 minutes.

### Step 5: 2-{2-[(3,5-dimethoxy-4-methylbenzoyl)(3-phenylpropyl)amino]ethoxy}-2-methylpropanoic Acid

To a solution of methyl 2-{2-[(3,5-dimethoxy-4-methylbenzoyl)(3-phenylpropyl)amino]ethoxy}-2-methylpropanoate (144 mg, 0.315 mmol) in dioxane (4 mL) was added aqueous lithium hydroxide solution. (1.0 N, 1.888 mL). The mixture was stirred at room temperature for 1 hour. The mixture was acidified with 1 N hydrochloric acid to adjust pH=3 and then extracted with ethyl acetate twice. The combined organic layers were concentrated to give a residue that was purified by preparative HPLC and lyophilized to give the titled compound. ¹H NMR (400 MHz, DMSO-d₆) δ ppm 7.36-6.93 (m, 5H), 6.54 (s, 2H), 3.76 (s, 6H), 3.51 (s, 2H), 3.40 (s, 4H), 2.50 (s, 2H), 2.00 (s, 3H), 1.95-1.80 (m, 2H), 1.23 (s, 6H), T=60° C.; LC-MS (ESI+) m/z 444.2 (M+H)⁺, RT=1.991 minutes.

### Example 56

### 3-{2-[(3,5-dimethoxy-4-methylbenzoyl)(3-phenylpropyl)amino]ethoxy}propanoic Acid

### Step 1: methyl 3-{2-[(3,5-dimethoxy-4-methylbenzoyl)(3-phenylpropyl)amino]ethoxy}propanoate

N-(2-Hydroxyethyl)-3,5-dimethoxy-4-methyl-N-(3-phenylpropyl)benzamide (150 mg, 0.420 mmol, Example 6—Step 4) was added to a mixture of dry tetrahydrofuran (3 mL) and sodium hydride (30.2 mg, 1.259 mmol). The mixture was stirred for 20 minutes at room temperature. Then methyl acrylate (181 mg, 2.098 mmol) was added, and the resultant mixture was stirred for another 3 hours at ambient temperature. The mixture was poured into water, and the mixture was extracted with ethyl acetate three times. The combined organic layers were washed with brine, dried over sodium sulfate, and concentrated. The residue was purified by flash column chromatography on silica gel eluted with ethyl acetate in hexanes (0-50%) to give the titled compound. LC-MS (ESI+) m/z 444.5 (M+H)⁺, RT=1.916 minutes.

### Step 2: 3-{2-[(3,5-dimethoxy-4-methylbenzoyl)(3-phenylpropyl)amino]ethoxy}propanoic Acid

Methyl 3-{2-[(3,5-dimethoxy-4-methylbenzoyl)(3-phenylpropyl)amino]ethoxy}propanoate (25 mg, 0.056 mmol) was dissolved in tetrahydrofuran. 1 N Lithium hydroxide (6.75 mg, 0.282 mmol) solution was added, and then the mixture was stirred at room temperature for 3 hours. The mixture was acidified with 1 N hydrochloric acid to adjust the pH=2-3. Then the mixture was extracted with ethyl acetate twice, and the combined organic layers were concentrated. The residue was purified by preparative HPLC and lyophilized to give the titled compound. ¹H NMR (400 MHz, DMSO-d₆) δ ppm 7.27-6.93 (m, 5H), 6.47 (s, 2H), 3.70 (s, 6H), 3.53 (t, J=6.0 Hz, 2H), 3.47 (s, 2H), 3.40 (s, 2H), 2.43 (m, 4H), 2.36 (t, J=6.2 Hz, 2H), 1.94 (s, 3H), 1.87-1.71 (m, 2H); LC-MS (ESI+) m/z 430.2 (M+H)⁺, RT=1.644 minutes.

### Example 57

### 5-[(3,5-dimethoxy-4-methylbenzoyl)(3-phenylpropyl)amino]-2,2-dimethylpentanoic Acid

### Step 1: methyl 5-[(3,5-dimethoxy-4-methylbenzoyl)(3-phenylpropyl)amino]-2,2-dimethylpentanoate

To a solution of diisopropylamine (0.267 mL, 1.87 mmol) in tetrahydrofuran (5 mL) was added n-butyllithium (1.87 mmol, 1.16 mL, 1.6 M in hexane) at 0° C. under a nitrogen atmosphere. The mixture was stirred at 0° C. for 0.5 hour, and cooled to −78° C. Then a solution of methyl 5-[(3,5-dimethoxy-4-methylbenzoyl)(3-phenylpropyl)amino]-2-methylpentanoate (376 mg, 0.852 mmol, prepared as an intermediate for the preparation of Example 54 using the procedure described for Example 52) in tetrahydrofuran (5 mL) was added to the mixture dropwise, and the mixture was stirred at −78° C. for 1 hour. Then methyl iodide (0.160 mL, 2.55 mmol) was added to the mixture, and the mixture was stirred at −78° C. to room temperature for 2 hours. Then saturated NH₄Cl was added to quench the reaction, and the mixture was extracted with ethyl acetate (30 mL). The organic layer was dried over Na₂SO₄, filtered and concentrated in vacuo to give a residue that was purified by chromatography on silica gel eluted with 0-50% ethyl acetate/hexanes to give the titled compound. LC-MS (ESI+) m/z 456 (M+H)⁺, RT=2.20 minutes.

### Step 2: 5-[(3,5-dimethoxy-4-methylbenzoyl)(3-phenylpropyl)amino]-2,2-dimethylpentanoic Acid

Methyl 5-[(3,5-dimethoxy-4-methylbenzoyl)(3-phenylpropyl)amino]-2,2-dimethylpentanoate (200 mg, 0.439 mmol) was dissolved in tetrahydrofuran (2 mL), and aqueous lithium hydroxide solution (1.3 mL, 1.31 mmol) was added. The mixture was stirred at room temperature for 12 hours. The mixture was acidified with 1 N hydrochloric acid to pH=2-3. Then the mixture was extracted with ethyl acetate twice. The combined organic layers were concentrated to give a residue that was purified by preparative HPLC and lyophilized to give the titled compound. ¹H NMR (400 MHz, DMSO-d₆) δ ppm 7.45-6.84 (m, 5H), 6.50 (s, 2H), 3.76 (s, 6H), 3.40 (m, 2H), 3.15 (s, 2H), 2.64 (s, 1H), 2.41 (s, 1H), 2.00 (s, 3H), 1.85 (m, 1H), 1.80 (m, 1H), 1.48 (m, 3H), 1.25 (s, 1H), 1.10 (s, 3H), 1.02 (s, 3H); LC-MS (ESI+) m/z 442.3 (M+H)⁺, RT=1.916 minutes.

### Example 58

### 5-[(2-fluoro-3,5-dimethoxybenzoyl)(3-phenylpropyl)amino]pentanoic Acid

Example 58 was prepared according to the procedure used for the preparation of Example 4, substituting 2-fluoro-3,5-dimethoxybenzoic acid for 3,5-dimethoxy-4-methylbenzoic acid, to provide the titled compound. ¹H NMR (400 MHz, DMSO-d₆) δ ppm 12.02 (s, 1H), 7.19 (m, 4H), 6.98 (d, J=7.3 Hz, 1H), 6.70 (dd, J=21.7, 6.9 Hz, 1H), 6.31 (d, J=12.6 Hz, 1H), 3.82 (d, J=4.1 Hz, 3H), 3.72 (d, J=7.3 Hz, 3H), 3.09 (m, 2H), 2.49 (s, 2H), 2.38 (t, J=7.4 Hz, 1H), 2.05 (d, J=5.7 Hz, 1H), 1.85 (m, 1H), 1.72 (t, J=8.5 Hz, 1H), 1.49 (m, 3H), 1.26 (m, 1H); LC-MS (ESI+) m/z 418.2 (M+H)⁺, RT=1.849 minutes.

### Example 59

### 1-({2-[(3,5-dimethoxy-4-methylbenzoyl)(3-phenylpropyl)amino]ethoxy}methyl)cyclopropane-1-carboxylic Acid

### Step 1: ethyl 1-(bromomethyl)cyclopropanecarboxylate

To the solution of ethyl 1-(hydroxymethyl)cyclopropanecarboxylate (520 mg, 3.61 mmol) in dichloromethane (25 mL) was added triphenylphosphine (1.14 g, 4.33 mmol) and carbon tetrabromide (1.79 g, 5.41 mmol) sequentially under a N₂ atmosphere. Then the solution was stirred at room temperature for 30 minutes, and then aqueous saturated NaHCO₃ was added to quench the reaction. The mixture was separated, and the organic layer was washed with brine once, dried over anhydrous Na₂SO₄, filtered and concentrated under vacuum to give a residue. The residue was purified by flash column chromatography on silica gel eluted with ethyl acetate in hexanes (0-20%) to give impure titled compound. The material was purified again by flash column chromatography on silica gel eluted with ethyl acetate in hexanes (0-20%) to give the titled compound. ¹H NMR (400 MHz, CDCl₃) δ ppm 4.19 (q, J=7.1 Hz, 2H), 3.62 (s, 2H), 1.53 (dd, J=5.2, 2.3 Hz, 2H), 1.28 (t, J=7.1 Hz, 3H), 1.03-1.00 (m, 2H).

### Step 2: 1-({2-[(3,5-dimethoxy-4-methylbenzoyl)(3-phenylpropyl)amino]ethoxy}methyl)cyclopropane-1-carboxylic Acid

The titled compound was prepared according to the procedure used for the preparation of Example 6, substituting ethyl 1-(bromomethyl)cyclopropanecarboxylate for methyl 2-chloroacetate. ¹H NMR (400 MHz, DMSO-d₆) δ ppm 12.40 (s, 1H), 7.33-6.99 (m, 5H), 6.52 (d, J=24.5 Hz, 2H), 3.74 (s, 6H), 3.55 (s, 3H), 3.45 (s, 3H), 3.30 (s, 1H), 3.21 (s, 1H), 2.61 (s, 1H), 2.38 (s, 1H), 1.98 (s, 3H), 1.86 (d, J=10.4 Hz, 2H), 0.91 (s, 2H), 0.64 (d, J=17.4 Hz, 2H), T=25° C.; LC-MS (ESI+) m/z 456.2 (M+H)⁺, RT=1.958 minutes.

### Example 60

### 3-({2-[(3,5-dimethoxy-4-methylbenzoyl)(3-phenylpropyl)amino]ethyl}sulfanyl)propanoic Acid

### Step 1: N-(2-chloroethyl)-3,5-dimethoxy-4-methyl-N-(3-phenylpropyl)benzamide

To a solution of N-(2-hydroxyethyl)-3,5-dimethoxy-4-methyl-N-(3-phenylpropyl)benzamide (100 mg, 0.280 mmol, Example 6—Step 4) in CH₂Cl₂ (3 mL) was added diisopropylethylamine (0.147 mL, 0.839 mmol). The solution was cooled down to 0° C. followed by addition of methanesulfonyl chloride (0.044 mL, 0.560 mmol) in dichloromethane (0.5 mL) dropwise. After addition, the solution was stirred at room temperature for 1 hour. The solution was concentrated, and the residue was purified by flash column chromatography on silica gel eluted with ethyl acetate in hexanes (0-60%) to give the titled compound. LC-MS (ESI+) m/z 376.2 (M+H)⁺, RT=2.136 minutes.

### Step 2: S-{2-[(3,5-dimethoxy-4-methylbenzoyl)(3-phenylpropyl)amino]ethyl}ethanethioate

To a solution of N-(2-chloroethyl)-3,5-dimethoxy-4-methyl-N-(3-phenylpropyl)benzamide (500 mg, 1.33 mmol) in N,N-dimethylformamide (10 mL) was added potassium ethanethioate (456 mg, 3.99 mmol) in one portion. Then the mixture was heated to 50° C. under a N₂ atmosphere for 3 hours. The mixture was cooled down, diluted with water (30 mL), and extracted with ethyl acetate (20 mL×3). The combined organic layers were washed with brine twice, dried over anhydrous Na₂SO₄, filtered and concentrated. The residue was purified by flash column chromatography on silica gel eluted with ethyl acetate in hexanes (0-60%) to give the titled compound. ¹H NMR (400 MHz, DMSO-d₆) δ ppm 7.21 (d, J=7.5 Hz, 2H), 7.13 (t, J=7.3 Hz, 3H), 6.52 (s, 2H), 3.76 (s, 6H), 3.47 (s, 2H), 3.33 (s, 2H), 3.11-3.01 (m, 2H), 2.51 (d, J=9.2 Hz, 2H), 2.30 (s, 3H), 2.00 (s, 3H), 1.86 (h, J=6.7, 5.7 Hz, 2H), T=60° C.; LC-MS (ESI+) m/z 416.2 (M+H)⁺, RT=2.122 minutes.

### Step 3: methyl 3-({2-[(3,5-dimethoxy-4-methylbenzoyl)(3-phenylpropyl)amino]ethyl}sulfanyl)propanoate

To a degassed solution of potassium hydroxide (27.0 mg, 0.481 mmol) in methanol (2 mL) was added S-{2-[(3,5-dimethoxy-4-methylbenzoyl)(3-phenylpropyl)amino]ethyl}ethanethioate (100 mg, 0.241 mmol), and the resulting mixture was stirred at 0° C. for 10 minutes under N₂. Then methyl 3-bromopropanoate (121 mg, 0.722 mmol) was added, and the mixture was heated to 50° C. for 2 hours. The mixture was concentrated giving a residue that was used directly in the next step. LC-MS (ESI+) m/z 460.2 (M+H)⁺, RT=2.103 minutes.

### Step 4: 3-({2-[(3,5-dimethoxy-4-methylbenzoyl)(3-phenylpropyl)amino]ethyl}sulfanyl)propanoic Acid

Aqueous lithium hydroxide solution. (1.0 N, 1.45 mL) was added to a mixture of methyl 3-({2-[(3,5-dimethoxy-4-methylbenzoyl)(3-phenylpropyl)amino]ethyl}sulfanyl)propanoate (111 mg, 0.241 mmol) in tetrahydrofuran (2 mL). The mixture was stirred at room temperature for 1 hour. The mixture was acidified with 1 N hydrochloric acid to adjust the pH=2-3. The mixture was then extracted with ethyl acetate twice, and the combined organic layers were concentrated to give a residue that was purified by preparative HPLC and lyophilized to give the titled compound. ¹H NMR (400 MHz, DMSO-d₆) δ ppm 12.24 (s, 1H), 7.32-6.99 (m, 5H), 6.52 (s, 2H), 3.74 (s, 6H), 3.53 (s, 1H), 3.43 (s, 1H), 3.32 (s, 1H), 3.19 (s, 1H), 2.73 (s, 2H), 2.64 (d, J=10.2 Hz, 2H), 2.53 (s, 1H), 2.39 (s, 2H), 2.34-2.26 (m, 1H), 1.98 (s, 3H), 1.86 (d, J=27.9 Hz, 2H), T=25° C.; LC-MS (ESI+) m/z 446.2 (M+H)⁺, RT=1.951 minutes.

### Example 61

### 1-[({2-[(3,5-dimethoxy-4-methylbenzoyl)(3-phenylpropyl)amino]ethyl}sulfanyl)methyl]cyclopropane-1-carboxylic Acid

The titled compound was prepared according to the procedure used for the preparation of Example 60, substituting ethyl 1-(bromomethyl)cyclopropanecarboxylate for methyl 3-bromopropanoate. ¹H NMR (400 MHz, DMSO-d₆) δ ppm 11.94 (s, 1H), 7.32-7.05 (m, 5H), 6.53 (s, 2H), 3.76 (s, 6H), 3.44 (s, 2H), 3.33 (s, 2H), 2.81-2.60 (m, 4H), 2.53 (s, 2H), 2.00 (s, 3H), 1.92-1.79 (m, 2H), 1.10 (q, J=3.9 Hz, 2H), 0.77 (s, 2H), T=60° C.; LC-MS (ESI+) m/z 472.2 (M+H)⁺, RT=2.002 minutes.

### Example 62

### 3-({2-[(3,5-dimethoxy-4-methylbenzoyl)(3-phenylpropyl)amino]ethyl}sulfanyl)butanoic Acid

The titled compound was prepared according to the procedure used for the preparation of Example 60, substituting methyl 3-bromobutanoate for methyl 3-bromopropanoate. ¹H NMR (400 MHz, DMSO-d₆) δ ppm 7.36-7.03 (m, 5H), 6.53 (s, 2H), 3.76 (s, 6H), 3.46 (s, 2H), 3.33 (s, 2H), 3.05 (s, 1H), 2.73 (s, 2H), 2.53 (s, 1H), 2.43 (s, 1H), 2.33 (s, 2H), 2.00 (s, 3H), 1.87 (d, J=9.4 Hz, 2H), 1.20 (s, 3H), T=60° C.; LC-MS (ESI+) m/z 460.2 (M+H)⁺, RT=1.998 minutes.

### Example 63

### 5-{[1-(5-methoxypyridin-2-yl)cyclopropane-1-carbonyl](3-phenylpropyl)amino}pentanoic Acid

The titled compound was prepared according to the procedure used for the preparation of Example 4, substituting 1-(5-methoxypyridin-2-yl)cyclopropanecarboxylic acid for 3,5-dimethoxy-4-methylbenzoic acid. ¹H NMR (400 MHz, DMSO-d₆) δ ppm 8.16 (dd, J=4.8, 3.0 Hz, 1H), 7.35-7.00 (m, 6H), 6.97 (d, J=7.3 Hz, 1H), 3.77 (d, J=2.3 Hz, 3H), 3.31-3.13 (m, 5H), 2.55 (t, J=7.8 Hz, 1H), 2.19 (dt, J=25.5, 6.9 Hz, 2H), 1.94 (s, 1H), 1.77 (q, J=7.8 Hz, 1H), 1.47-1.38 (m, 3H), 1.26 (q, J=4.0, 3.4 Hz, 1H), 1.19-1.11 (m, 4H); LC-MS (ESI+) m/z 411 (M+H)⁺, RT=1.677 minutes.

### Example 64

### 5-{(3-phenylpropyl)[1-(pyridin-4-yl)cyclopropane-1-carbonyl]amino}pentanoic Acid

The titled compound was prepared according to the procedure used for the preparation of Example 4, substituting 1-(pyridin-4-yl)cyclopropanecarboxylic acid for 3,5-dimethoxy-4-methylbenzoic acid. ¹H NMR (400 MHz, DMSO-d₆) δ ppm 8.47-8.39 (m, 2H), 7.33-7.11 (m, 4H), 7.12-6.89 (m, 3H), 3.31-3.19 (m, 2H), 3.09 (s, 2H), 2.54 (s, 1H), 2.28 (d, J=7.3 Hz, 1H), 2.06 (s, 1H), 1.85 (s, 1H), 1.77 (s, 1H), 1.50 (d, J=8.6 Hz, 1H), 1.46-1.34 (m, 2H), 1.24 (td, J=27.6, 4.1 Hz, 5H), 1.07-1.00 (m, 1H); LC-MS (ESI+) m/z 381 (M+H)⁺, RT=1.591 minutes.

### Example 65

### 5-[(6-methoxy-1H-indole-3-carbonyl)(3-phenylpropyl)amino]pentanoic Acid

The titled compound was prepared according to the procedure used for the preparation of Example 4, substituting 6-methoxy-1H-indole-3-carboxylic acid for 3,5-dimethoxy-4-methylbenzoic acid. ¹H NMR (400 MHz, CD₃OD) δ ppm 7.51 (d, J=8.8 Hz, 1H), 7.32 (s, 1H), 7.26-7.04 (m, 5H), 6.93 (d, J=2.3 Hz, 1H), 6.78 (dd, J=8.7, 2.3 Hz, 1H), 3.83 (s, 3H), 3.61-3.52 (m, 4H), 2.59 (s, 2H), 2.22 (s, 2H), 1.96 (s, 2H), 1.61 (d, J=38.5 Hz, 4H); LC-MS (ESI+) m/z 409 (M+H)⁺, RT=1.784 minutes.

### Example 66

### 5-{[(2R)-2-methoxy-2-(4-methoxyphenyl)acetyl](3-phenylpropyl)amino}pentanoic Acid

The titled compound was prepared according to the procedure used for the preparation of Example 4, substituting (S)-2-methoxy-2-(4-methoxyphenyl)acetic acid for 3,5-dimethoxy-4-methylbenzoic acid. ¹H NMR (400 MHz, DMSO-d₆) δ ppm 12.03 (s, 1H), 7.36-7.18 (m, 4H), 7.19-7.14 (m, 2H), 7.13-7.06 (m, 1H), 6.95-6.90 (m, 1H), 6.90-6.84 (m, 1H), 4.80 (s, 1H), 3.74 (s, 3H), 3.33 (m, 1H), 3.14 (s, 3H), 3.16-3.07 (m, 2H), 2.45 (m, 2H), 2.18 (dt, J=13.4, 6.9 Hz, 2H), 1.71 (q, J=7.3 Hz, 2H), 1.50-1.33 (m, 3H); LC-MS (ESI+) m/z 414.2 (M+H)⁺, RT=1.825 minutes.

### Example 67

### N-(3,5-dimethoxy-4-methylbenzoyl)-N-(3-phenylpropyl)-beta-alanine

### Step 1: methyl 3-[(3-phenylpropyl)amino]propanoate

A solution of 3-phenylpropan-1-amine (500 mg, 3.70 mmol) and methyl acrylate (500 μL 5.55 mmol) was heated to reflux at 90° C. for 1 hour. The mixture was cooled down and purified (loaded directly with CH₂Cl₂) by flash column chromatography (eluent: CH₂Cl₂/CH₃OH=0˜20%) to give the titled compound (668 mg, 3.02 mmol, 82% yield). LC-MS (ESI+) m/z 222.2 (M+H)⁺, RT=1.400 minutes.

### Step 2: methyl 3-[(3,5-dimethoxy-4-methylbenzoyl)(3-phenylpropyl)amino]propanoate

To a solution of 3,5-dimethoxy-4-methylbenzoic acid (380 mg, 1.937 mmol) and 1-[bis(dimethylamino)methylene]-1H-1,2,3-triazolo[4,5-b]pyridinium 3-oxid hexafluorophosphate (773 mg, 2.033 mmol, HATU) in N,N-dimethylformamide (8 mL) was added triethylamine (0.283 mL 2.033 mmol). The resulting solution was stirred at room temperature for 5 minutes. Then Example 67—Step 1 (450 mg, 2.033 mmol) was added in one portion. The solution was stirred at room temperature for 3 hours. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-100%) to give the titled compound (593 mg, 1.484 mmol, 77% yield). LC-MS (ESI+) m/z 400.2 (M+H)⁺, RT=1.990 minutes.

### Step 3: N-(3,5-dimethoxy-4-methylbenzoyl)-N-(3-phenylpropyl)-beta-alanine

To a solution of Example 67—Step 2 (593 mg, 1.484 mmol) in methanol (10 mL) was added 1 N lithium hydroxide (8.91 mL 8.91 mmol). The resulting mixture was stirred at room temperature overnight and then concentrated under reduced pressure. The resultant mixture was acidified with 1 N HCl to pH=2-3. The solid precipitate was extracted with ethyl acetate 3 times. The organic layers were combined and washed with brine once, dried over anhydrous Na₂SO₄, filtered and concentrated to give the titled compound (562 mg, 1.458 mmol, 98% yield). ¹H NMR (400 MHz, DMSO-d₆) δ ppm 12.30 (s, 1H), 7.17 (s, 4H), 7.03 (s, 1H), 6.52 (s, 2H), 3.74 (s, 6H), 3.57 (s, 1H), 3.41 (s, 2H), 3.19 (s, 1H), 2.54 (s, 4H), 1.98 (s, 3H), 1.83 (s, 2H); LC-MS (ESI+) m/z 386.2 (M+H)⁺, RT=1.856 minutes.

### Example 68

### 3,5-dimethoxy-4-methyl-N-{3-[(methylsulfamoyl)amino]-3-oxopropyl}-N-(3-phenylpropyl)benzamide

Example 67 (100 mg, 0.259 mmol) was dissolved in tetrahydrofuran (4 mL) and carbonyldiimidazole (63.1 mg, 0.389 mmol) was added. The mixture was heated to 60° C. for 1 hour. More carbonyldiimidazole (63.1 mg, 0.389 mmol) was added, and the resultant solution was stirred at 60° C. for 1 hour. The solution was cooled down to room temperature and was then added dropwise through a syringe to a solution of 1,8-diazabicyclo[5.4.0]undec-7-ene (0.117 mL 0.778 mmol, DBU) and N-methylsulfuric diamide (86 mg, 0.778 mmol) in 0.6 mL tetrahydrofuran. The resulting mixture was stirred at room temperature overnight. The mixture was acidified with 1 N HCl to pH=6-7 and then extracted with ethyl acetate 3 times. The organic layers were combined and concentrated. The residue was diluted with methanol to give a solution which was purified by preparative HPLC (0.1% CF₃CO₂H in H₂O/CH₃CN) and lyophilized to give the titled compound (72.5 mg, 0.152 mmol, 58.5% yield). ¹H NMR (400 MHz, DMSO-d₆) δ ppm 11.39 (s, 1H), 7.46 (q, J=4.9, 4.5 Hz, 1H), 7.14 (dd, J=56.8, 38.2 Hz, 5H), 6.50 (s, 2H), 3.74 (s, 6H), 3.67-3.58 (m, 1H), 3.46 (d, J=19.0 Hz, 4H), 3.19 (s, 1H), 2.60 (s, 3H), 2.40 (s, 2H), 1.98 (s, 3H), 1.82 (d, J=16.8 Hz, 2H); LC-MS (ESI+) m/z 478.2 (M+H)⁺, RT=1.919 minutes.

### Example 69

### 4-[(3,5-dimethoxy-4-methylbenzoyl)(3-phenylpropyl)amino]butanoic Acid

### Step 1: methyl (2E)-4-[(3-phenylpropyl)amino]but-2-enoate

A mixture of (E)-methyl 4-bromobut-2-enoate (300 mg, 1.676 mmol), 3-phenylpropan-1-amine (227 mg, 1.676 mmol) and potassium carbonate (463 mg, 3.35 mmol) in CH₃CN (5 mL) was stirred at room temperature for 1.5 hours. The mixture was concentrated to give the titled compound which was used directly in the next step. LC-MS (ESI+) m/z 234.2 (M+H)⁺, RT=1.470 minutes.

### Step 2: methyl (2E)-4-[(3,5-dimethoxy-4-methylbenzoyl)(3-phenylpropyl)amino]but-2-enoate

To a solution of 3,5-dimethoxy-4-methylbenzoic acid (329 mg, 1.676 mmol) and 1-[bis(dimethylamino)methylene]-1H-1,2,3-triazolo[4,5-b]pyridinium 3-oxid hexafluorophosphate (669 mg, 1.760 mmol, HATU) in N,N-dimethylformamide (6 mL) was added triethylamine (0.245 mL 1.760 mmol). The resulting solution was stirred at room temperature for 5 minutes. Then a solution of Example 69—Step 1 in N,N-dimethylformamide (2.0 mL) was added in one portion. The solution was stirred at room temperature for 30 minutes. The mixture was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (230 mg, 0.257 mmol, 15.31% yield). LC-MS (ESI+) m/z 412.2 (M+H)⁺, RT=2.060 minutes.

### Step 3: (2E)-4-[(3,5-dimethoxy-4-methylbenzoyl)(3-phenylpropyl)amino]but-2-enoic Acid

To a solution of Example 69—Step 2 (230 mg, 0.559 mmol) in 1,4-dioxane (5 mL) was added 1 N lithium hydroxide (2.236 mL 2.236 mmol). The solution was heated to 50° C. for 3 hours. The reaction mixture was acidified with 1 N HCl to pH=2-3. It was extracted with ethyl acetate twice. The combined organic layers were concentrated to give a residue which was purified twice by preparative HPLC (0.1% CF₃CO₂H in H₂O/CH₃CN) and lyophilized to give the titled compound (27 mg, 0.068 mmol, 12.15% yield). ¹H NMR (400 MHz, DMSO-d₆) δ ppm 12.17 (s, 1H), 7.36-6.98 (m, 5H), 6.76 (d, J=15.4 Hz, 1H), 6.55 (s, 2H), 5.87 (d, J=15.8 Hz, 1H), 4.11 (s, 2H), 3.75 (s, 6H), 3.30 (s, 2H), 2.50 (s, 2H), 2.00 (s, 3H), 1.95-1.76 (m, 2H); LC-MS (ESI) m/z 398.2 (M+H)⁺, RT=1.889 minutes.

### Step 4: 4-[(3,5-dimethoxy-4-methylbenzoyl)(3-phenylpropyl)amino]butanoic Acid

To a solution of Example 69—Step 3 (111 mg, 0.280 mmol) in CH₃OH (5 mL) was added palladium on carbon (2.98 mg, 0.028 mmol). The mixture was stirred at room temperature under a hydrogen atmosphere (balloon) for 1 hour. The reaction mixture was filtered. The filtrate was purified by preparative HPLC (0.1% CF₃CO₂H in H₂O/CH₃CN) and lyophilized to give the titled compound (43.7 mg, 0.109 mmol, 39.1% yield). ¹H NMR (400 MHz, DMSO-d₆) δ ppm 7.17 (dt, J=34.6, 8.0 Hz, 5H), 6.51 (s, 2H), 3.76 (s, 6H), 3.31 (s, 4H), 2.50 (s, 2H), 2.17 (d, J=7.9 Hz, 2H), 2.00 (s, 3H), 1.94-1.68 (m, 4H); LC-MS (ESI) m/z 400.2 (M+H)⁺, RT=1.914 minutes.

### Example 70

### 3,5-dimethoxy-4-methyl-N-{5-[(methylsulfamoyl)amino]-5-oxopentyl}-N-(3-phenylpropyl)benzamide

### Step 1: methyl 5-[(3-phenylpropyl)amino]pentanoate

A mixture of methyl 5-bromopentanoate (200 mg, 1.025 mmol), 3-phenylpropan-1-amine (139 mg, 1.025 mmol) and potassium carbonate (170 mg, 1.230 mmol) in CH₃CN (4 mL) was stirred at room temperature for 1.5 hours and then heated to reflux for 1 hour. The mixture was cooled down and filtered to give the titled compound which was used without further purification in the next step. LC-MS (ESI) m/z 250.2 (M+H)⁺, RT=1.515 minutes.

### Step 2: methyl 5-[(3,5-dimethoxy-4-methylbenzoyl)(3-phenylpropyl)amino]pentanoate

To a solution of 3,5-dimethoxy-4-methylbenzoic acid (201 mg, 1.025 mmol) and 1-[bis(dimethylamino)methylene]-1H-1,2,3-triazolo[4,5-b]pyridinium 3-oxid hexafluorophosphate (409 mg, 1.076 mmol, HATU) in N,N-dimethylformamide (6 mL) was added triethylamine (0.150 mL 1.076 mmol). The resulting solution was stirred at room temperature for 5 minutes. Then a solution of Example 70—Step 1 in N,N-dimethylformamide (2.0 mL) was added in one portion. The solution was stirred at room temperature for 1 hour. The mixture was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (10-70%) to give the titled compound (136 mg, 0.318 mmol, 31.0% yield). LC-MS (ESI) m/z 428.2 (M+H)⁺, RT=2.104 minutes.

### Step 3: 5-[(3,5-dimethoxy-4-methylbenzoyl)(3-phenylpropyl)amino]pentanoic Acid

To a solution of Example 70—Step 2 (136 mg, 0.318 mmol) in 1,4-dioxane (4 mL) was added 1 N lithium hydroxide (1.909 mL 1.909 mmol). The solution was heated to 50° C. for 1.5 hours. The solution was acidified with 1 N HCl to pH=2-3 and then extracted with ethyl acetate twice. The combined organic layers were concentrated to give a residue which was purified by preparative HPLC (0.1% CF₃CO₂H in H₂O/CH₃CN) and lyophilized to give the titled compound (118 mg, 0.286 mmol, 44.9% yield). ¹H NMR (400 MHz, DMSO-d₆) δ ppm 7.18 (dt, J=34.8, 7.6 Hz, 5H), 6.49 (s, 2H), 3.76 (s, 6H), 3.29 (s, 4H), 2.50 (s, 2H), 2.17 (s, 2H), 2.00 (s, 3H), 1.95-1.76 (m, 2H), 1.54 (d, J=8.7 Hz, 2H), 1.44 (s, 2H); LC-MS (ESI) m/z 414.2 (M+H)⁺, RT=1.943 minutes.

### Step 4: 3,5-dimethoxy-4-methyl-N-{5-[(methylsulfamoyl)amino]-5-oxopentyl}-N-(3-phenylpropyl)benzamide

Material from Example 70—Step 3 (59 mg, 0.143 mmol) was dissolved in tetrahydrofuran (4 mL), and carbonyldiimidazole (69.4 mg, 0.428 mmol) was added. The mixture was heated to 60° C. for 40 minutes. Additional carbonyldiimidazole (69.4 mg, 0.428 mmol) was added with continued stirring for 1 hour. N-Methylsulfuric diamide (47.1 mg, 0.428 mmol) and 1,8-diazabicyclo[5.4.0]undec-7-ene (0.065 mL 0.428 mmol, DBU) were added after the mixture was cooled down to room temperature, and the solution was stirred overnight at room temperature. The solution was acidified with 1 N HCl to pH=2-3 and extracted with ethyl acetate twice. The organic layers were combined and concentrated to give a residue which was purified by preparative HPLC (neutral phase, H₂O/CH₃CN) and lyophilized to give the titled compound (44.2 mg, 0.087 mmol, 61.3% yield). ¹H NMR (400 MHz, DMSO-d₆) δ ppm 11.06 (s, 1H), 7.40-6.91 (m, 6H), 6.49 (s, 2H), 3.76 (s, 6H), 3.29 (s, 4H), 2.51 (s, 2H), 2.48 (dd, J=2.1, 1.5 Hz, 3H), 2.23 (d, J=7.7 Hz, 2H), 2.00 (s, 3H), 1.94-1.76 (m, 2H), 1.52 (d, J=8.7 Hz, 4H); LC-MS (ESI) m/z 506.2 (M+H)⁺, RT=1.941 minutes.

### Example 71

### {4-[(3,5-dimethoxy-4-methylbenzoyl)(3-phenylpropyl)amino]butyl}phosphonic Acid and

### Example 72

{5-[(3,5-dimethoxy-4-methylbenzoyl)(3-phenylpropyl)amino]pentan-2-yl}phosphonic Acid

### Step 1: diethyl {4-[(3-phenylpropyl)amino]butyl}phosphonate

To a solution of diethyl (4-iodobutyl)phosphonate (2.0 g, 3.12 mmol) in tetrahydrofuran (10 mL) was added diisopropylethylamine (1.091 mL, 6.25 mmol) and 3-phenylpropan-1-amine (0.845 g, 6.25 mmol). The mixture was stirred at 70° C. under a nitrogen atmosphere for 1 hour. The solution was poured into water (10 mL), extracted with ethyl acetate (10 mL×3) and concentrated. The residue was purified by preparative-HPLC to the titled compound (600 mg, 1.833 mmol, 58.7% yield). ¹H NMR (400 MHz, CDCl₃) δ ppm 9.32 (s, 2H), 7.29 (dt, J=6.7, 1.3 Hz, 2H), 7.24-7.12 (m, 3H), 4.15-3.99 (m, 4H), 3.00-2.86 (m, 4H), 2.68 (t, J=7.6 Hz, 2H), 2.10 (p, J=7.6 Hz, 2H), 1.87 (t, J=7.4 Hz, 2H), 1.66 (d, J=7.6 Hz, 4H), 1.32 (q, J=7.0 Hz, 6H); MS (APCI⁺) m/z 328.2 (M+H)⁺.

### Step 2: diethyl {4-[(3,5-dimethoxy-4-methylbenzoyl)(3-phenylpropyl)amino]butyl}phosphonate

To a solution of 3,5-dimethoxy-4-methylbenzoic acid (1.226 g, 6.25 mmol) in dichloromethane (40 mL) was added 1-[bis(dimethylamino)methylene]-1H-1,2,3-triazolo[4,5-b]pyridinium 3-oxid hexafluorophosphate (2.376 g, 6.25 mmol, HATU) and diisopropylethylamine (2.183 mL, 12.50 mmol). The mixture was stirred at 25° C. under a nitrogen atmosphere for 1 hour. To this solution was added diethyl {4-[(3-phenylpropyl)amino]butyl}phosphonate (2.046 g, 6.25 mmol, Step 1), and the mixture was stirred for another 2 hours. The solution was poured into water (30 mL), extracted with ethyl acetate (40 mL×3) and concentrated. Flash chromatography on silica gel (50-100% ethyl acetate in n-hexane) gave the titled compound (2.4 g, 4.75 mmol, 76% yield). MS (APCI⁺) m/z 506.6 (M+H)⁺.

### Step 3: diethyl {5-[(3,5-dimethoxy-4-methylbenzoyl)(3-phenylpropyl)amino]pentan-2-yl}phosphonate and diethyl {4-[(3,5-dimethoxy-4-methylbenzoyl)(3-phenylpropyl)amino]butyl}phosphonate

To a solution of diethyl {4-[(3,5-dimethoxy-4-methylbenzoyl)(3-phenylpropyl)amino]butyl}phosphonate (250 mg, 0.494 mmol, Step 2) in tetrahydrofuran (30 mL) was added 1 M lithium diisopropylamide (1.483 mL, 1.483 mmol; tetrahydrofuran/hexanes, 1:7) dropwise at −78° C. The mixture was stirred at −78° C. for 1 hour. To the mixture was added methyl iodide (0.093 mL, 1.483 mmol), and the resulting mixture was stirred at −78° C. under a nitrogen atmosphere for 2 hours. The reaction was quenched with aqueous NH₄Cl. Then the mixture was poured into water (30 mL), extracted with ethyl acetate (30 mL×3) and concentrated. Flash chromatography (5-40% ethyl acetate in n-hexane followed by 2-7% methanol in dichloromethane) gave a mixture of diethyl {5-[(3,5-dimethoxy-4-methylbenzoyl)(3-phenylpropyl)amino]pentan-2-yl}phosphonate (200 mg, 0.192 mmol, 38.9% yield) MS (APCI⁺) m/z 520.6 (M+H)⁺ and diethyl {4-[(3,5-dimethoxy-4-methylbenzoyl)(3-phenylpropyl)amino]butyl}phosphonate (200 mg, 0.119 mmol, 24.00% yield) MS (APCI⁺) m/z 506.6 (M+H)⁺ that was used directly in the next step.

### Step 4: {4-[(3,5-dimethoxy-4-methylbenzoyl)(3-phenylpropyl)amino]butyl}phosphonic acid and {5-[(3,5-dimethoxy-4-methylbenzoyl)(3-phenylpropyl)amino]pentan-2-yl}phosphonic Acid

To a solution of diethyl {4-[(3,5-dimethoxy-4-methylbenzoyl)(3-phenylpropyl)amino]butyl}phosphonate (200 mg, 0.119 mmol) and diethyl {5-[(3,5-dimethoxy-4-methylbenzoyl)(3-phenylpropyl)amino]pentan-2-yl}phosphonate (200 mg, 0.192 mmol) in dichloromethane (10 mL) was added bromotrimethylsilane (459 mg, 3.0 mmol) dropwise at room temperature, and the resulting mixture was stirred overnight. To the mixture was added 5 mL of methanol and 0.5 mL of 28% ammonia in water, and the mixture was stirred for another 10 minutes. The mixture was concentrated, and the residue was purified by preparative-HPLC to give {4-[(3,5-dimethoxy-4-methylbenzoyl)(3-phenylpropyl)amino]butyl}phosphonic acid (21.6 mg, 0.047 mmol, 15.70% yield); ¹H NMR (400 MHz, DMSO-d₆) δ ppm 7.03-7.26 (5H, m, br), 6.48 (2H, s), 3.74 (6H, s), 3.38 (2H, s, br), 3.15 (2H, s, br), 2.61 (1H, s, br), 2.39 (1H, s, br), 1.98 (3H, s), 1.22-1.89 (6H, m), MS (APCI⁺) m/z 450.5 (M+H)⁺ and then {5-[(3,5-dimethoxy-4-methylbenzoyl)(3-phenylpropyl)amino]pentan-2-yl}phosphonic acid (17.2 mg, 0.036 mmol, 11.87% yield); ¹′H NMR (400 MHz, methanol-d₄) δ ppm 6.96-7.30 (5H, m), 6.47-6.64 (2H, m), 3.77-3.82 (6H, m), 3.51 (2H, s, br), 3.30 (3H, m), 3.28 (2H, m), 2.69 (1H, m), 2.45 (1H, m), 2.05 (3H, s), 1.03-1.89 (5H, m), MS (APCI⁺) m/z 464.5 (M+H)⁺.

### Example 73

### 1-(4-methoxyphenyl)-N-{5-[(methylsulfamoyl)amino]-5-oxopentyl}-N-(3-phenylpropyl)cyclopropane-1-carboxamide

### Step 1: 5-{[1-(4-methoxyphenyl)cyclopropane-1-carbonyl](3-phenylpropyl)amino}pentanoic Acid

1-(4-Methoxyphenyl)cyclopropanecarboxylic acid (100 mg, 0.521 mmol) and 1-[bis(dimethylamino)methylene]-1H-1,2,3-triazolo[4,5-b]pyridinium 3-oxid hexafluorophosphate (238 mg, 0.626 mmol, HATU) were dissolved in a mixture of dry N,N-dimethylformamide (1 mL), tetrahydrofuran (1.000 mL) and N,N-diisopropylethylamine (270 mg, 2.085 mmol). The mixture was stirred for 20 minutes at room temperature. Then Example 70—Step 1 (130 mg, 0.521 mmol) was added, and the solution was stirred overnight at room temperature. Then the mixture was poured into aqueous NH₄Cl and extracted with ethyl acetate 3 times. The combined organic fractions were washed with brine dried over Na₂SO₄, and concentrated. The residue was re-dissolved in tetrahydrofuran, and treated with 1 N LiOH for 2 hours. The mixture was purified by flash column chromatography on silica gel (loaded directly with CH₂Cl₂) eluted with hexanes and ethyl acetate (0-50%) to give the titled compound (63 mg, 0.154 mmol, 29.5% yield). ¹H NMR (400 MHz, DMSO-d₆) δ ppm 7.26 (t, J=7.6 Hz, 2H), 7.19 (d, J=7.8 Hz, 2H), 7.10 (d, J=7.9 Hz, 1H), 7.01 (dd, J=14.0, 7.9 Hz, 2H), 6.86 (t, J=6.4 Hz, 2H), 3.72 (s, 4H), 2.27 (d, J=8.1 Hz, 1H), 2.21 (s, 1H), 2.01 (s, 1H), 1.73 (s, 1H), 1.42 (s, 3H), 1.35 (s, 1H), 1.18 (s, 2H), 1.10 (d, J=23.5 Hz, 3H), 0.93 (d, J=5.2 Hz, 1H).

### Step 2: 1-(4-methoxyphenyl)-N-{5-[(methylsulfamoyl)amino]-5-oxopentyl}-N-(3-phenylpropyl)cyclopropane-1-carboxamide

Material from Example 73—Step 1 (40 mg, 0.098 mmol) was dissolved in tetrahydrofuran (5 mL) and 1,1′-carbonyldiimidazole (47.5 mg, 0.293 mmol) was added. The mixture was heated to 60° C. for 1 hour, then 1,8-diazabicyclo[5.4.0]undec-7-ene (44.6 mg, 0.293 mmol) and N-methylsulfuric diamide (32.3 mg, 0.293 mmol) were added followed by stirring at room temperature for 12 hours. The mixture was concentrated, and the residue was dissolved in CH₃OH (1.3 mL) to which a couple of drops of acetic acid were added to adjust the pH to 6-7. The filtered solution was purified by preparative HPLC (0.1% CF₃CO₂H/H₂O/CH₃CN) to give the titled compound (28 mg, 0.056 mmol, 57.1% yield). ¹H NMR (400 MHz, DMSO-d₆) δ ppm 11.25 (s, 1H), 7.42-7.36 (m, 1H), 7.33-7.08 (m, 4H), 7.06-6.96 (m, 2H), 6.86 (d, J=7.6 Hz, 2H), 3.73 (s, 2H), 3.26-3.16 (m, 3H), 2.46 (s, 2H), 2.32-2.20 (m, 2H), 2.09 (t, J=7.2 Hz, 1H), 1.73 (s, OH), 1.43 (s, 2H), 1.34 (s, 1H), 1.30-1.21 (m, 1H), 1.18 (s, 1H), 1.13 (s, 1H), 1.07 (s, 1H), 0.93 (s, 1H).

### Example 74

### 3,5-dimethoxy-N-{5-[(methylsulfamoyl)amino]-5-oxopentyl}-N-(3-phenylpropyl)benzamide

### Step 1: methyl 5-[(3,5-dimethoxybenzoyl)(3-phenylpropyl)amino]pentanoate

3,5-Dimethoxybenzoic acid (100 mg, 0.549 mmol) and 1-[bis(dimethylamino)methylene]-1H-1,2,3-triazolo[4,5-b]pyridinium 3-oxid hexafluorophosphate (250 mg, 0.659 mmol, HATU) were dissolved in a mixture of dry N,N-dimethylformamide (1 mL), tetrahydrofuran (1.000 mL) and N,N-diisopropylethylamine (213 mg, 1.647 mmol). The mixture was stirred for 20 minutes at room temperature. Then Example 70—Step 1 (137 mg, 0.549 mmol) was added, and the mixture was stirred for another 3 hours at room temperature. The mixture was poured into water and extracted with ethyl acetate 3 times. The combined organic fractions were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (160 mg, 0.387 mmol, 70.5% yield).

### Step 2: 5-[(3,5-dimethoxybenzoyl)(3-phenylpropyl)amino]pentanoic Acid

Step 1 (80 mg, 0.193 mmol) was dissolved in tetrahydrofuran (2 mL) and CH₃OH (0.2 mL), and then 1 N LiOH (1.0 mL) was added. The mixture was stirred at room temperature for 3 hours and then concentrated. The residue was treated with water (5 mL) followed by the addition of 1 N hydrochloric acid to adjust the pH to 5, and then the aqueous phase was extracted with ethyl acetate. The organic layer was washed with brine, dried, and concentrated. The residue was purified by preparative HPLC (0.1% CF₃CO₂H) to provide the titled compound (50 mg, 0.125 mmol, 64.7% yield). ¹H NMR (400 MHz, DMSO-d₆) δ ppm 7.23 (d, J=31.8 Hz, 4H), 7.05 (d, J=5.7 Hz, 1H), 6.51 (s, 1H), 6.40 (s, 2H), 3.75 (s, 6H), 3.14 (s, 3H), 2.62 (s, 1H), 2.23 (s, 1H), 2.05 (s, 1H), 1.83 (d, J=36.0 Hz, 3H), 1.53 (m, 4H), 1.26 (d, J=14.8 Hz, 2H); LC-MS (ESI) m/z 400.2 (M+H)⁺.

### Step 3: 3,5-dimethoxy-N-{5-[(methylsulfamoyl)amino]-5-oxopentyl}-N-(3-phenylpropyl)benzamide

The material from Example 74—Step 2 (40 mg, 0.100 mmol) was dissolved in tetrahydrofuran (5 mL), and 1,1′-carbonyldiimidazole (48.7 mg, 0.300 mmol) was added. The mixture was heated to 60° C. for 1 hour, and then 1,8-diazabicyclo[5.4.0]undec-7-ene (45.7 mg, 0.300 mmol) and N-methyl sulfonamide (33.1 mg, 0.300 mmol) were added followed by stirring at room temperature for 12 hours. The mixture was concentrated, and the residue was dissolved in CH₃OH (1.3 mL) to which a couple drops of acetic acid were added to adjust the pH to 6-7. The filtered solution was purified by preparative HPLC (0.1% CF₃CO₂H/H₂O/CH₃CN) to give the titled compound (26 mg, 0.053 mmol, 52.8% yield). ¹H NMR (400 MHz, DMSO-d₆) δ ppm 11.26 (d, J=16.9 Hz, 2H), 7.40 (d, J=5.9 Hz, 2H), 7.34-7.11 (m, 8H), 7.05 (d, J=7.3 Hz, 2H), 6.52 (s, 2H), 6.40 (s, 4H), 3.75 (s, 11H), 3.19-3.11 (m, 3H), 2.67-2.58 (m, 2H), 2.44 (dt, J=30.5, 6.4 Hz, 11H), 2.30 (s, 2H), 2.15 (s, 1H), 1.88 (s, 1H), 1.79 (s, 1H), 1.55 (s, 3H), 1.46 (s, 1H), 1.32 (s, 1H); LC-MS (ESI) m/z 492.2 (M+H)⁺.

### Example 75

### {4-[(3,5-dimethoxybenzoyl)(3-phenylpropyl)amino]butyl}phosphonic Acid and

### Example 76

### ethyl hydrogen {4-[(3,5-dimethoxybenzoyl)(3-phenylpropyl)amino]butyl}phosphonate

### Step 1: diethyl {4-[(3,5-dimethoxybenzoyl)(3-phenylpropyl)amino]butyl}phosphonate

To a stirred solution of 3,5-dimethoxybenzoic acid (107 mg, 0.589 mmol) in dichloromethane (2 mL) was added 1-[bis(dimethylamino)methylene]-1H-1,2,3-triazolo[4,5-b]pyridinium 3-oxid hexafluorophosphate (224 mg, 0.589 mmol, HATU), diisopropylethylamine (0.317 mL, 1.812 mmol) and diethyl {4-[(3-phenylpropyl)amino]butyl}phosphonate (200 mg, 0.453 mmol, Example 71/72—Step 1) at room temperature, and the resulting mixture was stirred overnight. The solution was poured into water (˜20 mL), extracted with ethyl acetate (2×20 mL) and concentrated. Preparative-HPLC gave the titled compound (202 mg, 0.390 mmol, 86% yield). ¹H NMR (400 MHz, DMSO-d₆) δ ppm 7.34-6.98 (m, 5H), 6.56-6.43 (m, 1H), 6.42-6.30 (m, 2H), 3.93 (s, 4H), 3.39 (s, 2H), 3.13 (s, 2H), 2.61 (s, 1H), 2.38 (s, 1H), 1.77 (s, 3H), 1.56 (d, J=36.2 Hz, 4H), 1.32-1.09 (m, 7H); MS (APCI⁺) m/z 492.2 (M+H)⁺.

### Step 2: {4-[(3,5-dimethoxybenzoyl)(3-phenylpropyl)amino]butyl}phosphonic acid and ethyl hydrogen {4-[(3,5-dimethoxybenzoyl)(3-phenylpropyl)amino]butyl}phosphonate

To a solution of diethyl {4-[(3,5-dimethoxybenzoyl)(3-phenylpropyl)amino]butyl}phosphonate (175 mg, 0.356 mmol) in dichloromethane (10 mL) was added bromotrimethylsilane (545 mg, 3.56 mmol) at 0° C. The mixture was stirred at 0° C. under a nitrogen atmosphere for 16 hours, and then it was warmed to room temperature with stirring continued for an additional 3 hours. To the reaction mixture was added methanol (10 mL) and 28% ammonia (0.5 mL), and resultant mixture was concentrated. Preparative-HPLC gave {4-[(3,5-dimethoxybenzoyl)(3-phenylpropyl)amino]butyl}phosphonic acid (51.9 mg, 0.117 mmol, 32.8% yield), ¹H NMR (400 MHz, DMSO-d₆) δ ppm 7.29-7.02 (m, 5H), 6.49 (s, 1H), 6.38 (d, J=8.2 Hz, 2H), 3.73 (s, 6H), 3.12 (s, 2H), 2.60 (d, J=8.2 Hz, 1H), 2.38 (s, 1H), 1.81 (d, J=35.0 Hz, 2H), 1.55 (d, J=39.8 Hz, 4H), 1.38-1.21 (m, 2H), m/z 436.4 (M+H)⁺; and then ethyl hydrogen {4-[(3,5-dimethoxybenzoyl)(3-phenylpropyl)amino]butyl}phosphonate (87.5 mg, 0.185 mmol, 52.0% yield), ¹H NMR (400 MHz, DMSO-d₆) δ ppm 7.30-7.02 (m, 5H), 6.50 (s, 1H), 6.38 (d, J=7.2 Hz, 2H), 3.87 (d, J=20.7 Hz, 2H), 3.73 (s, 6H), 3.38 (s, 2H), 3.12 (d, J=7.8 Hz, 2H), 2.61 (s, 1H), 2.38 (s, 1H), 1.77 (s, 2H), 1.60 (s, 5H), 1.30-1.11 (m, 4H), MS (APCI⁺) m/z 464.46 (M+H)⁺.

### Example 77

### (−)-(2R)-5-[(3,5-dimethoxy-4-methylbenzoyl)(3-phenylpropyl)amino]-2-methylpentanoic Acid

### Step 1: benzyl 5-[(3,5-dimethoxy-4-methylbenzoyl)(3-phenylpropyl)amino]-2-methylpentanoate

Thionyl chloride (40 mL, 548 mmol) was added to 5-[(3,5-dimethoxy-4-methylbenzoyl)(3-phenylpropyl)amino]-2-methylpentanoic acid (Example 54, 10.0 g, 23.39 mmol), and the resulting solution was heated at 75° C. for 2.5 hours. The solution was cooled to room temperature and concentrated under vacuum. The residue was dissolved in CH₂Cl₂ (40 mL) and concentrated again to remove excess SOCl₂. Finally, the crude acid chloride was dissolved in CH₂Cl₂ (40 mL) and stirred with ice cooling. Pyridine (5.68 mL, 70.2 mmol) was added, followed by benzyl alcohol (4.86 mL, 46.8 mmol). The cold bath was removed, and the resulting mixture was allowed to stir at room temperature for 12 hours. The reaction solution was diluted with CH₂Cl₂ (60 mL) and washed successively with 8% aqueous H₂SO₄ (80 mL) and 20% aqueous K₂CO₃ (40 mL). The organic phase was dried over Na₂SO₄ and concentrated under vacuum. The residue was purified by flash chromatography (120 g silica cartridge eluted with heptanes-ethyl acetate, 95:5-65:35) to provide the titled compound (9.52 g). Thin-layer chromatography R_{f}˜0.42 in heptanes-ethyl acetate, 67:33; MS (ESI+) m/z 518 (M+H)⁺.

### Step 2: benzyl 5-[(3,5-dimethoxy-4-methylbenzoyl)(3-phenylpropyl)amino]-2-methylpentanoate—Isomer 1 and Isomer 2

Benzyl 5-[(3,5-dimethoxy-4-methylbenzoyl)(3-phenylpropyl)amino]-2-methylpentanoate was separated into the individual enantiomers by preparative chiral HPLC, using a 30 mm ID×250 mm Chiralcel® OJ-H column and the following elution parameters: Mobile phase: A: Hexanes; B:

### Methanol-isopropanol (80:20)

Isocratic elution A/B=30:70 at 30 mL/minute

### Detection: UV at 254 nm

Sample loading (each injection): 159 mg in methanol (1 mL)

The fractions eluting at 6.2-7.9 minutes were pooled and concentrated to give benzyl 5-[(3,5-dimethoxy-4-methylbenzoyl)(3-phenylpropyl)amino]-2-methylpentanoate—isomer 1, 3.90 g.

The fractions eluting at 9-13.2 minutes were pooled and concentrated to give benzyl 5-[(3,5-dimethoxy-4-methylbenzoyl)(3-phenylpropyl)amino]-2-methylpentanoate—isomer 2, 2.82 g.

### Step 3: (−)-(2R)-5-[(3,5-dimethoxy-4-methylbenzoyl)(3-phenylpropyl)amino]-2-methylpentanoic Acid

Benzyl 5-(3,5-dimethoxy-4-methyl-N-(3-phenylpropyl)benzamido)-2-methylpentanoate (Step 2-isomer 1, 3.89 g) was dissolved in tetrahydrofuran (120 mL). 20% Pd(OH)₂/C (wet, 6.23 g) was added, and the mixture was agitated on a Parr shaker under H₂ (50 psi) for 50 hours until debenzylation was complete. The mixture was filtered through a polypropylene membrane, and the filtrate was concentrated under vacuum. The residue was purified by flash chromatography (80 g silica eluted with CH₂Cl₂—CH₃OH, 99:1-95:5) to provide a gum which was solidified from heptanes-ethyl acetate (8 mL, 60:40) to provide the titled compound (1.4 g). ¹H NMR (400 MHz, DMSO-d₆, T=120° C.) 6 ppm 7.28-7.18 (m, 2H), 7.16-7.06 (m, 3H), 6.50 (s, 2H), 3.76 (s, 6H), 3.36-3.27 (m, 4H), 2.54 (t, J=7.5 Hz, 2H), 2.29 (q, J=7.0 Hz, 1H), 2.02 (s, 3H), 1.87 (p, J=7.5 Hz, 2H), 1.67-1.43 (m, 3H), 1.42-1.19 (m, 1H), 1.04 (d, J=7.0 Hz, 3H); MS (APCI) m/z 428 (M+H)⁺; [α]_{D}^{22.1}=−6.7, c=1 (CH₃OH); Analytical chiral HPLC elution 4.084 minutes.

### Example 78

### (+)-(2S)-5-[(3,5-dimethoxy-4-methylbenzoyl)(3-phenylpropyl)amino]-2-methylpentanoic Acid

The title compound was prepared by a procedure similar to that described for Example 77, but starting with benzyl 5-[(3,5-dimethoxy-4-methylbenzoyl)(3-phenylpropyl)amino]-2-methylpentanoate Example 77—Step 2-isomer 2. ¹H NMR (400 MHz, DMSO-d₆, T=120° C.) 6 ppm 7.25-7.16 (m, 2H), 7.12 (t, J=6.1 Hz, 3H), 6.50 (s, 2H), 3.76 (s, 6H), 3.37-3.23 (m, 4H), 2.54 (t, J=7.6 Hz, 2H), 2.36-2.22 (m, 1H), 2.02 (s, 3H), 1.86 (p, J=7.7 Hz, 2H), 1.62-1.47 (m, 3H), 1.37-1.20 (m, 1H), 1.04 (d, J=7.0 Hz, 3H); MS (APCI) m/z 428 (M+H)⁺; [α]_{D}^{22.1}=+7.6, c=1 (CH₃OH); Analytical chiral HPLC elution 4.538 minutes.

### Example 79

### 5-[(3-fluoro-4-methoxybenzoyl)(3-phenylpropyl)amino]pentanoic Acid

### Step 1: methyl 5-[(3-fluoro-4-methoxybenzoyl)(3-phenylpropyl)amino]pentanoate

3-Fluoro-4-methoxybenzoic acid (68.2 mg, 0.401 mmol) and 1-[bis(dimethylamino)methylene]-1H-1,2,3-triazolo[4,5-b]pyridinium 3-oxid hexafluorophosphate (183 mg, 0.481 mmol, HATU) were dissolved in a mixture of dry N,N-dimethylformamide (1 mL), tetrahydrofuran (1.000 mL) and N,N-diisopropylethylamine (155 mg, 1.203 mmol). The mixture was stirred for 20 minutes at room temperature. Then Example 70—Step 1 (100 mg, 0.401 mmol) was added, and the mixture was stirred for another 3 hours at room temperature. The mixture was poured into water and extracted with ethyl acetate 3 times. The combined organic layers were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (120 mg, 0.299 mmol, 74.5% yield).

### Step 2: 5-[(3-fluoro-4-methoxybenzoyl)(3-phenylpropyl)amino]pentanoic Acid

Material from Example 79—Step 1 (120 mg, 0.299 mmol) was dissolved in tetrahydrofuran (2 mL), and 1 N lithium hydroxide (42.9 mg, 1.793 mmol, 2 mL) was added. The mixture was stirred for 2 hours at 45° C. The mixture was poured into water, the pH was adjusted to 7, and the mixture was extracted with ethyl acetate 3 times. The combined organic layers were washed with brine, dried over Na₂SO₄, and concentrated. The residue was purified by preparative HPLC with acetonitrile and water (0.5% CF₃CO₂H) to give the titled compound (86 mg, 0.222 mmol, 74.3% yield). ¹H NMR (400 MHz, DMSO-d₆) δ ppm 11.79 (s, 1H), 7.27-7.03 (m, 8H), 3.86 (s, 3H), 3.2(m, 1H), 2.52 (s, 1H), 2.16 (t, J=7.3 Hz, 2H), 1.83 (p, J=7.7 Hz, 2H), 1.58-1.46 (m, 2H), 1.42 (s, 2H); LC-MS (ESI) m/z 387.2 (M+H)⁺.

### Example 80

### 5-[(2H-1,3-benzodioxole-5-carbonyl)(3-phenylpropyl)amino]pentanoic Acid

### Step 1: methyl 5-[(2H-1,3-benzodioxole-5-carbonyl)(3-phenylpropyl)amino]pentanoate

To a solution of benzo[d][1,3]dioxole-5-carboxylic acid (95 mg, 0.572 mmol) and 1-[bis(dimethylamino)methylene]-1H-1,2,3-triazolo[4,5-b]pyridinium 3-oxid hexafluorophosphate (228 mg, 0.601 mmol, HATU) in N,N-dimethylformamide (3 mL) was added triethylamine (0.084 mL 0.601 mmol). The resulting solution was stirred at room temperature for 10 minutes. Then Example 70—Step 1 (143 mg, 0.572 mmol) was added in one portion. The solution was stirred at room temperature overnight. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (200 mg, 0.503 mmol, 88% yield). LC-MS (ESI) m/z 398.2 (M+H)⁺, RT=1.977 minutes.

### Step 2: 5-[(2H-1,3-benzodioxole-5-carbonyl)(3-phenylpropyl)amino]pentanoic Acid

To a solution of Example 80—Step 1 (200 mg, 0.503 mmol) in tetrahydrofuran (3 mL) was added 1 N lithium hydroxide (3.02 mL 3.02 mmol). It was stirred at room temperature for 2 hours. The mixture was cooled down and acidified with 1 N HCl to pH=2-3 and then extracted with ethyl acetate twice. The combined organic layers were concentrated to give a residue which was purified by preparative HPLC (0.1% CF₃CO₂H—H₂O/CH₃CN) and lyophilized to give the titled compound (119.6 mg, 0.312 mmol, 62.0% yield). ¹H NMR (400 MHz, DMSO-d₆) δ ppm 7.23 (dd, J=8.3, 6.5 Hz, 2H), 7.15 (td, J=7.8, 6.5, 3.5 Hz, 3H), 6.87 (d, J=7.9 Hz, 1H), 6.82 (d, J=1.6 Hz, 1H), 6.77 (dd, J=7.9, 1.6 Hz, 1H), 6.03 (s, 2H), 3.29 (s, 4H), 2.50 (s, 2H), 2.16 (t, J=7.1 Hz, 2H), 1.82 (p, J=7.7 Hz, 2H), 1.50 (d, J=8.4 Hz, 2H), 1.42 (s, 2H); LC-MS (ESI) m/z 384.2 (M+H)⁺, RT=1.831 minutes.

### Example 81

### 5-[(4-fluoro-3-methoxybenzoyl)(3-phenylpropyl)amino]pentanoic Acid

### Step 1: methyl 5-[(4-fluoro-3-methoxybenzoyl)(3-phenylpropyl)amino]pentanoate

To a solution of 4-fluoro-3-methoxybenzoic acid (97 mg, 0.572 mmol) and 1-[bis(dimethylamino)methylene]-1H-1,2,3-triazolo[4,5-b]pyridinium 3-oxid hexafluorophosphate (228 mg, 0.601 mmol, HATU) in N,N-dimethylformamide (3 mL) was added triethylamine (0.084 mL 0.601 mmol). The resulting solution was stirred at room temperature for 10 minutes. Then Example 70—Step 1 (143 mg, 0.572 mmol) was added in one portion. The solution was stirred at room temperature overnight. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (184 mg, 0.458 mmol, 80% yield). LC-MS (ESI) m/z 402.2 (M+H)⁺, RT=2.012 minutes.

### Step 2: 5-[(4-fluoro-3-methoxybenzoyl)(3-phenylpropyl)amino]pentanoic Acid

To a solution of Example 81—Step 1 (184 mg, 0.458 mmol) in tetrahydrofuran (3 mL) was added 1 N lithium hydroxide (2.75 mL 2.75 mmol). The mixture was stirred at room temperature for 2 hours. The mixture was cooled down and acidified with 1 N HCl to pH=2-3 and then extracted with ethyl acetate twice. The combined organic layers were concentrated to give a residue which was purified by preparative HPLC(0.1% CF₃CO₂H—H₂O/CH₃CN) and lyophilized twice to give the titled compound (135 mg, 0.348 mmol, 76% yield). ¹H NMR (400 MHz, DMSO-d₆) δ ppm 7.44-7.04 (m, 6H), 7.04 (dd, J=8.3, 2.0 Hz, 1H), 6.84 (ddd, J=8.2, 4.3, 1.9 Hz, 1H), 3.83 (s, 3H), 3.28 (s, 4H), 2.50 (s, 2H), 2.16 (s, 2H), 1.84 (s, 2H), 1.52 (d, J=5.7 Hz, 2H), 1.43 (s, 2H); LC-MS (ESI) m/z 388.2 (M+H)⁺, RT=1.861 minutes.

### Example 82

### 5-{[1-(3-methoxyphenyl)cyclopropane-1-carbonyl](3-phenylpropyl)amino}pentanoic Acid

### Step 1: methyl 5-{[1-(3-methoxyphenyl)cyclopropane-1-carbonyl](3-phenylpropyl)amino}pentanoate

To a solution of 1-(3-methoxyphenyl)cyclopropanecarboxylic acid (110 mg, 0.572 mmol) in N,N-dimethylformamide (3 mL) was added 1-[bis(dimethylamino)methylene]-1H-1,2,3-triazolo[4,5-b]pyridinium 3-oxid hexafluorophosphate (217 mg, 0.572 mmol, HATU) and diisopropylethylamine (0.500 mL 2.86 mmol), and the mixture was stirred at room temperature for 15 minutes. Then a solution of Example 70—Step 1 (143 mg, 0.572 mmol) in N,N-dimethylformamide (2 mL) was added to the above solution. The mixture was stirred at room temperature overnight. The mixture was partitioned between water (5 mL) and ethyl acetate (10 mL). The organic layer was dried over Na₂SO₄, filtered and concentrated to give a residue which was purified by flash chromatography on silica gel (loaded directly with CH₂Cl₂) eluted with hexanes and ethyl acetate (0-50%) to give the titled compound (200 mg, 0.472 mmol, 83% yield). LC-MS (ESI) m/z 424.5 (M+H)⁺, RT=2.09 minutes.

### Step 2: 5-{[1-(3-methoxyphenyl)cyclopropane-1-carbonyl](3-phenylpropyl)amino}pentanoic Acid

To a solution of the material from Example 82—Step 1 (200 mg, 0.472 mmol) in tetrahydrofuran (3 mL) was added 1 N LiOH (1.5 mL). The mixture was stirred at room temperature overnight. Then 1 N HCl was added to the mixture to adjust pH=2-3. The mixture was extracted with ethyl acetate (30 mL). The organic layer was dried over Na₂SO₄, filtered and concentrated. The residue was purified by high pressure liquid chromatography with acetonitrile and water (0.5% CF₃CO₂H) to give the titled compound (95 mg, 0.232 mmol, 49.1% yield). ¹H NMR (400 MHz, DMSO-d₆) δ ppm 11.76 (s, 1H), 7.20 (ddd, J=26.1, 12.4, 7.3 Hz, 5H), 7.03 (s, 1H), 6.77 (dd, J=8.2, 2.5 Hz, 1H), 6.66 (s, 2H), 3.71 (s, 3H), 3.22 (q, J=8.1, 7.7 Hz, 4H), 2.50-2.48 (m, 1H), 2.39-1.93 (m, 3H), 1.75 (brs, 1H), 1.44 (brs, 3H), 1.19 (brs, 4H), 1.03 (brs, 2H); LC-MS (ESI) m/z 410.5 (M+H)⁺, RT=1.93 minutes.

### Example 83

### 5-[(3,4-dimethoxybenzoyl)(3-phenylpropyl)amino]pentanoic Acid

### Step 1: methyl 5-[(3,4-dimethoxybenzoyl)(3-phenylpropyl)amino]pentanoate

To a solution of 3,4-dimethoxybenzoic acid (104 mg, 0.572 mmol) and 1-[bis(dimethylamino)methylene]-1H-1,2,3-triazolo[4,5-b]pyridinium 3-oxid hexafluorophosphate (228 mg, 0.601 mmol, HATU) in N,N-dimethylformamide (3 mL) was added triethylamine (0.084 mL 0.601 mmol). The resulting solution was stirred at room temperature for 10 minutes. Then Example 70—Step 1 (143 mg, 0.572 mmol) was added in one portion. The solution was stirred at room temperature overnight. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (86 mg, 0.208 mmol, 36.4% yield). LC-MS (ESI) m/z 414.2 (M+H)⁺, RT=1.937 minutes.

### Step 2: 5-[(3,4-dimethoxybenzoyl)(3-phenylpropyl)amino]pentanoic Acid

To a solution of Example 83—Step 1 (86 mg, 0.208 mmol) in tetrahydrofuran (3 mL) was added 1 N LiOH (1.2 mL). The mixture was stirred at room temperature overnight. Then the mixture was acidified with 1 N HCl, and the mixture was extracted with ethyl acetate (20 mL). The organic layer was dried over Na₂SO₄, filtered and concentrated to give the crude which was purified by high pressure liquid chromatography with acetonitrile and water (0.5% CF₃CO₂H) to give the titled compound (73 mg, 0.183 mmol, 88% yield). ¹H NMR (400 MHz, DMSO-d₆) δ ppm 7.23 (t, J=7.5 Hz, 2H), 7.14 (dd, J=7.5, 5.3 Hz, 3H), 6.93 (d, J=8.1 Hz, 1H), 6.89-6.80 (m, 2H), 3.78 (s, 3H), 3.75 (s, 3H), 3.30 (t, J=7.2 Hz, 4H), 2.51 (d, J=7.3 Hz, 2H), 2.16 (t, J=7.2 Hz, 2H), 1.84 (p, J=7.7 Hz, 2H), 1.53 (p, J=7.3 Hz, 2H), 1.43 (s, 2H); LC-MS (ESI) m/z 400.5 (M+H)⁺, RT=1.788 minutes.

### Example 84

### 5-[(4-methoxybenzoyl)(3-phenylpropyl)amino]pentanoic Acid

### Step 1: methyl 5-[(4-methoxybenzoyl)(3-phenylpropyl)amino]pentanoate

To a solution of 4-methoxybenzoic acid (76 mg, 0.500 mmol) in N,N-dimethylformamide (3 mL) was added 1-[bis(dimethylamino)methylene]-1H-1,2,3-triazolo[4,5-b]pyridinium 3-oxid hexafluorophosphate (190 mg, 0.500 mmol, HATU), and the mixture was stirred at room temperature for 15 minutes. Then a solution of Example 70—Step 1 (125 mg, 0.50 mmol) (CF₃CO₂H salt) and diisopropylethylamine (0.262 mL 1.500 mmol) in N,N-dimethylformamide (1 mL) was added to the mixture. The mixture was stirred at room temperature for 2 hours. Then water was added to the mixture, and the mixture was extracted with ethyl acetate (20 mL). The organic layer was washed with brine, dried over Na₂SO₄, filtered and concentrated. The residue was purified by flash chromatography with hexane and ethyl acetate (0-40%) to give the titled compound (157 mg, 0.409 mmol, 82% yield). LC-MS (ESI) m/z 384.5 (M+H)⁺, RT=2.000 minutes.

### Step 2: 5-[(4-methoxybenzoyl)(3-phenylpropyl)amino]pentanoic Acid

To a solution of material from Example 84—Step 1 (157 mg, 0.409 mmol) in 1,4-dioxane (3 mL) and CH₃OH (2 mL) was added 1 N LiOH (2 mL). The mixture was stirred at room temperature overnight. Then 1 N HCl was added to the mixture to adjust pH=3-4. Then the mixture was concentrated in vacuum, and the residue was extracted with ethyl acetate (30 mL). The organic layer was dried over Na₂SO₄, filtered and concentrated to give a residue which was purified by high pressure liquid chromatography with acetonitrile and water (0.5% CF₃CO₂H) to give the titled compound (100 mg, 0.271 mmol, 66.1% yield). ¹H NMR (400 MHz, DMSO-d₆) δ ppm 7.25-7.21 (m, 4H), 7.19-7.07 (m, 3H), 6.98-6.86 (m, 2H), 3.78 (s, 3H), 3.32-3.28 (m, 4H), 2.59-2.50 (m, 2H), 2.24-2.09 (m, 2H), 1.87-1.79 (m, 2H), 1.56-1.48 (m, 2H), 1.42 (brs, 2H); LC-MS (ESI) m/z 370.2 (M+H)⁺.

### Example 85

### 5-{[2-(4-methoxyphenyl)-2-methylpropanoyl](3-phenylpropyl)amino}pentanoic Acid

### Step 1: methyl 5-{[2-(4-methoxyphenyl)-2-methylpropanoyl](3-phenylpropyl)amino}pentanoate

To a solution of 2-(4-methoxyphenyl)-2-methylpropanoic acid (27.3 mg, 0.140 mmol) and 1-[bis(dimethylamino)methylene]-1H-1,2,3-triazolo[4,5-b]pyridinium 3-oxid hexafluorophosphate (53.4 mg, 0.140 mmol, HATU) in N,N-dimethylformamide (3 mL) was added diisopropylethylamine (0.025 mL 0.140 mmol). The resulting solution was stirred at room temperature for 10 minutes. Then Example 70—Step 1 (35 mg, 0.140 mmol) was added in one portion. The solution was stirred at room temperature overnight. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (50 mg, 0.117 mmol, 84% yield). LC-MS (ESI) m/z 426.2 (M+H)⁺, RT=1.944 minutes.

### Step 2: 5-{[2-(4-methoxyphenyl)-2-methylpropanoyl](3-phenylpropyl)amino}pentanoic Acid

To a solution of the material of Example 85—Step 1 (50 mg, 0.117 mmol) in 1,4-dioxane (0.5 mL) was added 1 N LiOH (0.5 mL 0.500 mmol). The resulting solution was stirred at 30° C. for 2 hours. The reaction was quenched with CF₃CO₂H until pH-7-8 and then purified by preparative HPLC (0.1% CF₃CO₂H—H₂O/CH₃CN) to give the titled compound (26 mg, 0.063 mmol, 53.8% yield). ¹H NMR (400 MHz, DMSO-d₆) δ ppm 7.32-6.83 (m, 9H), 3.72 (s, 3H), 3.16 (s, 2H), 2.81 (s, 2H), 2.10 (d, J=58.1 Hz, 3H), 1.71 (s, 2H), 1.46-1.20 (m, 9H), 0.98 (s, 2H); LC-MS (ESI) m/z 412.2 (M+H)⁺.

### Example 86

### 5-{[1-(4-methoxyphenyl)cyclobutane-1-carbonyl](3-phenylpropyl)amino}pentanoic Acid

### Step 1: methyl 5-{[1-(4-methoxyphenyl)cyclobutane-1-carbonyl](3-phenylpropyl)amino}pentanoate

The material of Example 70—Step 1 (0.143 mmol) and 1-(4-methoxyphenyl)cyclobutanecarboxylic acid (30 mg, 0.145 mmol) was dissolved in N,N-dimethylformamide (3 mL). 1-[Bis(dimethylamino)methylene]-1H-1,2,3-triazolo[4,5-b]pyridinium 3-oxid hexafluorophosphate (54.4 mg, 0.143 mmol, HATU) and diisopropylethylamine (0.050 mL 0.286 mmol) were added into the mixture. The resulting solution was stirred at room temperature for 1 hour. It was diluted with water and extracted with ethyl acetate twice. The combined organic layers were washed with brine 3 times, dried over anhydrous Na₂SO₄, filtered and concentrated to give the titled compound (50 mg, 0.114 mmol, 80% yield). LC-MS (ESI) m/z 438.2 (M+H)⁺.

### Step 2: 5-{[1-(4-methoxyphenyl)cyclobutane-1-carbonyl](3-phenylpropyl)amino}pentanoic Acid

To a solution of the material of Example 86—Step 1 (50 mg, 0.114 mmol) in 1,4-dioxane (0.5 mL) was added 1 N LiOH (0.5 mL 0.500 mmol) dropwise. The mixture was stirred at 30° C. for 1 hour. The reaction was quenched with CF₃CO₂H until pH-7-8 and then purified by preparative HPLC eluted with CH₃CN/H₂O/NH₄OH to give the titled compound (40 mg, 0.094 mmol, 83% yield). ¹H NMR (400 MHz, methanol-d₄) δ ppm 7.33-7.09 (m, 6H), 7.01-6.85 (m, 3H), 3.78 (s, 3H), 3.30-3.25 (m, 2H), 2.89 (q, J=7.2 Hz, 2H), 2.82-2.63 (m, 2H), 2.57 (t, J=7.8 Hz, 1H), 2.35 (ddd, J=12.3, 9.3, 3.7 Hz, 1H), 2.29-2.13 (m, 3H), 2.04-1.70 (m, 4H), 1.55 (p, J=3.0 Hz, 2H), 1.35-1.19 (m, 2H); LC-MS (ESI) m/z 424.2 (M+H)⁺.

### Example 87

### (2-{[1-(4-methoxyphenyl)cyclopropane-1-carbonyl](3-phenylpropyl)amino}ethoxy)acetic Acid

### Step 1: N-(2-hydroxyethyl)-1-(5-methoxypyridin-2-yl)-N-(3-phenylpropyl)cyclopropane-1-carboxamide

A solution of tert-butyl (2-hydroxyethyl)(3-phenylpropyl)carbamate (200 mg, 0.716 mmol, Accela ChemBio Co., Ltd) in hydrochloric acid/ethyl acetate (0.5 mL 2.000 mmol) was stirred at room temperature for 2 hours and then concentrated. The residue was dissolved in N,N-dimethylformamide (3 mL), and then 1-(5-methoxypyridin-2-yl)cyclopropanecarboxylic acid (138 mg, 0.716 mmol), 1-[bis(dimethylamino)methylene]-1H-1,2,3-triazolo[4,5-b]pyridinium 3-oxid hexafluorophosphate (272 mg, 0.716 mmol, HATU) and diisopropylethylamine (0.250 mL 1.432 mmol) were added thereto. The resulting solution was stirred at room temperature for 1 hour. It was diluted with water and extracted with ethyl acetate twice. The combined organic layers were washed with brine 3 times, dried over anhydrous Na₂SO₄, filtered and concentrated to give a residue. The residue was purified by flash chromatography on silica gel eluted with ethyl acetate in hexane from 30% to 100% to give the titled compound (150 mg, 0.423 mmol, 59.1% yield). LC-MS (ESI) m/z 354.2 (M+H)⁺.

### Step 2: methyl (2-{[1-(4-methoxyphenyl)cyclopropane-1-carbonyl](3-phenylpropyl)amino}ethoxy)acetate

To a solution of the material of Example 87—Step 1 (89 mg, 0.252 mmol) and methyl 2-chloroacetate (0.066 mL 0.755 mmol) in tetrahydrofuran (3.0 mL) was added potassium tert-butoxide (85 mg, 0.755 mmol) in one portion. The mixture was heated to reflux for 1 hour. The mixture was cooled down and quenched with saturated aqueous NH₄Cl. The mixture was extracted with ethyl acetate 3 times. The combined organic layers were washed with brine once, dried over anhydrous Na₂SO₄, filtered and concentrated to give the titled compound (80 mg, 0.188 mmol, 74.7% yield). LC-MS (ESI) m/z 426.2 (M+H)⁺.

### Step 3: (2-{[1-(4-methoxyphenyl)cyclopropane-1-carbonyl](3-phenylpropyl)amino}ethoxy)acetic Acid

To a solution of the material of Example 87—Step 2 (80 mg, 0.188 mmol) in 1,4-dioxane (0.5 mL) was added 1 N LiOH (0.5 mL 0.500 mmol) dropwise. The reaction mixture was stirred at 30° C. for 1 hour. The mixture was purified by preparative HPLC eluting with CH₃CN/H₂O/NH₄OH to give the titled compound (7 mg, 0.017 mmol, 3.40% yield). ¹H NMR (400 MHz, methanol-d₄) δ ppm 7.29-7.10 (m, 4H), 7.05-6.96 (m, 3H), 6.90-6.80 (m, 2H), 3.95 (s, 1H), 3.76 (d, J=1.3 Hz, 3H), 3.69-3.38 (m, 6H), 3.19 (t, J=6.0 Hz, 1H), 2.57 (t, J=7.8 Hz, 1H), 2.34 (t, J=7.5 Hz, 1H), 1.85 (p, J=7.8 Hz, 1H), 1.45 (s, 1H), 1.32 (t, J=3.4 Hz, 1H), 1.30-1.20 (m, 1H), 1.14 (q, J=4.7 Hz, 1H), 1.02-0.94 (m, 1H); LC-MS (ESI) m/z 412.2 (M+H)⁺.

### Example 88

### 5-{[4-(2-hydroxyethoxy)-3,5-dimethoxybenzoyl](3-phenylpropyl)amino}pentanoic Acid

### Step 1: methyl 4-(2-{[tert-butyl(dimethyl)silyl]oxy}ethoxy)-3,5-dimethoxybenzoate

A mixture of (2-bromoethoxy)(tert-butyl)dimethylsilane (451 mg, 1.885 mmol), methyl 4-hydroxy-3,5-dimethoxybenzoate (200 mg, 0.943 mmol) and potassium carbonate (391 mg, 2.83 mmol) in N,N-dimethylformamide (5 mL) were stirred at 110° C. for 1 hour. Then the mixture was cooled down to room temperature, diluted with water (15 mL), and extracted with ethyl acetate 3 times. The combined organic layers were dried over anhydrous Na₂SO₄, filtered and concentrated to give a residue which was purified by flash column chromatography on silica gel (loaded with CH₂Cl₂, elute: hexanes/ethyl acetate=0˜30%) to give the titled compound (340 mg, 0.918 mmol, 97% yield). LC-MS (ESI) m/z 371.2 (M+H)⁺, RT=2.283 minutes.

### Step 2: 4-(2-{[tert-butyl(dimethyl)silyl]oxy}ethoxy)-3,5-dimethoxybenzoic Acid

To a solution of the material of Example 88—Step 1 (340 mg, 0.918 mmol) in tetrahydrofuran (5 mL) was added 1 N lithium hydroxide (5.51 mL 5.51 mmol). The mixture was stirred at 60° C. for 7 hours. It was cooled down and acidified with 1 N HCl to pH=2-3 and then extracted with ethyl acetate twice. The combined organic layers were washed with brine twice, dried over anhydrous Na₂SO₄, filtered and concentrated to give the titled compound (250 mg, 0.701 mmol, 76% yield). LC-MS (ESI) m/z 357.2 (M+H)⁺, RT=2.083 minutes.

### Step 3: methyl 5-{[4-(2-{[tert-butyl(dimethyl)silyl]oxy}ethoxy)-3,5-dimethoxybenzoyl](3-phenylpropyl)amino}pentanoate

To a solution of the material of Example 88—Step 2 (150 mg, 0.421 mmol) and 1-[bis(dimethylamino)methylene]-1H-1,2,3-triazolo[4,5-b]pyridinium 3-oxid hexafluorophosphate (168 mg, 0.442 mmol, HATU) in N,N-dimethylformamide (3 mL) was added triethylamine (0.062 mL 0.442 mmol). The resulting solution was stirred at room temperature for 10 minutes. Then Example 70—Step 1 (440 mg, 0.884 mmol) was added in one portion. The solution was stirred at room temperature for 1 hour. The mixture was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (93 mg, 0.138 mmol, 32.7% yield). LC-MS (ESI) m/z 588.4 (M+H)⁺, RT=2.350 minutes.

### Step 4: methyl 5-{[4-(2-hydroxyethoxy)-3,5-dimethoxybenzoyl](3-phenylpropyl)amino}pentanoate

To a solution of the material of Example 88—Step 3 (93 mg, 0.158 mmol) in tetrahydrofuran (2 mL) was added tetra-n-butylammonium fluoride (165 mg, 0.633 mmol). The mixture was stirred at 30° C. for 1 hour. It was concentrated to give a residue which was diluted with water and extracted with tert-butyl methyl ether 3 times. The combined organic layers were washed with brine twice, dried over anhydrous Na₂SO₄, filtered and concentrated to give the titled compound (73 mg, 0.154 mmol, 97% yield). LC-MS (ESI) m/z 474.2 (M+H)⁺, RT=1.835 minutes.

### Step 5: 5-{[4-(2-hydroxyethoxy)-3,5-dimethoxybenzoyl](3-phenylpropyl)amino}pentanoic Acid

To a solution of the material of Example 88—Step 4 (73 mg, 0.154 mmol) in tetrahydrofuran (2 mL) was added 1 N lithium hydroxide (0.925 mL 0.925 mmol). The mixture was stirred at 30° C. for 1 hour. The mixture was cooled down and acidified with 1 N HCl to pH=2-3 and then extracted with ethyl acetate twice. The combined organic layers were washed with brine twice, dried over anhydrous Na₂SO₄, filtered and concentrated to give a residue which was purified by preparative HPLC (0.1% NH₃*H₂O/CH₃CN) and lyophilized to give the titled compound (35 mg, 0.076 mmol, 49.4% yield). ¹H NMR (400 MHz, DMSO-d₆) δ ppm 7.28-7.08 (m, 5H), 6.56 (s, 2H), 3.91 (t, J=5.7 Hz, 2H), 3.76 (s, 6H), 3.62 (t, J=5.7 Hz, 2H), 3.28 (s, 2H), 3.15 (s, 2H), 2.52 (s, 2H), 2.14 (s, 2H), 1.86 (q, J=7.5 Hz, 2H), 1.54 (s, 2H), 1.43 (s, 2H); LC-MS (ESI) m/z 460.2 (M+H)⁺, RT=1.700 minutes.

### Example 89

### 5-{[3-(4-methoxyphenyl)oxetane-3-carbonyl](3-phenylpropyl)amino}pentanoic Acid

### Step 1: methyl 5-{[3-(4-methoxyphenyl)oxetane-3-carbonyl](3-phenylpropyl)amino}pentanoate

To a solution of 3-(4-methoxyphenyl)oxetane-3-carboxylic acid (29.2 mg, 0.140 mmol) in N,N-dimethylformamide (8 mL) was added 1-[bis(dimethylamino)methylene]-1H-1,2,3-triazolo[4,5-b]pyridinium 3-oxid hexafluorophosphate (56.0 mg, 0.147 mmol, HATU). The resulting solution was stirred at room temperature for 10 minutes. Then a solution of Example 70—Step 1 (0.140 mmol) was added in one portion followed by the addition of diisopropylethylamine (0.025 mL 0.140 mmol). The solution was stirred at room temperature for 1 hour. The reaction mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (50 mg, 0.114 mmol, 81% yield). LC-MS (ESI) m/z 440.2 (M+H)⁺, RT=1.994 minutes.

### Step 2: 5-{[3-(4-methoxyphenyl)oxetane-3-carbonyl](3-phenylpropyl)amino}pentanoic Acid

To a solution of the material of Example 89—Step 1 (50 mg, 0.114 mmol) in dioxane (0.5 mL) was added 1 N LiOH (0.5 mL 0.500 mmol) dropwise. The reaction mixture was stirred at 30° C. for 1 hour. The mixture was purified by preparative HPLC eluting with CH₃CN/H₂O/NH₄OH to give the titled compound (25 mg, 0.059 mmol, 51.6% yield). ¹H NMR (400 MHz, methanol-d₄) δ ppm 7.42-7.34 (m, 1H), 7.31-7.11 (m, 5H), 6.97 (dd, J=10.9, 8.2 Hz, 3H), 5.19 (d, J=6.2 Hz, 1H), 5.07 (d, J=6.1 Hz, 1H), 4.76 (d, J=6.1 Hz, 1H), 4.43 (d, J=6.1 Hz, 1H), 3.81 (d, J=5.5 Hz, 3H), 3.42-3.33 (m, 2H), 2.65 (ddd, J=15.7, 10.8, 7.7 Hz, 3H), 2.36-2.22 (m, 2H), 1.93 (dt, J=28.2, 7.4 Hz, 2H), 1.60 (p, J=3.7 Hz, 2H), 1.54-1.42 (m, 1H), 1.27-1.08 (m, 2H); LC-MS (ESI) m/z 426.2 (M+H)⁺, RT=1.607 minutes.

### Example 90

### 5-{(3,5-dimethoxybenzoyl)[3-(3-fluorophenyl)propyl]amino}pentanoic Acid

### Step 1: methyl 5-{[3-(3-fluorophenyl)propyl]amino}pentanoate

To a solution of 3-(3-fluorophenyl)propan-1-amine (300 mg, 1.958 mmol) in acetonitrile (5 mL) was added methyl 5-bromopentanoate (382 mg, 1.958 mmol), and the resultant solution was heated to 80° C. for 1.5 hours. Then the mixture was cooled to room temperature and concentrated to give the titled compound. LC-MS (ESI) m/z 268.5 (M+H)⁺, RT=1.51 minutes.

### Step 2: methyl 5-{(3,5-dimethoxybenzoyl)[3-(3-fluorophenyl)propyl]amino}pentanoate

To a solution of 3,5-dimethoxybenzoic acid (357 mg, 1.958 mmol) in N,N-dimethylformamide (5 mL) was added 1-[bis(dimethylamino)methylene]-1H-1,2,3-triazolo[4,5-b]pyridinium 3-oxid hexafluorophosphate (744 mg, 1.958 mmol, HATU) and diisopropylethylamine (0.342 mL 1.958 mmol). The mixture was stirred at room temperature for 15 minutes. Then a solution of the material of Example 90—Step 1 (523 mg, 1.958 mmol) in acetonitrile (5 mL) was added to the mixture. The mixture was stirred at room temperature for 1 hour. Then the mixture was partitioned between water (20 mL) and ethyl acetate (20 mL). The organic layer was dried over Na₂SO₄, filtered and concentrated to give a residue that was purified by silica gel chromatography with hexane and ethyl acetate (0-30%) to give the titled compound (324 mg, 0.751 mmol, 38.3% yield). LC-MS (ESI) m/z 432.5 (M+H)⁺, RT=1.997 minutes.

### Step 3: 5-{(3,5-dimethoxybenzoyl)[3-(3-fluorophenyl)propyl]amino}pentanoic Acid

To a solution of the material of Example 90—Step 2 (324 mg, 0.751 mmol) in tetrahydrofuran (5 mL) and CH₃OH (2 mL) was added 1 N LiOH (3.75 mmol, 3.75 mL). The mixture was stirred at room temperature for 1 hour. Then the mixture was concentrated, and the residue was partitioned between water (5 mL) and diethyl ether (10 mL), and the mixture was washed with diethyl ether. The aqueous layer was acidified with 1 N HCl to pH=2-3 and extracted with ethyl acetate (20 mL). The organic layer was dried over Na₂SO₄, filtered and concentrated. The residue was purified by high pressure liquid column chromatography with acetonitrile and water to give the titled compound (190 mg, 0.455 mmol, 60.6% yield). ¹H NMR (400 MHz, DMSO-d₆) δ ppm 12.28 (brs, 1H), 7.42-6.77 (m, 4H), 6.48 (d, J=16.7 Hz, 1H), 6.37 (d, J=16.6 Hz, 2H), 3.73 (s, 3H), 3.71 (s, 3H), 3.37-3.35 (m, 2H), 3.11 (q, J=9.8, 6.5 Hz, 2H), 2.63 (t, J=8.0 Hz, 1H), 2.40 (t, J=7.6 Hz, 1H), 2.24 (d, J=6.5 Hz, 1H), 2.07 (d, J=9.0 Hz, 1H), 1.81 (dp, J=39.9, 7.5, 7.0 Hz, 2H), 1.65-1.38 (m, 3H), 1.27 (q, J=8.0, 7.4 Hz, 1H); LC-MS (ESI) m/z 418.5 (M+H)⁺, RT=1.860 minutes.

### Example 91

### 5-{[3-(3-chlorophenyl)propyl](3,5-dimethoxybenzoyl)amino}pentanoic Acid

### Step 1: methyl 5-{[3-(3-chlorophenyl)propyl]amino}pentanoate

To a solution of 3-(3-chlorophenyl)propan-1-amine (300 mg, 1.768 mmol) in acetonitrile (5 mL) was added methyl 5-bromopentanoate (345 mg, 1.768 mmol), and the solution was heated to 80° C. for 1 hour. Then K₂CO₃ (733 mg, 5.30 mmol) was added, and the mixture was heated to 80° C. for an additional 1 hour. Then the mixture was cooled to room temperature and concentrated. The residue was purified by flash column chromatography on silica gel (loaded directly with CH₂Cl₂) eluted with hexanes and ethyl acetate (0-50%) to give the titled compound.

### Step 2: methyl 5-{[3-(3-chlorophenyl)propyl](3,5-dimethoxybenzoyl)amino}pentanoate

To a solution of 3,5-dimethoxybenzoic acid (322 mg, 1.768 mmol) in N,N-dimethylformamide (5 mL) was added 1-[bis(dimethylamino)methylene]-1H-1,2,3-triazolo[4,5-b]pyridinium 3-oxid hexafluorophosphate (672 mg, 1.768 mmol, HATU) and diisopropylethylamine (0.309 mL 1.768 mmol). The mixture was stirred at room temperature for 15 minutes. Then a solution of the material from Example 91—Step 1 (502 mg, 1.768 mmol) in acetonitrile (5 mL) was added to the mixture. The mixture was stirred at room temperature for 1 hour. Then the mixture was partitioned between water (20 mL) and ethyl acetate (20 mL). The organic layer was dried over Na₂SO₄, filtered and concentrated to dryness. The residue was purified by chromatography with hexane and ethyl acetate (0-30%) to give the titled compound (110 mg, 0.246 mmol, 13.89% yield). LC-MS (ESI) m/z 448.5 (M+H)⁺, RT=2.060 minutes.

### Step 3: 5-{[3-(3-chlorophenyl)propyl](3,5-dimethoxybenzoyl)amino}pentanoic Acid

To a solution of the material from Example 91—Step 2 (110 mg, 0.246 mmol) in tetrahydrofuran (5 mL) and CH₃OH (2 mL) was added 1 N LiOH (1.23 mmol, 1.2 mL). The mixture was stirred at room temperature for 1 hour. Then the mixture was concentrated, and the residue was partitioned between water (5 mL) and diethyl ether (10 mL). The aqueous fraction was washed with diethyl ether, and the aqueous layer was acidified with 1 N HCl to pH=2-3 and then extracted with ethyl acetate (20 mL). The organic layer was dried over Na₂SO₄, filtered and concentrated. The residue was purified with high pressure liquid column chromatography eluted with acetonitrile and water (0.5% CF₃CO₂H) to give the titled compound (84 mg, 0.194 mmol, 79% yield). ¹H NMR (400 MHz, DMSO-d₆) δ ppm 12.04 (brs, 1H), 7.40-6.93 (m, 4H), 6.49 (d, J=14.2 Hz, 1H), 6.38 (d, J=16.3 Hz, 2H), 3.72 (s, 6H), 3.37 (d, J=7.5 Hz, 2H), 3.12 (q, J=8.7, 8.1 Hz, 2H), 2.62 (t, J=7.8 Hz, 1H), 2.39 (t, J=7.7 Hz, 1H), 2.26 (d, J=7.6 Hz, 1H), 2.06 (s, 1H), 1.81 (ddd, J=42.5, 12.0, 6.4 Hz, 2H), 1.61-1.40 (m, 3H), 1.36-1.19 (m, 1H); LC-MS (ESI) m/z 434.5 (M+H)⁺, RT=1.910 minutes.

### Example 92

### 5-{[3-(3-fluorophenyl)propyl][1-(4-methoxyphenyl)cyclopropane-1-carbonyl]amino}pentanoic Acid

### Step 1: methyl 5-{[3-(3-fluorophenyl)propyl][1-(4-methoxyphenyl)cyclopropane-1-carbonyl]amino}pentanoate

To a solution of 1-(4-methoxyphenyl)cyclopropanecarboxylic acid (259 mg, 1.347 mmol) in N,N-dimethylformamide (8 mL) was added 1-[bis(dimethylamino)methylene]-1H-1,2,3-triazolo[4,5-b]pyridinium 3-oxid hexafluorophosphate (538 mg, 1.414 mmol, HATU). The resulting solution was stirred at room temperature for 10 minutes. Then a solution of Example 90—Step 1 in N,N-dimethylformamide (2.0 mL) was added in one portion followed by the addition of diisopropylethylamine (0.235 mL 1.347 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on reverse phase silica C₁₈ eluting with CH₃CN in water (0.1% NH₄HCO₃) from 20% to 70% over 15 minutes to give the titled compound (100 mg, 0.226 mmol, 16.82% yield). ¹H NMR (400 MHz, methanol-d₄) δ ppm 7.37-7.19 (m, 2H), 7.19-7.12 (m, 1H), 7.02 (t, J=8.9 Hz, 2H), 6.98-6.80 (m, 4H), 3.77 (s, 3H), 3.63 (d, J=8.5 Hz, 3H), 3.35 (dd, J=8.2, 2.7 Hz, 2H), 3.29 (s, 1H), 2.60 (t, J=7.8 Hz, 1H), 2.39-2.30 (m, 3H), 2.10 (t, J=7.3 Hz, 1H), 1.90-1.78 (m, 1H), 1.54 (dq, J=5.9, 3.6 Hz, 3H), 1.39 (dt, J=15.9, 7.8 Hz, 2H), 1.25 (dq, J=21.5, 4.4, 3.4 Hz, 3H), 1.18-1.05 (m, 2H), 1.01 (q, J=4.6 Hz, 1H); LC-MS (ESI) m/z 442.5 (M+H)⁺, RT=2.079 minutes.

### Step 2: 5-{[3-(3-fluorophenyl)propyl][1-(4-methoxyphenyl)cyclopropane-1-carbonyl]amino}pentanoic Acid

To a solution of Example 92—Step 1 (100 mg, 0.226 mmol) in dioxane (0.5 mL) was added 1 N LiOH (0.5 mL 0.500 mmol) dropwise. The mixture was stirred at 30° C. for 1 hour. The reaction mixture was purified by preparative HPLC eluting with CH₃CN/H₂O/CF₃CO₂H to give the titled compound (42 mg, 0.098 mmol, 43.4% yield). ¹H NMR (400 MHz, methanol-d₄) δ ppm 7.32-7.13 (m, 2H), 7.06-6.80 (m, 5H), 6.74 (dd, J=10.1, 2.5 Hz, 1H), 3.76 (s, 3H), 3.40-3.32 (m, 3H), 2.59 (t, J=7.8 Hz, 1H), 2.32 (dt, J=13.7, 7.3 Hz, 2H), 2.07 (t, J=7.3 Hz, 1H), 1.84 (p, J=7.8 Hz, 1H), 1.56 (dd, J=7.2, 3.6 Hz, 2H), 1.46-1.17 (m, 5H), 1.19-1.06 (m, 2H), 1.01 (q, J=4.5 Hz, 1H); LC-MS (ESI) m/z 428.5 (M+H)⁺, RT=1.914 minutes.

### Example 93

### 2-{3-[(3,5-dimethoxy-4-methylbenzoyl)(3-phenylpropyl)amino]propyl}hexanoic Acid

### Step 1: methyl 2-{3-[(3,5-dimethoxy-4-methylbenzoyl)(3-phenylpropyl)amino]propyl}hexanoate

To a solution of diisopropylamine (0.362 mL 2.57 mmol) in tetrahydrofuran (5 mL) was added butyllithium (150 mg, 2.339 mmol) at 0° C. The mixture was stirred at 0-5° C. for 30 minutes, then a solution of Example 70—Step 2 (500 mg, 1.169 mmol) in tetrahydrofuran at −78° C. was added slowly, followed by 1-bromobutane (0.139 mL 1.286 mmol). The mixture was stirred at −78° C. and warmed-up to ambient temperature over 4 hours. The titled compound was obtained after purification by flash chromatography on silica gel (loaded directly with CH₂Cl₂) eluted with hexanes and ethyl acetate (0-50%). LC-MS (ESI) m/z 484.3 (M+H)⁺.

### Step 2: 2-{3-[(3,5-dimethoxy-4-methylbenzoyl)(3-phenylpropyl)amino]propyl}hexanoic Acid

Example 93—Step 1 (20 mg, 0.041 mmol) was dissolved in tetrahydrofuran (2 mL), and 1 N lithium hydroxide (5.94 mg, 0.248 mmol) solution (2 mL) was added. The mixture was stirred for 2 hours at room temperature. The mixture was poured into water. The pH was adjusted to 7, and the aqueous mixture was extracted with ethyl acetate three times. The combined organic layers were washed with brine, dried over Na₂SO₄, and concentrated. The residue was purified by preparative HPLC (0.1% CF₃CO₂H—H₂O/CH₃CN) to give the titled compound (9 mg, 0.019 mmol, 46.3% yield). ¹H NMR (400 MHz, DMSO-d₆) δ ppm 7.45-6.92 (m, 5H), 6.50 (s, 2H), 3.76 (s, 6H), 3.16 (s, 2H), 2.61 (s, 2H), 2.41 (s, 1H), 2.16 (s, 1H), 2.00 (s, 3H), 1.85 (d, J=31.9 Hz, 2H), 1.52 (m, 5H), 1.24 (s, 6H), 0.85 (s, 3H).

### Example 94

### 5-[benzyl(3,5-dimethoxy-4-methylbenzoyl)amino]pentanoic Acid

### Step 1: methyl 5-(benzylamino)pentanoate

Phenylmethanamine (200 mg, 1.866 mmol) and potassium carbonate (310 mg, 2.240 mmol) were dissolved in dry CH₃CN (6 mL) at room temperature. Methyl 5-bromopentanoate (400 mg, 2.053 mmol) dissolved in dry CH₃CN was added slowly to the mixture, and then the mixture was stirred at reflux for 3 hours. Then the mixture was cooled down and filtered to give the titled compound.

### Step 2: methyl 5-[benzyl(3,5-dimethoxy-4-methylbenzoyl)amino]pentanoate

3,5-Dimethoxy-4-methylbenzoic acid (432 mg, 2,200 mmol) and 1-[bis(dimethylamino)methylene]-1H-1,2,3-triazolo[4,5-b]pyridinium 3-oxid hexafluorophosphate (837 mg, 2,200 mmol, HATU) were dissolved in a mixture of dry N,N-dimethylformamide (1 mL), tetrahydrofuran (1.000 mL) and N,N-diisopropylethylamine (775 mg, 6.00 mmol). The mixture was stirred for 20 minutes at room temperature. Then Example 94—Step 1 (443 mg, 2.0 mmol) was added, the mixture was stirred for another 3 hours at room temperature. The mixture was poured into water and extracted with ethyl acetate 3 times. The combined organic fractions were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (230 mg, 0.576 mmol, 28.8% yield).

### Step 3: 5-[benzyl(3,5-dimethoxy-4-methylbenzoyl)amino]pentanoic Acid

Example 94—Step 2 (230 mg, 0.576 mmol) was dissolved in tetrahydrofuran (2 mL), and 1 N lithium hydroxide (83 mg, 3.45 mmol) solution (2 mL) was added. The mixture was stirred for 2 hours at room temperature. The mixture was poured into water, the pH was adjusted to 7, and the mixture was extracted with ethyl acetate 3 times. The combined organic layers were washed with brine, dried over Na₂SO₄, and concentrated. The residue was purified by preparative HPLC (0.1% CF₃CO₂H—H₂O/CH₃CN) to give the titled compound (106 mg, 0.275 mmol, 47.8% yield). ¹H NMR (400 MHz, DMSO-d₆) δ ppm 7.53-7.10 (m, 5H), 6.58 (s, 2H), 4.57 (d, J=76.0 Hz, 2H), 3.81 (s, 6H), 3.36 (s, 1H), 3.12 (s, 1H), 2.25 (s, 1H), 2.14-2.04 (m, 1H), 1.99 (d, J=16.7 Hz, 3H), 1.54 (s, 3H), 1.29 (s, 1H); LC-MS (ESI) m/z 386.2 (M+H)⁺.

### Example 95

### 5-[(3,5-dimethoxy-4-methylbenzoyl)(2-phenylethyl)amino]pentanoic Acid

### Step 1: methyl 5-[(2-phenylethyl)amino]pentanoate

2-Phenylethanamine (200 mg, 1.650 mmol) and potassium carbonate (274 mg, 1.981 mmol) were dissolved in dry CH₃CN (6 mL) at room temperature. Methyl 5-bromopentanoate (354 mg, 1.815 mmol) dissolved in dry CH₃CN was added slowly to the mixture, and then the mixture was stirred at reflux for 3 hours. Then the mixture was cooled down and filtered to give the titled compound.

### Step 2: methyl 5-[(3,5-dimethoxy-4-methylbenzoyl)(2-phenylethyl)amino]pentanoate

3,5-Dimethoxy-4-methylbenzoic acid (391 mg, 1.991 mmol) and 1-[bis(dimethylamino)methylene]-1H-1,2,3-triazolo[4,5-b]pyridinium 3-oxid hexafluorophosphate (757 mg, 1.991 mmol, HATU) were dissolved in a mixture of dry N,N-dimethylformamide (1 mL), tetrahydrofuran (1.000 mL) and N,N-diisopropylethylamine (702 mg, 5.43 mmol). The mixture was stirred for 20 minutes at room temperature. Then Example 95—Step 1 (426 mg, 1.81 mmol) was added, and the mixture was stirred for another 3 hours at room temperature. The mixture was poured into water and extracted with ethyl acetate 3 times. The combined organic layers were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (176 mg, 0.426 mmol, 23.52% yield).

### Step 3: 5-[(3,5-dimethoxy-4-methylbenzoyl)(2-phenylethyl)amino]pentanoic Acid

Example 95—Step 2 (160 mg, 0.387 mmol) was dissolved in tetrahydrofuran (2 mL) and 1 N lithium hydroxide (55.6 mg, 2.322 mmol) solution (2 mL) was added. The mixture was stirred for 2 hours at room temperature. The mixture was poured into water, the pH was adjusted to 7, and the aqueous mixture was extracted with ethyl acetate 3 times. The combined organic layers were washed with brine, dried over Na₂SO₄, and concentrated. The residue was purified by preparative HPLC (0.1% CF₃CO₂H—H₂O/CH₃CN) to give the titled compound (76 mg, 0.190 mmol, 49.2% yield). ¹H NMR (400 MHz, DMSO-d₆) δ ppm 7.43-7.13 (m, 4H), 7.01 (s, 1H), 6.43 (s, 2H), 3.60 (s, 1H), 3.43 (d, J=31.4 Hz, 2H), 3.08 (s, 1H), 2.87 (d, J=38.3 Hz, 2H), 2.30 (s, 1H), 2.16 (s, 1H), 2.00 (s, 3H), 1.53 (d, J=42.3 Hz, 3H), 1.29 (s, 1H); LC-MS (ESI) m/z 400.2 (M+H)⁺.

### Example 96

### 5-[(3,5-dimethoxy-4-methylbenzoyl)(4-phenylbutyl)amino]pentanoic Acid

### Step 1: methyl 5-[(4-phenylbutyl)amino]pentanoate

4-Phenylbutan-1-amine (200 mg, 1.340 mmol) and potassium carbonate (222 mg, 1.608 mmol) were dissolved in dry CH₃CN (6 mL) at room temperature. Methyl 5-bromopentanoate (288 mg, 1.474 mmol) dissolved in dry CH₃CN was added slowly to the mixture; then the mixture was stirred at reflux for 3 hours. Then the mixture was cooled down and filtered to give the titled compound. MS (ESI) m/z 264.2 (M+H)⁺.

### Step 2: methyl 5-[(3,5-dimethoxy-4-methylbenzoyl)(4-phenylbutyl)amino]pentanoate

3,5-Dimethoxy-4-methylbenzoic acid (317 mg, 1.617 mmol) and 1-[bis(dimethylamino)methylene]-1H-1,2,3-triazolo[4,5-b]pyridinium 3-oxid hexafluorophosphate (615 mg, 1.617 mmol, HATU) were dissolved in a mixture of dry N,N-dimethylformamide (1 mL), tetrahydrofuran (1.000 mL) and N,N-diisopropylethylamine (570 mg, 4.41 mmol). The mixture was stirred for 20 minutes at room temperature. Then Example 96—Step 1 (387 mg, 1.47 mmol) was added, and the mixture was stirred for another 3 hours at room temperature. The mixture was poured into water and extracted with ethyl acetate 3 times. The combined organic layers were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (312 mg, 0.707 mmol, 48.1% yield).

### Step 3: 5-[(3,5-dimethoxy-4-methylbenzoyl)(4-phenylbutyl)amino]pentanoic Acid

Example 96—Step 2 (270 mg, 0.611 mmol) was dissolved in tetrahydrofuran (2 mL), and 1 N lithium hydroxide (88 mg, 3.67 mmol) solution (2 mL) was added. The mixture was stirred for 2 hours at room temperature. The mixture was poured into water, the pH was adjusted to 7, and the aqueous mixture was extracted with ethyl acetate 3 times. The combined organic layers were washed with brine, and dried over Na₂SO₄, and concentrated. The residue was purified by preparative HPLC (0.1% CF₃CO₂H—H₂O/CH₃CN) to give the titled compound (76 mg, 0.178 mmol, 29.1% yield). ¹H NMR (400 MHz, DMSO-d₆) δ ppm 7.38-6.98 (m, 5H), 6.49 (s, 2H), 3.77 (m, 6H), 3.37 (s, 3H), 3.17 (s, 2H), 2.64 (s, 1H), 2.46 (s, 1H), 2.27 (s, 1H), 2.09 (d, J=9.3 Hz, 1H), 2.00 (s, 3H), 1.57 (d, J=28.0 Hz, 6H), 1.35 (d, J=37.6 Hz, 2H). MS (ESI) m/z 428.2 (M+H)⁺.

### Example 97

### 2-{3-[(3,5-dimethoxy-4-methylbenzoyl)(3-phenylpropyl)amino]propyl}-2-methylhexanoic Acid

### Step 1: methyl 5-[(3,5-dimethoxy-4-methylbenzoyl)(3-phenylpropyl)amino]-2-methylpentanoate

To a solution of diisopropylamine (1.467 mL, 10.29 mmol) in tetrahydrofuran (10 mL) was added n-butyllithium (10.29 mmol, 6.4 mL, 1.6 M in hexane) at 0° C. under N₂ atmosphere. The mixture was stirred at 0° C. for 0.5 hour, and cooled to −78° C. Then a solution of Example 70—Step 2 (2 g, 4.68 mmol) in tetrahydrofuran (10 mL) was added to the mixture dropwise, and the mixture was stirred at −78° C. for 10 minutes. Then methyl iodide (0.878 mL, 14.03 mmol) was added to the mixture with continued stirring at −78° C. to room temperature for 2 hours. Then the reaction was quenched with saturated NH₄Cl and the mixture was extracted with ethyl acetate (20 mL). The organic layer was dried over Na₂SO₄, filtered and concentrated to dryness. The residue was purified by flash column chromatography on silica gel (loaded directly with CH₂Cl₂) eluted with hexanes and ethyl acetate (0-50%) to give the titled compound (1.74 g, 3.94 mmol, 84% yield). LC-MS (ESI) m/z 442.2 (M+H)⁺, RT=2.15 minutes.

### Step 2: methyl 2-{3-[(3,5-dimethoxy-4-methylbenzoyl)(3-phenylpropyl)amino]propyl}-2-methylhexanoate

To a solution of diisopropylamine (0.177 mL, 1.25 mmol) in tetrahydrofuran (5 mL) was added n-butyllithium (1.19 mmol, 0.74 mL, 1.6 M in hexane) at 0° C. under N₂ atmosphere. The mixture was stirred at 0° C. for 0.5 hour, and cooled to −78° C. Then a solution of Example 97—Step 1 (250 mg, 0.566 mmol) in tetrahydrofuran (5 mL) was added to the mixture dropwise, and the mixture was stirred at −78° C. for 10 minutes. Then 1-bromobutane (233 mg, 1.699 mmol) was added to the mixture with continued stirring at −78° C. to room temperature for 2 hours. Then reaction was quenched with saturated NH₄Cl, and the mixture was extracted with ethyl acetate (20 mL). The organic layer was dried over Na₂SO₄, filtered and concentrated to dryness. The residue was purified by flash chromatography on silica gel eluted with hexane and ethyl acetate to give the titled compound (20 mg, 0.032 mmol, 5.68% yield). LC-MS (ESI) m/z 498.2 (M+H)⁺, RT=2.36 minutes.

### Step 3: 2-{3-[(3,5-dimethoxy-4-methylbenzoyl)(3-phenylpropyl)amino]propyl}-2-methylhexanoic Acid

To a solution of Example 97—Step 2 (20 mg, 0.040 mmol) in tetrahydrofuran (1 mL) was added 1 N LiOH (0.804 mmol, 0.8 mL), and the mixture was stirred at room temperature overnight. Then the mixture was heated to 50° C. overnight. Then 2 N KOH (1 mL) was added to the mixture which was then heated to 50° C. overnight. The mixture was cooled to room temperature and 1 N HCl was added to the mixture to adjust pH=2-3 followed by extraction with ethyl acetate (20 mL). The organic layer was dried over Na₂SO₄, filtered and concentrated. The residue was purified by high pressure liquid chromatography with acetonitrile and water (0.5% CF₃CO₂H) to give the titled compound (11 mg, 0.023 mmol, 56.6% yield). ¹H NMR (400 MHz, DMSO-d₆) δ ppm 12.07 (s, 1H), 7.16 (dt, J=54.4, 24.3 Hz, 5H), 6.48 (s, 2H), 3.74 (s, 6H), 3.13 (s, 2H), 2.62 (s, 1H), 2.39 (s, 1H), 1.98 (s, 3H), 1.93-1.67 (m, 2H), 1.62-1.05 (m, 10H), 0.98 (d, J=31.9 Hz, 3H), 0.90-0.71 (m, 3H); LC-MS (ESI) m/z 484.2 (M+H)⁺, RT=2.20 minutes.

### Example 98

### 5-[(3,5-dimethoxybenzoyl)(3-phenylpropyl)amino]-2-methylpentanoic Acid

### Step 1: methyl 5-[(3,5-dimethoxybenzoyl)(3-phenylpropyl)amino]-2-methylpentanoate

To a solution of diisopropylamine (325 mg, 3.21 mmol) in tetrahydrofuran (15 mL) was added butyllithium (1.882 mL, 3.01 mmol) at 0° C. under N₂ atmosphere. The mixture was stirred at 0° C. for 15 minutes, and cooled to −78° C. Then a solution of Example 74—Step 1 (830 mg, 2.007 mmol) in tetrahydrofuran (6 mL) was added to the mixture dropwise, and the mixture was stirred at −78° C. for 25 minutes. Then iodomethane (1425 mg, 10.04 mmol) was added to the mixture with continued stirring at −78° C. to −30° C. over 2 hours. Aqueous NH₄Cl was added to the mixture, and the mixture was extracted with ethyl acetate (60 mL). The organic fraction was dried and concentrated. The residue was purified by chromatography on silica gel eluted with hexanes and ethyl acetate (from 0-50%) to give the titled compound (720 mg, 1.684 mmol, 84% yield). ¹H NMR (400 MHz, CDCl₃) δ ppm 7.39-7.10 (m, 4H), 7.10-6.95 (m, 1H), 6.51-6.36 (m, 3H), 3.79 (s, 6H), 3.71-3.59 (m, 3H), 3.46 (brs, 2H), 3.21 (brs, 2H), 2.69 (brs, 1H), 2.56-2.21 (m, 2H), 1.98 (brs, 1H), 1.84 (brs, 1H), 1.63 (brs, 2H), 1.45 (d, J=15.9 Hz, 2H), 1.21-0.99 (m, 3H); LC-MS (ESI) m/z 428.2 (M+H)⁺, RT=2.08 minutes.

### Step 2: 5-[(3,5-dimethoxybenzoyl)(3-phenylpropyl)amino]-2-methylpentanoic Acid

To a solution of Example 98—Step 1 (220 mg, 0.515 mmol) in 1,4-dioxane (5 mL) was added 1 N LiOH (5.15 mL 5.15 mmol), and the mixture was heated to 70° C. overnight. Then the mixture was cooled to room temperature and concentrated. The residue was diluted with water and washed with ethyl ether (20 mL). The aqueous layer was acidified with 1 N HCl to pH=2-3 and extracted with ethyl acetate (30 mL). The organic layer was dried over Na₂SO₄, filtered and concentrated to give a residue which was purified by high pressure liquid chromatography eluted with acetonitrile and water (0.5% CF₃CO₂H) to give the titled compound (203 mg, 0.491 mmol, 95% yield). ¹H NMR (400 MHz, DMSO-d₆) δ ppm 12.08 (s, 1H), 7.40-6.92 (m, 5H), 6.50 (d, J=3.7 Hz, 1H), 6.38 (s, 2H), 3.73 (s, 6H), 3.43-3.30 (m, 2H), 3.10 (d, J=8.3 Hz, 2H), 2.61 (t, J=7.8 Hz, 1H), 2.38 (t, J=7.7 Hz, 1H), 2.16 (q, J=7.3, 6.5 Hz, 1H), 1.80 (dq, J=36.9, 8.5 Hz, 2H), 1.53 (d, J=10.0 Hz, 2H), 1.47-1.25 (m, 2H), 1.05 (d, J=6.8 Hz, 2H), 1.00-0.90 (m, 1H); LC-MS (ESI) m/z 414.2 (M+H)⁺, RT=1.92 minutes.

### Example 99

### 5-[(4-fluoro-3,5-dimethoxybenzoyl)(3-phenylpropyl)amino]pentanoic Acid

### Step 1: methyl 5-[(4-fluoro-3,5-dimethoxybenzoyl)(3-phenylpropyl)amino]pentanoate

To a solution of 4-fluoro-3,5-dimethoxybenzoic acid (150 mg, 0.749 mmol) and 1-[bis(dimethylamino)methylene]-1H-1,2,3-triazolo[4,5-b]pyridinium 3-oxid hexafluorophosphate (313 mg, 0.824 mmol, HATU) in N,N-dimethylformamide (3 mL) was added triethylamine (0.115 mL 0.824 mmol). The resulting solution was stirred at room temperature for 10 minutes. Then to a solution of Example 70—Step 1 (392 mg, 0.787 mmol) in CH₂Cl₂ (4 mL) was added triethylamine (2 mL) dropwise. After addition, the mixture was added to the above solution in one portion. The resulting solution was stirred at room temperature for 1 hour. Then the mixture was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-40%) to give the titled compound (230 mg, 0.533 mmol, 71.1% yield). LC-MS (ESI) m/z 432.2 (M+H)⁺, RT=2.00 minutes.

### Step 2: 5-[(4-fluoro-3,5-dimethoxybenzoyl)(3-phenylpropyl)amino]pentanoic Acid

To a solution of Example 99—Step 1 (230 mg, 0.533 mmol) in tetrahydrofuran (2 mL) was added 1 N lithium hydroxide (3.20 mL 3.20 mmol). The mixture was stirred at room temperature for 1 hour. The mixture was cooled down and acidified with 1 N HCl to pH=2-3. The mixture was extracted with ethyl acetate twice, and the combined organic layers were washed with brine twice, dried over anhydrous Na₂SO₄, filtered and concentrated to give a residue which was purified by preparative HPLC (0.1% CF₃CO₂H—H₂O/CH₃CN) and lyophilized to give an oil (150 mg). The oil was further purified with a second preparative HPLC (0.1% CF₃CO₂H—H₂O/CH₃CN) and lyophilized to give the titled compound (110 mg, 0.263 mmol, 49.4% yield). ¹H NMR (400 MHz, DMSO-d₆) δ ppm 7.21 (d, J=7.3 Hz, 2H), 7.14 (t, J=7.4 Hz, 3H), 6.64 (d, J=7.0 Hz, 2H), 3.81 (s, 6H), 3.27 (s, 4H), 2.51 (d, J=9.7 Hz, 2H), 2.17 (s, 2H), 1.84 (t, J=8.0 Hz, 2H), 1.59-1.50 (m, 2H), 1.44 (s, 2H); LC-MS (ESI) m/z 418.2 (M+H)⁺, RT=1.852 minutes.

### Example 100

### ({2-[(4-fluoro-3,5-dimethoxybenzoyl)(3-phenylpropyl)amino]ethyl}sulfanyl)acetic Acid

### Step 1: 2-[(3-phenylpropyl)amino]ethan-1-ol Hydrochloride

tert-Butyl (2-hydroxyethyl)(3-phenylpropyl)carbamate (1 g, 3.58 mmol, Accela ChemBio Co., Ltd) was added to 4 N HCl in 1,4-dioxane (10 mL), and the mixture was stirred at room temperature overnight. Then the mixture was concentrated to dryness to give the titled compound (0.772 g, 3.58 mmol, 100% yield).

### Step 2: 4-fluoro-N-(2-hydroxyethyl)-3,5-dimethoxy-N-(3-phenylpropyl)benzamide

To a solution of 4-fluoro-3,5-dimethoxybenzoic acid (150 mg, 0.749 mmol) and 1-[bis(dimethylamino)methylene]-1H-1,2,3-triazolo[4,5-b]pyridinium 3-oxid hexafluorophosphate (313 mg, 0.824 mmol, HATU) in N,N-dimethylformamide (3 mL) was added triethylamine (0.115 mL 0.824 mmol). The resultant mixture was stirred at room temperature for 30 minutes. Then Example 100—Step 1 (162 mg, 0.749 mmol) in tetrahydrofuran (2.0 mL) and triethylamine (0.5 mL) was added. The mixture was stirred at room temperature for 1 hour. It was diluted with water and extracted with ethyl acetate 3 times. The combined organic layers were washed with brine once, dried over anhydrous Na₂SO₄, filtered and concentrated to give a residue which was purified by flash column chromatography on silica gel (loaded directly with CH₂Cl₂) eluted with hexanes and ethyl acetate (50-100%) and then methanol (0-5%) in CH₂Cl₂ to give the titled compound (127 mg, 0.351 mmol, 46.9% yield). LC-MS (ESI) m/z 362.2 (M+H)⁺, RT=1.780 minutes.

### Step 3: N-(2-chloroethyl)-4-fluoro-3,5-dimethoxy-N-(3-phenylpropyl)benzamide

To a solution of Example 100—Step 2 (127 mg, 0.351 mmol) in CH₂Cl₂ (2 mL) was added triethylamine (0.098 mL 0.703 mmol) and methanesulfonyl chloride (0.055 mL 0.703 mmol), sequentially. Then the solution was stirred at room temperature overnight. The solvent was removed under vacuum to give the titled compound. LC-MS (ESI) m/z 380.2 (M+H)⁺, RT=2.008 minutes.

### Step 4: ethyl ({2-[(4-fluoro-3,5-dimethoxybenzoyl)(3-phenylpropyl)amino]ethyl}sulfanyl)acetate

The mixture of Example 100—Step 3 was diluted with N,N-dimethylformamide (2 mL) followed by addition of potassium carbonate (194 mg, 1.404 mmol) and ethyl 2-mercaptoacetate (0.077 mL 0.702 mmol). The mixture was heated to 50° C. for 1 hour. Then the mixture was cooled down and diluted with water (10 mL) and extracted with ethyl acetate twice. The combined organic layers were washed with brine twice, dried over anhydrous Na₂SO₄, filtered and concentrated to give a residue which was purified by flash column chromatography on silica gel eluted with ethyl acetate in hexanes (0-80%) to give the titled compound (56 mg, 0.100 mmol, 28.6% yield). LC-MS (ESI) m/z 464.2 (M+H)⁺, RT=2.044 minutes.

### Step 5: ({2-[(4-fluoro-3,5-dimethoxybenzoyl)(3-phenylpropyl)amino]ethyl}sulfanyl)acetic Acid

To a solution of Example 100—Step 4 (56 mg, 0.121 mmol) in tetrahydrofuran (2 mL) was added 1 N lithium hydroxide (0.725 mL 0.725 mmol). The mixture was stirred at room temperature for 1 hour. The mixture was acidified with 1 N HCl to pH=2-3 and then extracted with ethyl acetate twice. The combined organic layers were washed with brine twice, dried over anhydrous Na₂SO₄, filtered and concentrated to give a residue which was purified by preparative HPLC (0.1% CF₃CO₂H—H₂O/CH₃CN) and lyophilized to give the titled compound (27.6 mg, 0.063 mmol, 52.5% yield). ¹H NMR (400 MHz, DMSO-d₆) δ ppm 12.36 (s, 1H), 7.28-7.18 (m, 2H), 7.13 (t, J=7.5 Hz, 3H), 6.68 (d, J=7.0 Hz, 2H), 3.81 (s, 6H), 3.51 (s, 2H), 3.29 (s, 2H), 3.20 (s, 2H), 2.80 (t, J=7.3 Hz, 2H), 2.50 (d, J=5.2 Hz, 2H), 1.93-1.78 (m, 2H); LC-MS (ESI) m/z 436.2 (M+H)⁺, RT=1.843 minutes.

### Example 101

### ({2-[(3,5-dimethoxybenzoyl)(3-phenylpropyl)amino]ethyl}sulfanyl)acetic Acid

### Step 1: N-(2-hydroxyethyl)-3,5-dimethoxy-N-(3-phenylpropyl)benzamide

To a solution of 3,5-dimethoxybenzoic acid (200 mg, 1.098 mmol) and 1-[bis(dimethylamino)methylene]-1H-1,2,3-triazolo[4,5-b]pyridinium 3-oxid hexafluorophosphate (459 mg, 1.208 mmol, HATU) in N,N-dimethylformamide (5 mL) was added triethylamine (0.168 mL 1.208 mmol). The resultant solution was stirred at room temperature for 30 minutes. Then a solution of Example 100—Step 1 (237 mg, 1.098 mmol) in tetrahydrofuran (4 mL) and triethylamine (0.5 mL) was added. The mixture was stirred at room temperature for 1 hour. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (50-100%) to give the titled compound (300 mg, 0.874 mmol, 80% yield). LC-MS (ESI) m/z 344.2 (M+H)⁺, RT=1.783 minutes.

### Step 2: N-(2-chloroethyl)-3,5-dimethoxy-N-(3-phenylpropyl)benzamide

To a solution of Example 101—Step 1 (300 mg, 0.874 mmol) in CH₂Cl₂ (2 mL) was added triethylamine (0.244 mL 1.747 mmol) and methanesulfonyl chloride (0.136 mL 1.747 mmol), sequentially. Then the solution was stirred at room temperature overnight. The solvent was removed under vacuum to give the titled compound. LC-MS (ESI) m/z 362.2 (M+H)⁺, RT=2.019 minutes.

### Step 3: ethyl ({2-[(3,5-dimethoxybenzoyl)(3-phenylpropyl)amino]ethyl}sulfanyl)acetate

To a solution of Example 101—Step 2 (156 mg, 0.431 mmol) in N,N-dimethylformamide (2 mL) was added potassium carbonate (119 mg, 0.862 mmol) and ethyl 2-mercaptoacetate (0.095 mL 0.862 mmol). The mixture was heated to 50° C. for 1 hour. Then the mixture was cooled down and diluted with water (10 mL) and extracted with ethyl acetate twice. The combined organic layers were washed with brine twice, dried over anhydrous Na₂SO₄, filtered and concentrated to give a residue which was purified by flash column chromatography on silica gel eluted with ethyl acetate in hexanes(0-80%) to give the titled compound (125 mg, 0.281 mmol, 65.1% yield). LC-MS (ESI) m/z 446.2 (M+H)⁺, RT=2.056 minutes.

### Step 4: ({2-[(3,5-dimethoxybenzoyl)(3-phenylpropyl)amino]ethyl}sulfanyl)acetic Acid

To a solution of Example 101—Step 3 (125 mg, 0.281 mmol) in tetrahydrofuran (2 mL) was added 1 N lithium hydroxide (1.683 mL 1.683 mmol). The mixture was stirred at room temperature for 1 hour. The mixture was acidified with 1 N HCl to pH=2-3 and then extracted with ethyl acetate twice. The combined organic layers were washed with brine twice, dried over anhydrous Na₂SO₄, filtered and concentrated to give a residue which was purified by preparative HPLC (0.1% CF₃CO₂H—H₂O/CH₃CN) and lyophilized to give the titled compound (100 mg, 0.240 mmol, 85% yield). ¹H NMR (400 MHz, DMSO-d₆) δ ppm 12.36 (s, 1H), 7.23 (t, J=7.4 Hz, 2H), 7.18-6.91 (m, 3H), 6.51 (t, J=2.3 Hz, 1H), 6.42 (d, J=2.3 Hz, 2H), 3.74 (s, 6H), 3.53 (d, J=23.8 Hz, 2H), 3.29 (s, 2H), 3.15 (s, 2H), 2.78 (s, 2H), 2.50 (s, 2H), 1.84 (s, 2H); LC-MS (ESI) m/z 418.2 (M+H)⁺, RT=1.851 minutes.

### Example 102

### 5-{[3-(3-chlorophenyl)propyl][1-(4-methoxyphenyl)cyclopropane-1-carbonyl]amino}pentanoic Acid

### Step 1: methyl 5-{[3-(3-chlorophenyl)propyl][1-(4-methoxyphenyl)cyclopropane-1-carbonyl]amino}pentanoate

To a solution of 1-(4-methoxyphenyl)cyclopropanecarboxylic acid (237 mg, 1.233 mmol) and 1-[bis(dimethylamino)methylene]-1H-1,2,3-triazolo[4,5-b]pyridinium 3-oxid hexafluorophosphate (492 mg, 1.295 mmol, HATU) in N,N-dimethylformamide (8 mL) was added diisopropylethylamine (159 mg, 1.233 mmol). The resulting solution was stirred at room temperature for 5 minutes. Then Example 91—Step 1 (350 mg, 1.233 mmol) was added in one portion. The solution was stirred at room temperature for 3 hours. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-100%) to give the titled compound (200 mg, 0.437 mmol, 35.5% yield). LC-MS (ESI) m/z 458.2 (M+H)⁺, RT=2.138 minutes.

### Step 2: 5-{[3-(3-chlorophenyl)propyl][1-(4-methoxyphenyl)cyclopropane-1-carbonyl]amino}pentanoic Acid

To a solution of Example 102—Step 1 (100 mg, 0.218 mmol) in 1,4-dioxane (0.5 mL) was added 1 N LiOH (0.5 mL 0.500 mmol) dropwise. The mixture was stirred at 30° C. for 1 hour. The mixture was purified by preparative HPLC (0.1% CF₃CO₂H—H₂O/CH₃CN) to give the titled compound (26 mg, 0.059 mmol, 26.8% yield). ¹H NMR (400 MHz, methanol-d₄) δ ppm 7.27-7.09 (m, 4H), 7.04-6.94 (m, 2H), 6.86 (dd, J=11.1, 8.4 Hz, 2H), 3.76 (s, 3H), 3.38-3.29 (m, 4H), 2.57 (t, J=7.8 Hz, 1H), 2.38-2.26 (m, 2H), 2.07 (t, J=7.3 Hz, 1H), 1.89-1.76 (m, 1H), 1.55 (h, J=4.4, 3.8 Hz, 2H), 1.40 (p, J=7.7 Hz, 1H), 1.33-1.24 (m, 2H), 1.24-1.17 (m, 1H), 1.17-1.05 (m, 2H), 1.02-0.95 (m, 1H); LC-MS (ESI) m/z 444.2 (M+H)⁺, RT=1.974 minutes.

### Example 103

### 5-{[1-(2-fluoro-4-methoxyphenyl)cyclopropane-1-carbonyl](3-phenylpropyl)amino}pentanoic Acid

### Step 1: 1-(2-fluoro-4-methoxyphenyl)cyclopropane-1-carbonitrile

To a solution of 2-(2-fluoro-4-methoxyphenyl)acetonitrile (200 mg, 1.211 mmol) in N,N-dimethylformamide (1 mL) was added sodium hydride (60% in mineral oil) (145 mg, 3.63 mmol) at 0° C. under an atmosphere of nitrogen. The mixture was stirred at room temperature for 30 minutes. 1-bromo-2-chloroethane (347 mg, 2.422 mmol) was added to the mixture at 0° C. The reaction mixture was stirred at 30° C. for 3 hours. The mixture was cooled down to 0° C. and quenched with 1 N HCl. The mixture was extracted with ethyl acetate (2×20 mL), and the combined organic layers were washed with brine (2×20 mL), dried over Na₂SO₄ and concentrated. The residue was purified by silica flash column chromatography eluted with ethyl acetate in hexane from 0% to 50% over 20 minutes to give the titled compound (170 mg, 0.889 mmol, 73.4% yield). ¹H NMR (400 MHz, methanol-d₄) δ ppm 6.82-6.70 (m, 3H), 3.80 (d, J=1.4 Hz, 3H), 1.65-1.58 (m, 2H), 1.38-1.32 (m, 2H); LC-MS (ESI) m/z 192.2 (M+H)⁺, RT=1.788 minutes.

### Step 2: 1-(2-fluoro-4-methoxyphenyl)cyclopropane-1-carboxylic Acid

To a solution of Example 103—Step 1 (170 mg, 0.889 mmol) in ethanol (1 mL) was added 2 M KOH (1 mL 2.000 mmol). The mixture was heated to reflux and stirred for 16 hours. The mixture was cooled down and quenched with 1 N HCl slowly. The mixture was extracted with ethyl acetate (2×20 mL), and the combined organic layers were dried over Na₂SO₄ and concentrated to give the titled compound (130 mg, 0.618 mmol, 69.6% yield). ¹H NMR (400 MHz, methanol-d₄) δ ppm 6.74-6.60 (m, 3H), 3.78 (d, J=1.0 Hz, 3H), 1.56 (q, J=4.0 Hz, 2H), 1.14 (q, J=4.0 Hz, 2H); LC-MS (ESI) m/z 211.2 (M+H)⁺, RT=1.659 minutes.

### Step 3: methyl 5-{[1-(2-fluoro-4-methoxyphenyl)cyclopropane-1-carbonyl](3-phenylpropyl)amino}pentanoate

To a solution of Example 103—Step 2 (29.5 mg, 0.140 mmol) in N,N-dimethylformamide (1 mL) was added 1-[bis(dimethylamino)methylene]-1H-1,2,3-triazolo[4,5-b]pyridinium 3-oxid hexafluorophosphate (56.0 mg, 0.147 mmol, HATU). The resulting solution was stirred at room temperature for 10 minutes. Then Example 70—Step 1 (35 mg, 0.140 mmol) was added in one portion followed by the addition of diisopropylethylamine (0.049 mL 0.281 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (30 mg, 0.068 mmol, 48.4% yield). LC-MS (ESI) m/z 442 (M+H)⁺, RT=2.092 minutes.

### Step 4: 5-{[1-(2-fluoro-4-methoxyphenyl)cyclopropane-1-carbonyl](3-phenylpropyl)amino}pentanoic Acid

To a solution of Example 103—Step 3 (30 mg, 0.068 mmol) in dioxane (0.5 mL) was added 1 N LiOH (0.5 mL 0.500 mmol) dropwise. The mixture was stirred at 30° C. for 1 hour. The mixture was purified by preparative HPLC eluted with CH₃CN/H₂O/CF₃CO₂H to give the titled compound (20 mg, 0.047 mmol, 68.9% yield). ¹H NMR (400 MHz, methanol-d₄) δ ppm 7.25 (t, J=7.6 Hz, 2H), 7.16 (t, J=6.8 Hz, 2H), 7.02 (d, J=8.1 Hz, 2H), 6.70 (td, J=17.0, 16.6, 9.3 Hz, 2H), 3.77 (d, J=5.4 Hz, 3H), 3.39-3.32 (m, 2H), 3.25 (d, J=7.1 Hz, 2H), 2.55 (dd, J=9.2, 6.4 Hz, 1H), 2.29 (dt, J=13.1, 7.0 Hz, 2H), 2.06 (t, J=7.4 Hz, 1H), 1.85-1.76 (m, 1H), 1.51 (q, J=4.2 Hz, 2H), 1.31 (td, J=18.1, 16.0, 8.8 Hz, 4H), 1.16 (s, 1H), 1.07 (q, J=4.4 Hz, 1H), 0.99 (s, 1H); LC-MS (ESI) m/z 428 (M+H)⁺, RT=1.914 minutes.

### Example 104

### 1-{[N-(3,5-dimethoxy-4-methylbenzoyl)-N-(3-phenylpropyl)glycyl]amino}-4-ethylcyclohexane-1-carboxylic Acid

### Step 1: methyl [(3-phenylpropyl)amino]acetate

A mixture of methyl 2-chloroacetate (400 mg, 3.69 mmol), 3-phenylpropan-1-amine (498 mg, 3.69 mmol) and potassium carbonate (611 mg, 4.42 mmol) in CH₃CN (12 mL) was stirred at 50° C. overnight. The mixture was cooled down and filtered to give the titled compound. LC-MS (ESI) m/z 208.2 (M+H)⁺, RT=1.380 minutes.

### Step 2: methyl [(3,5-dimethoxy-4-methylbenzoyl)(3-phenylpropyl)amino]acetate

To a solution of 3,5-dimethoxy-4-methylbenzoic acid (724 mg, 3.69 mmol) and 1-[bis(dimethylamino)methylene]-1H-1,2,3-triazolo[4,5-b]pyridinium 3-oxid hexafluorophosphate (1473 mg, 3.87 mmol, HATU) in N,N-dimethylformamide (20 mL) was added triethylamine (0.540 mL 3.87 mmol). The resulting solution was stirred at room temperature for 5 minutes. Then a solution of the material of Example 104—Step 1 was added in one portion. The solution was stirred at room temperature for 1 hour. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-60%) to give the titled compound (640 mg, 1.660 mmol, 45.0% yield). LC-MS (ESI) m/z 386.2 (M+H)⁺, RT=2.036 minutes.

### Step 3: N-(3,5-dimethoxy-4-methylbenzoyl)-N-(3-phenylpropyl)glycine

To a solution of the material of Example 84—Step 2 (640 mg, 1.660 mmol) in 1,4-dioxane (4 mL) was added 1 N lithium hydroxide (4.98 mL 9.96 mmol). The solution was heated to 50° C. for 1.5 hours. The solution was acidified with 1 N HCl to pH=2-3. It was extracted with ethyl acetate twice. The combined organic layers were washed with brine twice, dried over anhydrous Na₂SO₄, filtered and concentrated to give the titled compound (634 mg, 1.707 mmol). ¹H NMR (400 MHz, DMSO-d₆) δ ppm 12.77 (s, 1H), 7.26 (dt, J=13.7, 7.3 Hz, 2H), 7.20-7.08 (m, 2H), 7.05 (d, J=7.4 Hz, 1H), 6.51 (d, J=3.0 Hz, 2H), 4.07 (s, 1H), 3.91 (s, 1H), 3.74 (d, J=7.5 Hz, 6H), 3.43 (t, J=7.7 Hz, 1H), 3.23 (t, J=7.8 Hz, 1H), 2.61 (t, J=7.9 Hz, 1H), 2.42 (t, J=7.7 Hz, 1H), 1.98 (d, J=10.2 Hz, 3H), 1.91-1.77 (m, 2H); LC-MS (ESI) m/z 372.2 (M+H)⁺, RT=1.897 minutes.

### Step 4: methyl 1-{2-[(3,5-dimethoxy-4-methylbenzoyl)(3-phenylpropyl)amino]acetamido}-4-ethylcyclohexane-1-carboxylate

Example 104—Step 3 (52 mg, 0.14 mmol) and 1-[bis(dimethylamino)methylene]-1H-1,2,3-triazolo[4,5-b]pyridinium 3-oxid hexafluorophosphate (58.6 mg, 0.154 mmol, HATU) was dissolved in a mixture of dry N,N-dimethylformamide (1 mL) and N,N-diisopropylethylamine (72.4 mg, 0.560 mmol). The mixture was stirred for 20 minutes at room temperature. Methyl 1-amino-4-ethylcyclohexanecarboxylate hydrochloride (31.0 mg, 0.140 mmol) in N,N-dimethylformamide (1 mL) was added, and the mixture was stirred for another 3 hours at room temperature. The mixture was poured into water and extracted with ethyl acetate 3 times. The combined organic layers were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (60 mg, 0.111 mmol, 80% yield).

### Step 5: 1-{[N-(3,5-dimethoxy-4-methylbenzoyl)-N-(3-phenylpropyl)glycyl]amino}-4-ethylcyclohexane-1-carboxylic Acid

To a solution of Example 104—Step 4 (60 mg, 0.111 mmol) in tetrahydrofuran (2 mL) was added aqueous lithium hydroxide solution (1.0 N, 0.70 mL). The mixture was stirred at room temperature for 3 hours. The mixture was acidified with 1 N hydrochloric acid to pH=3 and then extracted with ethyl acetate twice. The combined organic layers were concentrated to give a residue which was purified by preparative HPLC (0.1% CF₃CO₂H—H₂O/CH₃CN) and lyophilized to give the titled compound (45 mg, 0.086 mmol, 77% yield).

### Example 105

### 5-{[1-(3-fluoro-4-methoxyphenyl)cyclopropane-1-carbonyl](3-phenylpropyl)amino}pentanoic Acid

### Step 1: 1-(3-fluoro-4-methoxyphenyl)cyclopropane-1-carbonitrile

To a solution of 2-(2-fluoro-4-methoxyphenyl)acetonitrile (200 mg, 1.211 mmol) in N,N-dimethylformamide (1 mL) was added sodium hydride (60% in mineral oil) (145 mg, 3.63 mmol) at 0° C. under an atmosphere of nitrogen. The mixture was stirred at room temperature for 30 minutes. 1-Bromo-2-chloroethane (347 mg, 2.422 mmol) was added to the mixture at 0° C. The reaction mixture was stirred at 30° C. for 3 hours. The mixture was cooled down to 0° C. and quenched with 1 N HCl. The mixture was extracted with ethyl acetate (2×20 mL), and the combined organic layer was washed with brine (2×20 mL), dried over Na₂SO₄ and concentrated. The residue was purified by silica gel flash column chromatography eluted with ethyl acetate in hexane from 0% to 50% over 20 minutes to give the titled compound (170 mg, 0.889 mmol, 73.4% yield). ¹H NMR (400 MHz, methanol-d₄) δ ppm 6.82-6.70 (m, 3H), 3.80 (d, J=1.4 Hz, 3H), 1.65-1.58 (m, 2H), 1.38-1.32 (m, 2H); LC-MS (ESI) m/z 192 (M+H)⁺, RT=1.788 minutes.

### Step 2: 1-(3-fluoro-4-methoxyphenyl)cyclopropane-1-carboxylic Acid

To a solution of Example 105—Step 1 (170 mg, 0.889 mmol) in ethanol (1 mL) was added 2 M KOH (1 mL 2.000 mmol). The mixture was heated to reflux and stirred for 16 hours. The mixture was cooled down and quenched slowly with 1 N HCl. The mixture was extracted with ethyl acetate (2×20 mL), and the combined organic layer was dried over Na₂SO₄ and concentrated to give the titled compound (170 mg, 0.809 mmol, 91% yield). ¹H NMR (400 MHz, methanol-d₄) δ ppm 7.12-6.95 (m, 3H), 3.85 (s, 3H), 1.54 (q, J=3.9 Hz, 2H), 1.16 (q, J=3.9 Hz, 2H); LC-MS (ESI) m/z 211 (M+H)⁺, RT=1.650 minutes.

### Step 3: methyl 5-{[1-(3-fluoro-4-methoxyphenyl)cyclopropane-1-carbonyl](3-phenylpropyl)amino}pentanoate

To a solution of Example 105—Step 2 (29.5 mg, 0.140 mmol) in N,N-dimethylformamide (1 mL) was added 1-[bis(dimethylamino)methylene]-1H-1,2,3-triazolo[4,5-b]pyridinium 3-oxid hexafluorophosphate (56.0 mg, 0.147 mmol, HATU). The resulting solution was stirred at room temperature for 10 minutes. Then a solution of Example 70—Step 1 (35 mg, 0.14 mmol) was added in one portion followed by the addition of diisopropylethylamine (0.049 mL 0.281 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give the titled compound (50 mg, 0.113 mmol, 81% yield). LC-MS (ESI) m/z 442 (M+H)⁺, RT=2.075 minutes.

### Step 4: 5-{[1-(3-fluoro-4-methoxyphenyl)cyclopropane-1-carbonyl](3-phenylpropyl)amino}pentanoic Acid

To a solution of Example 105—Step 3 (60 mg, 0.136 mmol) in dioxane (0.5 mL) was added 1 N LiOH (0.5 mL 0.500 mmol) dropwise. The mixture was stirred at 30° C. for 1 hour. The reaction mixture was purified by preparative HPLC eluting with CH₃CN/H₂O/CF₃CO₂H to give the titled compound (16 mg, 0.037 mmol, 27.5% yield). ¹H NMR (400 MHz, DMSO-d₆) δ ppm 7.31-6.90 (m, 8H), 6.88-6.79 (m, 1H), 3.78 (s, 3H), 3.20 (q, J=9.6, 7.5 Hz, 4H), 2.28 (t, J=7.9 Hz, 2H), 2.23-2.15 (m, 1H), 2.01 (t, J=7.2 Hz, 1H), 1.72 (t, J=8.1 Hz, 1H), 1.38 (d, J=17.6 Hz, 4H), 1.25-1.07 (m, 4H), 0.94 (q, J=4.7 Hz, 1H); LC-MS (ESI) m/z 428 (M+H)⁺, RT=1.920 minutes.

### Example 106

### 5-{[1-(4-methoxyphenyl)cyclopentane-1-carbonyl](3-phenylpropyl)amino}pentanoic Acid

### Step 1: methyl 5-{[1-(4-methoxyphenyl)cyclopentane-1-carbonyl](3-phenylpropyl)amino}pentanoate

To a solution of 1-(4-methoxyphenyl)cyclopentanecarboxylic acid (38.0 mg, 0.172 mmol, Accela ChemBio Co., Ltd) in N,N-dimethylformamide (1 mL) was added 1-[bis(dimethylamino)methylene]-1H-1,2,3-triazolo[4,5-b]pyridinium 3-oxid hexafluorophosphate (68.8 mg, 0.181 mmol, HATU). The resulting solution was stirred at room temperature for 10 minutes. Then Example 70—Step 1 (43 mg, 0.172 mmol) in N,N-dimethylformamide (0.2 mL) was added in one portion followed by the addition of diisopropylethylamine (0.060 mL 0.345 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give the titled compound (60 mg, 0.133 mmol, 77% yield). LC-MS (ESI) m/z 452 (M+H)⁺, RT=2.061 minutes.

### Step 2: 5-{[1-(4-methoxyphenyl)cyclopentane-1-carbonyl](3-phenylpropyl)amino}pentanoic Acid

To a solution of Example 106—Step 1 (60 mg, 0.133 mmol) in dioxane (0.5 mL) was added 1 N LiOH (0.5 mL 0.500 mmol) dropwise. The mixture was stirred at 30° C. for 1 hour. The mixture was purified by preparative HPLC eluting with CH₃CN/H₂O/CF₃CO₂H to give the titled compound (38 mg, 0.087 mmol, 65.4% yield). ¹H NMR (400 MHz, methanol-d₄) δ ppm 7.30-7.10 (m, 5H), 7.03-6.99 (m, 1H), 6.98-6.94 (m, 1H), 6.91-6.84 (m, 2H), 3.77 (d, J=1.9 Hz, 3H), 3.26 (m, 2H), 2.99 (dt, J=12.8, 5.0 Hz, 2H), 2.59 (t, J=7.8 Hz, 1H), 2.34-2.14 (m, 4H), 2.06-1.76 (m, 4H), 1.73-1.50 (m, 6H), 1.31 (dq, J=11.6, 7.8 Hz, 1H), 1.13 (p, J=7.5 Hz, 1H), 1.04-0.91 (m, 1H); LC-MS (ESI) m/z 438 (M+H)⁺, RT=1.898 minutes.

### Example 107

### 5-{[1-(3-chloro-4-methoxyphenyl)cyclopropane-1-carbonyl](3-phenylpropyl)amino}pentanoic Acid

### Step 1: 1-(3-chloro-4-methoxyphenyl)cyclopropane-1-carbonitrile

To a solution of 2-(3-chloro-4-methoxyphenyl)acetonitrile (300 mg, 1.652 mmol) in N,N-dimethylformamide (1 mL) was added sodium hydride (60% in mineral oil) (198 mg, 4.96 mmol) at 0° C. under an atmosphere of nitrogen. The mixture was stirred at room temperature for 30 minutes. 1-Bromo-2-chloroethane (474 mg, 3.30 mmol) was added to the mixture at 0° C. The reaction mixture was stirred at 30° C. for 3 hours. The mixture was cooled down to 0° C. and quenched with 1 N HCl. The mixture was extracted with ethyl acetate (2×20 mL), and the combined organic layers were washed with brine (2×20 mL), dried over Na₂SO₄ and concentrated. The residue was purified by silica gel flash column chromatography eluted with ethyl acetate in hexane from 0% to 50% over 20 minutes to give the titled compound (250 mg, 1.204 mmol, 72.9% yield). ¹H NMR (400 MHz, methanol-d₄) δ ppm 7.34 (d, J=2.4 Hz, 1H), 7.27 (dd, J=8.6, 2.4 Hz, 1H), 7.06 (d, J=8.6 Hz, 1H), 3.88 (s, 3H), 1.71-1.62 (m, 2H), 1.46-1.37 (m, 2H); LC-MS (ESI) m/z 208 (M+H)⁺, RT=1.852 minutes.

### Step 2: 1-(3-chloro-4-methoxyphenyl)cyclopropane-1-carboxylic Acid

To a solution of Example 107—Step 1 (250 mg, 1.204 mmol) in ethanol (1 mL) was added 6 M KOH (1 mL, 6.00 mmol). The mixture was heated to 120° C. under microwave irradiation for 1.5 hours. The mixture was cooled down and slowly quenched with 1 N HCl. The mixture was extracted with ethyl acetate (2×20 mL), and the combined organic layers were dried over Na₂SO₄ and concentrated to give the titled compound (180 mg, 0.794 mmol, 66.0% yield). ¹H NMR (400 MHz, methanol-d₄) δ ppm 7.34 (d, J=2.2 Hz, 1H), 7.23 (dd, J=8.5, 2.2 Hz, 1H), 6.98 (d, J=8.5 Hz, 1H), 3.86 (s, 3H), 1.55 (q, J=4.0 Hz, 2H), 1.16 (q, J=4.0 Hz, 2H); LC-MS (ESI) m/z 227 (M+H)⁺, RT=1.701 minutes.

### Step 3: methyl 5-{[1-(3-chloro-4-methoxyphenyl)cyclopropane-1-carbonyl](3-phenylpropyl)amino}pentanoate

To a solution of Example 107—Step 2 (45.4 mg, 0.201 mmol) in N,N-dimethylformamide (2 mL) was added 1-[bis(dimethylamino)methylene]-1H-1,2,3-triazolo[4,5-b]pyridinium 3-oxid hexafluorophosphate (80 mg, 0.211 mmol, HATU). The resulting solution was stirred at room temperature for 10 minutes. Then a solution of Example 70—Step 1 was added in one portion followed by the addition of diisopropylethylamine (0.070 mL 0.401 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give the titled compound (60 mg, 0.131 mmol, 65.3% yield). LC-MS (ESI) m/z 458 (M+H)⁺, RT=2.123 minutes.

### Step 4: 5-{[1-(3-chloro-4-methoxyphenyl)cyclopropane-1-carbonyl](3-phenylpropyl)amino}pentanoic Acid

To a solution of Example 107—Step 3 (60 mg, 0.131 mmol) in dioxane (0.5 mL) was added 1 N LiOH (0.5 mL 0.500 mmol) dropwise. The mixture was stirred at 30° C. for 1 hour. The mixture was purified by preparative HPLC eluting with CH₃CN/H₂O/CF₃CO₂H to give the titled compound (42 mg, 0.095 mmol, 72.2% yield). ¹H NMR (400 MHz, methanol-d₄) δ ppm 7.31-7.11 (m, 5H), 7.10-6.95 (m, 4H), 3.85 (d, J=3.8 Hz, 3H), 3.36-3.32 (m, 4H), 2.59 (t, J=7.8 Hz, 1H), 2.34 (dt, J=29.6, 7.0 Hz, 2H), 2.09 (t, J=7.3 Hz, 1H), 1.85 (p, J=7.7 Hz, 1H), 1.52 (ddt, J=30.0, 8.1, 3.9 Hz, 4H), 1.35-1.27 (m, 1H), 1.23-1.09 (m, 3H), 0.99-0.91 (m, 1H); LC-MS (ESI) m/z 444 (M+H)⁺, RT=1.963 minutes.

### Example 108

### 5-[(3,5-dimethoxybenzoyl)(3-phenylpropyl)amino]-2,2-dimethylpentanoic Acid

### Step 1: methyl 5-[(3,5-dimethoxybenzoyl)(3-phenylpropyl)amino]-2,2-dimethylpentanoate

To a solution of diisopropylamine (0.197 mL, 1.385 mmol) in tetrahydrofuran (10 mL) was added butyllithium (0.811 mL, 1.298 mmol) at 0° C. under N₂ atmosphere. The mixture was stirred at 0° C. for 15 minutes and then cooled to −78° C. Then a solution of Example 98—Step 1 (370 mg, 0.865 mmol) in tetrahydrofuran (6 mL) was added to the mixture dropwise, and the mixture was stirred at −78° C. for 25 minutes. Then iodomethane (614 mg, 4.33 mmol) was added to the mixture with continued stirring at −78° C. to −30° C. over 2 hours. Aqueous NH₄Cl was added to the mixture, and the mixture was extracted with ethyl acetate (60 mL). The organic fraction was dried and concentrated. The residue was purified by chromatography on silica gel eluted with hexanes and ethyl acetate (from 0-50%) to give the titled compound (340 mg, 0.770 mmol, 89% yield). ¹H NMR (400 MHz, CDCl₃) δ ppm 7.25 (d, J=12.0 Hz, 4H), 7.03 (d, J=7.5 Hz, 1H), 6.56-6.37 (m, 3H), 3.79 (s, 6H), 3.69-3.56 (m, 3H), 3.47 (d, J=23.6 Hz, 2H), 3.19 (d, J=23.2 Hz, 2H), 2.70 (s, 1H), 2.44 (d, J=7.8 Hz, 1H), 1.98 (s, 1H), 1.82 (s, 1H), 1.74 (s, 1H), 1.62-1.49 (m, 2H), 1.48-1.35 (m, 1H), 1.22-1.03 (m, 6H); LC-MS (ESI) m/z 442 (M+H)⁺, RT=2.15 minutes.

### Step 2: 5-[(3,5-dimethoxybenzoyl)(3-phenylpropyl)amino]-2,2-dimethylpentanoic Acid

To a solution of Example 108—Step 1 (340 mg, 0.770 mmol) in 1,4-dioxane (5 mL) was added 1 N LiOH (7.70 mL 7.70 mmol), and the mixture was heated to 70° C. overnight. Then the mixture was cooled to room temperature and concentrated. The residue was diluted with water and washed with ethyl ether (20 mL). The aqueous layer was acidified with 1 N HCl to pH=2-3 and extracted with ethyl acetate (30 mL). The organic layer was dried over Na₂SO₄, filtered and concentrated to give a residue which was purified by high pressure liquid chromatography eluted with acetonitrile and water (0.5% CF₃CO₂H) to give the titled compound (242 mg, 0.566 mmol, 73.5% yield). ¹H NMR (400 MHz, DMSO-d₆) δ ppm 12.09 (s, 1H), 7.44-6.94 (m, 5H), 6.49 (d, J=3.0 Hz, 1H), 6.38 (d, J=2.3 Hz, 2H), 3.73 (s, 6H), 3.44-3.28 (m, 3H), 3.10 (t, J=7.8 Hz, 2H), 2.61 (t, J=7.6 Hz, 1H), 2.38 (t, J=7.5 Hz, 1H), 1.81 (dt, J=41.5, 7.8 Hz, 2H), 1.41 (d, J=32.4 Hz, 4H), 1.26-1.14 (m, 1H), 1.04 (d, J=35.0 Hz, 6H); LC-MS (ESI) m/z 428 (M+H)⁺, RT=1.98 minutes.

### Example 109

### (2-{[3-(3-chlorophenyl)propyl](3,5-dimethoxy-4-methylbenzoyl)amino}ethoxy)acetic Acid

### Step 1: N-(2-{[tert-butyl(dimethyl)silyl]oxy}ethyl)-3-(3-chlorophenyl)propan-1-amine

A mixture of (2-bromoethoxy)(tert-butyl)dimethylsilane (700 mg, 2.93 mmol), 3-(3-chlorophenyl)propan-1-amine (496 mg, 2.93 mmol), and potassium carbonate (485 mg, 3.51 mmol) in acetonitrile (6 mL) was refluxed for 16 hours. The mixture was cooled to room temperature and filtered to remove potassium carbonate to give the titled compound. LC-MS (ESI) m/z 328.2 (M+H)⁺, RT=1.67 minutes.

### Step 2: N-(2-{[tert-butyl(dimethyl)silyl]oxy}ethyl)-N-[3-(3-chlorophenyl)propyl]-3,5-dimethoxy-4-methylbenzamide

To a solution of 3,5-dimethoxy-4-methylbenzoic acid (0.575 g, 2.93 mmol) and 1-[bis(dimethylamino)methylene]-1H-1,2,3-triazolo[4,5-b]pyridinium 3-oxid hexafluorophosphate (1.170 g, 3.08 mmol, HATU) in N,N-dimethylformamide (10 mL) was added triethylamine (0.429 mL 0.311 mmol). The resulting solution was stirred at room temperature for 10 minutes. Then a solution of Example 109—Step 1 (1.19 g, 1.451 mmol) in CH₃CN (10 mL) was added in one portion. The reaction mixture was stirred at room temperature for 2 hours. The reaction mixture was diluted with water and extracted with ethyl acetate twice. The combined organic layers were washed with saturated NaHCO₃ solution (10 mL) and brine, dried over Na₂SO₄, filtered and concentrated. The residue was added to a silica gel column and was eluted with hexanes and ethyl acetate (0-50%) to give the titled compound (0.5 g, 0.889 mmol, 61.3% yield). LC-MS (ESI) m/z 506.2 (M+H)⁺, RT=2.50 minutes.

### Step 3: N-[3-(3-chlorophenyl)propyl]-N-(2-hydroxyethyl)-3,5-dimethoxy-4-methylbenzamide

To a solution of Example 109—Step 2 (0.5 g, 0.988 mmol) in tetrahydrofuran (10 mL) was added tetra-n-butylammonium fluoride (0.310 g, 1.185 mmol). The reaction mixture was stirred at 20° C. for 1 hour. The mixture was concentrated, and the residue was dissolved in t-butyl methyl ether (10 mL). This mixture was washed with water (5 mL) and brine (5 mL), dried over Na₂SO₄, filtered and concentrated to give the titled compound. LC-MS (ESI) m/z 392.2 (M+H)⁺, RT=1.98 minutes.

### Step 4: methyl (2-{[3-(3-chlorophenyl)propyl](3,5-dimethoxy-4-methylbenzoyl)amino}ethoxy)acetate

To a solution of Example 109—Step 3 (0.4 g, 1.021 mmol) in tetrahydrofuran (5 mL) was added methyl 2-chloroacetate (0.268 mL 3.06 mmol) and potassium tert-butoxide (0.344 g, 3.06 mmol) in one portion. The mixture was heated to reflux for 2 hours. The mixture was cooled down, quenched with saturated aqueous NH₄Cl, and extracted with ethyl acetate 3 times. The combined organic layers were washed with brine, dried over anhydrous Na₂SO₄, filtered and concentrated to give a residue which was purified by flash column chromatography on silica gel (eluent: hexanes:ethyl acetate from 100:0 to 50:50) to give the titled compound (0.15 g, 0.291 mmol, 28.5% yield). LC-MS (ESI) m/z 462.2 (M+H)⁺, RT=2.11 minutes.

### Step 5: (2-{[3-(3-chlorophenyl)propyl](3,5-dimethoxy-4-methylbenzoyl)amino}ethoxy)acetic Acid

To a solution of Example 109—Step 4 (0.15 g, 0.323 mmol) in tetrahydrofuran (4 mL) was added 1 N LiOH (1.940 mL 1.940 mmol). It was heated to 50° C. for 2 hours. The mixture was cooled down and acidified with 1 N HCl to pH=2-3 and then extracted with ethyl acetate twice. The combined organic layers were concentrated, and the residue was purified by preparative HPLC (0.1% aqueous ammonium bicarbonate/CH₃CN) and lyophilized to give the titled compound (55 mg, 0.120 mmol, 37.1% yield). ¹H NMR (400 MHz, DMSO-d₆) δ ppm 7.28-7.10 (m, 4H), 6.57 (s, 2H), 3.82 (m, 2H), 3.76 (s, 6H), 3.65-3.55 (m, 2H), 3.52-3.30 (m, 4H), 2.60-2.51 (m, 2H), 2.00 (s, 3H), 1.93-1.81 (m, 2H); LC-MS (ESI) m/z 450.2 (M+H)⁺, RT=1.96 minutes.

### Example 110

### (2-{(3,5-dimethoxy-4-methylbenzoyl)[3-(2-fluorophenyl)propyl]amino}ethoxy)acetic Acid

### Step 1: N-(2-{[tert-butyl(dimethyl)silyl]oxy}ethyl)-3-(2-fluorophenyl)propan-1-amine

In a 10 mL sealed tube, a mixture of (2-bromoethoxy)(tert-butyl)dimethylsilane (700 mg, 2.93 mmol), 3-(2-fluorophenyl)propan-1-amine (448 mg, 2.93 mmol), and potassium carbonate (485 mg, 3.51 mmol) in acetonitrile (6 mL) was stirred at reflux for 16 hours. The mixture was cooled to room temperature and filtered to remove potassium carbonate. The filtrate was concentrated to give the titled compound. LC-MS (ESI) m/z 312.2 (M+H)⁺, RT=1.84 minutes.

### Step 2: N-(2-{[tert-butyl(dimethyl)silyl]oxy}ethyl)-N-[3-(2-fluorophenyl)propyl]-3,5-dimethoxy-4-methylbenzamide

To a solution of 3,5-dimethoxy-4-methylbenzoic acid (0.510 g, 2.6 mmol) and 1-[bis(dimethylamino)methylene]-1H-1,2,3-triazolo[4,5-b]pyridinium 3-oxid hexafluorophosphate (0.879 g, 2.311 mmol, HATU) in N,N-dimethylformamide (10 mL) was added triethylamine (0.322 mL 2.311 mmol). The resulting solution was stirred at room temperature for 10 minutes. Then a solution of Example 110—Step 1 (1 g, 1.445 mmol) in CH₃CN (10 mL) was added in one portion. The reaction mixture was stirred at room temperature for 2 hours. The reaction mixture was diluted with water and extracted with ethyl acetate twice. The combined organic layers were washed with saturated NaHCO₃ solution (1×10 mL) and brine, dried over Na₂SO₄, filtered and concentrated. The residue was added to a silica gel column which was eluted with hexanes and ethyl acetate (0-50%) to give the titled compound (0.6 g, 1.103 mmol, 76% yield). LC-MS (ESI) m/z 490.2 (M+H)⁺, RT=2.49 minutes.

### Step 3: N-[3-(2-fluorophenyl)propyl]-N-(2-hydroxyethyl)-3,5-dimethoxy-4-methylbenzamide

To a solution of Example 110—Step 2 (0.6 g, 1.225 mmol) in tetrahydrofuran (10 mL) was added tetra-n-butylammonium fluoride (0.320 g, 1.225 mmol). The reaction mixture was stirred at 20° C. for 1 hour. The mixture was concentrated, and the residue was dissolved in t-butyl methyl ether (10 mL). This mixture was washed with water (5 mL) and brine (5 mL), dried over Na₂SO₄, filtered and concentrated. The residue was used to the next step without further purification. LC-MS (ESI) m/z 376.2 (M+H)⁺, RT=1.88 minutes.

### Step 4: methyl (2-{(3,5-dimethoxy-4-methylbenzoyl)[3-(2-fluorophenyl)propyl]amino}ethoxy)acetate

To a solution of Example 110—Step 3 (0.5 g, 1.332 mmol) in tetrahydrofuran (5 mL) was added methyl 2-chloroacetate (0.350 mL 4.00 mmol) and potassium tert-butoxide (0.448 g, 4.00 mmol) in one portion. The mixture was heated to reflux for 2 hours. The mixture was cooled down, quenched with saturated aqueous NH₄Cl, and extracted with ethyl acetate 3 times. The combined organic layers were washed with brine, dried over anhydrous Na₂SO₄, filtered and concentrated to give a residue which was purified by flash column chromatography on silica gel (eluent: hexanes:ethyl acetate from 100:0 to 50:50) to give the titled compound (0.15 g, 0.302 mmol, 22.65% yield). LC-MS (ESI) m/z 448.2 (M+H)⁺, RT=2.04 minutes.

### Step 5: (2-{(3,5-dimethoxy-4-methylbenzoyl)[3-(2-fluorophenyl)propyl]amino}ethoxy)acetic Acid

To a solution of Example 110—Step 4 (0.15 g, 0.335 mmol) in tetrahydrofuran (4 mL) was added LiOH (0.048 g, 2.011 mmol). It was heated to 50° C. for 2 hours. The mixture was cooled down and acidified with 1 N HCl to pH=2-3 and then extracted with ethyl acetate twice. The combined organic layers were concentrated, and the residue was purified by preparative HPLC (0.1% aqueous ammonium bicarbonate/CH₃CN) and lyophilized to give the titled compound (68 mg, 0.154 mmol, 45.9% yield). 1H NMR (400 MHz, DMSO-d₆, T=60° C.) d 7.23-7.03 (m, 4H), 6.65 (s, 2H), 3.81 (m, 2H), 3.75 (s, 6H), 3.64-3.52 (m, 2H), 3.50-3.35 (m, 4H), 2.60-2.52 (m, 2H), 1.99 (s, 3H), 1.90-1.80 (m, 2H); LC-MS (ESI) m/z 434.2 (M+H)⁺, RT=1.90 minutes.

### Example 111

### N-{5-[(methanesulfonyl)amino]-5-oxopentyl}-3,5-dimethoxy-4-methyl-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (43.1 mg, 0.266 mmol), in isopropyl acetate (2 mL) was stirred at room temperature for 10 minutes. Methanesulfonamide (25.3 mg, 0.266 mmol) and 1,8-diazabicyclo[5.4.0]undec-7-ene (0.058 mL, 0.387 mmol) were added, and the reaction mixture was stirred at room temperature. At 2 hours, LC-MS showed formation of the titled compound and ˜5% of unreacted starting material. 1,1′-Carbonyldiimidazole (22 mg, 0.13 mmol) in isopropyl acetate (1 mL) was added to the reaction mixture, and the resultant mixture was stirred at room temperature for 10 minutes. Methanesulfonamide (13 mg, 0.13 mmol) and 1,8-diazabicyclo[5.4.0]undec-7-ene (0.029 mL, 0.194 mmol) were then added, and the reaction mixture was agitated in an ultrasonic bath for 2 minutes and stirred at 40° C. overnight. The reaction mixture was diluted with 10 mL of ethyl acetate, washed with 10 mL of 1 N HCl and saturated NaCl, dried over MgSO₄, and concentrated. The residue was re-dissolved in isopropyl acetate, 1,1′-carbonyldiimidazole (43.1 mg, 0.266 mmol) was added, and the mixture was stirred at 40° C. for 30 minutes. Then methanesulfonamide (25.3 mg, 0.266 mmol) and 1,8-diazabicyclo[5.4.0]undec-7-ene (0.058 mL, 0.387 mmol) were added. The reaction mixture was stirred at 50° C. overnight, and then the mixture was cooled to room temperature, diluted with 10 mL of ethyl acetate, washed with 10 mL of 1 N HCl and saturated NaCl, dried over MgSO₄ and concentrated. Flash chromatography (100% ethyl acetate) gave the titled compound (36 mg, 30%). ¹H NMR (400 MHz, DMSO-d₆) δ ppm 1.54 (m, 4H), 1.82 (d, J=26.6 Hz, 2H), 1.98 (s, 3H), 2.15 (s, 1H), 2.26-2.45 (m, 2H), 2.61 (d, J=1.9 Hz, 1H), 3.19 (s, 5H), 3.37 (d, J=2.7 Hz, 2H), 3.74 (s, 6H), 6.48 (s, 2H), 6.87-7.38 (m, 5H), 11.62 (s, 1H); MS (DCI) m/z 491 (M+H)⁺.

### Example 112

### [(2-{[3-(2-chlorophenyl)propyl](3,5-dimethoxy-4-methylbenzoyl)amino}ethyl)sulfanyl]acetic Acid

### Step 1: 3-(2-chlorophenyl)propanamide

A mixture of 3-(2-chlorophenyl)propanoic acid (2.0 g, 10.83 mmol) in thionyl chloride (10 mL, 137 mmol) was heated to reflux for 2 hours. Then the mixture was concentrated, the residue was dissolved in anhydrous tetrahydrofuran (30 mL), and the solution was cooled to 0° C. Ammonia gas was bubbled into the system until pH>7. A solid was formed. Then the mixture was concentrated to give the titled compound (2.1 g, 10.29 mmol, 95% yield). ¹H NMR (400 MHz, DMSO-d₆) δ ppm 7.42-7.33 (m, 4H), 7.28-7.18 (m, 2H), 2.97-2.78 (m, 2H), 2.41-2.20 (m, 2H); LC-MS (ESI) m/z 184.0 (M+H)⁺, RT=0.289 minutes.

### Step 2: 3-(2-chlorophenyl)propan-1-amine

To a stirred solution of Example 112—Step 1 (2.1 g, 10.29 mmol) in tetrahydrofuran (60 mL) was added aluminum(III) lithium hydride (1.0 g, 26.3 mmol) in small portions at 0° C. Then the reaction mixture was heated to reflux for 2 hours. Then the mixture was quenched with 3.0 g of Na₂SO₄.10H₂O. To the mixture was added Mg₂SO₄ (5 g), and then the solid was removed by filtration. The filtrate was concentrated to give the titled compound (1.8 g, 8.49 mmol, 82% yield). ¹H NMR (400 MHz, DMSO-d₆) δ ppm 7.38 (dd, J=7.7, 1.5 Hz, 1H), 7.32 (dd, J=7.4, 1.9 Hz, 1H), 7.22 (dtd, J=21.4, 7.4, 1.7 Hz, 2H), 2.73-2.62 (m, 2H), 2.54 (t, J=6.9 Hz, 2H), 1.65-1.56 (m, 2H); LC-MS (ESI) m/z 172.2 (M+H)⁺, RT=1.349 minutes.

### Step 3: ethyl [(2-bromoethyl)sulfanyl]acetate

A mixture of ethyl 2-mercaptoacetate (10.0 g, 83 mmol), 1,2-dibromoethane (46.9 g, 250 mmol) and K₂CO₃ (34.5 g, 250 mmol) in acetonitrile (150 mL) was heated to reflux for 2 hours. Then the mixture was cooled to room temperature and filtered to remove solids. The filtrate was concentrated, and the residue was purified by chromatography on silica gel eluted with hexane and ethyl acetate (0-10%) to give the titled compound (14.0 g, 61.6 mmol, 74.1% yield).

### Step 4: ethyl [(2-{[3-(2-chlorophenyl)propyl]amino}ethyl)sulfanyl]acetate

A mixture of Example 112—Step 2 (250 mg, 1.474 mmol) and Example 112—Step 3 (335 mg, 1.474 mmol) in CH₃CN (3 mL) was stirred at reflux for 1 hour. The mixture was cooled to room temperature and filtered. The filtrate was concentrated to give the titled compound. LC-MS (ESI) m/z 316.2 (M+H)⁺, RT=1.664 minutes.

### Step 5: ethyl [(2-{[3-(2-chlorophenyl)propyl](3,5-dimethoxy-4-methylbenzoyl)amino}ethyl)sulfanyl]acetate

To a solution of 3,5-dimethoxy-4-methylbenzoic acid (289 mg, 1.474 mmol) and 1-[bis(dimethylamino)methylene]-1H-1,2,3-triazolo[4,5-b]pyridinium 3-oxid hexafluorophosphate (617 mg, 1.621 mmol, HATU) in N,N-dimethylformamide (5 mL) was added triethylamine (0.226 mL 1.621 mmol). The resultant solution was stirred at room temperature for 30 minutes. Then it was added to a solution of Example 112—Step 4 (466 mg, 1.474 mmol) in CH₃CN (3 mL) and triethylamine (0.5 mL). The mixture was stirred at room temperature for 1 hour. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (90 mg, 0.118 mmol, 8.03% yield). LC-MS (ESI) m/z 494.2 (M+H)⁺, RT=2.193 minutes.

### Step 6: 2-((2-(N-(3-(2-chlorophenyl)propyl)-3,5-dimethoxy-4-methylbenzamido)ethyl)thio)acetic Acid

To a solution of Example 112—Step 5 (90 mg, 0.182 mmol) in tetrahydrofuran (2 mL) was added 1 N lithium hydroxide (1.093 mL 1.093 mmol). The mixture was stirred at room temperature for 2 hours. The solution was concentrated under reduced pressure to remove most of the tetrahydrofuran. A solid precipitated from the aqueous mixture. The solids were collected by filtration and washed with water and hexanes to give the lithium salt of the titled compound. The salt was treated with 1 N HCl (4 mL) under ultra-sonic treatment. The solid was then collected by filtration and washed with water and hexanes to give the titled compound (42 mg, 0.090 mmol, 49.5% yield). ¹H NMR (400 MHz, DMSO-d₆) δ ppm 12.36 (s, 1H), 7.34 (d, J=7.1 Hz, 1H), 7.31-7.14 (m, 3H), 6.52 (s, 2H), 3.76 (s, 6H), 3.53 (s, 2H), 3.36 (s, 2H), 3.21 (s, 2H), 2.81 (t, J=7.4 Hz, 2H), 2.62 (s, 2H), 2.00 (s, 3H), 1.91-1.79 (m, 2H); LC-MS (ESI) m/z 466.2 (M+H)⁺, RT=1.988 minutes.

### Example 113

### [(2-{[3-(2,6-difluorophenyl)propyl](3,5-dimethoxy-4-methylbenzoyl)amino}ethyl)sulfanyl]acetic Acid

### Step 1: 3-(2,6-difluorophenyl)propanamide

A solution of 3-(2,6-difluorophenyl)propanoic acid (2.0 g, 10.74 mmol) in thionyl chloride (10 mL, 137 mmol) was heated to reflux for 2 hours. Then the mixture was concentrated, the residue was dissolved in anhydrous tetrahydrofuran (30 mL), and the solution was cooled to 0° C. Ammonia gas was bubbled into the system till pH>7, and a solid formed. Then the mixture was concentrated to give the titled compound (2.094 g, 10.74 mmol, 100% yield). ¹H NMR (400 MHz, methanol-d₄) δ ppm 7.24 (tt, J=8.4, 6.5 Hz, 1H), 6.98-6.86 (m, 2H), 3.02-2.93 (m, 2H), 2.52-2.43 (m, 2H); LC-MS (ESI) m/z 186.2 (M+H)⁺, RT=1.414 minutes.

### Step 2: 3-(2,6-difluorophenyl)propan-1-amine

To a solution of Example 113—Step 1 (2.1 g, 11.34 mmol) in tetrahydrofuran (60 mL) was added aluminum(III) lithium hydride (1.0 g, 26.3 mmol) in small portions at 0° C. Then the reaction mixture was heated to reflux for 2 hours. Then the mixture was quenched with 3.0 g of Na₂SO₄*10H₂0. To the mixture was added Mg₂SO₄ (5 g), and then the solids were removed by filtration. The filtrate was concentrated to give the titled compound (1.76 g, 8.22 mmol, 72.5% yield). ¹H NMR (400 MHz, CDCl₃) δ ppm 7.17-7.09 (m, 1H), 6.88-6.78 (m, 2H), 2.86-2.63 (m, 4H), 1.79 (h, J=7.4, 6.8 Hz, 2H); LC-MS (ESI) m/z 172.2 (M+H)⁺, RT=1.181 minutes.

### Step 3: ethyl [(2-{[3-(2,6-difluorophenyl)propyl]amino}ethyl)sulfanyl]acetate

A mixture of Example 113—Step 2 (250 mg, 1.460 mmol) and Example 112—Step 3 (332 mg, 1.460 mmol) in CH₃CN (3 mL) was stirred at reflux for 1.5 hours. Then the solution was cooled down to give the titled compound. LC-MS (ESI) m/z 318.2 (M+H)⁺, RT=1.606 minutes.

### Step 4: ethyl [(2-{[3-(2,6-difluorophenyl)propyl](3,5-dimethoxy-4-methylbenzoyl)amino}ethyl)sulfanyl]acetate

To a solution of 3,5-dimethoxy-4-methylbenzoic acid (286 mg, 1.460 mmol) and 1-[bis(dimethylamino)methylene]-1H-1,2,3-triazolo[4,5-b]pyridinium 3-oxid hexafluorophosphate (611 mg, 1.606 mmol, HATU) in N,N-dimethylformamide (5 mL) was added triethylamine (0.224 mL 1.606 mmol). The resultant mixture was stirred at room temperature for 30 minutes. A solution of Example 113—Step 3 (463 mg, 1.460 mmol) and triethylamine (0.5 mL) in CH₃CN (3 mL) was added into the mixture. The mixture was stirred at room temperature for 2 hours. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (330 mg, 0.360 mmol, 24.63% yield). LC-MS (ESI) m/z 496.2 (M+H)⁺, RT=2.147 minutes.

### Step 5: [(2-{[3-(2,6-difluorophenyl)propyl](3,5-dimethoxy-4-methylbenzoyl)amino}ethyl)sulfanyl]acetic Acid

To a solution of Example 113—Step 4 (330 mg, 0.666 mmol) in tetrahydrofuran (4 mL) was added 1 N lithium hydroxide (4.00 mL 4.00 mmol). The mixture was stirred at room temperature for 2 hours. The solution was concentrated under reduced pressure to remove most of the tetrahydrofuran. A solid precipitated which was collected by filtration and washed with water and hexanes to give the lithium salt of the titled compound. The salt was treated with 1 N HCl (4 mL) under ultra-sonic treatment. Then the solid was collected by filtration and washed with water and hexanes. This solid was dissolved in tetrahydrofuran (2 mL) and purified by preparative HPLC (0.1% CF₃CO₂H—H₂O/CH₃CN) and lyophilized to give the titled compound (147 mg, 0.314 mmol, 47.2% yield). ¹H NMR (400 MHz, DMSO-d₆) δ ppm 7.32-7.17 (m, 1H), 6.95 (t, J=7.9 Hz, 2H), 6.48 (s, 2H), 3.74 (s, 6H), 3.51 (s, 2H), 3.31 (s, 2H), 3.20 (s, 2H), 2.88-2.75 (m, 2H), 2.50 (s, 2H), 1.98 (s, 3H), 1.80 (dt, J=15.3, 7.4 Hz, 2H); LC-MS (ESI) m/z 468.2 (M+H)⁺, RT=1.951 minutes.

### Example 114

### [(2-{[3-(2,4-difluorophenyl)propyl](3,5-dimethoxy-4-methylbenzoyl)amino}ethyl)sulfanyl]acetic Acid

### Step 1: 3-(2,4-difluorophenyl)propanamide

A solution of 3-(2,4-difluorophenyl)propanoic acid (1.0 g, 5.37 mmol) in thionyl chloride (10 mL, 137 mmol) was heated at reflux for 2 hours. The mixture was then concentrated, the residue was dissolved in anhydrous tetrahydrofuran (30 mL), and the solution was cooled to 0° C. Ammonia gas was bubbled in until pH>7 producing a solid. Then the mixture was concentrated to give the titled compound (1.047 g, 5.37 mmol, 100% yield). ¹H NMR (400 MHz, methanol-d₄) δ ppm 7.29 (td, J=8.8, 6.4 Hz, 1H), 7.00-6.71 (m, 2H), 2.92 (t, J=7.7 Hz, 2H), 2.61-2.34 (m, 2H); LC-MS (ESI) m/z 186.0 (M+H)⁺, RT=1.471 minutes.

### Step 2: 3-(2,4-difluorophenyl)propan-1-amine

To a solution of Example 114—Step 1 (1.045 g, 5.64 mmol) in tetrahydrofuran (60 mL) was added aluminum(III) lithium hydride (1.0 g, 26.3 mmol) in small portions at 0° C. Then the reaction mixture was heated at reflux for 2 hours. Then the mixture was quenched with 3.0 g of Na₂SO₄. To the mixture was added 5 g of Mg₂SO₄, and then the solids were removed by filtration. The solution was concentrated to give the titled compound (800 mg, 3.74 mmol, 66.2% yield). ¹H NMR (400 MHz, CDCl₃) δ ppm 7.19-7.06 (m, 1H), 6.89-6.66 (m, 2H), 2.77 (t, J=7.1 Hz, 2H), 2.65 (q, J=7.9 Hz, 2H), 1.79 (q, J=7.4 Hz, 2H); LC-MS (ESI) m/z 172.2 (M+H)⁺, RT=1.215 minutes.

### Step 3: ethyl [(2-{[3-(2,4-difluorophenyl)propyl]amino}ethyl)sulfanyl]acetate

A mixture of Example 114—Step 2 (210 mg, 1.227 mmol) and Example 112—Step 3 (279 mg, 1.227 mmol) in CH₃CN (3 mL) was stirred at reflux for 1.5 hours to give the titled compound. LC-MS (ESI) m/z 318.2 (M+H)⁺, RT=1.604 minutes.

### Step 4: ethyl [(2-{[3-(2,4-difluorophenyl)propyl](3,5-dimethoxy-4-methylbenzoyl)amino}ethyl)sulfanyl]acetate

To a solution of 3,5-dimethoxy-4-methylbenzoic acid (241 mg, 1.227 mmol) and 1-[bis(dimethylamino)methylene]-1H-1,2,3-triazolo[4,5-b]pyridinium 3-oxid hexafluorophosphate (513 mg, 1.350 mmol, HATU) in N,N-dimethylformamide (5 mL) was added triethylamine (0.188 mL 1.350 mmol). The resultant mixture was stirred at room temperature for 30 minutes. A solution of Example 114—Step 3 (389 mg, 1.227 mmol) and triethylamine (0.5 mL) in CH₃CN (3.0 mL) was added. The mixture was stirred at room temperature for 1 hour. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (320 mg, 0.258 mmol, 21.05% yield). LC-MS (ESI) m/z 496.2 (M+H)⁺, RT=2.148 minutes.

### Step 5: [(2-{[3-(2,4-difluorophenyl)propyl](3,5-dimethoxy-4-methylbenzoyl)amino}ethyl)sulfanyl]acetic Acid

To a solution of Example 114—Step 4 (320 mg, 0.646 mmol) in tetrahydrofuran (4 mL) was added 1 N lithium hydroxide (3.87 mL 3.87 mmol). The mixture was stirred at room temperature for 2 hours. The solution was concentrated under reduced pressure to remove most of the tetrahydrofuran. A solid precipitated which was collected by filtration. The solid was washed with water and hexanes to give the lithium salt of the target molecule. The salt was treated with 1 N HCl (4 mL) under ultra-sonic treatment. The solid was collected by filtration and washed with water and hexanes. This solid was dissolved in 2 mL of tetrahydrofuran and purified by preparative HPLC (0.1% CF₃CO₂H—H₂O/CH₃CN) and lyophilized to give the titled compound (103 mg, 0.220 mmol, 34.1% yield). ¹H NMR (400 MHz, DMSO-d₆) δ ppm 7.23 (s, 1H), 7.09-6.98 (m, 1H), 6.90 (t, J=8.1 Hz, 1H), 6.49 (s, 2H), 3.75 (s, 6H), 3.51 (s, 2H), 3.30 (s, 2H), 3.20 (s, 2H), 2.81 (t, J=7.5 Hz, 2H), 2.49 (d, J=1.9 Hz, 2H), 1.99 (s, 3H), 1.89-1.76 (m, 2H); LC-MS (ESI) m/z 468.2 (M+H)⁺, RT=1.959 minutes.

### Example 115

### [(2-{[1-(4-methoxyphenyl)cyclopropane-1-carbonyl](3-phenylpropyl)amino}ethyl)sulfanyl]acetic Acid

### Step 1: ethyl ({2-[(3-phenylpropyl)amino]ethyl}sulfanyl)acetate

A mixture of 3-phenylpropan-1-amine (170 mg, 1.257 mmol) and ethyl [(2-bromoethyl)sulfanyl]acetate (286 mg, 1.257 mmol, Example 132—Step 4) in CH₃CN (6 mL) was stirred at reflux for 3 hours. Then the mixture was cooled down and filtered, the filtrate was concentrated to give the titled compound. LC-MS (ESI) m/z 282 (M+H)⁺, RT=1.754 minutes

### Step 2: ethyl [(2-{[1-(4-methoxyphenyl)cyclopropane-1-carbonyl](3-phenylpropyl)amino}ethyl)sulfanyl]acetate

To a solution of 1-(4-methoxyphenyl)cyclopropanecarboxylic acid (239 mg, 1.244 mmol) in N,N-dimethylformamide (3 mL) was added 1-[bis(dimethylamino)methylene]-1H-1,2,3-triazolo[4,5-b]pyridinium 3-oxid hexafluorophosphate (497 mg, 1.306 mmol, HATU). The resulting solution was stirred at room temperature for 10 minutes. A solution of ethyl ({2-[(3-phenylpropyl)amino]ethyl}sulfanyl)acetate (350 mg, 1.244 mmol, Example 115—Step 1) in N,N-dimethylformamide (1.0 mL) was added in one portion followed by the addition of diisopropylethylamine (0.434 mL 2.487 mmol). The solution was stirred at room temperature for 1 hour. The reaction mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (0.15 g, 0.329 mmol, 26.5% yield). LC-MS (ESI) m/z 456 (M+H)⁺, RT=2.12 minutes.

### Step 3: [(2-{[1-(4-methoxyphenyl)cyclopropane-1-carbonyl](3-phenylpropyl)amino}ethyl)sulfanyl]acetic Acid

To a solution of Example 115—Step 2 (0.15 g, 0.329 mmol) in dioxane (0.5 mL) was added 1 N LiOH (0.5 mL 0.500 mmol) dropwise. The mixture was stirred at 30° C. for 1 hour. The pH of the mixture was adjusted to about 6-7 and purified by preparative HPLC eluting with CH₃CN/H₂O/CF₃CO₂H to give the titled compound (60 mg, 0.140 mmol, 42.6% yield). ¹H NMR (400 MHz, methanol-d₄) δ ppm 7.28-7.13 (m, 4H), 7.07-6.97 (m, 3H), 6.86 (dd, J=15.8, 8.5 Hz, 2H), 3.76 (s, 3H), 3.61-3.47 (m, 2H), 3.41-3.32 (m, 2H), 3.25 (s, 1H), 3.02 (s, 1H), 2.79 (dd, J=8.2, 6.2 Hz, 1H), 2.58 (t, J=7.7 Hz, 1H), 2.34 (t, J=7.5 Hz, 1H), 2.26 (t, J=8.1 Hz, 1H), 1.85 (p, J=7.8 Hz, 1H), 1.49-1.39 (m, 1H), 1.34-1.11 (m, 3H), 0.98 (q, J=4.6 Hz, 1H); LC-MS (ESI) m/z 428 (M+H)⁺, RT=1.928 minutes.

### Example 116

### [(2-{(3,5-dimethoxy-4-methylbenzoyl)[3-(2-fluorophenyl)propyl]amino}ethyl)sulfanyl]acetic Acid

### Step 1: ethyl [(2-{[3-(2-fluorophenyl)propyl]amino}ethyl)sulfanyl]acetate

A mixture of 3-(2-fluorophenyl)propan-1-amine (300 mg, 1.958 mmol) and Example 112—Step 3 (445 mg, 1.958 mmol) in CH₃CN (2 mL) was stirred at reflux for 2 hours. The mixture was cooled and concentrated to give the titled compound. LC-MS (ESI) m/z 300 (M+H)⁺, RT=1.593 minutes.

### Step 2: ethyl [(2-{(3,5-dimethoxy-4-methylbenzoyl)[3-(2-fluorophenyl)propyl]amino}ethyl) sulfanyl]acetate

To a solution of 3,5-dimethoxy-4-methylbenzoic acid (360 mg, 1.837 mmol) in N,N-dimethylformamide (3 mL) was added 1-[bis(dimethylamino)methylene]-1H-1,2,3-triazolo[4,5-b]pyridinium 3-oxid hexafluorophosphate (733 mg, 1.929 mmol, HATU). The resulting solution was stirred at room temperature for 10 minutes. A solution of Example 116—Step 1 (550 mg, 1.837 mmol) in N,N-dimethylformamide (1.0 mL) was added in one portion followed by the addition of diisopropylethylamine (0.642 mL 3.67 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (0.2 g, 0.419 mmol, 22.80% yield). LC-MS (ESI) m/z 479 (M+H)⁺, RT=2.149 minutes.

### Step 3: [(2-{(3,5-dimethoxy-4-methylbenzoyl)[3-(2-fluorophenyl)propyl]amino}ethyl)sulfanyl]acetic Acid

To a solution of Example 116—Step 2 (0.2 g, 0.419 mmol) in dioxane (0.5 mL) was added 1 N LiOH (0.5 mL 0.500 mmol) dropwise. The mixture was stirred at 30° C. for 1 hour. The mixture was adjusted to pH about 6-7 and purified by preparative HPLC eluting with CH₃CN/H₂O/CF₃CO₂H to give the titled compound (65 mg, 0.145 mmol, 34.5% yield). ¹H NMR (400 MHz, methanol-d₄) δ ppm 7.39-6.86 (m, 4H), 6.53 (d, J=44.2 Hz, 2H), 3.80 (d, J=19.1 Hz, 8H), 3.64-3.45 (m, 2H), 3.40-3.32 (m, 2H), 2.95 (d, J=6.8 Hz, 2H), 2.80-2.72 (m, 1H), 2.54-2.46 (m, 1H), 2.04 (s, 4H), 1.88 (dd, J=14.2, 7.2 Hz, 1H); LC-MS (ESI) m/z 450 (M+H)⁺, RT=1.942 minutes.

### Example 117

### [(2-{(3,5-dimethoxy-4-methylbenzoyl)[3-(3-fluorophenyl)propyl]amino}ethyl)sulfanyl]acetic Acid

### Step 1: ethyl [(2-{[3-(3-fluorophenyl)propyl]amino}ethyl)sulfanyl]acetate

A mixture of 3-(3-fluorophenyl)propan-1-amine (300 mg, 1.958 mmol) and Example 112—Step 3 (445 mg, 1.958 mmol) in CH₃CN (2 mL) was stirred at reflux for 3 hours. The mixture was cooled down and concentrated to give the titled compound. LC-MS (ESI) m/z 300 (M+H)⁺, RT=1.537 minutes.

### Step 2: ethyl [(2-{(3,5-dimethoxy-4-methylbenzoyl)[3-(3-fluorophenyl)propyl]amino}ethyl) sulfanyl]acetate

To a solution of 3,5-dimethoxy-4-methylbenzoic acid (360 mg, 1.837 mmol) in N,N-dimethylformamide (3 mL) was added 1-[bis(dimethylamino)methylene]-1H-1,2,3-triazolo[4,5-b]pyridinium 3-oxid hexafluorophosphate (733 mg, 1.929 mmol, HATU). The resulting solution was stirred at room temperature for 10 minutes. A solution of Example 117—Step 1 (550 mg, 1.837 mmol) in N,N-dimethylformamide (1.0 mL) was added in one portion followed by the addition of diisopropylethylamine (0.642 mL 3.67 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (0.2 g, 0.419 mmol, 22.80% yield). LC-MS (ESI) m/z 478 (M+H)⁺, RT=2.145 minutes.

### Step 3: [(2-{(3,5-dimethoxy-4-methylbenzoyl)[3-(3-fluorophenyl)propyl]amino}ethyl)sulfanyl]acetic Acid

To a solution of Example 117—Step 2 (0.2 g, 0.419 mmol) in dioxane (0.5 mL) was added 1 N LiOH (0.5 mL 0.500 mmol) dropwise. The mixture was stirred at 30° C. for 1 hour. The mixture was adjusted to pH about 6-7 and then purified by preparative HPLC eluting with CH₃CN/H₂O/CF₃CO₂H to give the titled compound (0.14 g, 0.311 mmol, 74.4% yield). ¹H NMR (400 MHz, methanol-d₄) δ ppm 7.33-6.67 (m, 4H), 6.53 (d, J=31.8 Hz, 2H), 3.80 (d, J=18.9 Hz, 8H), 3.64-3.44 (m, 2H), 3.33 (d, J=2.9 Hz, 2H), 2.95 (d, J=7.6 Hz, 2H), 2.75 (d, J=28.1 Hz, 1H), 2.47 (d, J=7.6 Hz, 1H), 2.05 (s, 4H), 1.94-1.83 (m, 1H); LC-MS (ESI) m/z 450 (M+H)⁺, RT=1.95 minutes.

### Example 118

### [(2-{(3,5-dimethoxy-4-methylbenzoyl)[3-(4-fluorophenyl)propyl]amino}ethyl)sulfanyl]acetic Acid

### Step 1: ethyl [(2-{[3-(4-fluorophenyl)propyl]amino}ethyl)sulfanyl]acetate

A mixture of 3-(4-fluorophenyl)propan-1-amine (300 mg, 1.958 mmol) and Example 112—Step 3 (445 mg, 1.958 mmol) in CH₃CN (2 mL) was stirred at reflux for 3 hours. The mixture was cooled down and concentrated to give the titled compound. LC-MS (ESI) m/z 300 (M+H)⁺, RT=1.576 minutes.

### Step 2: ethyl [(2-{(3,5-dimethoxy-4-methylbenzoyl)[3-(4-fluorophenyl)propyl]amino}ethyl) sulfanyl]acetate

To a solution of 3,5-dimethoxy-4-methylbenzoic acid (360 mg, 1.837 mmol) in N,N-dimethylformamide (3 mL) was added 1-[bis(dimethylamino)methylene]-1H-1,2,3-triazolo[4,5-b]pyridinium 3-oxid hexafluorophosphate (733 mg, 1.929 mmol, HATU). The resulting solution was stirred at room temperature for 10 minutes. A solution of Example 118—Step 1 (550 mg, 1.837 mmol) in N,N-dimethylformamide (1.0 mL) was added in one portion followed by the addition of diisopropylethylamine (0.642 mL 3.67 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (0.2 g, 0.419 mmol, 22.80% yield). LC-MS (ESI) m/z 478 (M+H)⁺, RT=2.144 minutes.

### Step 3: [(2-{(3,5-dimethoxy-4-methylbenzoyl)[3-(4-fluorophenyl)propyl]amino}ethyl)sulfanyl]acetic Acid

To a solution of Example 118—Step 2 (0.2 g, 0.419 mmol) in dioxane (0.5 mL) was added 1 N LiOH (0.5 mL 0.500 mmol) dropwise. The mixture was stirred at 30° C. for 1 hour. The mixture pH was adjusted to about 6-7 and then purified by preparative HPLC eluting with CH₃CN/H₂O/CF₃CO₂H to give the titled compound (70 mg, 0.156 mmol, 37.2% yield). ¹H NMR (400 MHz, methanol-d₄) δ ppm 7.26 (s, 1H), 7.07-6.79 (m, 3H), 6.52 (d, J=33.8 Hz, 2H), 3.79 (d, J=13.7 Hz, 8H), 3.59-3.45 (m, 2H), 3.42-3.31 (m, 2H), 2.94 (d, J=7.7 Hz, 2H), 2.74 (d, J=37.0 Hz, 1H), 2.45 (t, J=6.8 Hz, 1H), 2.05 (s, 4H), 1.86 (q, J=7.7, 6.5 Hz, 1H); LC-MS (ESI) m/z 450 (M+H)⁺, RT=1.943 minutes.

### Example 119

### N-{5-[(cyclopropanesulfonyl)amino]-5-oxopentyl}-3,5-dimethoxy-4-methyl-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (58.8 mg, 0.363 mmol) in isopropyl acetate (2 mL) was stirred at 40° C. for 10 minutes, and then cyclopropanesulfonamide (32.2 mg, 0.266 mmol) and 1,8-diazabicyclo[5.4.0]undec-7-ene (72.9 μL, 0.484 mmol) were added. The reaction mixture was stirred at 40° C. for 18 hours. The reaction mixture was diluted with 10 mL of ethyl acetate, washed with 10 mL of 1 N HCl and saturated NaCl, dried over MgSO₄ and concentrated. Flash chromatography (100% ethyl acetate) gave the titled compound (76 mg) that was contaminated with a small amount of starting acid. This material was redissolved in isopropyl acetate (2 mL); 1,1′-carbonyldiimidazole (30 mg), 1,8-diazabicyclo[5.4.0]undec-7-ene (0.04 mL) and cyclopropane sulfonamide (25 mg) were added; and the resultant mixture was stirred at 50° C. for 48 hours. The reaction mixture was diluted with 10 mL of ethyl acetate, washed with 10 mL of 1 N HCl and saturated NaCl, dried over MgSO₄ and concentrated. Flash chromatography (100% ethyl acetate) gave the titled compound (55 mg, 44%). ¹H NMR (400 MHz, DMSO-d₆) δ ppm 0.69-0.90 (m, 2H), 1.03 (t, J=6.4 Hz, 4H), 1.22 (s, 2H), 1.54 (s, 3H), 1.82 (s, 2H), 2.07-2.42 (m, 3H), 2.61 (s, 1H), 2.90 (s, 1H), 3.15 (s, 2H), 3.37 (s, 2H), 3.74 (s, 6H), 6.48 (s, 2H), 6.88-7.53 (m, 5H), 11.57 (s, 1H); MS (DCI) m/z 517 (M+H)⁺.

### Example 120

### (4R)-5-[(3,5-dimethoxy-4-methylbenzoyl)(3-phenylpropyl)amino]-4-hydroxypentanoic Acid

### Step 1: ethyl (4R)-4-{[tert-butyl(dimethyl)silyl]oxy}-5-[(3-phenylpropyl)amino]pentanoate

To a solution of (2S,4R)-ethyl 4-((tert-butyldimethylsilyl)oxy)-1-(3-phenylpropyl)pyrrolidine-2-carboxylate (0.1 g, 0.255 mmol, Accela ChemBio Co., Ltd), HMPA (0.222 mL 1.277 mmol), and tetrahydrofuran (2 mL) were added dropwise samarium(II) iodide (2.043 mL 2.043 mmol) and pivalic acid (0.074 mL 0.638 mmol) in tetrahydrofuran (1 mL) at 0° C. The resulting solution was allowed to warm to room temperature. A stream of air was bubbled through the solution, and an excess of diatomaceous earth in diethyl ether and saturated aqueous NaHCO₃ (1 mL) were added. The mixture was filtered, and the filtrate was washed with brine. The organic layer was separated, dried and concentrated to give the titled compound. LC-MS (ESI) m/z 394.2 (M+H)⁺, RT=1.99 minutes.

### Step 2: ethyl (4R)-4-{[tert-butyl(dimethyl)silyl]oxy}-5-[(3,5-dimethoxy-4-methylbenzoyl)(3-phenylpropyl)amino]pentanoate

A mixture of 3,5-dimethoxy-4-methylbenzoic acid (0.025 g, 0.127 mmol), Example 120—Step 1 (50 mg, 0.079 mmol, 62.0% yield), 2-(3H-[1,2,3]triazolo[4,5-b]pyridin-3-yl)-1,1,3,3-tetramethylisouronium hexafluorophosphate(V) (0.048 g, 0.127 mmol), N-ethyl-N-isopropylpropan-2-amine (0.022 mL 0.127 mmol) and N,N-dimethylformamide (1 mL) was stirred at room temperature for 1 hour. The mixture was extracted with ethyl acetate (3×20 mL). The organic layer was combined, washed with brine, dried with Na₂SO₄, filtered and concentrated to dryness. The residue was purified by flash column (eluent, ethyl acetate/hexane from 0/100 to 20/80) to give the titled compound. ¹H NMR (400 MHz, CDCl₃) δ ppm 7.34-7.26 (m, 1H), 7.26-7.10 (m, 3H), 6.98 (d, J=7.3 Hz, 1H), 6.51 (s, 2H), 4.26-3.97 (m, 3H), 3.80 (d, J=5.3 Hz, 6H), 3.68-3.11 (m, 4H), 2.84-2.28 (m, 4H), 2.09 (s, 3H), 2.02-1.62 (m, 4H), 1.29-1.19 (m, 3H), 0.96-0.79 (m, 9H), 0.18-−0.12 (m, 6H); LC-MS (ESI) m/z 572.4 (M+H)⁺, RT=2.497 minutes.

### Step 3: ethyl (4R)-5-[(3,5-dimethoxy-4-methylbenzoyl)(3-phenylpropyl)amino]-4-hydroxypentanoate

A mixture of Example 120—Step 2 (80 mg, 0.140 mmol) and tetra-n-butylammonium fluoride (54.9 mg, 0.210 mmol) in tetrahydrofuran (2 mL) was stirred at room temperature for 1 hour. The mixture was extracted with ethyl acetate (3×20 mL). The organic layers were combined, washed with brine, dried with Na₂SO₄, filtered and concentrated to dryness. The residue was purified by flash column chromatography on silica gel (eluent, ethyl acetate/hexane from 0/100 to 40/60) to give the titled compound. ¹H NMR (400 MHz, CDCl₃) δ ppm 7.25-7.12 (m, 3H), 7.01 (s, 2H), 6.49 (d, J=12.1 Hz, 2H), 4.14 (q, J=7.1 Hz, 2H), 3.95 (d, J=14.6 Hz, 1H), 3.79 (s, 7H), 3.67-3.54 (m, 1H), 3.44-3.13 (m, 3H), 2.50 (d, J=13.9 Hz, 4H), 2.10 (s, 3H), 1.89 (s, 4H), 1.31-1.22 (m, 3H); LC-MS (ESI) m/z 572.4 (M+H)⁺, RT=2.497 minutes.

### Step 4: (4R)-5-[(3,5-dimethoxy-4-methylbenzoyl)(3-phenylpropyl)amino]-4-hydroxypentanoic Acid

A mixture of Example 120—Step 3 (50 mg, 0.109 mmol), lithium hydroxide hydrate (9.17 mg, 0.219 mmol), water (1 mL) and 1,4-dioxane (1 mL) was stirred at 40° C. for 2 hours. The mixture was acidified to pH=4 with 1 N HCl. The clear solution was purified by preparative HPLC (0.1% CF₃CO₂H as buffer). 90% of the material was eliminated to give Example 121. The mixture was purified again by preparative HPLC (ammonium acetate as buffer) to afford the titled compound and Example 121. ¹H NMR (400 MHz, methanol-d₄) δ ppm 7.31-7.04 (m, 4H), 6.95 (d, J=7.2 Hz, 1H), 6.69 (s, 1H), 6.54 (s, 1H), 3.96 (s, 1H), 3.80 (d, J=17.7 Hz, 6H), 3.63 (d, J=15.0 Hz, 1H), 3.45-3.35 (m, 2H), 2.70 (t, J=7.7 Hz, 1H), 2.40 (dt, J=26.1, 7.4 Hz, 2H), 2.20 (d, J=7.4 Hz, 1H), 2.05 (d, J=4.5 Hz, 3H), 1.95-1.78 (m, 2H), 1.77-1.66 (m, 1H), 1.58 (s, 1H), 1.46 (s, 1H); LC-MS (ESI) m/z 430.2 (M+H)⁺, RT=1.844 minutes.

### Example 121

### (3E)-5-[(3,5-dimethoxy-4-methylbenzoyl)(3-phenylpropyl)amino]pent-3-enoic Acid

See Example 120 for details. ¹H NMR (400 MHz, CDCl₃) δ ppm 7.29-6.91 (m, 5H), 6.48 (d, J=8.7 Hz, 2H), 4.91 (d, J=10.5 Hz, 1H), 4.03 (dd, J=68.0, 16.1 Hz, 1H), 3.79 (s, 6H), 3.53-3.13 (m, 3H), 2.62-2.37 (m, 4H), 2.10 (s, 3H); LC-MS (ESI) m/z 412.2 (M+H)⁺, RT=1.975 minutes.

### Example 122

### [(2-{[3-(3-chlorophenyl)propyl](3,5-dimethoxy-4-methylbenzoyl)amino}ethyl)sulfanyl]acetic Acid

### Step 1: ethyl [(2-{[3-(3-chlorophenyl)propyl]amino}ethyl)sulfanyl]acetate

A mixture of 3-(3-chlorophenyl)propan-1-amine (300 mg, 1.768 mmol) and Example 112—Step 3 (402 mg, 1.768 mmol) in CH₃CN (2 mL) was stirred at reflux for 3 hours. The mixture was cooled down and concentrated to give the titled compound. LC-MS (ESI) m/z 316.2 (M+H)⁺, RT=1.634 minutes.

### Step 2: ethyl [(2-{[3-(3-chlorophenyl)propyl](3,5-dimethoxy-4-methylbenzoyl)amino}ethyl)sulfanyl]acetate

To a solution of 3,5-dimethoxy-4-methylbenzoic acid (342 mg, 1.741 mmol) in N,N-dimethylformamide (1 mL) was added 1-[bis(dimethylamino)methylene]-1H-1,2,3-triazolo[4,5-b]pyridinium 3-oxid hexafluorophosphate (695 mg, 1.828 mmol, HATU). The resulting solution was stirred at room temperature for 10 minutes. A solution of Example 122—Step 1 (550 mg, 1.741 mmol) in N,N-dimethylformamide (1.0 mL) was added in one portion followed by the addition of diisopropylethylamine (0.608 mL 3.48 mmol). The solution was stirred at room temperature for 1 hour. The mixture was diluted with water (30 mL) and extracted with ethyl acetate (2×20 mL). The combined organic layers were washed with brine (3×20 mL), dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (0.2 g, 0.405 mmol, 23.25% yield). LC-MS (ESI) m/z 494.2 (M+H)⁺, RT=2.2 minutes.

### Step 3: [(2-{[3-(3-chlorophenyl)propyl](3,5-dimethoxy-4-methylbenzoyl)amino}ethyl)sulfanyl]acetic Acid

To a solution of Example 122—Step 2 (0.2 g, 0.417 mmol) in dioxane (0.5 mL) was added 1 N LiOH (0.5 mL 0.500 mmol) dropwise. The mixture was stirred at 30° C. for 1 hour. The pH of the mixture was adjusted to about 6-7 and then purified by preparative HPLC eluting with CH₃CN/H₂O/CF₃CO₂H to give the titled compound (70 mg, 0.150 mmol, 30.0% yield). ¹H NMR (400 MHz, methanol-d₄) δ ppm 7.37-6.85 (m, 4H), 6.53 (d, J=35.2 Hz, 2H), 3.80 (d, J=18.2 Hz, 8H), 3.54 (s, 2H), 3.34 (s, 2H), 2.96 (s, 1H), 2.75 (d, J=36.0 Hz, 1H), 2.46 (s, 1H), 2.05 (s, 4H), 1.94-1.77 (m, 1H); LC-MS (ESI) m/z 466.2 (M+H)⁺.

### Example 123

### N-{5-[(ethanesulfonyl)amino]-5-oxopentyl}-3,5-dimethoxy-4-methyl-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (62.7 mg, 0.387 mmol) in isopropyl acetate (2 mL) was stirred at 40° C. for 10 minutes. Ethanesulfonamide (34.3 mg, 0.314 mmol) and 1,8-diazabicyclo[5.4.0]undec-7-ene (72.9 μL, 0.484 mmol) were added, and the reaction mixture was stirred at 40° C. for 18 hours. The reaction mixture was diluted with 10 mL of ethyl acetate, washed with 10 mL of 1 N HCl and saturated NaCl, dried with MgSO₄ and concentrated. The residue was purified by flash chromatography on silica gel (100% ethyl acetate) to give the titled compound (70 mg, 57%). ¹H NMR (400 MHz, DMSO-d₆) δ ppm 1.17 (t, J=7.3 Hz, 4H), 1.42 (d, J=88.4 Hz, 5H), 1.81 (s, 2H), 1.98 (s, 3H), 2.09-2.42 (m, 3H), 2.61 (s, 1H), 3.15 (s, 2H), 3.56 (s, 2H), 3.74 (s, 6H), 6.48 (s, 2H), 7.14 (t, J=45.6 Hz, 5H), 11.51 (s, 1H); MS (DCI) m/z 505 (M+H)⁺.

### Example 124

### 3,5-dimethoxy-4-methyl-N-{5-oxo-5-[(propane-2-sulfonyl)amino]pentyl}-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (58.8 mg, 0.363 mmol) in isopropyl acetate (2 mL) was stirred at 40° C. for 10 minutes. Propane-2-sulfonamide (32.8 mg, 0.266 mmol) and 1,8-diazabicyclo[5.4.0]undec-7-ene (72.9 μL, 0.484 mmol) were added, and the reaction mixture was stirred at 40° C. for 18 hours. The reaction mixture was diluted with 10 mL of ethyl acetate, washed with 1 N HCl (10 mL) and saturated NaCl (10 mL), dried with MgSO₄ and concentrated. Flash chromatography on silica gel (100% ethyl acetate) gave the titled compound (94 mg, 75%). ¹H NMR (400 MHz, DMSO-d₆) δ ppm 1.21 (d, J=6.8 Hz, 6H), 1.40 (s, 4H), 1.80 (s, 2H), 1.96 (s, 3H), 2.06-2.42 (m, 3H), 2.61 (d, J=17.3 Hz, 1H), 3.13 (s, 2H), 3.35 (s, 2H), 3.54 (s, 1H), 3.72 (s, 6H), 6.46 (s, 2H), 6.84-7.42 (m, 5H), 11.42 (s, 1H); MS (DCI) m/z 519 (M+H)⁺.

### Example 125

### [(2-{[3-(3,5-difluorophenyl)propyl](3,5-dimethoxy-4-methylbenzoyl)amino}ethyl)sulfanyl]acetic Acid

### Step 1: 3-(3,5-difluorophenyl)prop-2-enamide

To a mixture of 1-bromo-3,5-difluorobenzene (5.00 g, 25.9 mmol) and acrylamide (1.842 g, 25.9 mmol) in N,N-dimethylformamide (50 mL) was added triethylamine (13.11 g, 130 mmol), tri-o-tolylphosphine (0.789 g, 2.59 mmol) and tris(dibenzylideneacetone)dipalladium(0) (1.186 g, 1.295 mmol, Pd₂(dba)₃). The mixture was stirred at 110° C. overnight. Then the mixture was cooled to room temperature, H₂O (30 mL) was added to the mixture, and the mixture was extracted with ethyl acetate (50 mL). The organic layer was dried over Na₂SO₄, filtered and concentrated to give the residue which was purified by chromatography with hexane and acetate (0-80%) to give the titled compound. ¹H NMR (400 MHz, CDCl₃) δ ppm 7.55 (d, J=15.5 Hz, 1H), 7.13-6.94 (m, 2H), 6.82 (td, J=8.7, 4.4 Hz, 1H), 6.48 (d, J=15.6 Hz, 1H), 5.82 (brs, 5.82 Hz, 2H).

### Step 2: 3-(3,5-difluorophenyl)propanamide

To a solution of Example 125—Step 1 (4.18 g, 22.82 mmol) in EtOH (50 mL) was added Pd-C(0.243 g, 2.282 mmol), and the mixture was stirred under a H₂ atmosphere at room temperature overnight. The catalyst was removed by filtration, and the filtrate was concentrated to give the titled compound (3.99 g, 21.55 mmol, 94% yield). LC-MS (ESI) m/z 186.2 (M+H)⁺, RT=1.42 minutes.

### Step 3: 3-(3,5-difluorophenyl)propan-1-amine

To a suspension of LiAlH₄ (1.734 g, 45.7 mmol) in tetrahydrofuran (50 mL) was added a solution of Example 125—Step 2 (4.23 g, 22.84 mmol) in tetrahydrofuran (20 mL). The mixture was stirred at reflux temperature overnight. Then the mixture was cooled to room temperature, and wet Na₂SO₄ was added to the mixture to quench the reaction. After stirring at room temperature for 15 minutes, the solid was filtered and the filtrate was concentrated to give the residue which was purified by flash chromatography on silica gel eluted with dichloromethane and methanol (0-60%) to give the titled compound (2.30 g, 10.75 mmol, 47.1% yield). ¹H NMR (400 MHz, CDCl₃) δ ppm 6.79-6.68 (m, 2H), 6.63 (ddd, J=9.1, 6.7, 2.4 Hz, 1H), 2.79-2.72 (m, 2H), 2.66 (dd, J=8.9, 6.7 Hz, 2H), 2.35 (s, 2H), 1.83-1.74 (m, 2H); LC-MS (ESI) m/z 172.2 (M+H)⁺, RT=0.19 minutes.

### Step 4: ethyl [(2-{[3-(3,5-difluorophenyl)propyl]amino}ethyl)sulfanyl]acetate

A mixture of Example 125—Step 3 (250 mg, 1.460 mmol) and Example 112—Step 3 (332 mg, 1.460 mmol) in CH₃CN (3 mL) was stirred at reflux for 1.5 hours. The solution was cooled down and concentrated to give the titled compound. LC-MS (ESI) m/z 318.2 (M+H)⁺, RT=1.614 minutes.

### Step 5: ethyl [(2-{[3-(3,5-difluorophenyl)propyl](3,5-dimethoxy-4-methylbenzoyl)amino}ethyl)sulfanyl]acetate

To a solution of 3,5-dimethoxy-4-methylbenzoic acid (289 mg, 1.474 mmol) and 1-[bis(dimethylamino)methylene]-1H-1,2,3-triazolo[4,5-b]pyridinium 3-oxid hexafluorophosphate (617 mg, 1.621 mmol, HATU) in N,N-dimethylformamide (6 mL) was added triethylamine (0.226 mL 1.621 mmol). The resultant mixture was stirred at room temperature for 30 minutes. A solution of Example 125—Step 4 (468 mg, 1.474 mmol) and triethylamine (0.5 mL) in CH₃CN (3 mL) was added to the reaction system. The mixture was stirred at room temperature overnight. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (364 mg, 0.261 mmol, 17.74% yield). LC-MS (ESI) m/z 496.2 (M+H)⁺, RT=2.150 minutes.

### Step 6: [(2-{[3-(3,5-difluorophenyl)propyl](3,5-dimethoxy-4-methylbenzoyl)amino}ethyl)sulfanyl]acetic Acid

To a solution of Example 125—Step 5 (364 mg, 0.734 mmol) in tetrahydrofuran (4 mL) was added 1 N lithium hydroxide (4.41 mL 4.41 mmol). The mixture was stirred at room temperature for 2 hours. The solution was concentrated under reduced pressure. A solid precipitated that was collected by filtration and washed with water and hexanes. The solid was purified by preparative HPLC (0.1% CF₃CO₂H in H₂O/CH₃CN) and lyophilized to give the titled compound (78 mg, 0.167 mmol, 22.71% yield). ¹H NMR (400 MHz, DMSO-d₆) δ ppm 12.58 (s, 1H), 7.02 (s, 1H), 6.92 (s, 1H), 6.77 (s, 1H), 6.53 (s, 1H), 6.47 (s, 1H), 3.84-3.66 (m, 6H), 3.58 (s, 1H), 3.40 (s, 1H), 3.32 (s, 2H), 3.17 (s, 1H), 3.03 (s, 1H), 2.82 (s, 1H), 2.75 (s, 1H), 2.66 (s, 1H), 2.42 (s, 1H), 1.97 (s, 3H), 1.88 (s, 1H), 1.80 (s, 1H); LC-MS (ESI) m/z 468.2 (M+H)⁺, RT=1.953 minutes.

### Example 126

### 5-{[3-(3,5-difluorophenyl)propyl](3,5-dimethoxy-4-methylbenzoyl)amino}pentanoic Acid

### Step 1: methyl 5-{[3-(3,5-difluorophenyl)propyl]amino}pentanoate

A mixture of Example 125—Step 3 (250 mg, 1.460 mmol) and methyl 5-bromopentanoate (285 mg, 1.460 mmol) in CH₃CN (3 mL) was stirred at reflux for 1.5 hours. The solution was cooled down and concentrated to give the titled compound. LC-MS (ESI) m/z 286.2 (M+H)⁺, RT=1.541 minutes.

### Step 2: methyl 5-{[3-(3,5-difluorophenyl)propyl](3,5-dimethoxy-4-methylbenzoyl)amino}pentanoate

To a solution of 3,5-dimethoxy-4-methylbenzoic acid (289 mg, 1.474 mmol) and 1-[bis(dimethylamino)methylene]-1H-1,2,3-triazolo[4,5-b]pyridinium 3-oxid hexafluorophosphate (617 mg, 1.621 mmol, HATU) in N,N-dimethylformamide (6 mL) was added triethylamine (0.226 mL 1.621 mmol). The resultant mixture was stirred at room temperature for 30 minutes. A solution of Example 126—Step 1 (421 mg, 1.474 mmol) and triethylamine (0.5 mL) in CH₃CN (3 mL) was added to the reaction system. The mixture was stirred at room temperature overnight. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (264 mg, 0.296 mmol, 20.09% yield). LC-MS (ESI) m/z 462.2 (M+H)⁺, RT=2.112 minutes.

### Step 3: 5-{[3-(3,5-difluorophenyl)propyl](3,5-dimethoxy-4-methylbenzoyl)amino}pentanoic Acid

To a solution of Example 126—Step 2 (264 mg, 0.570 mmol) in tetrahydrofuran (4 mL) was added 1 N lithium hydroxide (3.42 mL 3.42 mmol). The mixture was stirred at room temperature for 2 hours. The solution was concentrated under reduced pressure. The resultant solid was collected by filtration and washed with water and hexanes. This material was further purified by preparative HPLC (0.1% CF₃CO₂H—H₂O/CH₃CN) and lyophilized to give the titled compound (15 mg, 0.033 mmol, 5.86% yield). ¹H NMR (400 MHz, DMSO-d₆) δ ppm 12.04 (s, 1H), 6.97 (d, J=39.8 Hz, 2H), 6.76 (s, 1H), 6.49 (s, 1H), 6.44 (s, 1H), 3.74 (dd, J=11.1, 4.3 Hz, 6H), 3.38 (s, 2H), 3.14 (s, 2H), 2.65 (dd, J=3.8, 2.0 Hz, 1H), 2.42 (s, 1H), 2.25 (s, 1H), 2.07 (d, J=9.7 Hz, 1H), 1.97 (s, 3H), 1.88 (s, 1H), 1.77 (s, 1H), 1.53 (s, 3H), 1.29 (s, 1H); LC-MS (ESI) m/z 450.2 (M+H)⁺, RT=1.953 minutes.

### Example 127

### (2-{[3-(3,5-dichlorophenyl)propyl](3,5-dimethoxy-4-methylbenzoyl)amino}ethoxy)acetic Acid

### Step 1: 3-(3,5-dichlorophenyl)propanamide

A solution of 3-(3,5-dichlorophenyl)propanoic acid (2.0 g, 9.13 mmol) in thionyl chloride (10 mL, 137 mmol) was heated to reflux for 2 hours. The mixture was concentrate, the residue was dissolved in anhydrous tetrahydrofuran (30 mL), and the solution was cooled to 0° C. Ammonia gas was bubbled in until pH>7, and a solid was formed. Then the mixture was concentrated to give the titled compound (2.096 g, 9.13 mmol, 100% yield). ¹H NMR (400 MHz, methanol-d₄) δ ppm 7.26 (t, J=2.0 Hz, 1H), 7.22 (d, J=1.9 Hz, 2H), 2.90 (t, J=7.5 Hz, 2H), 2.50 (t, J=7.5 Hz, 2H); LC-MS (ESI) m/z 220.0 (M+H)⁺, RT=1.690 minutes.

### Step 2: 3-(3,5-dichlorophenyl)propan-1-amine

To a solution of Example 127—Step 1 (2.1 g, 9.63 mmol) in tetrahydrofuran (60 mL) was added lithium aluminum hydride (1.0 g, 26.3 mmol) in small portions at 0° C. Then the reaction mixture was heated to reflux for 2 hours. Then the reaction was quenched with 3.0 g of Na₂SO₄. To the mixture was added 5 g Mg₂SO₄, and then the solids were removed by filtration. The solution was concentrated to give the titled compound (1.76 g, 6.90 mmol, 71.6% yield). ¹H NMR (400 MHz, CDCl₃) δ ppm 7.19 (t, J=1.9 Hz, 1H), 7.14-7.01 (m, 2H), 2.77 (ddd, J=9.0, 4.9, 2.0 Hz, 2H), 2.68-2.58 (m, 2H), 1.82 (ddd, J=9.3, 5.2, 2.0 Hz, 2H); LC-MS (ESI) m/z 204.0 (M+H)⁺, RT=1.542 minutes.

### Step 3: N-(2-{[tert-butyl(dimethyl)silyl]oxy}ethyl)-3-(3,5-dichlorophenyl)propan-1-amine

A mixture of (2-bromoethoxy)(tert-butyl)dimethylsilane (0.5 g, 2.090 mmol), Example 127—Step 2 (0.427 g, 2.090 mmol) and potassium carbonate (0.347 g, 2.508 mmol) in acetonitrile (6 mL) was stirred at reflux for 16 hours. The solid was filtered off; and the filtrate was concentrated to give the titled compound. LC-MS (ESI) m/z 362.2 (M+H)⁺, RT=1.94 minutes.

### Step 4: N-(2-{[tert-butyl(dimethyl)silyl]oxy}ethyl)-N-[3-(3,5-dichlorophenyl)propyl]-3,5-dimethoxy-4-methylbenzamide

To a solution of 3,5-dimethoxy-4-methylbenzoic acid (0.224 g, 1.140 mmol) and 1-[bis(dimethylamino)methylene]-1H-1,2,3-triazolo[4,5-b]pyridinium 3-oxid hexafluorophosphate (0.433 g, 1.140 mmol, HATU) in N,N-dimethylformamide (5 mL) was added triethylamine (0.159 mL 1.140 mmol). The resulting solution was stirred at room temperature for 5 minutes. A solution of Example 127—Step 3 (0.551 g, 0.76 mmol) in N,N-dimethylformamide (1.0 mL) was added in one portion. The solution was stirred at room temperature for 3 hours. The mixture was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (0.38 g, 0.668 mmol, 88% yield). LC-MS (ESI) m/z 540.2 (M+H)⁺, RT=2.54 minutes.

### Step 5: N-[3-(3,5-dichlorophenyl)propyl]-N-(2-hydroxyethyl)-3,5-dimethoxy-4-methylbenzamide

To a solution of Example 127—Step 4 (0.38 g, 0.703 mmol) in tetrahydrofuran (5 mL) was added tetra-n-butylammonium fluoride (0.221 g, 0.844 mmol). The reaction mixture was stirred at 20° C. for 1 hour. The reaction mixture was concentrated. The residue was dissolved in t-butyl methyl ether (10 mL), washed with water (5 mL) and brine (5 mL), dried over Na₂SO₄, filtered and concentrated to give the titled compound. LC-MS (ESI) m/z 426.2 (M+H)⁺, RT=2.04 minutes.

### Step 6: methyl (2-{[3-(3,5-dichlorophenyl)propyl](3,5-dimethoxy-4-methylbenzoyl)amino}ethoxy)acetate

To a solution of Example 127—Step 5 (0.3 g, 0.704 mmol) in tetrahydrofuran (5 mL) was added methyl 2-chloroacetate (0.185 mL 2.111 mmol). Then potassium tert-butoxide (0.237 g, 2.111 mmol) was added in one portion, and the mixture was heated to reflux for 2 hours. The mixture was cooled down, quenched with saturated NH₄Cl, and extracted with ethyl acetate 3 times. The combined organic layers were washed with brine once, dried over anhydrous Na₂SO₄, filtered and concentrated to give a residue which was purified by flash column chromatography on silica gel (loaded directly with dichloromethane) eluted with hexanes and ethyl acetate (0-70%) to give the titled compound (0.15 g, 0.271 mmol, 38.5% yield). LC-MS (ESI) m/z 498.2 (M+H)⁺, RT=2.17 minutes.

### Step 7: (2-{[3-(3,5-dichlorophenyl)propyl](3,5-dimethoxy-4-methylbenzoyl)amino}ethoxy)acetic Acid

To a solution of Example 127—Step 6 (0.15 g, 0.301 mmol) in tetrahydrofuran (4 mL) was added 1 N lithium hydroxide (1.806 mL 1.806 mmol). The reaction mixture was stirred at 50° C. for 2 hours. The mixture was cooled down and acidified with 1 N HCl to pH=2-3. The mixture was extracted with ethyl acetate twice, and the combined organic layers were concentrated to give a crude product which was purified by preparative HPLC (0.1% ammonium bicarbonate/CH₃CN) and lyophilized to give the titled compound (90 mg, 0.182 mmol, 60.5% yield). ¹H NMR (400 MHz, DMSO-d₆) δ ppm 7.40-7.20 (m, 2H), 7.10 (s, 1H), 6.62-6.47 (m, 2H), 3.90-3.85 (m, 1H), 3.80-3.70 (m, 7H), 3.65-3.55 (m, 2H), 3.45-3.23 (m, 4H), 2.66-2.56 (m, 1H), 2.45-2.35 (m, 1H), 1.97 (s, 3H), 1.95-1.73 (m, 2H); LC-MS (ESI) m/z 484.2 (M+H)⁺, RT=2.03 minutes.

### Example 128

### (2-{[3-(2,6-difluorophenyl)propyl](3,5-dimethoxy-4-methylbenzoyl)amino}ethoxy)acetic Acid

### Step 1: N-(2-{[tert-butyl(dimethyl)silyl]oxy}ethyl)-3-(2,6-difluorophenyl)propan-1-amine

A mixture of (2-bromoethoxy)(tert-butyl)dimethylsilane (0.5 g, 2.090 mmol), Example 113—Step 2 (0.358 g, 2.090 mmol) and potassium carbonate (0.347 g, 2.508 mmol) in acetonitrile (6 mL) was stirred at reflux for 16 hours. The solid was filtered off and the filtrate was concentrated to give the titled compound. LC-MS (ESI) m/z 330.2 (M+H)⁺, RT=1.84 minutes.

### Step 2: N-(2-{[tert-butyl(dimethyl)silyl]oxy}ethyl)-N-[3-(2,6-difluorophenyl)propyl]-3,5-dimethoxy-4-methylbenzamide

To a solution of 3,5-dimethoxy-4-methylbenzoic acid (0.247 g, 1.256 mmol) and 1-[bis(dimethylamino)methylene]-1H-1,2,3-triazolo[4,5-b]pyridinium 3-oxid hexafluorophosphate (0.478 g, 1.256 mmol, HATU) in N,N-dimethylformamide (5 mL) was added triethylamine (0.175 mL 1.256 mmol). The resulting solution was stirred at room temperature for 5 minutes. A solution of Example 128—Step 1 (0.36 g, 0.674 mmol, 80% yield) in N,N-dimethylformamide (1.0 mL) was added in one portion. The solution was stirred at room temperature for 3 hours. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0˜50%) to give the titled compound (0.36 g, 0.674 mmol, 80% yield). LC-MS (ESI) m/z 508.4 (M+H)⁺, RT=2.47 minutes.

### Step 3: N-[3-(2,6-difluorophenyl)propyl]-N-(2-hydroxyethyl)-3,5-dimethoxy-4-methylbenzamide

To a solution of Example 128—Step 2 (0.36 g, 0.709 mmol) in tetrahydrofuran (5 mL) was added tetra-n-butylammonium fluoride (0.222 g, 0.851 mmol). The reaction mixture was stirred at 20° C. for 1 hour, and then the mixture was concentrated. The residue was dissolved in t-butyl methyl ether (10 mL), washed with water (5 mL) and brine (5 mL), dried over Na₂SO₄, filtered and concentrated to give the titled compound. LC-MS (ESI) m/z 394.2 (M+H)⁺, RT=1.88 minutes.

### Step 4: methyl (2-{[3-(2,6-difluorophenyl)propyl](3,5-dimethoxy-4-methylbenzoyl)amino}ethoxy)acetate

To a solution of Example 128—Step 3 (120 mg, 0.305 mmol) in tetrahydrofuran (3 mL) was added methyl 2-chloroacetate (99 mg, 0.915 mmol). Then potassium tert-butoxide (34.2 mg, 0.305 mmol) was added in one portion. The mixture was heated to reflux for 2 hours, and then the mixture was cooled down and quenched with saturated aqueous ammonium chloride. The mixture was extracted with ethyl acetate 3 times. The combined organic layers were washed with brine once, dried over anhydrous Na₂SO₄, filtered and concentrated to give a residue which was purified by flash column chromatography on silica gel (loaded directly with dichloromethane) eluted with hexanes and ethyl acetate (0-70%) to give the titled compound (80 mg, 0.155 mmol, 50.7% yield). LC-MS (ESI) m/z 466.2 (M+H)⁺, RT=2.04 minutes.

### Step 5: (2-{[3-(2,6-difluorophenyl)propyl](3,5-dimethoxy-4-methylbenzoyl)amino}ethoxy)acetic Acid

To a solution of Example 128—Step 4 (0.08 g, 0.172 mmol) in tetrahydrofuran (2 mL) was added 1 N lithium hydroxide (1.031 mL 1.031 mmol). The reaction mixture was heated to 50° C. for 2 hours. The mixture was cooled down, acidified with 1 N HCl to pH=2-3, and then extracted with ethyl acetate twice. The combined organic layers were concentrated to give a residue which was purified by preparative HPLC (0.1% ammonium bicarbonate/CH₃CN) and lyophilized to give the titled compound (15 mg, 0.032 mmol, 18.56% yield). ¹H NMR (400 MHz, DMSO-d₆, T=20° C.) 6 ppm 7.30-7.21 (m, 1H), 7.06-6.91 (m, 2H), 6.61-6.44 (m, 2H), 3.95-3.85 (m, 1H), 3.80-3.68 (m, 7H), 3.65-3.45 (m, 4H), 3.25-3.15 (m, 2H), 2.67-2.60 (m, 1H), 2.44-2.36 (m, 1H), 1.97 (s, 3H), 1.88-1.68 (m, 2H); LC-MS (ESI) m/z 452.2 (M+H)⁺, RT=1.90 minutes.

### Example 129

### (2-{(3,5-dimethoxy-4-methylbenzoyl)[3-(3-fluorophenyl)propyl]amino}ethoxy)acetic Acid

### Step 1: N-(2-{[tert-butyl(dimethyl)silyl]oxy}ethyl)-3-(3-fluorophenyl)propan-1-amine

A mixture of 3-(3-fluorophenyl)propan-1-amine (250 mg, 1.632 mmol), (2-bromoethoxy)(tert-butyl)dimethylsilane (390 mg, 1.632 mmol) and K₂CO₃ (451 mg, 3.26 mmol) in acetonitrile (10 mL) was heated to reflux overnight. Then the mixture was cooled to room temperature, and the solid was filtered and concentrated to give the titled compound. LC-MS (ESI) m/z 312.2 (M+H)⁺, RT=1.83 minutes.

### Step 2: N-(2-{[tert-butyl(dimethyl)silyl]oxy}ethyl)-N-[3-(3-fluorophenyl)propyl]-3,5-dimethoxy-4-methylbenzamide

To a solution of 3,5-dimethoxy-4-methylbenzoic acid (320 mg, 1.631 mmol) in N,N-dimethylformamide (5.00 mL) was added 1-[bis(dimethylamino)methylene]-1H-1,2,3-triazolo[4,5-b]pyridinium 3-oxid hexafluorophosphate (620 mg, 1.631 mmol, HATU) and triethylamine (0.682 mL, 4.89 mmol). The mixture was stirred at room temperature for 15 minutes. Then the mixture was added to a solution of Example 129—Step 1 in N,N-dimethylformamide (1.0 mL), and the mixture was stirred at room temperature for 2 hours. The mixture was concentrated, and the residue was diluted with water (20 mL) and extracted with ethyl acetate (30 mL). The organic layer was dried over Na₂SO₄, filtered and concentrated to give the residue which was purified by flash chromatography eluted with hexanes and ethyl acetate (0-30%) to give the titled compound (275 mg, 0.562 mmol, 34.4% yield). LC-MS (ESI) m/z 490.2 (M+H)⁺, RT=2.42 minutes.

### Step 3: N-[3-(3-fluorophenyl)propyl]-N-(2-hydroxyethyl)-3,5-dimethoxy-4-methylbenzamide

To a solution of Example 129—Step 2 (275 mg, 0.562 mmol) in tetrahydrofuran (5 mL) was added tetra-n-butylammonium fluoride (176 mg, 0.674 mmol) at room temperature. The mixture was stirred at room temperature overnight. The mixture was concentrated, and the residue was combined with ethyl acetate and H₂O and extracted with ethyl acetate (20 mL). The organic layer was dried over Na₂SO₄, filtered and concentrated. The residue was purified by flash chromatography eluted with hexane and ethyl acetate (0-50%) to give the titled compound (200 mg, 0.533 mmol, 95% yield). LC-MS (ESI) m/z 376.2 (M+H)⁺, RT=1.88 minutes.

### Step 4: methyl (2-{(3,5-dimethoxy-4-methylbenzoyl)[3-(3-fluorophenyl)propyl]amino}ethoxy)acetate

To a solution of Example 129—Step 3 (0.2 g, 0.533 mmol) in tetrahydrofuran (5 mL) was added methyl 2-chloroacetate (0.140 mL 1.598 mmol) and potassium tert-butoxide (0.060 g, 0.533 mmol) in one portion. The mixture was heated to reflux for 2 hours. The mixture was cooled down, quenched with saturated aqueous ammonium chloride, and extracted with ethyl acetate 3 times. The combined organic layers were washed with brine, dried over anhydrous Na₂SO₄, filtered and concentrated to give a residue which was purified by flash column chromatography on silica gel (eluent: hexanes:ethyl acetate from 100:0 to 50:50) to give the titled compound (0.1 g, 0.212 mmol, 39.9% yield). LC-MS (ESI) m/z 448.2 (M+H)⁺, RT=2.03 minutes.

### Step 5: (2-{(3,5-dimethoxy-4-methylbenzoyl)[3-(3-fluorophenyl)propyl]amino}ethoxy)acetic Acid

To a solution of Example 129—Step 4 (0.1 g, 0.223 mmol) in tetrahydrofuran (2 mL) was added 1 N lithium hydroxide (1.341 mL 1.341 mmol). It was heated to 50° C. for 2 hours. The mixture was cooled down and acidified with 1 N HCl to pH=2-3 and then extracted with ethyl acetate twice. The combined organic layers were concentrated to give a residue which was purified by preparative HPLC (0.1% ammonium bicarbonate/CH₃CN) and lyophilized to give the titled compound (0.03 g, 0.068 mmol, 30.4% yield). ¹H NMR (400 MHz, DMSO-d₆) δ ppm 7.34-7.09 (m, 4H), 6.63-6.50 (m, 2H), 3.76-3.73 (m, 6H), 3.72-3.66 (m, 2H), 3.64-3.60 (m, 4H), 3.39-3.15 (m, 2H), 2.67-2.61 (m, 1H), 2.45-2.35 (m, 1H), 1.97 (s, 3H), 1.93-1.75 (m, 2H); LC-MS (ESI) m/z 434.2 (M+H)⁺, RT=1.89 minutes.

### Example 130

### (2-{[3-(2,4-difluorophenyl)propyl](3,5-dimethoxy-4-methylbenzoyl)amino}ethoxy)acetic Acid

### Step 1: N-(2-{[tert-butyl(dimethyl)silyl]oxy}ethyl)-3-(2,4-difluorophenyl)propan-1-amine

A mixture of Example 114—Step 2 (250 mg, 1.460 mmol), (2-bromoethoxy)(tert-butyl)dimethylsilane (349 mg, 1.460 mmol) and K₂CO₃ (404 mg, 2.92 mmol) in acetonitrile (10 mL) was heated to reflux overnight. Then the mixture was cooled to room temperature, and resultant solid was collected by filtration. The filtrate was concentrated to give the titled compound. LC-MS (ESI) m/z 330.2 (M+H)⁺, RT=1.84 minutes.

### Step 2: N-(2-{[tert-butyl(dimethyl)silyl]oxy}ethyl)-N-[3-(3,4-difluorophenyl)propyl]-3,5-dimethoxy-4-methylbenzamide

To a solution of 3,5-dimethoxy-4-methylbenzoic acid (286 mg, 1.460 mmol) in N,N-dimethylformamide (5.00 mL) was added 1-[bis(dimethylamino)methylene]-1H-1,2,3-triazolo[4,5-b]pyridinium 3-oxid hexafluorophosphate (555 mg, 1.460 mmol, HATU) and triethylamine (0.610 mL, 4.38 mmol). The mixture was stirred at room temperature for 15 minutes. Then the mixture was combined with a solution of Example 130—Step 1 in N,N-dimethylformamide (1.0 mL), and the mixture was stirred at room temperature for 2 hours. The mixture was concentrated, and the residue was diluted with water (20 mL) and extracted with ethyl acetate (30 mL). The organic layer was dried over Na₂SO₄, filtered and concentrated to give the residue which was purified by flash chromatography with hexanes and ethyl acetate (0-30%) to give the titled compound (213 mg, 0.420 mmol, 28.7% yield). LC-MS (ESI) m/z 508.2 (M+H)⁺, RT=2.43 minutes.

### Step 3: N-[3-(3,4-difluorophenyl)propyl]-N-(2-hydroxyethyl)-3,5-dimethoxy-4-methylbenzamide

To a solution of Example 130—Step 2 (213 mg, 0.420 mmol) in tetrahydrofuran (5 mL) was added tetra-n-butylammonium fluoride (132 mg, 0.503 mmol) at room temperature. The mixture was stirred at room temperature overnight. The mixture was concentrated, and the residue was diluted with ethyl acetate and H₂O. The mixture was extracted with ethyl acetate (20 mL). The organic layer was dried over Na₂SO₄, filtered and concentrated. The residue was purified by flash chromatography eluted with hexane and ethyl acetate (0-50%) to give the titled compound (100 mg, 0.254 mmol, 60.6% yield). LC-MS (ESI) m/z 394.2 (M+H)⁺, RT=1.94 minutes.

### Step 4: methyl (2-{[3-(2,4-difluorophenyl)propyl](3,5-dimethoxy-4-methylbenzoyl)amino}ethoxy)acetate

To a solution of Example 130—Step 3 (0.12 g, 0.305 mmol) in tetrahydrofuran (5 mL) was added methyl 2-chloroacetate (0.080 mL 0.915 mmol) and potassium tert-butoxide (0.034 g, 0.305 mmol) in one portion. The mixture was heated to reflux for 2 hours. The mixture was cooled down and quenched with saturated aqueous ammonium chloride, and extracted with ethyl acetate 3 times. The combined organic layers were washed with brine, dried over anhydrous Na₂SO₄, filtered and concentrated to give a residue which was purified by flash column chromatography on silica gel (eluent: hexanes:ethyl acetate from 100:0 to 50:50) to give the titled compound (0.1 g, 0.204 mmol, 66.9% yield) as an oil. LC-MS (ESI) m/z 466.2 (M+H)⁺, RT=2.04 minutes.

### Step 5: (2-{[3-(2,4-difluorophenyl)propyl](3,5-dimethoxy-4-methylbenzoyl)amino}ethoxy)acetic Acid

To a solution of Example 130—Step 4 (0.08 g, 0.172 mmol) in tetrahydrofuran (2 mL) was added 1 N LiOH (1.031 mL 1.031 mmol). It was heated to 50° C. for 2 hours. The mixture was cooled down and acidified with 1 N HCl to pH=2-3 and then extracted with ethyl acetate twice. The combined organic layers were concentrated to give a residue which was purified by preparative HPLC (0.1% ammonium bicarbonate/CH₃CN) and lyophilized to give the titled compound (0.03 g, 0.065 mmol, 37.9% yield). ¹H NMR (400 MHz, DMSO-d₆) δ ppm 7.45-6.80 (m, 3H), 6.65-6.40 (m, 2H), 4.0-3.85 (m, 2H), 3.80-3.68 (m, 6H), 3.65-3.50 (m, 4H), 3.35-3.10 (m, 2H), 2.65-2.55 (m, 1H), 2.43-2.30 (m, 1H), 1.97 (s, 3H), 1.90-1.70 (m, 2H); LC-MS (ESI) m/z 452.2 (M+H)⁺, RT=1.90 minutes.

### Example 131

### (2-{[3-(2-chlorophenyl)propyl](3,5-dimethoxy-4-methylbenzoyl)amino}ethoxy)acetic Acid

### Step 1: N-(2-{[tert-butyl(dimethyl)silyl]oxy}ethyl)-3-(2-chlorophenyl)propan-1-amine

A mixture of (2-bromoethoxy)(tert-butyl)dimethylsilane (0.5 g, 2.090 mmol), 3-(2-chlorophenyl)propan-1-amine (0.355 g, 2.090 mmol) and potassium carbonate (0.347 g, 2.508 mmol) in acetonitrile (6 mL) was stirred at reflux for 16 hours. The solid was filtered off and the filtrate was concentrated to give the titled compound. LC-MS (ESI) m/z 328.2 (M+H)⁺, RT=1.87 minutes.

### Step 2: N-(2-{[tert-butyl(dimethyl)silyl]oxy}ethyl)-N-[3-(2-chlorophenyl)propyl]-3,5-dimethoxy-4-methylbenzamide

To a solution of 3,5-dimethoxy-4-methylbenzoic acid (0.246 g, 1.253 mmol) and 1-[bis(dimethylamino)methylene]-1H-1,2,3-triazolo[4,5-b]pyridinium 3-oxid hexafluorophosphate (0.476 g, 1.253 mmol, HATU) in N,N-dimethylformamide (5 mL) was added triethylamine (0.175 mL 1.253 mmol). The resulting solution was stirred at room temperature for 5 minutes. A solution of Example 131—Step 1 (0.685 g, 0.835 mmol) in N,N-dimethylformamide (1.0 mL) was added in one portion. The solution was stirred at room temperature for 3 hours. It was diluted with water and extracted with ethyl acetate twice. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (0.2 g, 0.375 mmol, 44.9% yield). LC-MS (ESI) m/z 506.2 (M+H)⁺, RT=2.49 minutes.

### Step 3: N-[3-(2-chlorophenyl)propyl]-N-(2-hydroxyethyl)-3,5-dimethoxy-4-methylbenzamide

To a solution of Example 131—Step 2 (0.2 g, 0.395 mmol) in tetrahydrofuran (5 mL) was added tetra-n-butylammonium fluoride (0.124 g, 0.474 mmol). The reaction mixture was stirred at 20° C. for 1 hour and then concentrated. The residue was dissolved in t-butyl methyl ether (10 mL) and washed with water (5 mL) and brine (5 mL), dried over Na₂SO₄, filtered and concentrated to give the titled compound. LC-MS (ESI) m/z 392.2 (M+H)⁺, RT=1.94 minutes.

### Step 4: methyl (2-{[3-(2-chlorophenyl)propyl](3,5-dimethoxy-4-methylbenzoyl)amino}ethoxy)acetate

To a solution of Example 131—Step 3 (0.2 g, 0.510 mmol) in tetrahydrofuran (5 mL) was added methyl 2-chloroacetate (0.134 mL 1.531 mmol) and potassium tert-butoxide (0.057 g, 0.510 mmol) in one portion. The mixture was heated to reflux for 2 hours. The mixture was cooled down, quenched with saturated aqueous ammonium chloride, and extracted with ethyl acetate 3 times. The combined organic layers were washed with brine, dried over anhydrous Na₂SO₄, filtered and concentrated to give a residue which was purified by flash column chromatography on silica gel (eluent: hexanes:ethyl acetate from 100:0 to 50:50) to give the titled compound (0.12 g, 0.233 mmol, 45.6% yield). LC-MS (ESI) m/z 464.2 (M+H)⁺, RT=2.08 minutes.

### Step 5: (2-{[3-(2-chlorophenyl)propyl](3,5-dimethoxy-4-methylbenzoyl)amino}ethoxy)acetic Acid

To a solution of Example 131—Step 4 (0.12 g, 0.259 mmol) in tetrahydrofuran (4 mL) was added 1 N lithium hydroxide (1.552 mL 1.552 mmol). The reaction mixture was heated to 50° C. for 2 hours. The mixture was cooled down and acidified with 1 N HCl to pH=2-3. The mixture was extracted with ethyl acetate twice, and the combined organic layers were concentrated. The residue was purified by preparative HPLC (0.1% ammonium bicarbonate/CH₃CN) and lyophilized to give the titled compound (40 mg, 0.087 mmol, 33.7% yield). ¹H NMR (400 MHz, DMSO-d₆, T=20° C.) 6 ppm 7.45-7.35 (m, 1H), 7.32-7.10 (m, 3H), 6.61-6.48 (m, 2H), 3.98-3.89 (m, 2H), 3.75-3.74 (m, 6H), 3.65-3.49 (m, 2H), 3.39-3.20 (m, 4H), 2.75-2.66 (m, 1H), 2.50-2.48 (m, 1H), 1.97 (s, 3H), 1.90-1.72 (m, 2H); LC-MS (ESI) m/z 450.2 (M+H)⁺, RT=1.94 minutes.

### Example 132

### [(2-{(3,5-dimethoxy-4-methylbenzoyl)[3-(5-methylfuran-2-yl)propyl]amino}ethyl)sulfanyl]acetic Acid

### Step 1: 2-(3-chloropropyl)-5-methylfuran

To a cooled stirred solution of 2-methylfuran (3.0 g, 36.5 mmol) in tetrahydrofuran (60 mL) was added butyllithium (27.4 mL, 43.8 mmol) dropwise at −78° C. Then the mixture was stirred at 0° C. for 1.5 hour. 1-Chloro-3-iodopropane (9.71 g, 47.5 mmol) was added dropwise, and the resulting solution was stirred for 16 hours at room temperature. Then the solution was concentrated to give the titled compound. ¹H NMR (400 MHz, CDCl₃) δ ppm 5.90 (d, J=3.0 Hz, 1H), 5.85 (d, J=3.0 Hz, 1H), 3.56 (t, J=6.5 Hz, 2H), 2.75 (t, J=7.2 Hz, 2H), 2.25 (s, 3H), 2.09 (p, J=6.9 Hz, 2H).

### Step 2: 2-[3-(5-methylfuran-2-yl)propyl]-1H-isoindole-1,3(2H)-dione

To a stirred solution of Example 132—Step 1 (5.8 g, 36.6 mmol) in CH₃CN (80 mL) was added isoindoline-1,3-dione (8.07 g, 54.8 mmol), sodium iodide (5.48 g, 36.6 mmol) and potassium carbonate (15.16 g, 110 mmol), and then the mixture was stirred at 85° C. over a weekend. The mixture was concentrated, and the residue was partitioned between water (100 mL) and ethyl acetate (100 mL). The organic layer was separated and concentrated. The residue was purified by flash column (0-45% ethyl acetate in n-hexane) to give the titled compound (4.0 g, 13.67 mmol, 37.4% yield). ¹H NMR (400 MHz, CDCl₃) δ ppm 7.92-7.78 (m, 2H), 7.78-7.64 (m, 2H), 5.90 (d, J=3.0 Hz, 1H), 5.78 (dq, J=3.1, 1.1 Hz, 1H), 3.84-3.64 (m, 2H), 2.65 (t, J=7.5 Hz, 2H), 2.19 (d, J=0.9 Hz, 3H), 2.07-1.93 (m, 2H); LC-MS (ESI) m/z 270.2 (M+H)⁺, RT=1.994 minutes.

### Step 3: 3-(5-methylfuran-2-yl)propan-1-amine

To a stirred solution of Example 132—Step 2 (4.0 g, 14.85 mmol) in CH₃OH (40 mL) was added hydrazine hydrate (1.957 g, 37.1 mmol) dropwise. Then the mixture was stirred at 80° C. for 3 hours. The solution was cooled, and the solid was removed by filtration. The filtrate was concentrated, and the residue was dissolved in ethyl ether (100 mL). The produced solid was removed again by filtration. The filtrate was concentrated, and the residue was dried under vacuum to give the titled compound (1.58 g, 10.22 mmol, 68.8% yield). ¹H NMR (400 MHz, CDCl₃) δ ppm 5.92-5.76 (m, 2H), 2.75 (td, J=7.0, 1.1 Hz, 2H), 2.62 (t, J=7.5 Hz, 2H), 2.24 (d, J=0.9 Hz, 3H), 1.85-1.72 (m, 2H), 1.70 (s, 2H); LC-MS (ESI) m/z 140.2 (M+H)⁺, RT=0.922 minutes.

### Step 4: ethyl [(2-bromoethyl)sulfanyl]acetate

A mixture of ethyl 2-mercaptoacetate (10.0 g, 83 mmol), 1,2-dibromoethane (46.9 g, 250 mmol) and K₂CO₃ (34.5 g, 250 mmol) in acetonitrile (150 mL) was heated to reflux for 2 hours. Then the mixture was cooled to room temperature, the solid was removed by filtration, and the filtrate was concentrated to give a residue which was purified by chromatography on silica gel eluted with hexane and ethyl acetate (0-10%) to give the titled compound (14.0 g, 61.6 mmol, 74.1% yield).

### Step 5: ethyl [(2-{[3-(5-methylfuran-2-yl)propyl]amino}ethyl)sulfanyl]acetate

A mixture of Example 132—Step 3 (300 mg, 2.155 mmol), Example 132—Step 4 (489 mg, 2.155 mmol) and potassium carbonate (447 mg, 3.23 mmol) in CH₃CN (7 mL) was stirred at reflux for 2 hours. The solution was cooled down, filtered and concentrated to give the titled compound. LC-MS (ESI) m/z 286.2 (M+H)⁺, RT=1.523 minutes.

### Step 6: ethyl [(2-{(3,5-dimethoxy-4-methylbenzoyl)[3-(5-methylfuran-2-yl)propyl]amino}ethyl)sulfanyl]acetate

To a solution of 3,5-dimethoxy-4-methylbenzoic acid (423 mg, 2.155 mmol) and 1-[bis(dimethylamino)methylene]-1H-1,2,3-triazolo[4,5-b]pyridinium 3-oxid hexafluorophosphate (901 mg, 2.371 mmol, HATU) in N,N-dimethylformamide (8 mL) was added triethylamine (0.330 mL 2.371 mmol). The resultant mixture was stirred at room temperature for 30 minutes. A solution of Example 132—Step 5 (615 mg, 2.155 mmol) in CH₃CN was added to the reaction system. The mixture was stirred at room temperature overnight. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (719 mg, 0.775 mmol, 36.0% yield). LC-MS (ESI) m/z 464.2 (M+H)⁺, RT=2.109 minutes.

### Step 7: [(2-{(3,5-dimethoxy-4-methylbenzoyl)[3-(5-methylfuran-2-yl)propyl]amino}ethyl)sulfanyl]acetic Acid

To a solution of Example 132—Step 6 (719 mg, 1.551 mmol) in tetrahydrofuran (4 mL) was added 1 N lithium hydroxide (9.31 mL 9.31 mmol). The mixture was stirred at room temperature for 1 hour. The solution was concentrated under reduced pressure. A solid precipitated from the aqueous mixture. The solid was collected by filtration and washed with water and hexanes to give the titled compound. A portion was purified by preparative HPLC (0.1% CF₃CO₂H—H₂O/CH₃CN) and lyophilized to give the titled compound (35 mg, 0.080 mmol, 5.18% yield). ¹H NMR (400 MHz, DMSO-d₆) δ ppm 12.59 (s, 1H), 6.53 (s, 2H), 5.96 (d, J=30.7 Hz, 1H), 5.78 (d, J=21.6 Hz, 1H), 3.75 (s, 6H), 3.57 (s, 1H), 3.40 (s, 2H), 3.33 (s, 1H), 3.21 (s, 1H), 3.03 (s, 1H), 2.82 (s, 1H), 2.74 (s, 1H), 2.67-2.51 (m, 1H), 2.37 (s, 1H), 2.19 (s, 1H), 2.09 (s, 2H), 1.98 (s, 3H), 1.83 (d, J=18.3 Hz, 2H); LC-MS (ESI) m/z 436.2 (M+H)⁺, RT=1.849 minutes.

### Example 133

### 5-{(3,5-dimethoxybenzoyl)[3-(5-methylfuran-2-yl)propyl]amino}pentanoic Acid

### Step 1: methyl 5-{[3-(5-methylfuran-2-yl)propyl]amino}pentanoate

A solution of Example 132—Step 3 (500 mg, 3.59 mmol) and methyl 5-bromopentanoate (701 mg, 3.59 mmol) in CH₃CN (10 mL) was stirred at reflux. The mixture was cooled down and concentrated to give the titled compound. LC-MS (ESI) m/z 254.2 (M+H)⁺, RT=1.517 minutes.

### Step 2: methyl 5-{(3,5-dimethoxybenzoyl)[3-(5-methylfuran-2-yl)propyl]amino}pentanoate

To a solution of 3,5-dimethoxybenzoic acid (327 mg, 1.795 mmol) and 1-[bis(dimethylamino)methylene]-1H-1,2,3-triazolo[4,5-b]pyridinium 3-oxid hexafluorophosphate (751 mg, 1.975 mmol, HATU) in N,N-dimethylformamide (8 mL) was added triethylamine (0.275 mL 1.975 mmol). The resultant mixture was stirred at room temperature for 30 minutes. A solution of Example 133—Step 1 (455 mg, 1.795 mmol) and triethylamine (2 mL) in CH₃CN was added to the reaction system. The mixture was stirred at room temperature overnight. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (315 mg, 0.626 mmol, 34.9% yield). LC-MS (ESI) m/z 418.2 (M+H)⁺, RT=1.991 minutes.

### Step 3: 5-{(3,5-dimethoxybenzoyl)[3-(5-methylfuran-2-yl)propyl]amino}pentanoic Acid

To a solution of Example 133—Step 2 (315 mg, 0.754 mmol) in tetrahydrofuran (6 mL) was added 1 N lithium hydroxide (4.53 mL 4.53 mmol). The mixture was stirred at room temperature for 1 hour. The solution was concentrated under reduced pressure. The aqueous mixture was washed with t-butyl methyl ether once. Then the aqueous mixture was acidified with 1 N HCl to pH=2-3 and extracted with ethyl acetate once. The organic layer was concentrated. The residue was purified by preparative HPLC (0.1% CF₃CO₂H—H₂O/CH₃CN) and lyophilized to give the titled compound (25 mg, 0.062 mmol, 8.21% yield). ¹H NMR (400 MHz, DMSO-d₆) δ ppm 12.03 (s, 1H), 6.50 (d, J=2.8 Hz, 1H), 6.39 (d, J=3.1 Hz, 2H), 6.02-5.89 (m, 1H), 5.78 (d, J=28.8 Hz, 1H), 3.73 (s, 6H), 3.37 (d, J=7.4 Hz, 2H), 3.13 (s, 2H), 2.56 (d, J=7.8 Hz, 1H), 2.35 (t, J=7.1 Hz, 1H), 2.24 (d, J=6.3 Hz, 1H), 2.16 (d, J=31.3 Hz, 3H), 2.07 (t, J=8.0 Hz, 1H), 1.84 (t, J=7.3 Hz, 1H), 1.79-1.69 (m, 1H), 1.53 (s, 3H), 1.33-1.22 (m, 1H); LC-MS (ESI) m/z 404.2 (M+H)⁺, RT=1.853 minutes.

### Example 134

### 5-{(3,5-dimethoxybenzoyl)[3-(2-fluorophenyl)propyl]amino}pentanoic Acid

### Step 1: methyl 5-{[3-(2-fluorophenyl)propyl]amino}pentanoate

In a 10 mL sealed tube was added 3-(2-fluorophenyl)propan-1-amine (0.5 g, 3.26 mmol), methyl 5-bromopentanoate (0.637 g, 3.26 mmol), K₂CO₃ (0.541 g, 3.92 mmol) and acetonitrile (5 mL). The reaction mixture was stirred at reflux for 1 hour. The mixture was cooled to room temperature and filtered to remove the solids. The filtrate was concentrated to give the titled compound. LC-MS (ESI) m/z 268.2 (M+H)⁺, RT=1.51 minutes.

### Step 2: methyl 5-{(3,5-dimethoxybenzoyl)[3-(2-fluorophenyl)propyl]amino}pentanoate

To a solution of 3,5-dimethoxybenzoic acid (0.267 g, 1.464 mmol) and 1-[bis(dimethylamino)methylene]-1H-1,2,3-triazolo[4,5-b]pyridinium 3-oxid hexafluorophosphate (0.594 g, 1.562 mmol, HATU) in N,N-dimethylformamide (5 mL) was added triethylamine (0.218 mL 1.562 mmol). The resulting solution was stirred at room temperature for 10 minutes. Then a solution of Example 134—Step 1 (0.87 g, 0.976 mmol) in CH₃CN (5 mL) was added in one portion. The reaction mixture was stirred at room temperature for 2 hours. The reaction mixture was diluted with water and extracted with ethyl acetate twice. The combined organic layers were washed with saturated NaHCO₃ solution (1×10 mL) and brine, dried over Na₂SO₄, filtered and concentrated. The residue was added to a silica gel column and was eluted with hexanes and ethyl acetate (0-50%) to give the titled compound (0.26 g, 0.542 mmol, 55.5% yield). LC-MS (ESI) m/z 432.2 (M+H)⁺, RT=2.0 minutes.

### Step 3: 5-{(3,5-dimethoxybenzoyl)[3-(2-fluorophenyl)propyl]amino}pentanoic Acid

To a solution of Example 134—Step 2 (0.26 g, 0.603 mmol) in tetrahydrofuran (6 mL) was added 1 N LiOH (3.62 mL 3.62 mmol). The reaction mixture was heated to 50° C. for 2 hours. The mixture was concentrated, and the resultant mixture was extracted with t-butyl methyl ether (5 mL). The water layer was separated and acidified with 1 N HCl to pH=2-3. This was then extracted with ethyl acetate twice, and the combined organic layers were concentrated to give a residue which was purified by preparative HPLC (0.1% CF₃CO₂H in water/CH₃CN) and lyophilized to give the titled compound (0.036 g, 0.085 mmol, 14.03% yield). ¹H NMR (400 MHz, DMSO-d₆, T=20° C.) 6 ppm 7.40-7.0 (m, 4H), 6.51-6.46 (m, 1H), 6.39-6.35 (m, 2H), 3.73-3.72 (m, 6H), 3.45-3.33 (m, 2H), 3.17-3.07 (m, 2H), 2.67-2.60 (m, 1H), 2.43-2.35 (m, 1H), 2.27-2.20 (m, 1H), 2.10-2.02 (m, 1H), 1.90-1.70 (m, 2H), 1.60-1.40 (m, 3H), 1.32-1.20 (m, 1H); LC-MS (ESI) m/z 418.2 (M+H)⁺, RT=1.86 minutes.

### Example 135

### 5-{[3-(2-chlorophenyl)propyl](3,5-dimethoxybenzoyl)amino}pentanoic Acid

### Step 1: methyl 5-{[3-(2-chlorophenyl)propyl](3,5-dimethoxybenzoyl)amino}pentanoate

To a solution of 3,5-dimethoxybenzoic acid (0.274 g, 1.506 mmol) and 1-[bis(dimethylamino)methylene]-1H-1,2,3-triazolo[4,5-b]pyridinium 3-oxid hexafluorophosphate (0.611 g, 1.607 mmol, HATU) in N,N-dimethylformamide (5 mL) was added triethylamine (0.224 mL 1.607 mmol). The resulting solution was stirred at room temperature for 10 minutes. Then a solution of methyl 5-{[3-(2-chlorophenyl)propyl]amino}pentanoate (Prepared using the procedure described for Example 134—Step 1 substituting 3-(2-chlorophenyl)propan-1-amine for 3-(2-fluorophenyl)propan-1-amine.) (0.95 g, 1.004 mmol) in CH₃CN (5 mL) was added in one portion. The reaction mixture was stirred at room temperature for 2 hours. The reaction mixture was diluted with water and extracted with ethyl acetate twice. The combined organic layers were washed with saturated NaHCO₃ solution (1×10 mL) and brine, dried over Na₂SO₄, filtered and concentrated to give a residue. The residue was applied to a silica gel column and eluted with hexanes and ethyl acetate (0-50%) to give the titled compound (0.15 g, 0.301 mmol, 30.0% yield). LC-MS (ESI) m/z 448.2 (M+H)⁺, RT=2.05 minutes.

### Step 2: 5-{[3-(2-chlorophenyl)propyl](3,5-dimethoxybenzoyl)amino}pentanoic Acid

To a solution of Example 135—Step 1 (0.15 g, 0.335 mmol) in tetrahydrofuran (4 mL) was added 1 N LiOH (2.009 mL 2.009 mmol). The reaction mixture was heated to 50° C. for 2 hours. The mixture was concentrated, and the resultant aqueous mixture was extracted with t-butyl methyl ether (5 mL). The water layer was separated and acidified with 1 N HCl to pH=2-3. The acidic aqueous mixture was extracted with ethyl acetate twice, and the combined organic layers were concentrated. The residue was purified by preparative HPLC (0.1% CF₃CO₂H in water/CH₃CN) and lyophilized to give the titled compound (0.03 g, 0.068 mmol, 20.23% yield). ¹H NMR (400 MHz, DMSO-d₆, T=20° C.) 6 ppm 12.02 (brs, 1H), 7.44-7.10 (m, 4H), 6.54-6.44 (m, 1H), 6.44-6.30 (m, 2H), 3.72 (s, 6H), 3.48-3.32 (m, 2H), 3.22-3.04 (m, 2H), 2.78-2.66 (m, 1H), 2.45-2.40 (m, 1H), 2.30-2.20 (m, 1H), 2.12-2.00 (m, 1H), 1.90-1.68 (m, 2H), 1.62-1.40 (m, 3H), 1.34-1.20 (m, 1H); LC-MS (ESI) m/z 434.2 (M+H)⁺, RT=1.88 minutes.

### Example 136

### 5-{[3-(3,5-difluorophenyl)propyl](3,5-dimethoxybenzoyl)amino}pentanoic Acid

### Step 1: methyl 5-{[3-(3,5-difluorophenyl)propyl](3,5-dimethoxybenzoyl)amino}pentanoate

To a solution of 3,5-dimethoxybenzoic acid (0.250 g, 1.372 mmol) and 1-[bis(dimethylamino)methylene]-1H-1,2,3-triazolo[4,5-b]pyridinium 3-oxid hexafluorophosphate (0.556 g, 1.464 mmol, HATU) in N,N-dimethylformamide (5 mL) was added triethylamine (0.204 mL 1.464 mmol). The resulting solution was stirred at room temperature for 10 minutes. Then a solution of Example 126—Step 1 (0.87 g, 0.915 mmol) in CH₃CN (5 mL) was added in one portion. The reaction mixture was stirred at room temperature for 2 hours. The reaction mixture was diluted with water and extracted with ethyl acetate twice. The combined organic fractions were washed with saturated NaHCO₃ solution (1×10 mL) and brine, dried over Na₂SO₄, filtered and concentrated to give a residue. The residue was added to a silica gel column and was eluted with hexanes and ethyl acetate (0-50%) to give the titled compound (0.12 g, 0.240 mmol, 26.3% yield). LC-MS (ESI) m/z 450.2 (M+H)⁺, RT=1.98 minutes.

### Step 2: 5-{[3-(3,5-difluorophenyl)propyl](3,5-dimethoxybenzoyl)amino}pentanoic Acid

To a solution of Example 136—Step 1 (0.12 g, 0.267 mmol) in tetrahydrofuran (3 mL) was added 1 N LiOH (1.602 mL 1.602 mmol). The reaction mixture was heated to 50° C. for 2 hours. The mixture was concentrated, and the remaining aqueous mixture was extracted with t-butyl methyl ether (5 mL). The water layer was separated and acidified with 1 N HCl to pH=2-3. The acidic aqueous fraction was extracted with ethyl acetate twice. The combined organic layers were concentrated to give a residue which was purified by preparative HPLC (0.1% CF₃CO₂H in water/CH₃CN) and lyophilized to give the titled compound (0.025 g, 0.056 mmol, 21.07% yield). ¹H NMR (400 MHz, DMSO-d₆, T=20° C.) 6 ppm 12.02 (brs, 1H), 7.10-6.90 (m, 2H), 6.82-6.70 (m, 1H), 6.55-6.30 (m, 3H), 3.73-3.71 (m, 6H), 3.45-3.30 (m, 2H), 3.20-3.00 (m, 2H), 2.70-2.60 (m, 1H), 2.45-2.35 (m, 1H), 2.30-2.15 (m, 1H), 2.10-2.00 (m, 1H), 1.92-1.70 (m, 2H), 1.60-1.40 (m, 3H), 1.35-1.20 (m, 1H); LC-MS (ESI) m/z 436.2 (M+H)⁺, RT=1.87 minutes.

### Example 137

### 5-{[3-(2,6-difluorophenyl)propyl](3,5-dimethoxybenzoyl)amino}pentanoic Acid

### Step 1: methyl 5-{[3-(2,6-difluorophenyl)propyl]amino}pentanoate

In a 10 mL sealed tube was added Example 113—Step 2 (0.6 g, 3.50 mmol), methyl 5-bromopentanoate (0.684 g, 3.50 mmol), K₂CO₃ (0.581 g, 4.21 mmol) and acetonitrile (5 mL). The reaction mixture was stirred at reflux for 1 hour. The mixture was cooled to room temperature and filtered to remove potassium carbonate. The filtrate was concentrated to give the titled compound. LC-MS (ESI) m/z 286.2 (M+H)⁺, RT=0.21 minutes.

### Step 2: methyl 5-{[3-(2,6-difluorophenyl)propyl](3,5-dimethoxybenzoyl)amino}pentanoate

To a solution of 3,5-dimethoxybenzoic acid (0.259 g, 1.419 mmol) and 1-[bis(dimethylamino)methylene]-1H-1,2,3-triazolo[4,5-b]pyridinium 3-oxid hexafluorophosphate (0.576 g, 1.514 mmol, HATU) in N,N-dimethylformamide (5 mL) was added triethylamine (0.211 mL 1.514 mmol). The resulting solution was stirred at room temperature for 10 minutes. Then a solution of Example 137—Step 1 (0.9 g, 0.946 mmol) in CH₃CN (5 mL) was added in one portion. The reaction mixture was stirred at room temperature for 2 hours. The reaction mixture was diluted with water and extracted with ethyl acetate twice. The combined organic fractions were washed with saturated NaHCO₃ solution (1×10 mL) and brine, dried over Na₂SO₄, filtered and concentrated to give a residue. The residue was added to a silica gel column and was eluted with hexanes and ethyl acetate (0-50%) to give the titled compound (0.2 g, 0.445 mmol, 47.0% yield). LC-MS (ESI) m/z 450.2 (M+H)⁺, RT=2.02 minutes.

### Step 3: 5-{[3-(2,6-difluorophenyl)propyl](3,5-dimethoxybenzoyl)amino}pentanoic Acid

To a solution of Example 137—Step 2 (0.2 g, 0.445 mmol) in tetrahydrofuran (5 mL) was added 1 N LiOH (2.67 mL 2.67 mmol). The reaction mixture was heated to 50° C. for 2 hours. The reaction mixture was concentrated, and the remaining aqueous mixture was extracted with t-butyl methyl ether (5 mL). The water layer was separated and acidified with 1 N HCl to pH=2-3. The acidic aqueous fraction was extracted with ethyl acetate twice, and the combined organic layers were concentrated to give a residue which was precipitated from CH₃CN (2 mL) to give the titled compound (0.05 g, 0.109 mmol, 24.52% yield). ¹H NMR (400 MHz, DMSO-d₆, T=20° C.) 6 ppm 12.0 (s, 1H), 7.32-7.20 (m, 1H), 7.10-6.90 (m, 2H), 6.55-6.25 (m, 3H), 3.73-3.70 (m, 6H), 3.45-3.34 (m, 2H), 3.20-3.05 (m, 2H), 2.70-2.60 (m, 1H), 2.5-2.38 (m, 1H), 2.27-2.20 (m, 1H), 2.10-2.00 (m, 1H), 1.87-1.65 (m, 2H), 1.60-1.40 (m, 3H), 1.35-1.20 (m, 1H); LC-MS (ESI) m/z 436.2 (M+H)⁺, RT=1.87 minutes.

### Example 138

### 5-{(3,5-dimethoxy-4-methylbenzoyl)[3-(5-methylfuran-2-yl)propyl]amino}pentanoic Acid

### Step 1: methyl 5-{(3,5-dimethoxy-4-methylbenzoyl)[3-(5-methylfuran-2-yl)propyl]amino}pentanoate

To a solution of 3,5-dimethoxy-4-methylbenzoic acid (352 mg, 1.795 mmol) and 1-[bis(dimethylamino)methylene]-1H-1,2,3-triazolo[4,5-b]pyridinium 3-oxid hexafluorophosphate (751 mg, 1.975 mmol, HATU) in N,N-dimethylformamide (8 mL) was added triethylamine (0.275 mL 1.975 mmol). The resultant mixture was stirred at room temperature for 30 minutes. A solution of Example 133—Step 1 (455 mg, 1.795 mmol) and triethylamine (2 mL) in CH₃CN was added to the reaction system. The mixture was stirred at room temperature overnight. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-35%) to give the titled compound (313 mg, 0.508 mmol, 28.3% yield). LC-MS (ESI) m/z 432.2 (M+H)⁺, RT=2.055 minutes.

### Step 2: 5-{(3,5-dimethoxy-4-methylbenzoyl)[3-(5-methylfuran-2-yl)propyl]amino}pentanoic Acid

To a solution of Example 138—Step 1 (313 mg, 0.725 mmol) in tetrahydrofuran (4 mL) was added 1 N lithium hydroxide (4.35 mL 4.35 mmol). The mixture was stirred at room temperature for 1 hour and then concentrated. The remaining aqueous mixture was washed with t-butyl methyl ether once. Then the aqueous fraction was acidified with 1 N HCl to pH=2-3 and extracted with ethyl acetate once. The organic layer was concentrated. The residue was purified by preparative HPLC (0.1% CF₃CO₂H in water/CH₃CN) and lyophilized twice to give the titled compound (25 mg, 0.060 mmol, 8.26% yield). ¹H NMR (400 MHz, DMSO-d₆) δ ppm 12.02 (s, 1H), 6.50 (s, 2H), 5.96 (d, J=29.2 Hz, 1H), 5.77 (d, J=23.1 Hz, 1H), 3.75 (s, 6H), 3.37 (s, 2H), 3.16 (s, 2H), 2.58 (s, 1H), 2.37 (s, 1H), 2.25 (s, 1H), 2.19 (s, 2H), 2.09 (s, 2H), 1.98 (s, 3H), 1.86 (s, 1H), 1.77 (s, 1H), 1.53 (s, 3H), 1.29 (s, 1H); LC-MS (ESI) m/z 418.2 (M+H)⁺, RT=1.925 minutes.

### Example 139

### (2-{(3,5-dimethoxy-4-methylbenzoyl)[3-(4-fluorophenyl)propyl]amino}ethoxy)acetic Acid

### Step 1: N-(2-{[tert-butyl(dimethyl)silyl]oxy}ethyl)-3-(4-fluorophenyl)propan-1-amine

In a 10 mL sealed tube was added (2-bromoethoxy)(tert-butyl)dimethylsilane (0.468 g, 1.958 mmol), 3-(4-fluorophenyl)propan-1-amine (0.3 g, 1.958 mmol), potassium carbonate (0.325 g, 2.350 mmol) and acetonitrile (6 mL). The reaction mixture was stirred at reflux for 16 hours. The mixture was cooled to room temperature and filtered to remove potassium carbonate. The filtrate was concentrated to give the titled compound. LC-MS (ESI) m/z 312.2 (M+H)⁺, RT=1.83 minutes.

### Step 2: N-(2-{[tert-butyl(dimethyl)silyl]oxy}ethyl)-N-[3-(4-fluorophenyl)propyl]-3,5-dimethoxy-4-methylbenzamide

To a solution of 3,5-dimethoxy-4-methylbenzoic acid (0.346 g, 1.762 mmol) and 1-[bis(dimethylamino)methylene]-1H-1,2,3-triazolo[4,5-b]pyridinium 3-oxid hexafluorophosphate (0.670 g, 1.762 mmol, HATU) in N,N-dimethylformamide (10 mL) was added triethylamine (0.246 mL 1.762 mmol). The resulting solution was stirred at room temperature for 10 minutes. Then a solution of Example 139—Step 1 (0.61 g, 1.175 mmol) in CH₃CN (10 mL) was added in one portion. The reaction mixture was stirred at room temperature for 2 hours. The reaction mixture was diluted with water and extracted with ethyl acetate twice. The combined organic fractions were washed with saturated NaHCO₃ solution (1×10 mL) and brine, dried over Na₂SO₄, filtered and concentrated to give a residue. The residue was added to a silica gel column and was eluted with hexanes and ethyl acetate (0-50%) to give the titled compound (0.44 g, 0.764 mmol, 65.0% yield). LC-MS (ESI) m/z 490.4 (M+H)⁺, RT=2.38 minutes.

### Step 3: N-[3-(4-fluorophenyl)propyl]-N-(2-hydroxyethyl)-3,5-dimethoxy-4-methylbenzamide

To a solution of Example 139—Step 2 (0.44 g, 0.899 mmol) in tetrahydrofuran (10 mL) was added tetra-n-butylammonium fluoride (0.235 g, 0.899 mmol). The reaction mixture was stirred at 20° C. for 1 hour. The reaction mixture was concentrated. The residue was dissolved in t-butyl methyl ether (10 mL), washed with water (5 mL) and brine (5 mL), dried over Na₂SO₄, filtered and concentrated to give the titled compound. LC-MS (ESI) m/z 376.2 (M+H)⁺, RT=1.88 minutes.

### Step 4: methyl (2-{(3,5-dimethoxy-4-methylbenzoyl)[3-(4-fluorophenyl)propyl]amino}ethoxy)acetate

To a solution of Example 139—Step 3 (0.338 g, 0.899 mmol) in tetrahydrofuran (5 mL) was added methyl 2-chloroacetate (0.293 g, 2.70 mmol) and potassium tert-butoxide (0.303 g, 2.70 mmol) in one portion. The mixture was heated to reflux for 2 hours. The mixture was cooled down, quenched with saturated aqueous ammonium chloride, and extracted with ethyl acetate 3 times. The combined organic layers were washed with brine, dried over anhydrous Na₂SO₄, filtered and concentrated to give a residue which was purified by flash column chromatography on silica gel (eluent: hexanes:ethyl acetate from 100:0 to 50:50) to give the titled compound (0.2 g, 0.402 mmol, 44.7% yield). LC-MS (ESI) m/z 448.2 (M+H)⁺, RT=2.02 minutes.

### Step 5: (2-{(3,5-dimethoxy-4-methylbenzoyl)[3-(4-fluorophenyl)propyl]amino}ethoxy)acetic Acid

To a solution of Example 139—Step 4 (0.2 g, 0.447 mmol) in tetrahydrofuran (6 mL) was added 1 N LiOH (2.68 mL 2.68 mmol). The reaction mixture was heated to 50° C. for 2 hours. The reaction mixture was concentrated, and the remaining aqueous mixture was extracted with t-butyl methyl ether (5 mL). The water layer was separated and acidified with 1 N HCl to pH=2-3. The acidic aqueous fraction was extracted with ethyl acetate twice. The combined organic layers were concentrated to give a crude product, which was purified by preparative HPLC (0.1% CF₃CO₂H in water/CH₃CN) and lyophilized to give the titled compound (0.052 g, 0.118 mmol, 26.3% yield). ¹H NMR (400 MHz, DMSO-d₆) δ ppm 7.35-7.22 (m, 1H), 7.15-6.90 (m, 3H), 6.65-6.45 (m, 2H), 4.07-3.90 (m, 2H), 3.79-3.69 (m, 6H), 3.68-3.49 (m, 3H), 3.48-3.31 (m, 2H), 3.21-3.14 (m, 1H), 2.68-2.56 (m, 1H), 2.45-2.31 (m, 1H), 1.98 (s, 3H), 1.91-1.71 (m, 2H); LC-MS (ESI) m/z 434.2 (M+H)⁺, RT=1.89 minutes.

### Example 140

### N-{5-[(4-fluorobenzene-1-sulfonyl)amino]-5-oxopentyl}-3,5-dimethoxy-4-methyl-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (62.7 mg, 0.387 mmol) in isopropyl acetate (2 mL) was stirred at 40° C. for 10 minutes. 4-Fluorobenzenesulfonamide (55.1 mg, 0.314 mmol) and 1,8-diazabicyclo[5.4.0]undec-7-ene (72.9 μL, 0.484 mmol) were added, and the reaction mixture was stirred at 40° C. for 18 hours. The reaction mixture was diluted with 10 mL of ethyl acetate, washed with 10 mL each of 1 N HCl and saturated NaCl, dried with MgSO₄ and concentrated. Flash chromatography on silica gel (100% ethyl acetate) gave the titled compound (91 mg, 66%). ¹H NMR (400 MHz, DMSO-d₆) δ ppm 1.30 (d, J=80.5 Hz, 5H), 1.77 (d, J=21.1 Hz, 2H), 1.96 (s, 3H), 2.02-2.43 (m, 3H), 2.57 (s, 1H), 3.07 (s, 2H), 3.71 (s, 6H), 6.43 (s, 2H), 6.84-7.36 (m, 5H), 7.36-7.49 (m, 2H), 7.94 (dd, J=8.7, 5.1 Hz, 2H), 12.07 (s, 1H); MS (DCI) m/z 571 (M+H)⁺.

### Example 141

### 3,5-dimethoxy-4-methyl-N-{5-oxo-5-[(pyridine-2-sulfonyl)amino]pentyl}-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (62.7 mg, 0.387 mmol) in isopropyl acetate (2 mL) was stirred at 40° C. for 10 minutes. Pyridine-2-sulfonamide (49.7 mg, 0.314 mmol) and 1,8-diazabicyclo[5.4.0]undec-7-ene (72.9 μL, 0.484 mmol) were added, and the reaction mixture was stirred at 40° C. for 18 hours. The reaction mixture was diluted with 10 mL of ethyl acetate; washed with 10 mL each of 1 N HCl, 1 N NaOH and saturated NaCl; dried (MgSO4) and concentrated. Flash chromatography on silica gel (0-10% ethanol in ethyl acetate) gave the titled compound (12 mg, 9%). ¹H NMR (400 MHz, DMSO-d₆, 90° C.) 6 ppm 1.41 (dq, J=38.8, 7.6 Hz, 4H), 1.75-1.89 (m, 2H), 2.00 (d, J=8.2 Hz, 5H), 2.47-2.60 (m, 2H), 3.25 (dt, J=16.2, 7.5 Hz, 5H), 3.73 (s, 6H), 6.46 (s, 2H), 7.03-7.16 (m, 3H), 7.16-7.27 (m, 2H), 7.40 (td, J=5.2, 3.0 Hz, 1H), 7.80-7.91 (m, 2H), 8.50 (d, J=4.7 Hz, 1H); MS (DCI) m/z 554 (M+H)⁺.

### Example 142

### 3,5-dimethoxy-4-methyl-N-{5-oxo-5-[(pyridine-3-sulfonyl)amino]pentyl}-N-(3-phenylpropyl)benzamide

A mixture of 5-[(3,5-dimethoxy-4-methylbenzoyl)(3-phenylpropyl)amino]pentanoic acid (103 mg, 0.249 mmol, Example 4) and 1,1′-carbonyldiimidazole (64.6 mg, 0.399 mmol) in isopropyl acetate (2 mL) was stirred at 40° C. for 10 minutes. Pyridine-3-sulfonamide (51.2 mg, 0.324 mmol) and 1,8-diazabicyclo[5.4.0]undec-7-ene (75 μL, 0.498 mmol) were added, and the reaction mixture was stirred at 40° C. for 18 hours. The reaction mixture was diluted with 10 mL of ethyl acetate, washed with 10 mL of 1 N HCl and saturated NaCl, dried with MgSO₄ and concentrated. Flash chromatography on silica gel (0-10% ethanol in ethyl acetate) gave the titled compound (32 mg, 23%). ¹H NMR (400 MHz, DMSO-d₆) δ ppm 1.42 (s, 6H), 1.98 (d, J=1.8 Hz, 3H), 2.15 (d, J=59.1 Hz, 2H), 2.38 (q, J=6.5, 5.1 Hz, 1H), 2.59 (s, 1H), 3.09 (s, 5H), 3.72 (s, 6H), 6.46 (d, J=11.4 Hz, 2H), 7.14 (t, J=46.6 Hz, 5H), 7.60 (dd, J=8.1, 4.8 Hz, 1H), 8.22 (s, 1H), 8.79 (dd, J=4.8, 1.6 Hz, 1H), 8.99 (s, 1H); MS (DCI) m/z 554 (M+H)⁺.

### Example 143

### 3,5-dimethoxy-4-methyl-N-{5-oxo-5-[(pyridine-4-sulfonyl)amino]pentyl}-N-(3-phenylpropyl)benzamide

A mixture of 5-[(3,5-dimethoxy-4-methylbenzoyl)(3-phenylpropyl)amino]pentanoic acid (108 mg, 0.261 mmol, Example 4) and 1,1′-carbonyldiimidazole (67.8 mg, 0.418 mmol) in isopropyl acetate (2 mL) was stirred at 40° C. for 10 minutes. Pyridine-4-sulfonamide (53.7 mg, 0.340 mmol) and 1,8-diazabicyclo[5.4.0]undec-7-ene (79 μL, 0.522 mmol) were added, and the reaction mixture was stirred at 40° C. for 18 hours. The reaction mixture was diluted with 10 mL of ethyl acetate, washed with 10 mL of 1 N HCl and saturated NaCl, dried with MgSO₄ and concentrated. Flash chromatography on silica gel (0-10% ethanol in ethyl acetate) gave the titled compound (59 mg, 41%). ¹H NMR (400 MHz, DMSO-d₆) δ ppm 1.01-1.27 (m, 2H), 1.46 (d, J=37.5 Hz, 4H), 1.84 (s, 3H), 1.97 (d, J=3.0 Hz, 5H), 2.38 (s, 1H), 2.60 (s, 1H), 3.09 (s, 2H), 3.72 (s, 6H), 6.46 (s, 2H), 6.86-7.40 (m, 5H), 7.60 (s, 2H), 8.54-8.67 (m, 2H); MS (DCI) m/z 554 (M+H)⁺.

### Example 144

### N-{5-[(benzenesulfonyl)amino]-5-oxopentyl}-3,5-dimethoxy-4-methyl-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (62.7 mg, 0.387 mmol) in isopropyl acetate (2 mL) was stirred at 40° C. for 10 minutes. Benzenesulfonamide (49.4 mg, 0.314 mmol) and 1,8-diazabicyclo[5.4.0]undec-7-ene (72.9 μL, 0.484 mmol) were added, and the reaction mixture was stirred at 40° C. for 18 hours. The reaction mixture was diluted with 10 mL of ethyl acetate, washed with 10 mL of 1 N HCl and saturated NaCl, dried with MgSO₄ and concentrated. Flash chromatography on silica gel (0-50% ethyl acetate in heptane) gave the titled compound (15 mg, 11%). ¹H NMR (400 MHz, DMSO-d₆) δ ppm 1.3-1.52 (m, 4H), 1.79 (d, J=24.6 Hz, 2H), 1.98 (s, 3H), 2.05-2.43 (m, 4H), 2.58 (s, 1H), 3.08 (s, 2H), 3.72 (s, 6H), 6.44 (s, 2H), 7.13 (t, J=47.0 Hz, 6H), 7.50-7.77 (m, 3H), 7.88 (d, J=7.7 Hz, 2H), 12.03 (s, 1H); MS (DCI) m/z 553 (M+H)⁺.

### Example 145

### 3-[(3,5-dimethoxy-4-methylbenzoyl)(3-phenylpropyl)amino]propyl (4-fluorobenzene-1-sulfonyl)carbamate

### Step 1: N-(3-hydroxypropyl)-3,5-dimethoxy-4-methyl-N-(3-phenylpropyl)benzamide

A mixture of 3-phenylpropylamine (0.563 mL, 3.95 mmol) and (3-bromopropoxy)(tert-butyl)dimethylsilane (0.897 mL, 3.95 mmol) in acetonitrile (20 mL) was heated to reflux for 2 hours and then cooled to room temperature to give 3-((tert-butyldimethylsilyl)oxy)-N-(3-phenylpropyl)propan-1-amine. Meanwhile, to a solution of 3,5-dimethoxy-4-methylbenzoic acid (0.775 g, 3.95 mmol) in N,N-dimethylformamide (10 mL) was added 1-[bis(dimethylamino)methylene]-1H-1,2,3-triazolo[4,5-b]pyridinium 3-oxid hexafluorophosphate (1.501 g, 3.95 mmol, HATU) followed by Hunig's base (1.000 mL, 5.73 mmol). After stirring for 15 minutes, this solution was added to the solution of 3-((tert-butyldimethylsilyl)oxy)-N-(3-phenylpropyl)propan-1-amine. The reaction mixture was stirred at room temperature overnight. The crude mixture was concentrated and then partitioned between 1 M HCl (˜50 mL) and ethyl acetate (˜50 mL). The aqueous phase was extracted twice with ethyl acetate. The combined organic phases were washed with HCl (2×), aqueous NaHCO₃ (2×) and brine, dried over MgSO₄ and concentrated. Flash chromatography on silica gel eluted with 0-100% t-butyl methyl ether in hexanes gave the titled compound (500 mg, 34.1%) that was used directly in the next step. MS (DCI) m/z 372 (M+H)⁺.

### Step 2: 3-[(3,5-dimethoxy-4-methylbenzoyl)(3-phenylpropyl)amino]propyl (4-fluorobenzene-1-sulfonyl)carbamate

To a solution of N-(3-hydroxypropyl)-3,5-dimethoxy-4-methyl-N-(3-phenylpropyl)benzamide (150 mg, 0.404 mmol,) in t-butyl methyl ether (10 mL) was added 4-fluorobenzenesulfonyl isocyanate (81 mg, 0.404 mmol) dropwise. The solution was stirred at room temperature overnight. The reaction mixture was diluted with ethyl acetate, washed with brine, dried and concentrated. Flash chromatography on silica gel (0-100% t-butyl methyl ether/heptanes) gave the titled compound (173 mg, 75%). ¹H NMR (400 MHz, DMSO-d₆) δ ppm 0.67-0.91 (m, 2H), 1.17 (m, 2H), 1.78 (m, 4H), 1.96 (s, 3H), 2.36 (s, 1H), 3.12 (m, 2H), 3.71 (s, 6H), 4.00 (m, 1H), 6.46 (s, 2H), 6.85-7.33 (m, 5H), 7.33-7.51 (m, 2H), 7.80-8.07 (m, 2H), 12.04 (s, 1H); MS (DCI) m/z 573 (M+H)⁺.

### Example 146

### 5-{[3-(2,4-difluorophenyl)propyl](3,5-dimethoxy-4-methylbenzoyl)amino}pentanoic Acid

### Step 1: methyl 5-{[3-(2,4-difluorophenyl)propyl]amino}pentanoate

In a 10 mL sealed tube was added Example 114—Step 2 (0.3 g, 1.752 mmol), methyl 5-bromopentanoate (0.410 g, 2.103 mmol), K₂CO₃ (0.291 g, 2.103 mmol) and acetonitrile (5 mL). The reaction mixture was stirred at reflux for 1 hour. The mixture was cooled to room temperature and filtered to remove potassium carbonate. The filtrate was concentrated to give the titled compound.

### Step 2: methyl 5-{[3-(2,4-difluorophenyl)propyl](3,5-dimethoxy-4-methylbenzoyl)amino}pentanoate

To a solution of 3,5-dimethoxy-4-methylbenzoic acid (0.206 g, 1.051 mmol) and 1-[bis(dimethylamino)methylene]-1H-1,2,3-triazolo[4,5-b]pyridinium 3-oxid hexafluorophosphate (0.426 g, 1.122 mmol, HATU) in N,N-dimethylformamide (5 mL) was added triethylamine (0.156 mL 1.122 mmol). The resulting solution was stirred at room temperature for 10 minutes. Then a solution of Example 146—Step 1 (0.5 g, 0.701 mmol) in CH₃CN (5 mL) was added in one portion. The reaction mixture was stirred at room temperature for 2 hours. The reaction mixture was diluted with water and extracted with ethyl acetate twice. The combined organic fractions were washed with saturated NaHCO₃ solution (1×10 mL) and brine, dried over Na₂SO₄, filtered and concentrated to give a residue. The residue was added to a silica gel column that was eluted with hexanes and ethyl acetate (0-50%) to give the titled compound (0.25 g, 0.485 mmol, 69.3% yield). LC-MS (ESI) m/z 464.2 (M+H)⁺, RT=2.11 minutes.

### Step 3: 5-{[3-(2,4-difluorophenyl)propyl](3,5-dimethoxy-4-methylbenzoyl)amino}pentanoic Acid

To a solution of Example 146—Step 2 (0.25 g, 0.539 mmol) in tetrahydrofuran (6 mL) was added 1 N LiOH (3.24 mL 3.24 mmol). The reaction mixture was heated to 50° C. for 2 hours. The reaction mixture was concentrated, and the remaining aqueous mixture was extracted with t-butyl methyl ether (5 mL). The aqueous mixture was cooled down and acidified with 1 N HCl to pH=2-3. The aqueous mixture was extracted with ethyl acetate twice, and the combined organic layers were concentrated to give a residue which was purified by preparative HPLC (0.1% CF₃CO₂H as buffer) and lyophilized to give the titled compound (0.105 g, 0.229 mmol, 42.4% yield). ¹H NMR (400 MHz, DMSO-d₆, T=20° C.) 6 ppm 11.99 (brs, 1H), 7.45-6.80 (m, 3H), 6.55-6.35 (m, 2H), 3.80-3.68 (m, 6H), 3.62-3.55 (m, 2H), 3.24-3.02 (m, 2H), 2.68-2.54 (m, 1H), 2.44-2.32 (m, 1H), 2.28-2.20 (m, 1H), 2.14-2.02 (m, 1H), 1.97 (s, 3H), 1.90-1.67 (m, 2H), 1.62-1.42 (m, 3H), 1.41-1.21 (m, 1H); LC-MS (ESI) m/z 450.2 (M+H)⁺, RT=1.96 minutes.

### Example 147

### 5-{[3-(2,6-difluorophenyl)propyl](3,5-dimethoxy-4-methylbenzoyl)amino}pentanoic Acid

### Step 1: methyl 5-{[3-(2,6-difluorophenyl)propyl](3,5-dimethoxy-4-methylbenzoyl)amino}pentanoate

To a solution of 3,5-dimethoxy-4-methylbenzoic acid (0.248 g, 1.262 mmol) and 1-[bis(dimethylamino)methylene]-1H-1,2,3-triazolo[4,5-b]pyridinium 3-oxid hexafluorophosphate (0.512 g, 1.346 mmol, HATU) in N,N-dimethylformamide (5 mL) was added triethylamine (0.188 mL 1.346 mmol). The resulting solution was stirred at room temperature for 10 minutes. Then a solution of Example 137—Step 1 (0.6 g, 0.841 mmol) in CH₃CN (5 mL) was added in one portion. The reaction mixture was stirred at room temperature for 2 hours. The reaction mixture was diluted with water and extracted with ethyl acetate twice. The combined organic fractions were washed with saturated NaHCO₃ solution (1×10 mL) and brine, dried over Na₂SO₄, filtered and concentrated to give a residue. The residue was added to a silica gel column that was eluted with hexanes and ethyl acetate (0-50%) to give the titled compound (0.3 g, 0.583 mmol, 69.3% yield). LC-MS (ESI) m/z 464.2 (M+H)⁺, RT=2.11 minutes.

### Step 2: 5-{[3-(2,6-difluorophenyl)propyl](3,5-dimethoxy-4-methylbenzoyl)amino}pentanoic Acid

To a solution of Example 147—Step 1 (0.3 g, 0.647 mmol) in tetrahydrofuran (6 mL) was added 1 N LiOH (3.88 mL 3.88 mmol). The reaction mixture was heated to 50° C. for 2 hours. The reaction mixture was concentrated, and the remaining aqueous mixture was extracted with t-butyl methyl ether (5 mL). The mixture was cooled down and acidified with 1 N HCl to pH=2-3. The acidic aqueous mixture was extracted with ethyl acetate twice, and the combined organic layers were concentrated to give a residue which was precipitated from CH₃CN (2 mL) to give the titled compound (0.15 g, 0.320 mmol, 49.5% yield). ¹H NMR (400 MHz, DMSO-d₆, T=20° C.) 6 ppm 12.01 (s, 1H), 7.36-7.16 (m, 1H), 7.12-6.82 (m, 1H), 6.54-6.36 (m, 2H), 3.78-3.66 (m, 6H), 3.46-3.34 (m, 2H), 3.22-3.06 (m, 2H), 2.72-2.60 (m, 1H), 2.46-2.36 (m, 1H), 2.30-2.20 (m, 1H), 2.14-2.02 (m, 1H), 1.96 (s, 3H), 1.88-1.68 (m, 2H), 1.62-1.42 (m, 3H), 1.34-1.22 (m, 1H); LC-MS (ESI) m/z 450.2 (M+H)⁺, RT=1.95 minutes.

### Example 148

### 5-{(3,5-dimethoxybenzoyl)[3-(4-fluorophenyl)propyl]amino}pentanoic Acid

### Step 1: methyl 5-{[3-(4-fluorophenyl)propyl]amino}pentanoate

To a 10 mL sealed tube was added 3-(4-fluorophenyl)propan-1-amine (0.3 g, 1.958 mmol), methyl 5-bromopentanoate (0.382 g, 1.958 mmol), K₂CO₃ (0.271 g, 1.958 mmol) and acetonitrile (5 mL). The reaction mixture was stirred at reflux for 1 hour. The mixture was cooled to room temperature and filtered to remove potassium carbonate. The filtrate was concentrated to give the titled compound. LC-MS (ESI) m/z 268.2 (M+H)⁺, RT=1.52 minutes.

### Step 2: methyl 5-{(3,5-dimethoxybenzoyl)[3-(4-fluorophenyl)propyl]amino}pentanoate

To a solution of 3,5-dimethoxybenzoic acid (0.153 g, 0.842 mmol) and 1-[bis(dimethylamino)methylene]-1H-1,2,3-triazolo[4,5-b]pyridinium 3-oxid hexafluorophosphate (0.341 g, 0.898 mmol, HATU) in N,N-dimethylformamide (5 mL) was added triethylamine (0.125 mL 0.898 mmol). The resulting solution was stirred at room temperature for 10 minutes. Then a solution of Example 148—Step 1 (0.5 g, 0.561 mmol) in CH₃CN (5 mL) was added in one portion. The reaction mixture was stirred at room temperature for 2 hours. The reaction mixture was diluted with water and extracted with ethyl acetate twice. The combined organic fractions were washed with saturated NaHCO₃ solution (1×10 mL) and brine, dried over Na₂SO₄, filtered and concentrated to give a residue. The residue was added to a silica gel column that was eluted with hexanes and ethyl acetate (0-50%) to give the titled compound (0.25 g, 0.521 mmol, 93% yield). LC-MS (ESI) m/z 432.2 (M+H)⁺, RT=2.01 minutes.

### Step 3: 5-{(3,5-dimethoxybenzoyl)[3-(4-fluorophenyl)propyl]amino}pentanoic Acid

To a solution of Example 148—Step 2 (0.25 g, 0.579 mmol) in tetrahydrofuran (3 mL) was added 1 N LiOH (0.579 mL 0.579 mmol). The reaction mixture was heated to 50° C. for 2 hours. The reaction mixture was concentrated, and the remaining aqueous mixture was extracted with t-butyl methyl ether (5 mL). The aqueous mixture was then acidified with 1 N HCl to pH=2-3. The acidic aqueous mixture was extracted with ethyl acetate twice, and the combined organic layers were concentrated to give a residue which was purified by preparative HPLC (0.1% CF₃CO₂H as buffer) and lyophilized to give the titled compound (0.085 g, 0.200 mmol, 34.4% yield). ¹H NMR (400 MHz, DMSO-d₆) δ ppm 12.01 (s, 1H), 7.27 (t, J=6.8 Hz, 1H), 7.04 (ddd, J=28.9, 20.5, 8.7 Hz, 3H), 6.49 (d, J=13.5 Hz, 1H), 6.36 (d, J=19.1 Hz, 2H), 3.89-3.57 (m, 6H), 3.50-3.37 (m, 2H), 3.18-3.02 (m, 2H), 2.64-2.54 (m, 1H), 2.42-2.33 (m, 1H), 2.28-2.20 (m, 1H), 2.12-2.02 (m, 1H), 1.90-1.68 (m, 2H), 1.62-1.35 (m, 3H), 1.37-1.14 (m, 1H); LC-MS (ESI) m/z 418.2 (M+H)⁺, RT=1.87 minutes.

### Example 149

### 5-{[2-ethoxy-1-(4-methoxyphenyl)cyclopropane-1-carbonyl](3-phenylpropyl)amino}pentanoic Acid

### Step 1: methyl 5-[(tert-butoxycarbonyl)(3-phenylpropyl)amino]pentanoate

A solution of methyl 5-bromopentanoate (4.00 g, 20.51 mmol) in acetonitrile (40 mL) was heated to reflux, and then a solution of 3-phenylpropan-1-amine (2.77 g, 20.51 mmol) in acetonitrile (5 mL) was added to the mixture. The mixture was heated to reflux for 1 hour. The mixture was cooled to 0° C., di-tert-butyl dicarbonate (4.48 g, 20.51 mmol) was added to the mixture and triethylamine (2.075 g, 20.51 mmol) was added subsequently. The mixture was stirred at room temperature for 30 minutes and then concentrated to dryness. The residue was purified by flash chromatography with 0-20% ethyl acetate/hexane to give the titled compound (3.00 g, 8.58 mmol, 41.9% yield). LC-MS (ESI) m/z 250.5 (M-CO₂C(CH₃)₃+H)⁺, RT=2.18 minutes.

### Step 2: methyl 5-[(3-phenylpropyl)amino]pentanoate-hydrogen chloride

A solution of Example 149—Step 1 (1.20 g, 3.43 mmol) in 4 N HCl in 1,4-dioxane (12 mL) was stirred at room temperature for 30 minutes. Then the mixture was concentrated to dryness under vacuum to give the titled compound (980 mg, 3.43 mmol, 100% yield) as a HCl salt. LC-MS (ESI) m/z 250.5 (M+H)⁺, RT=1.48 minutes.

### Step 3: methyl 2-ethoxy-1-(4-methoxyphenyl)cyclopropane-1-carboxylate

To a solution of ethoxyethene (0.157 g, 2.182 mmol) and diacetoxyrhodium (0.026 g, 0.058 mmol) in 5 mL of 2,2-dimethyl butane was added dropwise methyl 2-diazo-2-(4-methoxyphenyl)acetate (0.15 g, 0.727 mmol) in 2,2-dimethylbutane (50 mL) treated with 2 mg of diacetoxyrhodium (2.144 mg, 4.85 μmol). The mixture was allowed to stir at 25° C. for 30 minutes and was then concentrated under reduced pressure. Chromatography of the resulting residue on flash column (eluent, ethyl acetate/hexane from 0/100 to 20/80) gave the titled compound. ¹H NMR (400 MHz, CDCl₃) δ ppm 7.35-7.20 (m, 2H), 6.94-6.76 (m, 2H), 3.93-3.86 (m, 1H), 3.81 (s, 3H), 3.63 (s, 3H), 3.58 (pd, J=5.4, 4.7, 2.1 Hz, 2H), 1.78 (dd, J=7.1, 5.7 Hz, 1H), 1.57 (d, J=10.3 Hz, 1H), 1.03 (t, J=7.0 Hz, 3H).

### Step 4: 2-ethoxy-1-(4-methoxyphenyl)cyclopropane-1-carboxylic Acid

A mixture of Example 149—Step 3 (30 mg, 0.120 mmol), lithium hydroxide hydrate (20.12 mg, 0.479 mmol), water (1 mL) and tetrahydrofuran (1 mL) was stirred at 40° C. for 2 hours. The mixture was acidified to pH=4 with 1 N HCl. The mixture was extracted with ethyl acetate (3×20 mL). The organic layers were combined, washed with brine, dried with Na₂SO₄ and concentrated to dryness to give the titled compound. ¹H NMR (400 MHz, CDCl₃) δ ppm 7.30-7.27 (m, 2H), 6.92-6.84 (m, 2H), 3.97-3.92 (m, 1H), 3.81 (d, J=1.6 Hz, 3H), 3.62-3.55 (m, 2H), 1.84 (dd, J=7.1, 5.8 Hz, 1H), 1.65 (dd, J=5.8, 4.8 Hz, 1H), 1.03 (t, J=7.0 Hz, 3H).

### Step 5: methyl 5-{[2-ethoxy-1-(4-methoxyphenyl)cyclopropane-1-carbonyl](3-phenylpropyl)amino}pentanoate

A mixture of Example 149—Step 4 (35 mg, 0.148 mmol), 2-(3H-[1,2,3]triazolo[4,5-b]pyridin-3-yl)-1,1,3,3-tetramethylisouronium hexafluorophosphate(V) (56.3 mg, 0.148 mmol), Example 149—Step 2 (50.8 mg, 0.178 mmol), N,N-dimethylformamide (2 mL) and N-ethyl-N-isopropylpropan-2-amine (57.4 mg, 0.444 mmol) was stirred at 25° C. for 2 hours. The reaction was quenched with water. The mixture was extracted with ethyl acetate (2×20 mL). The combined organic fractions were washed with brine (2×30 mL), and dried over Na₂SO₄, and concentrated to give the titled compound. LC-MS (ESI) m/z 468.4 (M+H)⁺, RT=2.096 minutes.

### Step 6: 5-{[2-ethoxy-1-(4-methoxyphenyl)cyclopropane-1-carbonyl](3-phenylpropyl)amino}pentanoic Acid

A mixture of Example 149—Step 5 (35 mg, 0.075 mmol), lithium hydroxide hydrate (12.57 mg, 0.299 mmol), water (1 mL) and tetrahydrofuran (1 mL) was stirred at 25° C. for 2 hours. The mixture was acidified to pH=4 with 1 N HCl. The mixture was purified by preparative HPLC (0.1% CF₃CO₂H in water/CH₃CN) to give the titled compound. ¹H NMR (400 MHz, methanol-d₄) δ ppm 7.25 (ddd, J=7.5, 5.5, 3.4 Hz, 3H), 7.21-7.12 (m, 2H), 7.10-7.06 (m, 1H), 7.01-6.97 (m, 1H), 6.92-6.83 (m, 2H), 3.83-3.75 (m, 4H), 3.42 (ddt, J=14.2, 9.2, 7.1 Hz, 1H), 3.29-3.15 (m, 4H), 2.57 (t, J=7.8 Hz, 1H), 2.40-2.22 (m, 1H), 2.18-2.12 (m, 1H), 1.96 (p, J=7.7, 7.1 Hz, 1H), 1.81 (ddt, J=19.6, 8.9, 6.4 Hz, 1H), 1.63-1.41 (m, 4H), 1.32 (td, J=9.8, 9.2, 4.1 Hz, 1H), 1.28-1.09 (m, 2H), 0.82 (dt, J=13.9, 7.0 Hz, 4H); LC-MS (ESI) m/z 454.4 (M+H)⁺, RT=1.940 minutes.

### Example 150

### 3,5-dimethoxy-4-methyl-N-{5-oxo-5-[(trifluoromethanesulfonyl)amino]pentyl}-N-(3-phenylpropyl)benzamide

A mixture of 5-[(3,5-dimethoxy-4-methylbenzoyl)(3-phenylpropyl)amino]pentanoic acid (100 mg, 0.242 mmol, Example 4) and 1,1′-carbonyldiimidazole (62.7 mg, 0.387 mmol) in isopropyl acetate (2 mL) was stirred at 50° C. for 20 minutes. Trifluoromethanesulfonamide (46.9 mg, 0.314 mmol) and 1,8-diazabicyclo[5.4.0]undec-7-ene (72.9 μL, 0.484 mmol) were added, and the reaction mixture was stirred at 50° C. for 18 hours. Additional trifluoromethanesulfonamide (5 mg, 0.03 mmol) was added, and the reaction mixture was stirred at 60° C. for 1 hour. The reaction mixture was diluted with 10 mL of ethyl acetate; washed with 10 mL of 1 N HCl, 1N NaOH, and saturated NaCl; dried with MgSO₄ and concentrated. Two rounds of flash chromatography on silica gel (100% ethyl acetate) gave the titled compound (12 mg, 9%). ¹H NMR (501 MHz, DMSO-d₆) δ ppm 1.27 (s, 1H), 1.51 (s, 3H), 1.89 (s, 2H), 1.97 (d, J=5.4 Hz, 3H), 2.10 (d, J=4.0 Hz, 1H), 2.34 (m, 2H), 2.62 (m, 1H), 3.13 (m, 2H), 3.37 (m, 3H), 3.74 (s, 6H), 6.47 (s, 2H), 6.93-7.39 (m, 5H); MS (DCI) m/z 544 (M+H)⁺.

### Example 151

### 5-[(3,5-dimethoxy-4-methylbenzoyl)(3-phenylpropyl)amino]-2-hydroxypentanoic Acid

### Step 1: methyl 2-(acetyloxy)-5-iodopentanoate

To a mixture of methyl tetrahydrofuran-2-carboxylate (1 g, 7.68 mmol) and sodium iodide (2.304 g, 15.37 mmol) in dry CH₃CN (10 mL) cooled in an ice-water bath was added, dropwise, acetyl chloride (1.093 mL 15.37 mmol). Then the mixture was stirred for 24 hours at room temperature. The mixture was quenched with saturated NaHCO₃ (10 mL), much bubbling occurred, and the suspension became clear. The aqueous fraction was separated and extracted with t-butyl methyl ether 3 times. The combined organic layers were washed with brine and saturated NaHSO₃ once, dried, and concentrated to give the titled compound (1.989 g, 6.63 mmol, 86% yield) as a light yellow oil. ¹H NMR (400 MHz, CDCl₃) δ ppm 5.07-5.00 (m, 1H), 3.76 (s, 3H), 3.27-3.15 (m, 2H), 2.15 (s, 3H), 1.99-1.87 (m, 4H).

### Step 2: methyl 2-(acetyloxy)-5-[(3-phenylpropyl)amino]pentanoate

A mixture of 3-phenylpropan-1-amine (162 mg, 1.196 mmol) and Example 151—Step 1 (359 mg, 1.196 mmol) in CH₃CN (3 mL) was stirred at reflux for 40 minutes. The solution was cooled down and concentrated to give the titled compound. LC-MS (ESI) m/z 308.2 (M+H)⁺, RT=1.541 minutes.

### Step 3: methyl 2-(acetyloxy)-5-[(3,5-dimethoxy-4-methylbenzoyl)(3-phenylpropyl)amino]pentanoate

To a solution of 3,5-dimethoxy-4-methylbenzoic acid (235 mg, 1.196 mmol) and 1-[bis(dimethylamino)methylene]-1H-1,2,3-triazolo[4,5-b]pyridinium 3-oxid hexafluorophosphate (500 mg, 1.316 mmol, HATU) in N,N-dimethylformamide (6 mL) was added triethylamine (0.183 mL 1.316 mmol). The resultant mixture was stirred at room temperature for 30 minutes. A solution of Example 151—Step 2 (368 mg, 1.196 mmol) and triethylamine (1 mL) in CH₃CN (3 mL) was added to the reaction system. The mixture was stirred at room temperature for 1 hour. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (127 mg, 0.201 mmol, 16.84% yield). LC-MS (ESI) m/z 486.2 (M+H)⁺, RT=2.084 minutes.

### Step 4: 5-[(3,5-dimethoxy-4-methylbenzoyl)(3-phenylpropyl)amino]-2-hydroxypentanoic Acid

To a solution of Example 151—Step 3 (127 mg, 0.262 mmol) in tetrahydrofuran (4 mL) was added 1 N lithium hydroxide (2.62 mL 2.62 mmol). The mixture was stirred at room temperature overnight. The mixture was acidified with 1 N HCl to pH=2-3 and extracted with ethyl acetate once. The organic layer was concentrated. The residue was purified by preparative HPLC (0.1% NH₃H₂O/CH₃CN) and lyophilized to give the titled compound (45 mg, 0.105 mmol, 40.1% yield). ¹H NMR (400 MHz, DMSO-d₆) δ ppm 7.22 (t, J=7.6 Hz, 2H), 7.19-7.00 (m, 3H), 6.49 (s, 2H), 3.76 (s, 6H), 3.64 (s, 1H), 3.28 (s, 4H), 2.51 (d, J=12.8 Hz, 2H), 2.00 (s, 3H), 1.85 (t, J=7.7 Hz, 2H), 1.61 (dd, J=15.2, 7.3 Hz, 2H), 1.52 (s, 1H), 1.37 (s, 1H); LC-MS (ESI) m/z 430.2 (M+H)⁺, RT=1.849 minutes.

### Example 152

### 5-{[1-(4-methoxyphenyl)-3-oxocyclobutane-1-carbonyl](3-phenylpropyl)amino}pentanoic Acid

### Step 1: methyl 5-[(tert-butoxycarbonyl)(3-phenylpropyl)amino]pentanoate

A solution of methyl 5-bromopentanoate (25 g, 128 mmol) in acetonitrile (200 mL) was heated to reflux, and then a solution of 3-phenylpropan-1-amine (19.06 g, 141 mmol) in acetonitrile (5 mL) was added dropwise to the mixture. The mixture was heated to reflux for 0.5 hour, and then was cooled to 0° C. Di-tert-butyl dicarbonate (30.8 g, 141 mmol) was added to the mixture and triethylamine (12.97 g, 128 mmol) was subsequently added. The mixture was stirred at room temperature for 30 minutes and then concentrated to dryness. The residue was diluted with H₂O (100 mL) and extracted with ethyl acetate (300 mL). The organic layer was dried over Na₂SO₄, filtered and concentrated. The residue was purified by flash chromatography with hexane and ethyl acetate (0-10%) to give the titled compound (14.0 g, 40.1 mmol, 31.3% yield). LC-MS (ESI) m/z 250 (M+H)⁺, RT=2.19 minutes.

### Step 2: methyl 5-[(3-phenylpropyl)amino]pentanoate

To a solution of Example 152—Step 1 (2.0 g, 5.72 mmol) in CH₂Cl₂ (6.5 mL) was added CF₃CO₂H (2.126 mL 28.6 mmol). The solution was stirred at room temperature for 1.5 hours. Then triethylamine (8 mL) was added to quench the solution to pH>9. The resulting solution was concentrated to give the titled compound. LC-MS (ESI) m/z 250.2 (M+H)⁺, RT=1.474 minutes.

### Step 3: methyl 5-{[1-(4-methoxyphenyl)-3-oxocyclobutane-1-carbonyl](3-phenylpropyl)amino}pentanoate

Example 152—Step 2 (50 mg, 0.143 mmol) and 1-(4-methoxyphenyl)-3-oxocyclobutanecarboxylic acid (31.5 mg, 0.143 mmol) were dissolved in N,N-dimethylformamide (3 mL), and 1-[bis(dimethylamino)methylene]-1H-1,2,3-triazolo[4,5-b]pyridinium 3-oxid hexafluorophosphate (54.4 mg, 0.143 mmol, HATU) and diisopropylethylamine (0.050 mL 0.286 mmol) were added thereto. The resulting solution was stirred at room temperature for 1 hour. It was diluted with water and extracted with ethyl acetate twice. The combined organic layers were washed with brine 3 times, dried over anhydrous Na₂SO₄, filtered and concentrated to give the titled compound (50 mg, 0.111 mmol, 77% yield). LC-MS (ESI) m/z 452 (M+H)⁺, RT=2.036 minutes.

### Step 4: 5-{[1-(4-methoxyphenyl)-3-oxocyclobutane-1-carbonyl](3-phenylpropyl)amino}pentanoic Acid

A mixture of Example 152—Step 3 (30 mg, 0.066 mmol), 1 N lithium hydroxide (0.5 mL 0.500 mmol), and dioxane (0.5 mL) was stirred at 30° C. for 2 hours. The mixture was acidified to pH=6-7 with 1 N HCl, and then the product was purified by preparative HPLC (0.1% CF₃CO₂H in water/CH₃CN) to give the titled compound (2.5 mg, 5.71 μmol, 8.60% yield). ¹H NMR (400 MHz, methanol-d₄) δ ppm 7.34-7.03 (m, 6H), 7.03-6.83 (m, 3H), 3.93-3.69 (m, 4H), 3.43-3.24 (m, 4H), 3.17-3.07 (m, 1H), 3.04-2.88 (m, 2H), 2.59 (q, J=9.8, 8.8 Hz, 1H), 2.29 (dq, J=13.4, 6.9 Hz, 2H), 2.01 (t, J=7.4 Hz, 1H), 1.86 (dp, J=14.2, 7.8 Hz, 1H), 1.57 (dt, J=12.1, 5.8 Hz, 2H), 1.37 (p, J=7.8 Hz, 1H), 1.19 (p, J=7.4 Hz, 1H), 0.99 (tt, J=11.6, 6.2 Hz, 1H); LC-MS (ESI) m/z 438 (M+H)⁺, RT=1.876 minutes.

### Example 153

### 5-{(3,5-dimethoxy-4-methylbenzoyl)[(2R)-4-phenylbutan-2-yl]amino}pentanoic Acid

### Step 1: methyl 5-{[(2R)-4-phenylbutan-2-yl]amino}pentanoate

(R)-4-Phenylbutan-2-amine (200 mg, 1.340 mmol) and potassium carbonate (222 mg, 1.608 mmol) were dissolved in dry CH₃CN (6 mL) at room temperature. Methyl 5-bromopentanoate (288 mg, 1.474 mmol) dissolved in dry CH₃CN was added slowly to the mixture, and then the mixture was stirred at reflux for 3 hours. The mixture was cooled down and filtered. The filtrate was concentrated to give the titled compound (230 mg, 0.873 mmol, 65.2% yield).

### Step 2: methyl 5-{(3,5-dimethoxy-4-methylbenzoyl)[(2R)-4-phenylbutan-2-yl]amino}pentanoate

To a solution of 3,5-dimethoxy-4-methylbenzoic acid (235 mg, 1.196 mmol) and 1-[bis(dimethylamino)methylene]-1H-1,2,3-triazolo[4,5-b]pyridinium 3-oxid hexafluorophosphate (500 mg, 1.316 mmol, HATU) in N,N-dimethylformamide (6 mL) was added triethylamine (0.183 mL 1.316 mmol). The resultant mixture was stirred at room temperature for 30 minutes. A solution of Example 153—Step 1 (314 mg, 1.196 mmol) and triethylamine (1 mL) in CH₃CN (3 mL) was added to the reaction system. The mixture was stirred at room temperature for 1 hour. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (88 mg, 0.201 mmol, 16.8% yield).

### Step 3: 5-{(3,5-dimethoxy-4-methylbenzoyl) [(2R)-4-phenylbutan-2-yl]amino}pentanoic Acid

Example 153—Step 2 (260 mg, 0.589 mmol) was dissolved in tetrahydrofuran (2 mL) and lithium hydroxide (85 mg, 3.53 mmol) solution (2 mL) was added. The mixture was stirred for 12 hours at room temperature. The mixture was poured into water; the pH was adjusted to 4 with 1 N HCl and extracted with ethyl acetate 3 times. The combined organic fractions were washed with brine, dried over Na₂SO₄, and concentrated. The residue was purified by preparative HPLC (0.1% CF₃CO₂H in water/CH₃CN) to give the titled compound (200 mg, 0.468 mmol, 79% yield). ¹H NMR (400 MHz, DMSO-d₆) δ ppm 7.36-6.96 (m, 5H), 6.48 (s, 2H), 3.75 (s, 6H), 3.14 (s, 1H), 2.38 (s, 2H), 2.26 (s, 2H), 1.98 (s, 3H), 1.85-1.46 (m, 5H), 1.15 (d, J=6.6 Hz, 4H); LC-MS (ESI) m/z 428.2 (M+H)⁺.

### Example 154

### 5-{(3,5-dimethoxy-4-methylbenzoyl) [(2S)-4-phenylbutan-2-yl]amino}pentanoic Acid

### Step 1: methyl 5-{[(2S)-4-phenylbutan-2-yl]amino}pentanoate

(S)-4-Phenylbutan-2-amine (200 mg, 1.340 mmol) and potassium carbonate (222 mg, 1.608 mmol) were dissolved in dry CH₃CN (6 mL) at room temperature. Methyl 5-bromopentanoate (288 mg, 1.474 mmol) dissolved in dry CH₃CN was added slowly to the mixture, and then the mixture was stirred at reflux for 3 hours. The mixture was cooled down and filtered. The filtrate was concentrated to give the titled compound. LC-MS (ESI) m/z 264.2 (M+H)⁺.

### Step 2: methyl 5-{(3,5-dimethoxy-4-methylbenzoyl)[(2S)-4-phenylbutan-2-yl]amino}pentanoate

3,5-Dimethoxy-4-methylbenzoic acid (263 mg, 1.340 mmol) and 1-[bis(dimethylamino)methylene]-1H-1,2,3-triazolo[4,5-b]pyridinium 3-oxid hexafluorophosphate (611 mg, 1.608 mmol, HATU) were dissolved in a mixture of dry N,N-dimethylformamide (1 mL), tetrahydrofuran (1.000 mL) and N,N-diisopropylethylamine (520 mg, 4.02 mmol). The mixture was stirred for 20 minutes at room temperature. Then Example 154—Step 1 (353 mg, 1.34 mmol) was added, and the mixture was stirred for another 3 hours at room temperature. The mixture was poured into water and extracted with ethyl acetate 3 times. The combined organic fractions were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (271 mg, 0.614 mmol, 45.8% yield).

### Step 3: 5-{(3,5-dimethoxy-4-methylbenzoyl) [(2S)-4-phenylbutan-2-yl]amino}pentanoic Acid

Example 154—Step 2 (265 mg, 0.600 mmol) was dissolved in tetrahydrofuran (2 mL) and lithium hydroxide (86 mg, 3.60 mmol) solution (2 mL) was added. The mixture was stirred for 12 hours at room temperature. The mixture was poured into water, the pH was adjusted to 5, and the mixture was extracted with ethyl acetate 3 times. The combined organic fractions were washed with brine, dried over Na₂SO₄, and concentrated. The residue was purified by preparative HPLC (0.1% CF₃CO₂H in water/CH₃CN) to give the titled compound (186 mg, 0.435 mmol, 72.5% yield). ¹H NMR (400 MHz, DMSO-d₆) δ ppm 7.36-6.96 (m, 5H), 6.48 (s, 2H), 3.75 (s, 6H), 3.14 (s, 1H), 2.38 (s, 2H), 2.26 (s, 2H), 1.98 (s, 3H), 1.85-1.46 (m, 5H), 1.15 (d, J=6.6 Hz, 4H); LC-MS (ESI) m/z 428.2 (M+H)⁺.

### Example 155

### 5-[(3,5-dimethoxy-4-methylbenzoyl)(3-phenylpropyl)amino]-2-hydroxy-2-methylpentanoic Acid

### Step 1: methyl 2-(acetyloxy)-5-iodo-2-methylpentanoate

To a mixture of methyl 2-methyltetrahydrofuran-2-carboxylate (300 mg, 2.081 mmol) and sodium iodide (624 mg, 4.16 mmol) in dry CH₃CN (3 mL) cooled in an ice-water bath was added, dropwise, acetyl chloride (0.296 mL 4.16 mmol). Then the mixture was stirred for 24 hours at room temperature. The mixture was quenched with saturated NaHCO₃ (4 mL) and much bubbling was noted as the suspension became clear. The aqueous fraction was separated and extracted with t-butyl methyl ether 3 times. The combined organic fractions were washed with brine and saturated NaHSO₃ once, dried, and concentrated to give the titled compound (376 mg, 1.197 mmol, 57.5% yield). ¹H NMR (400 MHz, CDCl₃) δ ppm 3.74 (s, 3H), 3.18 (dt, J=6.5, 3.0 Hz, 2H), 2.07 (s, 3H), 1.95-1.88 (m, 4H), 1.58 (s, 3H).

### Step 2: methyl 2-(acetyloxy)-2-methyl-5-[(3-phenylpropyl)amino]pentanoate

A mixture of 3-phenylpropan-1-amine (232 mg, 1.714 mmol) and Example 155—Step 1 (359 mg, 1.143 mmol) in CH₃CN (3 mL) was stirred at reflux for 30 minutes. The solution was cooled down and concentrated to give the titled compound. LC-MS (ESI) m/z 322.2 (M+H)⁺, RT=1.550 minutes.

### Step 3: methyl 2-(acetyloxy)-5-[(3,5-dimethoxy-4-methylbenzoyl)(3-phenylpropyl)amino]-2-methylpentanoate

To a solution of 3,5-dimethoxy-4-methylbenzoic acid (336 mg, 1.714 mmol) and 1-[bis(dimethylamino)methylene]-1H-1,2,3-triazolo[4,5-b]pyridinium 3-oxid hexafluorophosphate (717 mg, 1.885 mmol, HATU) in N,N-dimethylformamide (8 mL) was added triethylamine (0.263 mL 1.885 mmol). The resultant mixture was stirred at room temperature for 30 minutes. A solution of Example 155—Step 2 (551 mg, 1.714 mmol) and triethylamine (1 mL) in CH₃CN (3.0 mL) was added to the reaction system. The mixture was stirred at room temperature for 1 hour. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-50%) to give the titled compound (224 mg, 0.448 mmol, 26.2% yield). LC-MS (ESI) m/z 500.2 (M+H)⁺, RT=2.124 minutes.

### Step 4: 5-[(3,5-dimethoxy-4-methylbenzoyl)(3-phenylpropyl)amino]-2-hydroxy-2-methylpentanoic Acid

To a solution of Example 155—Step 3 (224 mg, 0.448 mmol) in tetrahydrofuran (4 mL) was added 1 N lithium hydroxide (4.48 mL 4.48 mmol). The mixture was stirred at room temperature overnight. The mixture was concentrated under vacuum and washed with t-butyl methyl ether 3 times. Then the aqueous fraction was acidified with 1 N HCl to pH=2-3 and extracted with ethyl acetate once. The organic layer was concentrated. The residue was purified by preparative HPLC (0.1% NH₃ in H₂O/CH₃CN) and lyophilized to give the titled compound (110 mg, 0.248 mmol, 55.3% yield). ¹H NMR (400 MHz, DMSO-d₆) δ ppm 7.30-7.11 (m, 4H), 7.02 (s, 1H), 6.91 (s, 1H), 6.47 (s, 2H), 3.75 (d, J=4.1 Hz, 6H), 3.32 (d, J=29.5 Hz, 2H), 3.10 (d, J=19.5 Hz, 2H), 2.61 (s, 1H), 2.39 (s, 1H), 1.98 (s, 3H), 1.86 (s, 1H), 1.78 (s, 1H), 1.60 (s, 1H), 1.37 (s, 2H), 1.14 (s, 2H), 1.06 (s, 2H); LC-MS (ESI) m/z 444.2 (M+H)⁺, RT=1.891 minutes.

### Example 156

### N-[2-({1-[(methanesulfonyl)amino]-2-methyl-1-oxopropan-2-yl}oxy)ethyl]-3,5-dimethoxy-4-methyl-N-(3-phenylpropyl)benzamide

A mixture of 2-{2-[(3,5-dimethoxy-4-methylbenzoyl)(3-phenylpropyl)amino]ethoxy}-2-methylpropanoic acid (20 mg, 0.045 mmol, Example 55) and 1,1′-carbonyldiimidazole (11.70 mg, 0.072 mmol) in isopropyl acetate (2 mL) was stirred at 50° C. for 25 minutes. Methanesulfonamide (6.86 mg, 0.072 mmol) and 1,8-diazabicyclo[5.4.0]undec-7-ene (10.87 μL, 0.072 mmol) were added, and the reaction mixture was stirred at 50° C. overnight. The reaction mixture was diluted with 10 mL of ethyl acetate, washed with 10 mL of 1 N HCl and saturated NaCl, dried with MgSO₄ and concentrated. The residue was re-dissolved in isopropyl acetate (2 mL) and treated with 1,1′-carbonyldiimidazole (11.70 mg, 0.072 mmol) and stirred at 40° C. for 30 minutes. Then more methanesulfonamide (6.86 mg, 0.072 mmol) and 1,8-diazabicyclo[5.4.0]undec-7-ene (10.87 μL, 0.072 mmol) were added, and the mixture was stirred at 50° C. overnight. The reaction mixture was diluted with 10 mL of ethyl acetate, washed with 10 mL of 1 N HCl and saturated NaCl, dried with MgSO₄ and concentrated. Flash chromatography on silica gel (100% ethyl acetate) gave the titled compound (4 mg, 18%). ¹H NMR (501 MHz, CDCl₃) δ ppm 0.81-1.02 (m, 6H), 1.37 (d, J=84.7 Hz, 2H), 1.94 (s, 2H), 2.13 (s, 3H), 2.53 (s, 2H), 3.34 (s, 2H), 3.71 (d, J=29.8 Hz, 4H), 3.83 (s, 7H), 6.54 (s, 2H), 7.07 (d, J=9.2 Hz, 2H), 7.16-7.29 (m, 3H), 10.02 (s, 1H); MS (DCI) m/z 521 (M+H)⁺

### Example 157

### N-(2-{2-[(methanesulfonyl)amino]-2-oxoethoxy}ethyl)-3,5-dimethoxy-4-methyl-N-(3-phenylpropyl)benzamide

A 4 mL vial was charged with {2-[(3,5-dimethoxy-4-methylbenzoyl)(3-phenylpropyl)amino]ethoxy}acetic acid (0.1 g, 0.241 mmol, Example 6), tetrahydrofuran (2 mL) and 1,1′-carbonyldiimidazole (0.047 g, 0.289 mmol). The vial was capped and heated at 50° C. for 1 hour. Methanesulfonamide (0.025 g, 0.265 mmol) and 1,8-diazabicyclo[5.4.0]undec-7-ene (0.073 mL, 0.481 mmol) were added and heating was continued for 2 hours. The mixture was diluted with CHC1₃ (10 mL) and shaken with 1 M HCl (10 mL). The lower organic layer was isolated using a Biotage® phase separator column (#120-1905C) and concentrated. The product was isolated using preparative HPLC followed by flash chromatography on silica gel (30-100% ethyl acetate/heptane) to give the titled compound (0.048 g, 0.097 mmol, 40.5% yield). ¹H NMR (400 MHz, DMSO-d₆) δ ppm 7.16-7.24 (3H, m), 7.01 (2H, s, br), 6.51 (2H, s), 4.09 (2H, s, br), 3.81 (6H, s), 3.70 (2H, s, br), 3.34 (2H, s, br), 3.28 (3H, s), 2.51 (2H, s, br), 2.10 (3H, s), 1.91 (2H, s, br); MS (APCI⁺) m/z 493.3 (M+H)⁺.

### Example 158

### 5-{[cis-3-methoxy-1-(4-methoxyphenyl)cyclobutane-1-carbonyl](3-phenylpropyl)amino}pentanoic Acid

### Step 1: methyl 5-{[1-(4-methoxyphenyl)-3-oxocyclobutane-1-carbonyl](3-phenylpropyl)amino}pentanoate

A mixture of 1-(4-methoxyphenyl)-3-oxocyclobutanecarboxylic acid (100 mg, 0.485 mmol), 2-(3H-[1,2,3]triazolo[4,5-b]pyridin-3-yl)-1,1,3,3-tetramethylisouronium hexafluorophosphate(V) (184 mg, 0.485 mmol), Example 70—Step 1 (139 mg, 0.485 mmol), N,N-dimethylformamide (2 mL) and N-ethyl-N-isopropylpropan-2-amine (188 mg, 1.455 mmol) was stirred at 25° C. for 2 hours. The reaction was quenched with water, and the mixture was extracted with ethyl acetate (2×20 mL). The combined organic fractions were washed with brine (2×30 mL), dried over Na₂SO₄, and concentrated to give a residue which was purified by flash column chromatography on silica gel (eluent, ethyl acetate/hexane from 0/10 to 1/2) to afford the titled compound. ¹H NMR (400 MHz, CDCl₃) δ ppm 7.31-7.27 (m, 2H), 7.25-7.17 (m, 3H), 7.07-7.03 (m, 1H), 6.97-6.94 (m, 1H), 6.90-6.84 (m, 2H), 3.81 (d, J=3.4 Hz, 3H), 3.65 (d, J=11.5 Hz, 3H), 3.35 (ddd, J=14.2, 7.0, 4.2 Hz, 2H), 3.15-2.99 (m, 2H), 2.93-2.85 (m, 2H), 2.63 (t, J=7.8 Hz, 1H), 2.32 (dt, J=24.7, 7.0 Hz, 3H), 2.05 (t, J=7.4 Hz, 1H), 1.94-1.80 (m, 1H), 1.44-1.35 (m, 2H), 1.27-1.15 (m, 2H), 1.02 (p, J=8.8, 8.0 Hz, 2H); LC-MS (ESI) m/z 452.2 (M+H)⁺, RT=2.042 minutes.

### Step 2: methyl 5-{[3-hydroxy-1-(4-methoxyphenyl)cyclobutane-1-carbonyl](3-phenylpropyl)amino}pentanoate

To a mixture of Example 158—Step 1 (0.05 g, 0.100 mmol) in CH₃OH (5 mL) was added portionwise sodium tetrahydroborate (3.77 mg, 0.100 mmol) at 0° C. The mixture was stirred at 0° C. for 1.5 hours. The mixture was quenched with water and extracted with ethyl acetate (3×20 mL). The combined organic phases were washed with brine (2×30 mL), dried over Na₂SO₄, and concentrated to give the titled compound. LC-MS (ESI) m/z 454.2 (M+H)⁺, RT=1.928 minutes.

### Step 3: 5-{[cis-3-methoxy-1-(4-methoxyphenyl)cyclobutane-1-carbonyl](3-phenylpropyl)amino}pentanoic Acid

To a mixture of Example 158—Step 2 (55 mg, 0.121 mmol) in N,N-dimethylformamide (2 mL) was added portionwise sodium hydride (9.70 mg, 0.243 mmol) at 0° C. The mixture was stirred at 0° C. for 10 minutes. Iodomethane (0.023 mL 0.364 mmol) was added to the mixture at 0° C. The mixture was stirred at room temperature for 3 hours. The mixture was quenched with water (2 mL). To the mixture was added lithium hydroxide (11.62 mg, 0.485 mmol) portion wise. The mixture was stirred for additional 2 hours. The mixture was neutralized with 1 N HCl solution, and the result mixture was concentrated to ˜2 mL. The mixture was purified by preparative HPLC (0.1% CF₃CO₂H in water/CH₃CN) to afford the titled compound and Example 159. ¹H NMR (400 MHz, CDCl₃) δ ppm 8.28 (s, 2H), 7.33-7.20 (m, 4H), 7.17 (dd, J=9.2, 7.3 Hz, 1H), 7.09 (d, J=8.3 Hz, 1H), 7.03-6.96 (m, 1H), 6.87 (dd, J=11.9, 8.2 Hz, 2H), 3.83 (d, J=6.8 Hz, 1H), 3.80 (d, J=4.1 Hz, 3H), 3.33-3.24 (m, 2H), 3.22 (d, J=11.4 Hz, 3H), 2.87 (t, J=7.8 Hz, 2H), 2.69 (t, J=9.0 Hz, 1H), 2.54 (dt, J=27.4, 9.4 Hz, 3H), 2.35 (dt, J=22.8, 6.9 Hz, 2H), 2.12 (t, J=7.3 Hz, 1H), 1.81 (dd, J=10.0, 5.3 Hz, 1H), 1.61-1.34 (m, 4H), 1.25 (p, J=7.3 Hz, 1H), 1.04 (td, J=9.6, 8.9, 5.2 Hz, 1H); LC-MS (ESI) m/z 454.2 (M+H)⁺, RT=1.929 minutes

### Example 159

### 5-{[trans-3-methoxy-1-(4-methoxyphenyl)cyclobutane-1-carbonyl](3-phenylpropyl)amino}pentanoic Acid

See Example 158 for details. ¹H NMR (400 MHz, CDCl₃) δ ppm 8.35 (s, 2H), 7.31-7.12 (m, 5H), 7.06 (d, J=8.2 Hz, 1H), 6.95-6.90 (m, 1H), 6.85 (d, J=8.1 Hz, 2H), 3.86 (dt, J=13.0, 6.7 Hz, 1H), 3.78 (d, J=3.9 Hz, 3H), 3.31 (q, J=6.6 Hz, 2H), 3.24 (d, J=6.2 Hz, 3H), 3.08-2.93 (m, 2H), 2.87 (q, J=9.0 Hz, 2H), 2.61 (t, J=7.8 Hz, 1H), 2.43-2.28 (m, 2H), 2.21 (dt, J=18.9, 7.4 Hz, 2H), 2.03 (t, J=7.4 Hz, 1H), 1.85 (q, J=7.8, 7.4 Hz, 1H), 1.60 (d, J=5.8 Hz, 2H), 1.28-1.09 (m, 2H), 0.85 (ddt, J=15.9, 11.6, 6.2 Hz, 1H); LC-MS (ESI) m/z 454.2 (M+H)⁺, RT=1.913 minutes.

### Example 160

### 5-[(2-methyl-4-oxo-3,4-dihydroquinazoline-8-carbonyl)(3-phenylpropyl)amino]pentanoic Acid

A 4 mL scintillation vial was charged with 210 μL of a 0.6 mM solution of 2-methyl-4-oxo-3,4-dihydroquinazoline-8-carboxylic acid in dimethylacetamide (25.7 mg, 1.2 equivalents, 0.13 mmol), 500 μL of a solution of 1-[bis(dimethylamino)methylene]-1H-1,2,3-triazolo[4,5-b]pyridinium 3-oxid hexafluorophosphate (HATU) in dimethylacetamide (43.9 mg, 1.1 equivalents, 0.12 mmol), 500 μL of a solution of methyl 5-[(3-phenylpropyl)amino]pentanoate in dimethylacetamide (30.0 mg, 0.10 mmol, Example 70—Step 1), neat triethylamine (44.2 μL, 3 equivalents, 0.31 mmol), and a stir bar. This was capped with a white seal and microwave cap for an Anton Paar microwave reactor. The vial was placed in an Anton Paar Synthos 3000 parallel microwave optimizer and heated for 15 minutes at 120° C. Upon completion, the mixture was filtered, and the filtrate was concentrated to dryness under reduced pressure. The residue was then dissolved in 1000 μL of dioxane. 1000 μL of a 1 M LiOH aqueous solution in 75% methanol was added. The mixture was then heated at 60° C. for 1 hour. The reacting mixture was then filtered once more and concentrated to dryness under reduced pressure. The residue was re-dissolved in dimethyl sulfoxide/methanol and purified using preparative-HPLC to give the titled compound (22.5 mg, 53.7%). ¹H NMR (400 MHz, DMSO-d₆) δ ppm 8.03 (dd, J=7.9, 1.5 Hz, 1H), 7.54 (dd, J=7.3, 1.6 Hz, 1H), 7.42 (t, J=7.6 Hz, 1H), 7.32-7.24 (m, 2H), 7.07-6.99 (m, 2H), 6.80 (dd, J=7.7, 1.8 Hz, 1H), 3.54-3.47 (m, 1H), 2.97 (t, J=7.0 Hz, 1H), 2.89 (t, J=7.9 Hz, 1H), 2.74 (t, J=7.9 Hz, 1H), 2.31-2.24 (m, 5H), 1.93 (q, J=8.2, 7.8 Hz, 2H), 1.71 (q, J=7.5, 6.8 Hz, 1H), 1.63 (dd, J=6.6, 3.3 Hz, 2H), 1.39 (q, J=8.6, 7.3 Hz, 1H), 1.19 (p, J=7.4 Hz, 1H); MS (APCI+) m/z 422.0 (M+H)⁺.

### Example 161

### N-{5-[(methanesulfonyl)amino]-4-methyl-5-oxopentyl}-3,5-dimethoxy-4-methyl-N-(3-phenylpropyl)benzamide

A mixture of 5-[(3,5-dimethoxy-4-methylbenzoyl)(3-phenylpropyl)amino]-2-methylpentanoic acid (100 mg, 0.234 mmol, Example 54) and 1,1′-carbonyldiimidazole (45.5 mg, 0.281 mmol) in tetrahydrofuran (2 mL) was stirred at 50° C. for 60 minutes. Methanesulfonamide (26.7 mg, 0.281 mmol) and 1,8-diazabicyclo[5.4.0]undec-7-ene (0.056 mL, 0.374 mmol) were added, and the reaction mixture was stirred at 50° C. The reaction mixture was diluted with 20 mL of ethyl acetate, washed with 20 mL each of 1 N HCl and saturated NaCl, dried with MgSO₄ and concentrated. Flash chromatography on silica gel (5-100% ethyl acetate/heptanes) gave the titled compound (15 mg, 13%). ¹H NMR (400 MHz, DMSO-d₆) δ ppm 1.13 (m, 3H), 1.53 (m, 3H) (m, 2H), 2.01 (s, 3H), 2.42 (m, 2H), 2.53 (t, J=7.6 Hz, 2H), 3.15 (s, 3H), 3.29 (q, J=7.0, 6.3 Hz, 4H), 3.76 (s, 6H), 6.49 (s, 2H), 7.16 (m, 5H), 11.07 (s, 1H); MS (DCI) m/z 504 (M+H)⁺.

### Example 162

### N-{5-[(methanesulfonyl)amino]-4,4-dimethyl-5-oxopentyl}-3,5-dimethoxy-4-methyl-N-(3-phenylpropyl)benzamide

A mixture of 5-[(3,5-dimethoxy-4-methylbenzoyl)(3-phenylpropyl)amino]-2,2-dimethylpentanoic acid (90 mg, 0.204 mmol, Example 57) and 1,1′-carbonyldiimidazole (39.7 mg, 0.245 mmol), in tetrahydrofuran (2 mL) was stirred at 50° C. for 60 minutes. Methanesulfonamide (29.1 mg, 0.306 mmol) and 1,8-diazabicyclo[5.4.0]undec-7-ene (0.049 mL, 0.326 mmol) were added, and the reaction mixture was stirred at 50° C. overnight. The reaction mixture was diluted with 20 mL of ethyl acetate, washed with 20 mL each of 1 N HCl and saturated NaCl, dried with MgSO₄ and concentrated. Flash chromatography on silica gel (100% ethyl acetate) gave the titled compound (30 mg, 90%). ¹H NMR (400 MHz, DMSO-d₆) δ ppm 1.09 (d, J=1.4 Hz, 6H), 1.45 (d, J=3.2 Hz, 4H), 1.86 (p, J=7.6 Hz, 2H), 2.01 (s, 3H), 2.54 (t, J=7.7 Hz, 2H), 3.09 (d, J=1.4 Hz, 3H), 3.26 (t, J=7 Hz, 2H), 3.32 (t, J=7 Hz, 2H), 3.76 (d, J=1.4 Hz, 6H), 6.50 (s, 2H), 7.04-7.32 (m, 5H), 10.61 (s, 1H); MS (DCI) m/z 519 (M+H)⁺.

### Example 163

### 5-[(2,6-dimethoxypyrimidine-4-carbonyl)(3-phenylpropyl)amino]pentanoic Acid

### Step 1: 2,6-dimethoxypyrimidine-4-carbonyl chloride

Oxalyl chloride (0.092 mL, 1.051 mmol) was added dropwise to a stirred mixture of 2,6-dimethoxypyrimidine-4-carboxylic acid (168 mg, 0.910 mmol) and catalytic N,N-dimethylformamide (5.42 μL, 0.070 mmol) in dichloromethane (14 mL). After stirring for 2.3 hours, volatiles were removed under reduced pressure to give a white solid. The solid was treated with t-butyl methyl ether. Any insolubles were removed by filtration and the filtrate was concentrated under reduced pressure (bath temperature: 25° C.) to give the titled compound that was dissolved in CH₂Cl₂ (10 mL).

### Step 2: methyl 5-[(2,6-dimethoxypyrimidine-4-carbonyl)(3-phenylpropyl)amino]pentanoate

The stirred solution above was treated with a suspension of methyl 5-[(3-phenylpropyl)amino]pentanoate-hydrogen chloride (200 mg, 0.7 mmol, Example 149—Step 2) in CH₂Cl₂ (20 mL). Next, triethylamine (0.293 mL, 2.100 mmol) was added. Finally a solution of 4-(dimethylamino)pyridine (42.8 mg, 0.350 mmol) in CH₂Cl₂ (3 mL) was added. The reaction mixture was then stirred at ambient temperature for 16.5 hours. Volatiles were removed under reduced pressure to give a residue that was purified by flash chromatography (100% CH₂Cl₂ to 60:40 CH₂Cl₂/ethyl acetate) to give the titled compound (226 mg 78% yield). ¹H NMR (CDCl₃) δ ppm 7.32-7.14 (m, 4H), 7.06-7.01 (m, 1H), 6.50 (d, J=22.6 Hz, 1H), 4.01-3.93 (m, 6H), 3.66 (d, J=10.7 Hz, 3H), 3.48 (dt, J=13.6, 6.9 Hz, 2H), 3.32-3.24 (m, 2H), 2.70 (t, J=7.9 Hz, 1H), 2.50 (t, J=7.5 Hz, 1H), 2.40-2.33 (m, 1H), 2.23 (t, J=7.2 Hz, 1H), 2.05-1.88 (m, 2H), 1.70-1.65 (m, 2H), 1.54 (ddt, J=49.6, 15.1, 7.2 Hz, 2H); MS (ESI+) m/z 416 (M+H)⁺.

### Step 3: 5-[(2,6-dimethoxypyrimidine-4-carbonyl)(3-phenylpropyl)amino]pentanoic Acid

A solution of methyl 5-[(2,6-dimethoxypyrimidine-4-carbonyl)(3-phenylpropyl)amino]pentanoate (210 mg, 0.505 mmol, Step 2) in tetrahydrofuran (4.2 mL) and methanol (0.85 mL) was stirred at ambient temperature. Lithium hydroxide, 0.5 M aqueous (3.033 mL, 1.517 mmol) was added, and the reaction mixture was stirred at ambient temperature for 27 hours. The reaction mixture was brought to pH=4 with aqueous citric acid, diluted with brine and extracted with ethyl acetate. The organic layer was dried (MgSO₄) and filtered. The filtrate was concentrated under reduced pressure to give a residue. Flash chromatography on silica gel (100% CH₂Cl₂ to 97:3 CH₂Cl₂/CH₃OH) gave the titled compound (151 mg, 74.4% yield). ¹H NMR (CDCl₃) δ ppm 7.31-7.26 (m, 1H), 7.25-7.14 (m, 3H), 7.04 (dd, J=6.9, 1.7 Hz, 1H), 6.50 (d, J=29.6 Hz, 1H), 3.97 (d, J=21.5 Hz, 6H), 3.52-3.46 (m, 2H), 3.28 (ddd, J=9.4, 6.6, 1.9 Hz, 2H), 2.72-2.67 (m, 1H), 2.50 (t, J=7.5 Hz, 1H), 2.44-2.39 (m, 1H), 2.27 (t, J=7.3 Hz, 1H), 2.03-1.89 (m, 2H), 1.69 (p, J=3.7 Hz, 2H), 1.66-1.59 (m, 1H), 1.49 (p, J=7.4 Hz, 1H); MS (ESI+) m/z 402 (M+H)⁺, MS (ESI−) m/z 400 (M−H)⁻.

### Example 164

### 5-[(3,5-dimethoxy-4-methylbenzoyl)(3-phenylpropyl)amino]hexanoic Acid

### Step 1: methyl 5-[(3-phenylpropyl)amino]hexanoate

3-Phenylpropan-1-amine (200 mg, 1.479 mmol) and potassium carbonate (245 mg, 1.775 mmol) were dissolved in dry CH₃CN (6 mL) at room temperature. Methyl 5-bromohexanoate (340 mg, 1.627 mmol) dissolved in dry CH₃CN was added slowly to the mixture, and then the reaction was stirred at reflux for 3 hours. Then the mixture was cooled down and filtered. The filtrate was concentrated to give the titled compound.

### Step 2: methyl 5-[(3,5-dimethoxy-4-methylbenzoyl)(3-phenylpropyl)amino]hexanoate

3,5-Dimethoxy-4-methylbenzoic acid (281 mg, 1.430 mmol) and 1-[bis(dimethylamino)methylene]-1H-1,2,3-triazolo[4,5-b]pyridinium 3-oxid hexafluorophosphate (652 mg, 1.716 mmol, HATU) were dissolved in a mixture of dry N,N-dimethylformamide (1 mL), tetrahydrofuran (1.000 mL) and N,N-diisopropylethylamine (554 mg, 4.29 mmol). The mixture was stirred for 20 minutes at room temperature. Then Example 164—Step 1 (377 mg, 1.43 mmol) was added, and the mixture was stirred for another 3 hours at room temperature. The mixture was poured into water and extracted with ethyl acetate 3 times. The combined organic layers were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (233 mg, 0.528 mmol, 36.9% yield). LC-MS (ESI) m/z 442.2 (M+H)⁺.

### Step 3: 5-[(3,5-dimethoxy-4-methylbenzoyl)(3-phenylpropyl)amino]hexanoic Acid

Example 164—Step 2 (45 mg, 0.102 mmol) was dissolved in tetrahydrofuran (2 mL) and lithium hydroxide (14.64 mg, 0.611 mmol) in water (2 mL) was added. The mixture was stirred for 12 hours at room temperature. The mixture was poured into water, the pH was adjusted to 5, and the acidic mixture was extracted with ethyl acetate 3 times. The combined organic layers were washed with brine, dried over Na₂SO₄, and concentrated. The residue was purified by preparative HPLC (0.1% CF₃CO₂H in water/CH₃CN) to give the titled compound (20 mg, 0.047 mmol, 45.9% yield). ¹H NMR (400 MHz, DMSO-d₆) δ ppm 12.02 (s, 2H), 7.44-7.02 (m, 5H), 6.46 (s, 2H), 3.60 (s, 1H), 3.08 (s, 1H), 2.62 (t, J=8.2 Hz, 2H), 2.06 (q, J=6.0, 5.2 Hz, 2H), 1.98 (s, 5H), 1.59-0.88 (m, 8H); LC-MS (ESI) m/z 428.2 (M+H)⁺.

### Example 165

### 5-[(4-cyclopropyl-3,5-dimethoxybenzoyl)(3-phenylpropyl)amino]-2-hydroxypentanoic Acid

### Step 1: methyl 2-(acetyloxy)-5-[(4-cyclopropyl-3,5-dimethoxybenzoyl)(3-phenylpropyl)amino]pentanoate

To a solution of 4-cyclopropyl-3,5-dimethoxybenzoic acid (164 mg, 0.740 mmol) and 1-[bis(dimethylamino)methylene]-1H-1,2,3-triazolo[4,5-b]pyridinium 3-oxid hexafluorophosphate (310 mg, 0.814 mmol, HATU) in N,N-dimethylformamide (3 mL) was added triethylamine (0.113 mL 0.814 mmol). The resultant mixture was stirred at room temperature for 10 minutes. Then a solution of Example 151—Step 2 (227 mg, 0.740 mmol) in N,N-dimethylformamide (1.0 mL) was added to the reaction system. The mixture was stirred at room temperature for 40 minutes. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-30%) to give the titled compound (128 mg, 0.228 mmol, 30.8% yield). LC-MS (ESI) m/z 512.2 (M+H)⁺, RT=2.149 minutes.

### Step 2: 5-[(4-cyclopropyl-3,5-dimethoxybenzoyl)(3-phenylpropyl)amino]-2-hydroxypentanoic Acid

To a solution of Example 165—Step 1 (128 mg, 0.250 mmol) in tetrahydrofuran (2 mL) was added 1 N lithium hydroxide (2.502 mL, 2.502 mmol). The mixture was stirred at room temperature for 2 hours. The mixture was concentrated, and the aqueous residue was washed with ethyl ether twice and then acidified with 1 N HCl to pH=2-3. The acidic aqueous mixture was extracted with ethyl acetate 3 times. The combined organic layers were dried and concentrated. The residue was purified by preparative HPLC (0.1% NH₃ in H₂O/CH₃CN) and lyophilized to give the titled compound (65 mg, 0.143 mmol, 57.0% yield). ¹H NMR (400 MHz, DMSO-d₆) δ ppm 7.32-7.01 (m, 5H), 6.73 (s, 1H), 6.44 (s, 2H), 3.71 (s, 6H), 3.46 (s, 1H), 3.36 (s, 2H), 3.13 (s, 2H), 2.60 (s, 1H), 2.40 (s, 1H), 1.84 (tt, J=8.8, 5.7 Hz, 3H), 1.57 (s, 3H), 1.40 (s, 1H), 0.99-0.89 (m, 2H), 0.74 (dt, J=8.5, 3.0 Hz, 2H); LC-MS (ESI) m/z 456.2 (M+H)⁺, RT=1.912 minutes.

### Example 166

### 3,5-dimethoxy-4-methyl-N-(3-phenylpropyl)-N-[4-(1H-tetrazol-5-yl)butyl]benzamide

### Step 1: N-(5-amino-5-oxopentyl)-3,5-dimethoxy-4-methyl-N-(3-phenylpropyl)benzamide

A solution of 5-[(3,5-dimethoxy-4-methylbenzoyl)(3-phenylpropyl)amino]pentanoic acid (100 mg, 0.242 mmol, Example 4) in tetrahydrofuran (5 mL) was treated with ((3H-[1,2,3]triazolo[4,5-b]pyridin-3-yl)oxy)tri(pyrrolidin-1-yl)phosphonium hexafluorophosphate(V) (126 mg, 0.242 mmol) followed by N-ethyl-N-isopropylpropan-2-amine (31.3 mg, 0.242 mmol), and the reaction mixture was stirred for 1 hour. NH₄OH (0.49 mL, 3.76 mmol) was added, and the mixture was stirred for 18 hours. The mixture was concentrated, and the residue was partitioned between ethyl acetate and 1 N HCl. The organic fraction was washed with 1 N NaOH and brine, dried (MgSO₄) and concentrated to the titled compound that was used directly in the next step.

### Step 2: N-(4-cyanobutyl)-3,5-dimethoxy-4-methyl-N-(3-phenylpropyl)benzamide

N-(5-amino-5-oxopentyl)-3,5-dimethoxy-4-methyl-N-(3-phenylpropyl)benzamide (430 mg, 1.042 mmol, Step 1) in dioxane/pyridine (5 mL/0.5 mL) was cooled to 0° C. and treated with trifluoromethanesulfonic anhydride (0.15 mL, 1.04 mmol). The reaction mixture was warmed to room temperature and stirred for 4 hours. The mixture was partitioned between ethyl acetate and brine. The organic layer was washed with water (2×30 mL), dried (MgSO₄) and concentrated to give the titled compound (410 mg) that was used directly in the next step.

### Step 3: 3,5-dimethoxy-4-methyl-N-(3-phenylpropyl)-N-[4-(1H-tetrazol-5-yl)butyl]benzamide

A solution of N-(4-cyanobutyl)-3,5-dimethoxy-4-methyl-N-(3-phenylpropyl)benzamide (300 mg, 0.760 mmol) in N,N-dimethylformamide was treated with sodium azide (222 mg, 3.42 mmol) and ammonium chloride (61.0 mg, 1.141 mmol) and then heated to 105° C. for 72 hours. The reaction mixture was cooled to room temperature and poured into water. The aqueous mixture was acidified with HCl, and extracted with ethyl acetate. The organic fraction was washed with brine (3×25 mL), dried (MgSO₄) and concentrated. Flash chromatography on silica gel (100% ethyl acetate) gave the titled compound (74 mg, 22%). ¹H NMR (400 MHz, DMSO-d₆) δ ppm 1.49-1.77 (m, 4H), 1.85 (t, J=7.6 Hz, 2H), 2.01 (s, 3H), 2.52 (t, J=7.6 Hz, 3H), 2.83 (t, J=7.1 Hz, 2H), 3.26-3.38 (m, 4H), 3.74 (d, J=0.9 Hz, 6H), 6.49 (s, 2H), 7.11 (t, J=7.2 Hz, 3H), 7.20 (dd, J=8.3, 6.5 Hz, 2H); MS (DCI) m/z 438 (M+H)⁺.

### Example 167

### 5-[(5-chloro-2-methylpyrimidine-4-carbonyl)(3-phenylpropyl)amino]pentanoic Acid

A 4 mL Wheaton scintillation vial was charged with 280 μL of a 0.6 mM solution of 5-chloro-2-methylpyrimidine-4-carboxylic acid in N,N-dimethylacetamide (30.0 mg, 1.2 equivalents, 0.2 mmol), a 500 μL solution of 1-[bis(dimethylamino)methylene]-1H-1,2,3-triazolo[4,5-b]pyridinium 3-oxid hexafluorophosphate in N,N-dimethylacetamide (58.5 mg, 1.1 equivalents, 0.15 mmol, HATU), a 500 μL solution of methyl 5-[(3-phenylpropyl)amino]pentanoate hydrochloride in N,N-dimethylacetamide (40.0 mg, 0.14 mmol, Example 149—Step 2), neat triethylamine (58.9 μL, 3 equivalents, 0.42 mmol), and a stir bar. This was capped with a white seal and microwave cap for an Anton Paar microwave reactor. The vial was heated in an Anton Paar Synthos 3000 parallel microwave optimizer for 15 minutes at 120° C. Upon completion, the reaction mixture was filtered, and the filtrate was concentrated to dryness under reduced pressure. The residue was then dissolved in 1000 μL of dioxane. To this, 1000 μL of a 1 M LiOH aqueous solution in 75% CH₃OH was added. The mixture was then heated at 60° C. for 1 hour. The reaction mixture was then filtered once more and concentrated under reduced pressure. The residue was re-dissolved in dimethyl sulfoxide/methanol and purified using preparative-HPLC to give the titled compound (42.3 mg, 73.6%). ¹H NMR (400 MHz, DMSO-d₆) δ ppm 8.72 (d, J=35.5 Hz, 1H), 7.31-7.08 (m, 4H), 6.97 (d, J=7.4 Hz, 1H), 3.52-3.41 (m, 2H), 3.09-3.05 (m, 1H), 2.66 (t, J=7.6 Hz, 1H), 2.59 (d, J=23.0 Hz, 3H), 2.43 (t, J=7.3 Hz, 1H), 2.26 (t, J=6.9 Hz, 1H), 2.06 (t, J=7.2 Hz, 1H), 1.94 (p, J=7.5, 6.9 Hz, 1H), 1.80 (dt, J=13.0, 6.2 Hz, 2H), 1.70-1.56 (m, 3H), 1.50 (q, J=8.2, 7.8 Hz, 1H), 1.35 (p, J=6.8 Hz, 1H); MS (APCI⁺) m/z 390.0 (M+H)⁺.

### Example 168

### 5-[(6-fluoro-2-oxo-1,2,3,4-tetrahydroquinoline-4-carbonyl)(3-phenylpropyl)amino]pentanoic Acid

A 4 mL Wheaton scintillation vial was charged with 280 μL of a 0.6 mM solution of 6-fluoro-2-oxo-1,2,3,4-tetrahydroquinoline-4-carboxylic acid in N,N-dimethylacetamide (35.13 mg, 1.2 equivalents, 0.2 mmol), a 500 μL solution of 1-[bis(dimethylamino)methylene]-1H-1,2,3-triazolo[4,5-b]pyridinium 3-oxid hexafluorophosphate in N,N-dimethylacetamide (58.5 mg, 1.1 equivalents, 0.15 mmol, HATU), a 500 μL solution of methyl 5-[(3-phenylpropyl)amino]pentanoate hydrochloride in N,N-dimethylacetamide (40.0 mg, 0.14 mmol, Example 149—Step 2), neat triethylamine (58 μL, 3 equivalents, 0.42 mmol), and a stir bar. This was capped with a white seal and microwave cap for an Anton Paar microwave reactor. The vial was heated in an Anton Paar Synthos 3000 parallel microwave optimizer for 15 minutes at 120° C. Upon completion, the reaction mixture was filtered, and the filtrate was concentrated to dryness under reduced pressure. The residue was then dissolved in 1000 μL of dioxane. To this, 1000 μL of a 1 M LiOH aqueous solution in 75% CH₃OH was added. The mixture was then heated at 60° C. for 1 hour. The reaction mixture was then filtered once more and concentrated under reduced pressure. The residue was re-dissolved in dimethyl sulfoxide/methanol and purified using preparative-HPLC to give the titled compound (46.3 mg, 77.7%). ¹H NMR (400 MHz, DMSO-d₆) δ ppm 7.27-7.21 (m, 2H), 7.19-7.10 (m, 3H), 6.94 (td, J=8.6, 2.7 Hz, 1H), 6.89 (dd, J=8.7, 5.2 Hz, 1H), 6.71 (d, J=9.8 Hz, 1H), 4.25-4.17 (m, 1H), 3.43 (dt, J=14.9, 8.0 Hz, 2H), 3.31 (dt, J=14.5, 7.4 Hz, 2H), 2.62-2.51 (m, 4H), 2.21 (t, J=6.9 Hz, 2H), 1.91-1.76 (m, 2H), 1.61-1.48 (m, 4H); MS (APCI⁺) m/z 427.1 (M+H)⁺.

### Example 169

### 5-[(2-methyl-1-oxo-1,2-dihydroisoquinoline-4-carbonyl)(3-phenylpropyl)amino]pentanoic Acid

A 4 mL Wheaton scintillation vial was charged with 280 μL of a 0.6 mM solution of 2-methyl-1-oxo-1,2-dihydroisoquinoline-4-carboxylic acid in N,N-dimethylacetamide (34.1 mg, 1.2 equivalents, 0.2 mmol), a 500 μL solution of 1-[bis(dimethylamino)methylene]-1H-1,2,3-triazolo[4,5-b]pyridinium 3-oxid hexafluorophosphate in N,N-dimethylacetamide (58.5 mg, 1.1 equivalents, 0.15 mmol, HATU), a 500 μL solution of methyl 5-[(3-phenylpropyl)amino]pentanoate hydrochloride in N,N-dimethylacetamide (40.0 mg, 0.14 mmol, Example 149—Step 2), neat triethylamine (58.9 μL, 3 equivalents, 0.42 mmol), and a stir bar. This was capped with a white seal and microwave cap for an Anton Paar microwave reactor. The vial was heated in an Anton Paar Synthos 3000 parallel microwave optimizer for 15 minutes at 120° C. Upon completion, the reaction mixture was filtered, and the filtrate was concentrated to dryness under reduced pressure. The residue was then dissolved in 1000 μL of dioxane. To this, 1000 μL of a 1 M LiOH aqueous solution in 75% CH₃OH was added. The mixture was then heated at 60° C. for 1 hour. The reaction mixture was then filtered once more and concentrated under reduced pressure. The residue was re-dissolved in dimethyl sulfoxide/methanol and purified using preparative-HPLC to give the titled compound (24.8 mg, 51.1%). ¹H NMR (400 MHz, DMSO-d₆) δ ppm 8.26 (dd, J=8.1, 1.4 Hz, 1H), 7.67 (ddd, J=8.4, 7.2, 1.5 Hz, 1H), 7.51 (ddd, J=8.2, 7.2, 1.2 Hz, 1H), 7.41 (d, J=8.1 Hz, 1H), 7.32 (s, 1H), 7.19-7.13 (m, 2H), 7.11-7.06 (m, 1H), 7.02 (d, J=7.4 Hz, 2H), 3.33 (dt, J=18.2, 7.3 Hz, 4H), 2.51 (d, J=7.5 Hz, 2H), 2.16 (t, J=7.1 Hz, 2H), 1.85 (p, J=7.5 Hz, 2H), 1.59 (p, J=7.1 Hz, 2H), 1.48 (p, J=7.2 Hz, 2H); MS (APCI⁺) m/z 421.1 (M+H)⁺.

### Example 170

### 5-[(7-fluoro-2-oxo-1,2,3,4-tetrahydroquinoline-4-carbonyl)(3-phenylpropyl)amino]pentanoic Acid

A 4 mL Wheaton scintillation vial was charged with 280 μL of a 0.6 mM solution of 7-fluoro-2-oxo-1,2,3,4-tetrahydroquinoline-4-carboxylic acid in N,N-dimethylacetamide (35.1 mg, 1.2 equivalents, 0.2 mmol), a 500 μL solution of 1-[bis(dimethylamino)methylene]-1H-1,2,3-triazolo[4,5-b]pyridinium 3-oxid hexafluorophosphate in N,N-dimethylacetamide (58.5 mg, 1.1 equivalents, 0.15 mmol, HATU), a 500 μL solution of methyl 5-[(3-phenylpropyl)amino]pentanoate hydrochloride in N,N-dimethylacetamide (40.0 mg, 0.14 mmol, Example 149—Step 2), neat triethylamine (58.9 μL, 3 equivalents, 0.42 mmol), and a stir bar. This was capped with a white seal and microwave cap for an Anton Paar microwave reactor. The vial was heated in an Anton Paar Synthos 3000 parallel microwave optimizer for 15 minutes at 120° C. Upon completion, the reaction mixture was filtered, and the filtrate was concentrated to dryness under reduced pressure. The residue was then dissolved in 1000 μL of dioxane. To this, 1000 μL of a 1 M LiOH aqueous solution in 75% CH₃OH was added. The mixture was then heated at 60° C. for 1 hour. The reaction mixture was then filtered once more and concentrated under reduced pressure. The residue was re-dissolved in dimethyl sulfoxide/methanol and purified using preparative-HPLC to give the titled compound (13.8 mg, 27.2%). ¹H NMR (400 MHz, DMSO-d₆) δ ppm 7.23 (t, J=7.5 Hz, 2H), 7.14 (dd, J=15.6, 7.6 Hz, 3H), 6.94 (t, J=7.2 Hz, 1H), 6.70-6.61 (m, 2H), 4.17 (s, 1H), 3.42 (dt, J=14.8, 7.3 Hz, 2H), 3.30 (p, J=7.1 Hz, 2H), 2.56 (dt, J=12.3, 6.7 Hz, 4H), 2.21 (t, J=6.7 Hz, 2H), 1.91-1.76 (m, 2H), 1.60-1.45 (m, 4H); MS (APCI⁺) m/z 427.0 (M+H)⁺.

### Example 171

### 5-[(2-acetamidopyridine-4-carbonyl)(3-phenylpropyl)amino]pentanoic Acid

A 4 mL Wheaton scintillation vial was charged with 280 μL of a 0.6 mM solution of 2-acetamidoisonicotinic acid in N,N-dimethylacetamide (30.3 mg, 1.2 equivalents, 0.2 mmol), a 500 μL solution of 1-[bis(dimethylamino)methylene]-1H-1,2,3-triazolo[4,5-b]pyridinium 3-oxid hexafluorophosphate in N,N-dimethylacetamide (58.5 mg, 1.1 equivalents, 0.15 mmol, HATU), a 500 μL solution of methyl 5-[(3-phenylpropyl)amino]pentanoate hydrochloride in N,N-dimethylacetamide (40.0 mg, 0.14 mmol, Example 149—Step 2), neat triethylamine (58.9 μL, 3 equivalents, 0.42 mmol), and a stir bar. This was capped with a white seal and microwave cap for an Anton Paar microwave reactor. The vial was heated in an Anton Paar Synthos 3000 parallel microwave optimizer for 15 minutes at 120° C. Upon completion, the reaction mixture was filtered, and the filtrate was concentrated to dryness under reduced pressure. The residue was then dissolved in 1000 μL of dioxane. To this, 1000 μL of a 1 M LiOH aqueous solution in 75% CH₃OH was added. The mixture was then heated at 60° C. for 1 hour. The reaction mixture was then filtered once more and concentrated under reduced pressure. The residue was re-dissolved in dimethyl sulfoxide/methanol and purified using preparative-HPLC to give the titled compound (11.9 mg, 19.1%). ¹H NMR (400 MHz, DMSO-d₆) δ ppm 8.27 (d, J=5.0 Hz, 1H), 7.88 (d, J=1.2 Hz, 1H), 7.20 (t, J=7.5 Hz, 2H), 7.11 (dd, J=9.0, 6.6 Hz, 3H), 6.92 (dd, J=5.0, 1.4 Hz, 1H), 3.28 (s, 4H), 2.52 (d, J=7.5 Hz, 1H), 2.16 (t, J=7.2 Hz, 2H), 2.10 (s, 3H), 1.92-1.79 (m, 2H), 1.55 (p, J=7.5 Hz, 2H), 1.46 (dd, J=15.2, 6.9 Hz, 3H); MS (APCI⁺) m/z 398.0 (M+H)⁺.

### Example 172

### 5-[(3,6-dimethyl[1,2]oxazolo[5,4-b]pyridine-4-carbonyl)(3-phenylpropyl)amino]pentanoic Acid

A 4 mL Wheaton scintillation vial was charged with 280 μL of a 0.6 mM solution of 3,6-dimethylisoxazolo[5,4-b]pyridine-4-carboxylic acid in N,N-dimethylacetamide (32.7 mg, 1.2 equivalents, 0.2 mmol), a 500 μL solution of 1-[bis(dimethylamino)methylene]-1H-1,2,3-triazolo[4,5-b]pyridinium 3-oxid hexafluorophosphate in N,N-dimethylacetamide (58.5 mg, 1.1 equivalents, 0.15 mmol, HATU), a 500 μL solution of methyl 5-[(3-phenylpropyl)amino]pentanoate hydrochloride in N,N-dimethylacetamide (40.0 mg, 0.14 mmol, Example 149—Step 2), neat triethylamine (58.9 μL, 3 equivalents, 0.42 mmol), and a stir bar. This was capped with a white seal and microwave cap for an Anton Paar microwave reactor. The vial was heated in an Anton Paar Synthos 3000 parallel microwave optimizer for 15 minutes at 120° C. Upon completion, the reaction mixture was filtered, and the filtrate was concentrated to dryness under reduced pressure. The residue was then dissolved in 1000 μL of dioxane. To this, 1000 μL of a 1 M LiOH aqueous solution in 75% CH₃OH was added. The mixture was then heated at 60° C. for 1 hour. The reaction mixture was then filtered once more and concentrated under reduced pressure. The residue was re-dissolved in dimethyl sulfoxide/methanol and purified using preparative-HPLC to give the titled compound (9.8 mg, 20.2%). ¹H NMR (400 MHz, DMSO-d₆) δ ppm 7.36-7.23 (m, 2H), 7.22-7.16 (m, 1H), 7.14-7.05 (m, 2H), 6.87 (d, J=4.5 Hz, 1H), 3.56-3.48 (m, 3H), 3.17-3.05 (m, 2H), 2.75-2.66 (m, 1H), 2.62 (d, J=22.0 Hz, 3H), 2.41-2.35 (m, 4H), 2.34-2.26 (m, 1H), 2.05-1.95 (m, 1H), 1.83-1.58 (m, 4H), 1.50-1.39 (m, 1H), 1.34-1.23 (m, 1H); MS (APCI⁺) m/z 410.1 (M+H)⁺.

### Example 173

### 5-{(3,5-dimethoxy-4-methylbenzoyl)[3-(furan-2-yl)propyl]amino}pentanoic Acid

### Step 1: methyl 5-{[3-(furan-2-yl)propyl]amino}pentanoate

A mixture of 3-(furan-2-yl)propan-1-amine (100 mg, 0.799 mmol), methyl 5-bromopentanoate (156 mg, 0.799 mmol) and potassium carbonate (132 mg, 0.959 mmol) in CH₃CN (4 mL) was stirred at reflux for 1 hour. The mixture was cooled down and filtered. The filtrate was concentrated to give the titled compound. LC-MS (ESI) m/z 240.2 (M+H)⁺, RT=1.343 minutes.

### Step 2: methyl 5-{(3,5-dimethoxy-4-methylbenzoyl)[3-(furan-2-yl)propyl]amino}pentanoate

To a solution of 3,5-dimethoxy-4-methylbenzoic acid (157 mg, 0.799 mmol) and 1-[bis(dimethylamino)methylene]-1H-1,2,3-triazolo[4,5-b]pyridinium 3-oxid hexafluorophosphate (334 mg, 0.879 mmol, HATU) in N,N-dimethylformamide (4 mL) was added triethylamine (0.122 mL 0.879 mmol). The resultant mixture was stirred at room temperature for 10 minutes. A solution of Example 173—Step 1 (191 mg, 0.799 mmol) in N,N-dimethylformamide (1.0 mL) was added to the reaction system. The mixture was stirred at room temperature for 40 minutes. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-40%) to give the titled compound (108 mg, 0.259 mmol, 32.4% yield). LC-MS (ESI) m/z 418.2 (M+H)⁺, RT=2.051 minutes.

### Step 3: 5-{(3,5-dimethoxy-4-methylbenzoyl)[3-(furan-2-yl)propyl]amino}pentanoic Acid

To a solution of Example 173—Step 2 (108 mg, 0.259 mmol) in tetrahydrofuran (1 mL) was added 1 N lithium hydroxide (1.552 mL 1.552 mmol). The mixture was stirred at room temperature for 1 hour. The mixture was concentrated under reduced pressure, and the aqueous residue was washed with ethyl ether twice and then acidified with 1 N HCl to pH=2-3. The mixture was extracted with ethyl acetate 3 times. The combined organic fractions were washed with brine once, dried and concentrated. The residue was purified by preparative HPLC (0.1% CF₃CO₂H in water/CH₃CN) and lyophilized to give the titled compound (79 mg, 0.196 mmol, 76% yield). ¹H NMR (400 MHz, DMSO-d₆) δ ppm 12.10 (s, 1H), 7.43 (d, J=54.6 Hz, 1H), 6.50 (s, 2H), 6.39-6.21 (m, 1H), 6.03 (d, J=99.7 Hz, 1H), 3.75 (s, 6H), 3.38 (s, 2H), 3.15 (s, 2H), 2.66 (s, 1H), 2.43 (s, 1H), 2.25 (s, 1H), 2.08 (s, 1H), 1.98 (s, 3H), 1.84 (d, J=30.1 Hz, 2H), 1.52 (s, 3H), 1.29 (s, 1H); LC-MS (ESI) m/z 404.2 (M+H)⁺, RT=1.882 minutes.

### Example 174

### 5-{(3,5-dimethoxy-4-methylbenzoyl)[3-(5-methylfuran-2-yl)propyl]amino}-2-hydroxy-2-methylpentanoic Acid

### Step 1: methyl 2-(acetyloxy)-2-methyl-5-{[3-(5-methylfuran-2-yl)propyl]amino}pentanoate

A mixture of 3-(5-methylfuran-2-yl)propan-1-amine (100 mg, 0.718 mmol) and Example 155—Step 1 (226 mg, 0.718 mmol) in CH₃CN (3 mL) was stirred at reflux for 50 minutes. The solution was cooled down and concentrated to give the titled compound. LC-MS (ESI) m/z 326.2 (M+H)⁺, RT=1.555 minutes.

### Step 2: methyl 2-(acetyloxy)-5-{(3,5-dimethoxy-4-methylbenzoyl)[3-(5-methylfuran-2-yl)propyl]amino}-2-methylpentanoate

To a solution of 3,5-dimethoxy-4-methylbenzoic acid (141 mg, 0.718 mmol) and 1-[bis(dimethylamino)methylene]-1H-1,2,3-triazolo[4,5-b]pyridinium 3-oxid hexafluorophosphate (300 mg, 0.790 mmol, HATU) in N,N-dimethylformamide (3 mL) was added triethylamine (0.110 mL 0.790 mmol). The resultant mixture was stirred at room temperature for 10 minutes. A solution of Example 174—Step 1 (234 mg, 0.718 mmol) in N,N-dimethylformamide (1.0 mL) was added to the reaction system. The mixture was stirred at room temperature for 40 minutes. It was diluted with water and extracted with ethyl acetate 3 times. The combined organic layers were washed with brine 3 times, dried over anhydrous Na₂SO₄, filtered and concentrated to give a residue which was purified by flash column chromatography on silica gel (loaded directly with CH₂Cl₂) eluted with hexanes and ethyl acetate (0-40%) to give the titled compound (146 mg, 0.217 mmol, 30.3% yield). LC-MS (ESI) m/z 504.2 (M+H)⁺, RT=2.131 minutes.

### Step 3: 5-{(3,5-dimethoxy-4-methylbenzoyl)[3-(5-methylfuran-2-yl)propyl]amino}-2-hydroxy-2-methylpentanoic Acid

To a solution of Example 174—Step 2 (146 mg, 0.290 mmol) in tetrahydrofuran (2 mL) was added 1 N lithium hydroxide (1.740 mL 1.740 mmol). The mixture was stirred at room temperature for 1 hour. The mixture was concentrated under reduced pressure, and the aqueous residue was washed with ethyl ether twice and then acidified with 1 N HCl to pH=2-3. The acidic aqueous mixture was extracted with ethyl acetate 3 times. The combined organic fractions were washed with brine once, dried and concentrated. The residue was purified by preparative HPLC (0.1% NH₃ in H₂O/CH₃CN) and lyophilized to give the titled compound (37 mg, 0.083 mmol, 28.5% yield). ¹H NMR (400 MHz, DMSO-d₆) δ ppm 7.18 (s, 1H), 6.48 (s, 2H), 5.96 (d, J=30.2 Hz, 1H), 5.77 (d, J=20.7 Hz, 1H), 3.76 (s, 6H), 3.32 (s, 2H), 3.12 (d, J=25.3 Hz, 2H), 2.58 (s, 1H), 2.36 (s, 1H), 2.14 (d, J=42.1 Hz, 3H), 1.98 (s, 3H), 1.80 (d, J=33.2 Hz, 2H), 1.61 (s, 2H), 1.38 (d, J=16.4 Hz, 2H), 1.12 (d, J=35.0 Hz, 3H); LC-MS (ESI) m/z 448.2 (M+H)⁺, RT=1.893 minutes.

### Example 175

### 5-{(2,4-difluoro-3,5-dimethoxybenzoyl)[3-(5-methylfuran-2-yl)propyl]amino}pentanoic Acid

### Step 1: methyl 5-{(2,4-difluoro-3,5-dimethoxybenzoyl)[3-(5-methylfuran-2-yl)propyl]amino}pentanoate

2,4-Difluoro-3,5-dimethoxybenzoic acid (51.7 mg, 0.237 mmol) and 1-[bis(dimethylamino)methylene]-1H-1,2,3-triazolo[4,5-b]pyridinium 3-oxid hexafluorophosphate (108 mg, 0.284 mmol, HATU) were dissolved in a mixture of dry N,N-dimethylformamide (1 mL), tetrahydrofuran (1.000 mL) and N,N-diisopropylethylamine (92 mg, 0.711 mmol). The mixture was stirred for 20 minutes at room temperature. Then Example 133—Step 1 (60 mg, 0.237 mmol) was added, and the mixture was stirred for another 3 hours at room temperature. The mixture was poured into water and extracted with ethyl acetate 3 times. The combined organic fractions were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (100 mg, 0.221 mmol, 93% yield). LC-MS (ESI) m/z 454.2 (M+H)⁺.

### Step 2: 5-{(2,4-difluoro-3,5-dimethoxybenzoyl)[3-(5-methylfuran-2-yl)propyl]amino}pentanoic Acid

The material of Example 175—Step 1 (100 mg, 0.221 mmol) was dissolved in tetrahydrofuran (2 mL) and lithium hydroxide (31.7 mg, 1.323 mmol) solution (2 mL) was added. The mixture was stirred for 2 hours at room temperature. The mixture was poured into water, the pH was adjusted to 3, and the mixture was extracted with ethyl acetate 3 times. The combined organic fractions were washed with brine, dried over Na₂SO₄, and concentrated. The residue was purified by preparative HPLC (0.1% CF₃CO₂H in H₂O/CH₃CN) to give the titled compound (65 mg, 0.148 mmol, 67.1% yield). ¹H NMR (400 MHz, DMSO-d₆) δ ppm 6.80 (ddd, J=8.9, 5.7, 3.4 Hz, 1H), 5.76 (dd, J=26.0, 2.9 Hz, 2H), 3.91 (d, J=14.0 Hz, 3H), 3.81 (d, J=10.0 Hz, 3H), 3.10 (t, J=6.9 Hz, 2H), 2.57 (t, J=7.7 Hz, 1H), 2.38 (t, J=7.0 Hz, 1H), 2.25 (t, J=6.9 Hz, 1H), 2.20 (s, 1H), 2.08 (d, J=13.1 Hz, 2H), 1.85 (p, J=7.6 Hz, 1H), 1.69 (t, J=7.7 Hz, 1H), 1.54 (ddd, J=17.6, 14.2, 7.7 Hz, 2H), 1.42 (q, J=7.6 Hz, 1H), 1.29 (p, J=7.4 Hz, 1H); LC-MS (ESI) m/z 440.2 (M+H)⁺.

### Example 176

### 5-{(3,5-diethoxybenzoyl)[3-(5-methylfuran-2-yl)propyl]amino}pentanoic Acid

### Step 1: methyl 5-{(3,5-diethoxybenzoyl)[3-(5-methylfuran-2-yl)propyl]amino}pentanoate

3,5-Diethoxybenzoic acid (49.8 mg, 0.237 mmol) and 1-[bis(dimethylamino)methylene]-1H-1,2,3-triazolo[4,5-b]pyridinium 3-oxid hexafluorophosphate (108 mg, 0.284 mmol, HATU) was dissolved in a mixture of dry N,N-dimethylformamide (1 mL), tetrahydrofuran (1.000 mL) and N,N-diisopropylethylamine (92 mg, 0.711 mmol). The mixture was stirred for 20 minutes at room temperature. Then Example 133—Step 1 (60 mg, 0.237 mmol) was added, and the solution was stirred for another 3 hours at room temperature. The mixture was poured into water and extracted with ethyl acetate 3 times. The combined organic layers were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (80 mg, 0.180 mmol, 76% yield). LC-MS (ESI) m/z 446.2 (M+H)⁺.

### Step 2: 5-{(3,5-diethoxybenzoyl)[3-(5-methylfuran-2-yl)propyl]amino}pentanoic Acid

Example 176—Step 1 (96 mg, 0.215 mmol) was dissolved in tetrahydrofuran (2 mL) and lithium hydroxide (31.0 mg, 1.293 mmol) solution (2 mL) was added. The mixture was stirred for 2 hours at room temperature. The mixture was poured into water, the pH was adjusted to 3, and the mixture was extracted with ethyl acetate 3 times. The combined organic layers were washed with brine, dried over Na₂SO₄, and concentrated. The residue was purified by preparative HPLC (0.1% CF₃CO₂H in H₂O/CH₃CN) to give the titled compound (72 mg, 0.167 mmol, 77% yield). ¹H NMR (400 MHz, DMSO-d₆) δ ppm 11.99 (s, 1H), 6.41 (d, J=43.8 Hz, 3H), 3.99 (d, J=7.4 Hz, 3H), 3.36 (s, 2H), 3.14 (s, 2H), 2.57 (s, 1H), 2.35 (s, 1H), 2.30-1.99 (m, 4H), 1.78 (d, J=45.9 Hz, 2H), 1.48 (d, J=32.6 Hz, 3H), 1.29 (t, J=6.9 Hz, 5H); LC-MS (ESI) m/z 432.2 (M+H)⁺.

### Example 177

### 5-{[3,5-dimethoxy-4-(trifluoromethyl)benzoyl][3-(5-methylfuran-2-yl)propyl]amino}pentanoic Acid

### Step 1: methyl 5-{[3,5-dimethoxy-4-(trifluoromethyl)benzoyl][3-(5-methylfuran-2-yl)propyl]amino}pentanoate

3,5-Dimethoxy-4-(trifluoromethyl)benzoic acid (59.3 mg, 0.237 mmol) and 1-[bis(dimethylamino)methylene]-1H-1,2,3-triazolo[4,5-b]pyridinium 3-oxid hexafluorophosphate (108 mg, 0.284 mmol, HATU) were dissolved in a mixture of dry N,N-dimethylformamide (1 mL), tetrahydrofuran (1.000 mL) and N,N-diisopropylethylamine (92 mg, 0.711 mmol). The mixture was stirred for 20 minutes at room temperature. Then Example 133—Step 1 (60 mg, 0.237 mmol) was added, and the mixture was stirred for another 3 hours at room temperature. The mixture was poured into water and extracted with ethyl acetate 3 times. The combined organic layers were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (98 mg, 0.202 mmol, 85% yield).

### Step 2: 5-{[3,5-dimethoxy-4-(trifluoromethyl)benzoyl][3-(5-methylfuran-2-yl)propyl]amino}pentanoic Acid

Example 177—Step 1 (96 mg, 0.198 mmol) was dissolved in tetrahydrofuran (2 mL) and lithium hydroxide (28.4 mg, 1.186 mmol) solution (2 mL) was added. The mixture was stirred for 2 hours at room temperature. The mixture was poured into water, the pH was adjusted to 3, and the mixture was extracted with ethyl acetate 3 times. The combined organic layers were washed with brine, dried over Na₂SO₄, and concentrated. The residue was purified by preparative HPLC (0.1% CF₃CO₂H in H₂O/CH₃CN) to give the titled compound (75 mg, 0.159 mmol, 80% yield). ¹H NMR (400 MHz, DMSO-d₆) δ ppm 6.68 (d, J=6.4 Hz, 2H), 5.96 (dd, J=28.2, 2.9 Hz, 1H), 5.74 (dd, J=22.2, 2.9 Hz, 1H), 3.82 (d, J=12.5 Hz, 6H), 3.39 (t, J=7.7 Hz, 2H), 3.24-3.03 (m, 2H), 2.60 (t, J=7.6 Hz, 1H), 2.40 (t, J=7.1 Hz, 1H), 2.26 (q, J=8.1, 7.6 Hz, 1H), 2.20 (s, 1H), 2.08 (s, 3H), 1.88 (dd, J=10.7, 5.0 Hz, 1H), 1.77 (p, J=7.4 Hz, 1H), 1.54 (ddd, J=29.5, 13.9, 7.7 Hz, 4H), 1.31 (q, J=7.6 Hz, 1H); LC-MS (ESI) m/z 472.2 (M+H)⁺.

### Example 178

### 5-{(4-cyclopropyl-3,5-dimethoxybenzoyl)[3-(5-methylfuran-2-yl)propyl]amino}pentanoic Acid

### Step 1: methyl 5-{(4-cyclopropyl-3,5-dimethoxybenzoyl)[3-(5-methylfuran-2-yl)propyl]amino}pentanoate

4-Cyclopropyl-3,5-dimethoxybenzoic acid (52.6 mg, 0.237 mmol) and 1-[bis(dimethylamino)methylene]-1H-1,2,3-triazolo[4,5-b]pyridinium 3-oxid hexafluorophosphate (108 mg, 0.284 mmol, HATU) were dissolved in a mixture of dry N,N-dimethylformamide (1 mL), tetrahydrofuran (1.000 mL) and N,N-diisopropylethylamine (92 mg, 0.711 mmol). The mixture was stirred for 20 minutes at room temperature. Then Example 133—Step 1 (60 mg, 0.237 mmol) was added, and the mixture was stirred for another 3 hours at room temperature. The mixture was poured into water and extracted with ethyl acetate 3 times. The combined organic fractions were washed with brine, dried over Na₂SO₄, and concentrated. The residue was purified by flash columnchromatography on silica gel (loaded directly with CH₂Cl₂) eluted with hexanes and ethyl acetate (0-50%) to give the titled compound (96 mg, 0.210 mmol, 89% yield).

### Step 2: 5-{(4-cyclopropyl-3,5-dimethoxybenzoyl)[3-(5-methylfuran-2-yl)propyl]amino}pentanoic Acid

Example 178—Step 1 (100 mg, 0.219 mmol) was dissolved in tetrahydrofuran (2 mL) and lithium hydroxide (31.4 mg, 1.311 mmol) solution (2 mL) was added. The mixture was stirred for 2 hours at room temperature. The mixture was poured into water, the pH was adjusted to 3, and the mixture was extracted with ethyl acetate 3 times. The combined organic fractions were washed with brine, dried over Na₂SO₄, and concentrated. The residue was purified by preparative HPLC (0.1% CF₃CO₂H in H₂O/CH₃CN) to give the titled compound (75 mg, 0.169 mmol, 77% yield). ¹H NMR (400 MHz, DMSO-d₆) δ ppm 6.47 (s, 2H), 5.86 (d, J=42.1 Hz, 2H), 3.71 (s, 6H), 3.26-3.00 (m, 2H), 2.57 (s, 1H), 2.38 (s, 1H), 2.15 (d, J=28.6 Hz, 5H), 1.84 (tt, J=8.8, 5.9 Hz, 3H), 1.53 (s, 3H), 1.30 (s, 1H), 0.94 (dt, J=5.8, 2.9 Hz, 2H), 0.74 (dt, J=8.8, 3.0 Hz, 2H); LC-MS (ESI) m/z 444.2 (M+H)⁺.

### Example 179

### 5-{(3,5-dimethoxy-4-methylbenzoyl)[3-(5-methylfuran-2-yl)propyl]amino}-2-methylpentanoic Acid

### Step 1: methyl 5-{(3,5-dimethoxy-4-methylbenzoyl)[3-(5-methylfuran-2-yl)propyl]amino}pentanoate

To a solution of 3,5-dimethoxy-4-methylbenzoic acid (1.110 g, 5.66 mmol) in N,N-dimethylformamide (15 mL) was added 1-[bis(dimethylamino)methylene]-1H-1,2,3-triazolo[4,5-b]pyridinium 3-oxid hexafluorophosphate (2.152 g, 5.66 mmol, HATU) and diisopropylethylamine (4.94 mL, 28.3 mmol) at room temperature, and the mixture was stirred at room temperature for 10 minutes. Then Example 133—Step 1 (3.37 g, 5.66 mmol) was added and the resultant mixture was stirred at room temperature for another 2 hours. Water (20 mL) was added to the mixture followed by extraction with ethyl acetate (50 mL) twice. The combined organic fractions were dried over Na₂SO₄ and concentrated. The residue was purified by chromatography on silica gel eluted with hexanes and ethyl acetate (0-20%) to give the titled compound (2.19 g, 5.08 mmol, 90% yield). LC-MS (ESI) m/z 432.2 (M+H)⁺, RT=2.11 minutes.

### Step 2: methyl 5-{(3,5-dimethoxy-4-methylbenzoyl)[3-(5-methylfuran-2-yl)propyl]amino}-2-methylpentanoate

To a solution of diisopropylamine (0.875 mL, 6.14 mmol) in tetrahydrofuran (20 mL) was added n-butyllithium (3.84 mL, 6.14 mmol) at 0° C. under a N₂ atmosphere. The mixture was stirred at 0° C. for 0.5 hour, and then cooled to −78° C. Then a solution of Example 179—Step 1 (1.06 g, 2.456 mmol) in tetrahydrofuran (5 mL) was added to the mixture drop wise, and the mixture was stirred at −78° C. for 1 hour. Then methyl iodide (0.461 mL, 7.37 mmol) was added to the mixture with continued stirring at −78° C. to room temperature over 2 hours. Then the reaction was quenched with saturated NH₄Cl, and the mixture was extracted with ethyl acetate (30 mL). The organic layer was dried over Na₂SO₄, filtered and concentrated to give a residue which was purified by chromatography on silica gel eluted with hexanes and ethyl acetate from 0-20% to give the titled compound (400 mg, 0.898 mmol, 36.5% yield) and Example 180—Step 1 (200 mg, 0.435 mmol, 17.72% yield). LC-MS (ESI) m/z 446.2 (M+H)⁺, RT=2.17 minutes.

### Step 3: 5-{(3,5-dimethoxy-4-methylbenzoyl)[3-(5-methylfuran-2-yl)propyl]amino}-2-methylpentanoic Acid

To a solution of Example 179—Step 2 (60 mg, 0.135 mmol) in tetrahydrofuran (5 mL) was added 1 N LiOH (1.347 mL 1.347 mmol), and the mixture was heated to 50° C. for 4 hours. Then the mixture was cooled to room temperature and concentrated. The residue was diluted with water (10 mL) and washed with ethyl ether (20 mL). The aqueous layer was acidified with 1 N HCl to pH=2-3, and extracted with ethyl acetate (30 mL). The organic layer was dried over Na₂SO₄, filtered and concentrated to give a residue which was purified by high pressure liquid chromatography with water (0.5% CF₃CO₂H) and acetonitrile to give the titled compound (40 mg, 0.093 mmol, 68.8% yield). ¹H NMR (400 MHz, DMSO-d₆) δ ppm 12.14 (brs, 1H), 6.51 (d, J=4.7 Hz, 2H), 5.96 (d, J=27.5 Hz, 1H), 5.77 (d, J=23.4 Hz, 1H), 3.76 (d, J=3.8 Hz, 6H), 3.33 (d, J=30.7 Hz, 2H), 3.14 (s, 2H), 2.58 (s, 1H), 2.45-2.29 (m, 1H), 2.29-2.04 (m, 3H), 1.94-1.66 (m, 2H), 1.66-1.41 (m, 3H), 1.35 (s, 2H), 1.15-0.85 (m, 3H); LC-MS (ESI) m/z 432.2 (M+H)⁺, RT=2.00 minutes.

### Example 180

### 5-{(3,5-dimethoxy-4-methylbenzoyl)[3-(5-methylfuran-2-yl)propyl]amino}-2,2-dimethylpentanoic Acid

### Step 1: methyl 5-{(3,5-dimethoxy-4-methylbenzoyl)[3-(5-methylfuran-2-yl)propyl]amino}-2,2-dimethylpentanoate

The titled compound was prepared as described in Example 179—Step 2. LC-MS (ESI) m/z 460 (M+H)⁺, RT=2.23 minutes

### Step 2: 5-{(3,5-dimethoxy-4-methylbenzoyl)[3-(5-methylfuran-2-yl)propyl]amino}-2,2-dimethylpentanoic Acid

To a solution of Example 180—Step 1 (200 mg, 0.435 mmol) in 1,4-dioxane (5 mL) was added 1 N LiOH (4.35 mL 4.35 mmol) in H₂O, and the mixture was heated to 70° C. overnight. Then the mixture was cooled to room temperature and concentrated. The residue was diluted with water and washed with ethyl ether (20 mL). The aqueous layer was acidified with 1 N HCl to pH=2-3, and extracted with ethyl acetate (30 mL). The organic layer was dried over Na₂SO₄, filtered and concentrated to give a residue which was purified by high pressure liquid chromatography eluted with acetonitrile and water (0.5% CF₃CO₂H) to give the titled compound (143 mg, 0.321 mmol, 73.8% yield). ¹H NMR (400 MHz, DMSO-d₆) δ ppm 12.15 (brs, 1H), 6.50 (s, 2H), 5.96 (d, J=28.0 Hz, 1H), 5.77 (d, J=25.0 Hz, 1H), 3.75 (s, 6H), 3.34-3.35 (m, 2H), 3.22-3.04 (m, 2H), 2.56 (s, 1H), 2.34 (d, J=21.6 Hz, 1H), 2.14 (d, J=39.7 Hz, 3H), 1.81 (d, J=36.5 Hz, 2H), 1.47 (s, 3H), 1.23 (d, J=6.8 Hz, 1H), 1.16-0.89 (m, 6H); LC-MS (ESI) m/z 446.2 (M+H)⁺, RT=2.08 minutes.

### Example 181

### 3,5-dimethoxy-4-methyl-N-{[(2R)-5-oxooxolan-2-yl]methyl}-N-(3-phenylpropyl)benzamide

### Step 1: ethyl (2S,4R)-4-{[tert-butyl(dimethyl)silyl]oxy}pyrrolidine-2-carboxylate

To a solution of (2S,4R)-ethyl 4-hydroxypyrrolidine-2-carboxylate (0.68 g, 4.28 mmol) in CH₂Cl₂ (10 mL) were added tert-butylchlorodimethylsilane (0.847 g, 5.62 mmol) and 1H-imidazole (0.696 g, 10.22 mmol). The mixture was stirred overnight at room temperature and the solids were filtered off. The filtrate was concentrated under reduced pressure, and the residue was partitioned between ethyl acetate and water. The organic phase was separated, and the aqueous layer was back extracted with ethyl acetate. The combined organic fractions were washed with brine, dried over magnesium sulfate end concentrated under reduced pressure. The residue was dissolved in hexane (100 mL) and solids were filtered off. The filtrate was concentrated under vacuum to give the titled compound. ¹H NMR (400 MHz, CDCl₃) δ ppm 4.42 (tt, J=5.0, 2.6 Hz, 1H), 4.22 (dq, J=14.3, 7.1 Hz, 2H), 4.10 (t, J=8.1 Hz, 1H), 3.28 (dd, J=11.6, 4.6 Hz, 1H), 2.94 (ddd, J=11.7, 2.6, 1.3 Hz, 1H), 2.11 (dddd, J=11.7, 7.9, 2.6, 1.2 Hz, 1H), 2.06-1.94 (m, 1H), 1.29 (dd, J=7.7, 6.7 Hz, 3H), 0.89-0.86 (m, 9H), 0.09-0.04 (m, 6H).

### Step 2: ethyl (2S,4R)-4-{[tert-butyl(dimethyl)silyl]oxy}-1-(3-phenylpropyl)pyrrolidine-2-carboxylate

A mixture of Example 181—Step 1 (1 g, 3.66 mmol), (3-bromopropyl)benzene (0.946 g, 4.75 mmol) and acetonitrile (10 mL) was stirred at 70° C. for 0.5 hour. The mixture was concentrated to dryness. The residue was extracted with ethyl acetate, washed with brine, dried with Na₂SO₄, filtered and concentrated to dryness. The residue was purified by flash column chromatography (eluent, ethyl acetate/hexane from 0/100 to 20/80) to give the titled compound. ¹H NMR (400 MHz, CDCl₃) δ ppm 7.29-7.25 (m, 2H), 7.21-7.13 (m, 3H), 4.44 (p, J=5.2 Hz, 1H), 4.18 (q, J=7.1 Hz, 2H), 3.44 (s, 2H), 2.89-2.46 (m, 4H), 2.35 (s, 1H), 2.17 (dt, J=14.0, 7.2 Hz, 1H), 2.02 (ddd, J=12.8, 7.6, 3.9 Hz, 1H), 1.25 (t, J=7.1 Hz, 3H), 0.87 (s, 9H), 0.04 (d, J=4.0 Hz, 6H); LC-MS (ESI) m/z 392.2 (M+H)⁺.

### Step 3: ethyl (4R)-4-{[tert-butyl(dimethyl)silyl]oxy}-5-[(3-phenylpropyl)amino]pentanoate

To a solution of Example 181—Step 2 (0.3 g, 0.766 mmol), hexamethylphosphoramide (0.666 mL 3.83 mmol), and tetrahydrofuran (5 mL) were added dropwise samarium(II) iodide (38.3 mL 3.83 mmol) and pivalic acid (0.222 mL 1.915 mmol) in tetrahydrofuran(2 mL) at 0° C. The resulting solution was allowed to warm to room temperature. A stream of air was bubbled through the solution, and an excess of diatomaceous earth in diethyl ether and saturated aqueous NaHCO₃ (1 mL) were added. The solution was filtered, and the filtrate was washed with brine. The organic layer was separated, dried and concentrated to give the titled compound. LC-MS (ESI) m/z 394.2 (M+H)⁺.

### Step 4: ethyl (4R)-4-{[tert-butyl(dimethyl)silyl]oxy}-5-[(3,5-dimethoxy-4-methylbenzoyl)(3-phenylpropyl)amino]pentanoate

A mixture of 3,5-dimethoxy-4-methylbenzoic acid (0.050 g, 0.254 mmol), Example 181—Step 3 (0.1 g, 0.157 mmol, 62.0% yield), 2-(3H-[1,2,3]triazolo[4,5-b]pyridin-3-yl)-1,1,3,3-tetramethylisouronium hexafluorophosphate(V) (0.097 g, 0.254 mmol), N-ethyl-N-isopropylpropan-2-amine (0.089 mL 0.508 mmol) and N,N-dimethylformamide (1 mL) was stirred at room temperature for 1 hour. The mixture was extracted with ethyl acetate (3×20 mL). The combined organic fractions were washed with brine, dried with Na₂SO₄, filtered and concentrated to dryness. The residue was purified by flash column chromatography on silica gel (eluent, ethyl acetate/hexane from 0/100 to 20/80) to give the titled compound. ¹H NMR (400 MHz, CDCl₃) δ ppm 7.24-7.13 (m, 3H), 6.96 (dd, J=14.5, 6.8 Hz, 2H), 6.51 (s, 2H), 4.19-4.06 (m, 3H), 3.82-3.73 (m, 6H), 3.55 (s, 1H), 3.42-3.21 (m, 3H), 2.70 (s, 1H), 2.44 (d, J=10.6 Hz, 3H), 2.10 (s, 3H), 1.90 (s, 4H), 1.26 (d, J=7.8 Hz, 3H), 0.93-0.82 (m, 9H), 0.10 (tt, J=7.7, 5.6, 4.6 Hz, 4H), 0.00-0.11 (m, 2H); LC-MS (ESI) m/z 572.4 (M+H)⁺.

### Step 5: 3,5-dimethoxy-4-methyl-N-{[(2R)-5-oxooxolan-2-yl]methyl}-N-(3-phenylpropyl)benzamide

A mixture of Example 181—Step 4 (40 mg, 0.070 mmol), tetra-n-butylammonium fluoride (36.6 mg, 0.140 mmol) and tetrahydrofuran (2 mL) was stirred at room temperature overnight. The mixture was extracted with ethyl acetate (3×20 mL). The combined organic layers were washed with brine, dried with Na₂SO₄, filtered and concentrated to dryness. The residue was purified by flash column chromatography on silica gel (eluent, ethyl acetate/hexane from 0/100 to 40/60) to give the titled compound. ¹H NMR (400 MHz, CDCl₃) δ ppm 7.18 (dq, J=14.1, 7.3, 6.9 Hz, 3H), 7.00 (s, 2H), 6.47 (s, 2H), 4.91 (s, 1H), 4.13 (d, J=14.2 Hz, 1H), 3.80 (s, 6H), 3.43 (s, 2H), 3.24 (dd, J=14.6, 7.4 Hz, 1H), 2.62-2.53 (m, 2H), 2.51-2.34 (m, 3H), 2.10 (s, 3H), 1.91 (d, J=21.4 Hz, 3H); LC-MS (ESI) m/z 412.4 (M+H)⁺.

### Example 182

### 5-[(3,5-dimethoxy-4-methylbenzoyl)(3-phenylbutyl)amino]pentanoic Acid

### Step 1: methyl 5-[(3-phenylbutyl)amino]pentanoate

3-Phenylbutan-1-amine (200 mg, 1.340 mmol) and potassium carbonate (222 mg, 1.608 mmol) were dissolved in dry CH₃CN (6 mL) at room temperature. Methyl 5-bromopentanoate (288 mg, 1.474 mmol) dissolved in dry CH₃CN was added slowly to the mixture, and then the mixture was stirred at reflux for 3 hours. Then the mixture was cooled down and filtered. The filtrate was concentrated to give the titled compound (81 mg, 0.308 mmol, 23% yield).

### Step 2: methyl 5-[(3,5-dimethoxy-4-methylbenzoyl)(3-phenylbutyl)amino]pentanoate

3,5-Dimethoxy-4-methylbenzoic acid (263 mg, 1.340 mmol) and 1-[bis(dimethylamino)methylene]-1H-1,2,3-triazolo[4,5-b]pyridinium 3-oxid hexafluorophosphate (611 mg, 1.608 mmol, HATU) were dissolved in a mixture of dry N,N-dimethylformamide (1 mL), tetrahydrofuran (1.000 mL) and N,N-diisopropylethylamine (520 mg, 4.02 mmol). The mixture was stirred for 20 minutes at room temperature. Then Example 182—Step 1 (353 mg, 1.34 mmol) was added, and the mixture was stirred for another 3 hours at room temperature. The mixture was poured into water and extracted with ethyl acetate 3 times. The combined organic fractions were washed with brine, dried over Na₂SO₄, and concentrated. The residue was purified by flash column chromatography on silica gel (loaded directly with CH₂Cl₂) eluted with hexanes and ethyl acetate (0-50%) to give the titled compound (65 mg, 0.147 mmol, 10.99% yield).

### Step 3: 5-[(3,5-dimethoxy-4-methylbenzoyl)(3-phenylbutyl)amino]pentanoic Acid

Example 182—Step 2 (75 mg, 0.170 mmol) was dissolved in tetrahydrofuran (2 mL) and lithium hydroxide (24.41 mg, 1.019 mmol) solution (2 mL) was added. The mixture was stirred for 12 hours at room temperature. The mixture was poured into water, the pH was adjusted to 5, and the mixture was extracted with ethyl acetate 3 times. The combined organic fractions were washed with brine, dried over Na₂SO₄, and concentrated. The residue was purified by preparative HPLC (0.1% CF₃CO₂H in H₂O/CH₃CN) to give the titled compound (56 mg, 0.131 mmol, 77% yield). ¹H NMR (400 MHz, DMSO-d₆) δ ppm 12.02 (s, 1H), 7.46-6.84 (m, 5H), 6.56-6.30 (m, 2H), 3.73 (d, J=8.2 Hz, 7H), 3.22-2.84 (m, 3H), 2.23 (s, 1H), 2.12-2.02 (m, 1H), 1.98 (s, 3H), 1.78 (d, J=49.8 Hz, 2H), 1.45 (d, J=31.3 Hz, 3H), 1.23 (s, 2H), 1.03 (d, J=6.8 Hz, 2H); LC-MS (ESI) m/z 428.2 (M+H)⁺.

### Example 183

### 2-[(3,5-dimethoxy-4-methylbenzoyl)(3-phenylpropyl)amino]ethyl (4-fluorobenzene-1-sulfonyl)carbamate

To a solution of N-(2-hydroxyethyl)-3,5-dimethoxy-4-methyl-N-(3-phenylpropyl)benzamide (300 mg, 0.839 mmol, Example 6—Step 4) in diethyl ether (10 mL) was added 4-fluorobenzenesulfonyl isocyanate (169 mg, 0.839 mmol) dropwise. The solution was stirred at room temperature overnight. The reaction mixture was diluted with ethyl acetate, washed with brine, dried (MgSO₄) and concentrated. Flash chromatography on silica gel (0-100% t-butyl methyl ether in heptanes) gave the titled compound (190 mg, 40%). ¹H NMR (400 MHz, DMSO-d₆) δ ppm 1.87 (p, J=7.7 Hz, 2H), 1.99 (s, 3H), 2.53 (t, J=7.7 Hz, 2H), 3.32-3.44 (m, 4H), 3.53 (t, J=6.1 Hz, 2H), 3.75 (s, 6H), 6.54 (s, 2H), 6.95-7.27 (m, 6H), 7.27-7.40 (m, 2H), 7.77-8.00 (m, 2H); MS (DCI) m/z 559 (M+H)⁺.

### Example 184

### 2-[(3,5-dimethoxy-4-methylbenzoyl)(3-phenylpropyl)amino]ethyl (methanesulfonyl)carbamate

To a solution of N-(2-hydroxyethyl)-3,5-dimethoxy-4-methyl-N-(3-phenylpropyl)benzamide (200 mg, 0.560 mmol, Example 6—Step 4) in t-butyl methyl ether (10 mL) was added freshly prepared methanesulfonyl isocyanate (1.119 mL, 1.119 mmol) (in toluene) dropwise. The mixture was stirred at room temperature overnight and then concentrated. The residue was partitioned between ethyl acetate and brine. The organic layer was dried (MgSO₄) and concentrated. Flash chromatography on silica gel (0-100% t-butyl methyl ether/heptanes) gave the titled compound (34 mg, 13%). ¹H NMR (400 MHz, DMSO-d₆) δ ppm 1.86 (m, 2H), 1.97 (s, 3H), 2.47 (m, 1H), 2.89 (s, 3H), 3.36 (m, 3H), 3.53 (t, J=6.2 Hz, 2H), 3.61 (m, 1H), 3.75 (s, 6H), 4.41 (s, 1H), 6.54 (m, 2H), 7.05-7.21 (m, 5H), 7.21 (s, 1H); MS (DCI) m/z 479 (M+H)⁺.

### Example 185

### 5-{[3-(5-chlorofuran-2-yl)propyl](3,5-dimethoxy-4-methylbenzoyl)amino}pentanoic Acid

### Step 1: methyl 5-{[3-(5-chlorofuran-2-yl)propyl](3,5-dimethoxy-4-methylbenzoyl)amino}pentanoate

To a solution of Example 173—Step 2 (240 mg, 0.575 mmol) in CCl₄ (1.5 mL) was added N-chlorosuccinimide (84 mg, 0.632 mmol) in one portion. Then the mixture was stirred at 70° C. for 15 minutes, and then the mixture was heated for an additional 25 minutes. The mixture was diluted with CH₂Cl₂ and washed with water once, dried over anhydrous Na₂SO₄, filtered and concentrated to give a residue which was purified by flash column chromatography on silica gel eluted with ethyl acetate in hexanes (0-40%) to give the titled compound (117 mg, 0.236 mmol, 41.0% yield). LC-MS (ESI) m/z 452.4 (M+H)⁺, RT=2.453 minutes.

### Step 2: 5-{[3-(5-chlorofuran-2-yl)propyl](3,5-dimethoxy-4-methylbenzoyl)amino}pentanoic Acid

To a solution of Example 185—Step 1 (117 mg, 0.259 mmol) in tetrahydrofuran (2 mL) was added 1 N lithium hydroxide (1.553 mL 1.553 mmol). The mixture was stirred at room temperature for 1 hour. The mixture was concentrated under vacuum, and the aqueous residue was washed with ethyl ether twice and then acidified with 1 N HCl to pH=2-3. The acid aqueous mixture was extracted with ethyl acetate 3 times. The combined organic layers were dried and concentrated. The residue was purified by preparative HPLC (0.1% CF₃CO₂H in H₂O/CH₃CN) and lyophilized to give the titled compound (51 mg, 0.116 mmol, 45.0% yield). ¹H NMR (400 MHz, DMSO-d₆) δ ppm 12.09 (s, 1H), 6.50 (s, 2H), 6.30 (d, J=25.2 Hz, 1H), 6.11 (d, J=76.1 Hz, 1H), 3.75 (s, 6H), 3.37 (s, 2H), 3.17 (s, 2H), 2.63 (s, 1H), 2.41 (s, 1H), 2.25 (s, 1H), 2.09 (s, 1H), 1.98 (s, 3H), 1.87 (s, 1H), 1.77 (s, 1H), 1.64-1.40 (m, 3H), 1.28 (s, 1H); LC-MS (ESI) m/z 438.2 (M+H)⁺, RT=1.964 minutes.

### Example 186

### 3-[(3,5-dimethoxy-4-methylbenzoyl)(3-phenylpropyl)amino]propyl (methanesulfonyl)carbamate

To a solution of N-(3-hydroxypropyl)-3,5-dimethoxy-4-methyl-N-(3-phenylpropyl)benzamide (150 mg, 0.404 mmol, Example 145—Step 1) in t-butyl methyl ether (10 ml) was added a solution of methanesulfonyl isocyanate (48.9 mg, 0.404 mmol) dropwise in toluene, and the mixture was stirred at room temperature overnight. The reaction mixture was diluted with ethyl acetate, washed with brine, dried (MgSO₄) and concentrated. Flash chromatography on silica gel (0-100% t-butyl methyl ether/heptanes) gave the titled compound (60 mg, 28%). ¹H NMR (400 MHz, DMSO-d₆) δ ppm 1.80 (m, 4H), 1.96 (s, 3H), 2.34 (m, 2H), 2.68 (m, 2H), 3.19 (m, 4H), 3.39-3.53 (m, 1H), 3.72 (s, 6H), 3.85-4.29 (m, 2H), 6.49 (s, 2H), 7.08 (m, 5H), 11.55 (s, 1H); MS (DCI) m/z 493 (M+H)⁺.

### Determination of Biological Activity

Abbreviations: BSA for bovine serum albumin; CRC for concentration-response curve; dFBS for dialyzed fetal bovine serum; DMEM for Dulbecco's modified Eagle's medium; DMSO for dimethyl sulfoxide; FLIPR for fluorescence imaging plate reader; FRET for fluorescence resonance energy transfer; HBSS for Hank's buffered salt solution; HEPES for 4-(2-hydroxyethyl)-1-piperazineethanesulfonic acid; HTRF for heterogeneous time resolved fluorescence; LPA for lysophosphatidic acid; PDL for poly-D-lysine; po for per os (by mouth); rpm for revolutions per minute; and TC-treated for tissue culture treated.

### LPAR1 Arrestin Assay.

One day prior to the experiment, frozen U2OS cells stably expressing the LPAR1 receptor (Life Technologies, Grand Island, N.Y., #K1519A) were thawed, centrifuged at 1,000 rpm for 5 minutes to remove DMSO, and resuspended in FreeStyle™ media (Gibco, Grand Island, N.Y.). Forty microliters of cells were plated into PDL-coated 384-well plates (Greiner Bio-One, Monroe, N.C.) at 10,000 cells/well, and then the plates were incubated overnight in humidified 5% CO₂ at 37° C. Test compounds were dissolved in DMSO, and 12-point, 1:3 dilution CRCs were prepared in duplicate; daughter plates were prepared by aliquoting 0.8 μL/well of test compound dilutions into 384-well plates. On the day of the assay, the daughter plates were diluted to 6× stocks using HEPES-buffered HBSS, then 10 μL of 6× test compounds were added to the cells, and the plates were incubated at 37° C. for one hour. After this 1 hour incubation, 10 μL of a 6× stock of the LPAR1 agonist, 18:1 LPA (Avanti Polar Lipids, Alabaster, Ala.), was prepared in HEPES-buffered HBSS+0.1% fatty acid-free BSA (Sigma, St. Louis, Mo.). The 6×LPA was added to all wells for a final concentration of 200 nM, which corresponds to the 18:1 LPA EC₈₀ in this arrestin assay. The final DMSO concentration in the assay plate was 0.1%, and the final BSA concentration was 0.02%. After addition of LPA to all wells, the assay plates were incubated in humidified 5% CO₂ at 37° C. for five hours. During the incubation period, 6× dye from the LiveBLAzer™-FRET B/G with CCF4-AM kit (Life Technologies, Grand Island, N.Y.) was prepared according to manufacturers' instructions. At the end of the 5 hour compound incubation, 10 μL of 6× dye was added to each well, and the assay plate was incubated for two hours in the dark at room temperature. Throughout the assay procedure, all volume additions were performed with a Biomek® NX liquid handler (Beckman Coulter, Indianapolis, Ind.). After the 2 hour dye-load, fluorescence was measured on an EnVision® Multilabel Plate Reader (Perkin Elmer, Waltham, Mass.) with an excitation filter at 409/20 nm and emission filters at 460/40 nm and 530/30 nm. Data processing was performed as recommended by the LiveBLAzer™-FRET B/G with CCF4-AM kit manual; all test compound CRCs were performed in duplicate with n≥3.

TABLE 1
Arrestin Reporter Assay Data

| | | Maximum |
|---|---|---|
| | | Inhibition |
| Example | IC50 (μM) | (%) |
| 3 | 2.233 | 110 |
| 4 | 0.027 | 101 |
| 6 | 0.126 | 130 |
| 7 | 0.713 | 108 |
| 8 | 0.691 | 112 |
| 10 | 0.674 | 114 |
| 20 | 0.043 | 101 |
| 30 | 0.618 | 118 |
| 32 | 0.076 | 112 |
| 33 | 0.071 | 110 |
| 34 | 0.391 | 115 |
| 35 | 0.013 | 103 |
| 37 | 0.231 | 122 |
| 38 | 0.026 | 114 |
| 39 | 1.214 | 99 |
| 44 | 0.069 | 114 |
| 45 | 0.494 | 108 |
| 48 | 3.323 | 113 |
| 50 | 0.001 | 103 |
| 57 | 0.004 | 103 |
| 58 | 0.636 | 112 |
| 60 | 0.308 | 110 |
| 62 | 0.039 | 114 |
| 63 | 3.066 | 118 |
| 77 | 0.005 | 102 |
| 78 | 0.011 | 103 |
| 89 | 0.445 | 114 |
| 90 | 0.675 | 108 |
| 91 | 0.293 | 120 |
| 99 | 3.248 | 112 |
| 105 | 0.301 | 120 |
| 109 | 0.139 | 111 |
| 110 | 0.706 | 110 |
| 111 | 0.093 | 116 |
| 115 | 1.083 | 104 |
| 116 | 0.281 | 108 |
| 117 | 0.103 | 116 |
| 122 | 0.152 | 127 |
| 129 | 0.218 | 124 |
| 130 | 0.760 | 119 |
| 131 | 2.137 | 120 |
| 132 | 0.490 | 126 |
| 134 | 2.239 | 125 |
| 139 | 0.464 | 125 |
| 147 | 0.178 | 122 |
| 151 | 0.665 | 114 |
| 155 | 0.401 | 125 |
| 157 | 0.108 | 128 |
| 173 | 0.295 | 122 |
| 174 | 0.991 | 118 |
| 175 | 5.681 | 119 |
| 178 | 1.556 | 122 |
| 184 | 1.55 | 99 |
| 185 | 0.288 | 122 |

### LPAR1 FLIPR® Assay

U2OS cells stably expressing LPAR1 (Life Technologies #K1519A) were plated at 12000 cells/well in growth medium (McCoy's 5A medium(Life Technologies 16600-082)) with 10% dFBS (Life Technologies 26400-036), 0.1 mM non-essential amino acids, 25 mM HEPES, 1 mM sodium pyruvate, 1% penicillin/streptomycin, 50 μg/mL hygromycin, and 100 μg/mL Geneticin® in black-walled, clear-bottom 384-well plates (Corning #3683) and incubated in humidified 5% CO₂ at 37° C. overnight. On the day of the assay, medium was removed and 30 μL of 5% Calcium 6 FLIPR dye in assay buffer-HBSS (Life Technologies #14025) containing 20 mM HEPES (Life Technologies #15630-080) was added to the cells. Cells were incubated at 37° C. in the dark for 2 hours. Compounds were 3-fold serial diluted from 10 mM for 11-points using dimethyl sulfoxide. 1 μL of compound in DMSO was diluted in 82.3 μL assay buffer immediately before the assay. Using a FLIPR® TETRA® system, 15 μL of the (4×) compound solutions were first added to the cells, then 15 μL of LPA (EC₈₀) diluted in of assay buffer supplemented with 0.1% BSA was added 3 minutes later to activate the receptor. Changes in fluorescence were measured at wavelengths of 515-575 nm over the course of the experimental run. IC₅₀ values were calculated from curve fits of the ratio data using a four-parameter logistic Hill equation (Assay Explorer 3.3 Client).

TABLE 2
LPAR1 FLIPR ®Assay Data

| | | Maximum |
|---|---|---|
| | | Inhibition |
| Example | IC₅₀ (μM) | (%) |
| 1 | 0.045 | 74 |
| 2 | 0.114 | 72 |
| 3 | 0.071 | 52 |
| 4 | 0.014 | 98 |
| 5 | 0.027 | 97 |
| 6 | 0.011 | 94 |
| 7 | 0.032 | 82 |
| 8 | 0.017 | 86 |
| 9 | 0.695 | 75 |
| 10 | 0.064 | 92 |
| 11 | 0.015 | 96 |
| 12 | 3.241 | 55 |
| 13 | 6.165 | 48 |
| 14 | 0.942 | 41 |
| 15 | 0.290 | 59 |
| 16 | 0.985 | 70 |
| 17 | 3.982 | 61 |
| 18 | 0.050 | 89 |
| 19 | 0.071 | 98 |
| 20 | 0.020 | 99 |
| 21 | 0.224 | 82 |
| 22 | 0.054 | 89 |
| 23 | 12.643 | 53 |
| 24 | 0.481 | 58 |
| 25 | 29.261 | 48 |
| 26 | >30.000 | 50 |
| 27 | 1.061 | 65 |
| 28 | 0.398 | 72 |
| 29 | 0.309 | 79 |
| 30 | 0.042 | 96 |
| 31 | 0.045 | 87 |
| 32 | 0.018 | 98 |
| 33 | 0.019 | 99 |
| 34 | 0.024 | 96 |
| 35 | 0.032 | 98 |
| 36 | 0.600 | 83 |
| 37 | 0.032 | 96 |
| 38 | 0.014 | 99 |
| 39 | 0.054 | 96 |
| 40 | 29.302 | 45 |
| 41 | 0.022 | 97 |
| 42 | 0.236 | 90 |
| 43 | 23.283 | 55 |
| 44 | 0.008 | 96 |
| 45 | 0.051 | 91 |
| 46 | 0.128 | 66 |
| 47 | 0.079 | 88 |
| 48 | 0.052 | 81 |
| 49 | 24.262 | 58 |
| 50 | 0.003 | 101 |
| 51 | 0.238 | 62 |
| 52 | 0.124 | 87 |
| 53 | 0.239 | 63 |
| 54 | 0.015 | 100 |
| 55 | 0.022 | 100 |
| 56 | 0.021 | 95 |
| 57 | 0.035 | 100 |
| 58 | 0.032 | 93 |
| 59 | 0.023 | 100 |
| 60 | 0.020 | 95 |
| 61 | 0.020 | 98 |
| 62 | 0.029 | 97 |
| 63 | 0.080 | 87 |
| 64 | 1.226 | 54 |
| 65 | 5.023 | 49 |
| 66 | 0.312 | 71 |
| 67 | >30.000 | 46 |
| 68 | 0.902 | 52 |
| 69 | >30.000 | 38 |
| 70 | 0.056 | 98 |
| 71 | 0.012 | 91 |
| 72 | 0.026 | 99 |
| 73 | 0.060 | 65 |
| 74 | 0.079 | 67 |
| 75 | 0.020 | 72 |
| 76 | 0.048 | 76 |
| 77 | 0.020 | 100 |
| 78 | 0.033 | 100 |
| 79 | >30.000 | 13 |
| 80 | >30.000 | 37 |
| 81 | >30.000 | 31 |
| 82 | >30.000 | 39 |
| 83 | >30.000 | 25 |
| 84 | >30.000 | 7 |
| 85 | >30.000 | 14 |
| 86 | 0.044 | 88 |
| 87 | 0.081 | 71 |
| 88 | 0.293 | 91 |
| 89 | 0.036 | 95 |
| 90 | 0.037 | 88 |
| 91 | 0.025 | 89 |
| 92 | 0.060 | 90 |
| 93 | 0.043 | 96 |
| 94 | >30.000 | 2 |
| 95 | >30.000 | 10 |
| 96 | 19.923 | 62 |
| 97 | 0.116 | 97 |
| 98 | 0.029 | 87 |
| 99 | 0.067 | 82 |
| 100 | >30.000 | 60 |
| 101 | 6.946 | 59 |
| 102 | 0.047 | 85 |
| 103 | 0.068 | 75 |
| 104 | 0.094 | 95 |
| 105 | 0.025 | 90 |
| 106 | 0.766 | 55 |
| 107 | 0.078 | 85 |
| 108 | 0.026 | 94 |
| 109 | 0.015 | 93 |
| 110 | 0.016 | 90 |
| 111 | 0.028 | 95 |
| 112 | 0.315 | 77 |
| 113 | 0.032 | 87 |
| 114 | 0.671 | 76 |
| 115 | 0.030 | 78 |
| 116 | 0.020 | 88 |
| 117 | 0.010 | 93 |
| 118 | 0.247 | 78 |
| 119 | 0.097 | 92 |
| 120 | 0.039 | 81 |
| 121 | 0.503 | 86 |
| 122 | 0.010 | 95 |
| 123 | 0.046 | 94 |
| 124 | 0.059 | 92 |
| 125 | >10.000 | 32 |
| 126 | 0.119 | 74 |
| 127 | >10.000 | 7 |
| 128 | 0.023 | 89 |
| 129 | 0.020 | 93 |
| 130 | 0.041 | 83 |
| 131 | 0.021 | 86 |
| 132 | 0.010 | 86 |
| 133 | 0.046 | 78 |
| 134 | 0.074 | 77 |
| 135 | >10.000 | 32 |
| 136 | >10.000 | 15 |
| 137 | 0.092 | 64 |
| 138 | 0.013 | 89 |
| 139 | 0.046 | 78 |
| 140 | 0.143 | 89 |
| 141 | 0.028 | 94 |
| 142 | 0.076 | 88 |
| 143 | 0.081 | 83 |
| 144 | 0.099 | 92 |
| 145 | 0.437 | 51 |
| 146 | 0.046 | 89 |
| 147 | 0.031 | 93 |
| 148 | >10.000 | 47 |
| 149 | 0.838 | 72 |
| 150 | 0.045 | 73 |
| 151 | 0.037 | 79 |
| 152 | 0.085 | 88 |
| 153 | 0.127 | 83 |
| 154 | 0.225 | 69 |
| 155 | 0.027 | 90 |
| 156 | 0.021 | 99 |
| 157 | 0.016 | 92 |
| 158 | 0.511 | 98 |
| 159 | 0.302 | 85 |
| 160 | 8.020 | 63 |
| 161 | 0.099 | 99 |
| 162 | 0.076 | 100 |
| 163 | 0.244 | 57 |
| 164 | 0.237 | 76 |
| 165 | 0.045 | 83 |
| 166 | 0.042 | 96 |
| 167 | 0.635 | 58 |
| 168 | 1.102 | 62 |
| 169 | 3.094 | 71 |
| 170 | 0.333 | 81 |
| 171 | 0.933 | 70 |
| 172 | 1.463 | 63 |
| 173 | 0.029 | 74 |
| 174 | 0.033 | 73 |
| 175 | 0.048 | 73 |
| 176 | 0.084 | 64 |
| 177 | 0.235 | 57 |
| 178 | 0.045 | 84 |
| 179 | 0.046 | 97 |
| 180 | 0.048 | 100 |
| 181 | 0.486 | 80 |
| 182 | 0.047 | 91 |
| 183 | >10.000 | 30 |
| 184 | 0.131 | 66 |
| 185 | 0.013 | 91 |
| 186 | 0.065 | 83 |

### LPAR2 FLIPR® Assay

U2OS cells stably expressing LPAR2 (Life Technologies #K1442) were plated at 12000 cells/well in growth medium (McCoy's 5A(Life Technologies 16600-082)) with 10% dFBS (Life Technologies 26400-036), 0.1 mM non-essential amino acids, 25 mM HEPES, 1 mM sodium pyruvate, 1% penicillin/streptomycin, 50 μg/mL hygromycin, and 100 μg/mL Geneticin® in black-walled, clear-bottom 384-well plates (Corning #3683) and incubated in humidified 5% CO₂ at 37° C. overnight. On the day of the assay, medium was removed and 30 μL of 5% Calcium 6 FLIPR dye in assay buffer-HBSS (Life Technologies #14025) containing 20 mM HEPES (Life Technologies #15630-080) was added to the cells. Cells were incubated at 37° C. in the dark for 2 hours. Compounds were 3-fold serial diluted from 10 mM for 11-points using dimethyl sulfoxide. 1 μL of compound in DMSO was diluted in 82.3 μL of assay buffer immediately before the assay. Using a FLIPR® TETRA® system, 15 μL of the (4×) compound solutions were first added to the cells, then 15 μL of LPA (EC₈₀) diluted in assay buffer supplemented with 0.1% BSA were added 3 minutes later to activate the receptor. Changes in fluorescence were measured at wavelengths of 515-575 nm over the course of the experimental run. IC₅₀ values were calculated from curve fits of the ratio data using a four-parameter logistic Hill equation (Assay Explorer 3.3 Client).

### LPAR3 FLIPR® Assay

U2OS cells stably expressing LPAR3 (Life Technologies #K1849A) were plated at 12000 cells/well in growth medium (McCoy's 5A(Life Technologies 16600-082)) with 10% dFBS (Life Technologies 26400-036), 0.1 mM non-essential amino acids, 25 mM HEPES, 1 mM sodium pyruvate, 1% penicillin/streptomycin, 50 μg/mL hygromycin, and 100 μg/mL Geneticin® in black-walled, clear-bottom 384-well plates (Corning #3683) and incubated in humidified 5% CO₂ at 37° C. overnight. On the day of the assay, medium was removed and 30 μL of 5% Calcium 6 FLIPR dye in assay buffer-HBSS (Life Technologies #14025) containing 20 mM HEPES (Life Technologies #15630-080) was added to the cells. Cells were incubated at 37° C. in the dark for 2 hours. Compounds were 3-fold serial diluted from 10 mM for 11-points using dimethyl sulfoxide. 1 μL of compound in DMSO was diluted in 82.3 μL assay buffer immediately before the assay. Using a FLIPR® TETRA® system, 15 μL of the (4×) compound solutions were first added to the cells, then 15 μL of LPA (EC₈₀) diluted in assay buffer supplemented with 0.1% BSA were added 3 minutes later to activate the receptor. Changes in fluorescence were measured at wavelengths of 515-575 nm over the course of the experimental run. IC₅₀ values were calculated from curve fits of the ratio data using a four-parameter logistic Hill equation (Assay Explorer 3.3 Client).

### IP-One HTRF® Assay

U2OS cells stably expressing LPAR1 (Life Technologies #K1519A) were plated at 20000 cells/well in growth medium (McCoy's 5A(Life Technologies #16600-082)) with 10% dFBS (Life Technologies #26400-036), 0.1 mM non-essential amino acids, 25 mM HEPES, 1 mM sodium pyruvate, 1% penicillin/streptomycin, 50 μg/mL hygromycin, and 100 μg/mL Geneticin® in tissue culture treated 384-well white plates (Greiner #781080) in humidified 5% CO₂ at 37° C. overnight. On the day of the assay, culture medium was replaced with 30 μL/well of McCoy's 5A with 0.1% BSA. Cells were incubated in humidified 5% CO₂ at 37° C. for 3 more hours. Compounds were 3-fold serial diluted from 2 mM for 11-points using dimethyl sulfoxide. 1 μL of compound in DMSO was diluted in 50 μL stimulation buffer from an IP-One Tb HTRF kit (Cisbio #62IPAPEB) immediately before assay. 15 μL/well of 1.33× compound in stimulation buffer was added to cells after removal of culture medium. After 20 minutes of incubation at 37° C., 5 μL/well of 8 μM LPA diluted in stimulation buffer with 0.1% fatty acid free BSA was added to cells. High control wells used stimulation buffer for substitution. To EC₁₀₀ wells were added 4×20 μM LPA. To the cells were added 5 μL/well IP1-d2 conjugate and 5 μLwell IP1-K antibody after 2 hours of incubation at 37° C. Cells were incubated at room temperature for 1 hour (protected from light). The plate was then read at 665 nm and 615 nm using an EnVision® Multilabel Plate Reader. IC₅₀ values were calculated from curve fits of the ratio data using a four-parameter logistic Hill equation (Assay Explorer 3.3 Client).

TABLE 3
IP-one HTRF ® Assay Data

| | | Maximum |
|---|---|---|
| | | Inhibition |
| Example | IC₅₀ (μM) | (%) |
| 1 | 1.210 | 75 |
| 2 | 4.827 | 84 |
| 3 | 4.273 | 76 |
| 4 | 0.115 | 93 |
| 5 | 0.313 | 105 |
| 6 | 0.183 | 97 |
| 7 | 1.745 | 75 |
| 8 | 2.246 | 84 |
| 9 | 3.041 | 57 |
| 10 | 0.333 | 77 |
| 11 | 0.253 | 88 |
| 20 | 0.050 | 102 |
| 32 | 0.321 | 88 |
| 33 | 0.166 | 76 |
| 34 | 0.880 | 70 |
| 35 | 0.059 | 94 |
| 37 | 0.069 | 79 |
| 38 | 0.194 | 90 |
| 41 | 0.191 | 100 |
| 44 | 0.223 | 80 |
| 50 | 0.003 | 85 |
| 54 | 0.022 | 91 |
| 55 | 0.089 | 98 |
| 56 | 0.424 | 100 |
| 57 | 0.062 | 95 |
| 59 | 0.449 | 88 |
| 60 | 0.208 | 82 |
| 61 | 0.114 | 85 |
| 62 | 0.291 | 75 |
| 68 | 13.252 | 67 |
| 69 | 3.824 | 85 |
| 73 | >30.000 | 44 |
| 74 | 1.179 | 63 |
| 75 | 1.070 | 77 |
| 76 | 5.830 | 65 |
| 77 | 0.077 | 84 |
| 91 | 0.285 | 70 |
| 98 | 0.836 | 76 |
| 105 | 1.683 | 46 |
| 108 | 0.230 | 82 |
| 109 | 0.809 | 85 |
| 110 | 0.902 | 82 |
| 111 | 0.570 | 80 |
| 115 | 2.915 | 71 |
| 116 | 0.344 | 81 |
| 117 | 0.473 | 85 |
| 122 | 0.134 | 78 |
| 128 | 1.796 | 101 |
| 129 | 1.311 | 96 |
| 131 | 1.485 | 101 |
| 132 | 0.220 | 80 |
| 138 | 0.201 | 87 |
| 141 | 0.200 | 76 |
| 155 | 0.663 | 83 |
| 157 | 0.920 | 75 |
| 173 | 0.523 | 75 |
| 185 | 0.325 | 73 |

### LPA-Induced MCP-1 AlphaLISA® Assay

Two days prior to the experiment, NRK-49F cells were plated at 8,000 cells/well in 100 μL of growth medium [DMEM (Life Technologies #11995) with 5% dFBS (Life Technologies #30067-334), 100 units/mL penicillin/streptomycin (Life Technologies #15140122)] in 96-well clear flat bottom polystyrene TC-treated microplate (Corning® #3599) and incubated in humidified 10% CO₂ at 37° C. One day prior to the experiment, the cell medium was removed and serum free medium [100 μL/well, DMEM (Life Technologies #11995) with 0.1% fatty acid free BSA (Sigma #A6003)] was added with continued incubation in humidified 10% CO₂ at 37° C. overnight. On the day of the assay, test compounds were 1:3 serially diluted in 100% DMSO using a BioTek Precision™ system and then transferred (equivalent volume of DMSO as controls) to a plate in serum free medium [DMEM with 0.1% fatty acid free BSA] using a Biomek® NX workstation to give a 2× compound solution. The cell culture medium was removed, and cells were pre-treated with 50 μL/well of 2× compound in serum free medium [DMEM with 0.1% fatty acid free BSA] at 37° C. for 30 minutes. Following the incubation, 50 μL/well of 20 μM LPA diluted in serum free medium [DMEM with 0.1% fatty acid free BSA] was added to the cells. Serum free medium [DMEM with 0.1% fatty acid free BSA] was used as substitution for low control wells while 20 M LPA was added to high control wells and test compound wells. 200 M LPA was added to EC₁₀₀ wells. Final DMSO concentration was 1% in every well. Final LPA concentration in test compound wells and high control wells was 10 μM. Final LPA concentration in EC₁₀₀ wells was 100 M. The cell plate was incubated for 7 hours in humidified 10% CO₂ at 37° C. After the incubation, cell supernatant was transferred to a new 96 well polypropylene plate (Greiner #651201) where it was sealed and stored at −80° C. On the detection day, 5 μL of thawed cell supernatant was transferred to a new white opaque 384-well microplate (Perkin Elmer #6007299). An AlphaLISA® mouse/rat CCL2/MCP1, Immunoassay Research kit (PerkinElmer #AL509F) was used. A mixture (20 μL/well) of AlphaLISA® Anti-Analyte Accept beads (10 μg/mL final) and Biotinylated Antibody Anti-Analyte (freshly prepared, 1 nM final) was added. The mixture in the wells was mixed and then incubated at room temperature for 1 hour. Then SA-Donor beads (25 μL/well, 40 μg/mL final) were added giving a final volume of 50 μL/well. The contents of the wells were mixed and then incubated at room temperature for 0.5 hour (protect from light). The plate was read using the default Envision™ Alpha protocol (PerkinElmer). IC₅₀ values were calculated from curve fits of the ratio data using a four-parameter logistic Hill equation (Assay Explorer 3.3 Client).

TABLE 4
LPA-induced MCP-1 AlphaLISA ® assay data

| | | Maximum |
|---|---|---|
| | | Inhibition |
| Example | IC₅₀ (μM) | (%) |
| 1 | 1.541 | 66 |
| 3 | 3.124 | 74 |
| 4 | 0.188 | 91 |
| 5 | 0.286 | 97 |
| 6 | 0.723 | 80 |
| 7 | 2.626 | 71 |
| 8 | 1.829 | 84 |
| 10 | 4.841 | 70 |
| 11 | 0.247 | 91 |
| 17 | >1.296 | 65 |
| 18 | 1.634 | 86 |
| 19 | 0.783 | 84 |
| 20 | 0.169 | 89 |
| 22 | >9.350 | 42 |
| 30 | 0.743 | 74 |
| 31 | 5.319 | 68 |
| 32 | 0.473 | 89 |
| 33 | 0.215 | 84 |
| 34 | 0.786 | 77 |
| 35 | 0.107 | 92 |
| 37 | 0.479 | 81 |
| 38 | 0.108 | 91 |
| 39 | 0.318 | 80 |
| 41 | 0.335 | 88 |
| 44 | 0.410 | 91 |
| 45 | 5.545 | 71 |
| 47 | 4.701 | 58 |
| 48 | >4.771 | 62 |
| 50 | 0.002 | 98 |
| 52 | 1.006 | 77 |
| 54 | 0.055 | 96 |
| 55 | 0.111 | 93 |
| 56 | 1.239 | 79 |
| 57 | 0.039 | 97 |
| 58 | 2.389 | 78 |
| 59 | 0.127 | 93 |
| 60 | 1.547 | 84 |
| 61 | 0.147 | 92 |
| 62 | 0.184 | 94 |
| 63 | 5.530 | 51 |
| 67 | 3.715 | 57 |
| 69 | 1.663 | 62 |
| 70 | 0.637 | 90 |
| 73 | 5.155 | 79 |
| 74 | 3.877 | 77 |
| 75 | 3.250 | 65 |
| 76 | >10.000 | 36 |
| 77 | 0.060 | 92 |
| 78 | 0.078 | 95 |
| 79 | >10.000 | 36 |
| 80 | >10.000 | 37 |
| 81 | >10.000 | 33 |
| 82 | >10.000 | 2 |
| 83 | >10.000 | 11 |
| 84 | >10.000 | 31 |
| 85 | >10.000 | 12 |
| 86 | 2.977 | 74 |
| 87 | 5.437 | 62 |
| 89 | 4.697 | 65 |
| 90 | 2.498 | 76 |
| 91 | 2.093 | 84 |
| 92 | 4.489 | 72 |
| 93 | 0.819 | 84 |
| 94 | >10.000 | 13 |
| 95 | >10.000 | 18 |
| 96 | >10.000 | 52 |
| 97 | 0.036 | 94 |
| 98 | 4.576 | 66 |
| 99 | 7.854 | 56 |
| 102 | 1.550 | 80 |
| 103 | 5.464 | 62 |
| 104 | 2.898 | 74 |
| 105 | 2.126 | 75 |
| 107 | 3.590 | 71 |
| 108 | 0.668 | 83 |
| 109 | 1.554 | 91 |
| 110 | 1.258 | 83 |
| 111 | 0.787 | 91 |
| 112 | 0.996 | 78 |
| 113 | 1.014 | 86 |
| 115 | 3.540 | 67 |
| 116 | 0.173 | 85 |
| 117 | 0.180 | 91 |
| 119 | 1.171 | 80 |
| 120 | 5.239 | 65 |
| 122 | 0.419 | 97 |
| 123 | 0.793 | 85 |
| 124 | 1.147 | 88 |
| 125 | >10.000 | 53 |
| 126 | 3.894 | 75 |
| 127 | >10.000 | 14 |
| 128 | 1.082 | 78 |
| 129 | 0.968 | 90 |
| 130 | 1.790 | 83 |
| 131 | 1.730 | 71 |
| 132 | 0.783 | 80 |
| 133 | 3.187 | 68 |
| 134 | >10.000 | 37 |
| 135 | >10.000 | 25 |
| 136 | >10.000 | 47 |
| 137 | >10.000 | 28 |
| 138 | 1.008 | 85 |
| 139 | 3.151 | 72 |
| 141 | 0.143 | 84 |
| 142 | 0.831 | 90 |
| 143 | 1.351 | 66 |
| 144 | 1.093 | 96 |
| 145 | 3.963 | 59 |
| 146 | 0.807 | 79 |
| 147 | 0.665 | 86 |
| 148 | >10.000 | 40 |
| 150 | 2.028 | 67 |
| 151 | 1.634 | 79 |
| 152 | 6.174 | 64 |
| 155 | 0.763 | 81 |
| 157 | 0.460 | 77 |
| 161 | 0.079 | 88 |
| 162 | 0.007 | 94 |
| 165 | 2.731 | 70 |
| 166 | 1.063 | 80 |
| 167 | >10.000 | 25 |
| 170 | >10.000 | 50 |
| 171 | >2.177 | 30 |
| 173 | 4.535 | 64 |
| 174 | 5.429 | 65 |
| 175 | 3.943 | 67 |
| 176 | >8.186 | 53 |
| 178 | 1.974 | 77 |
| 179 | 1.983 | 74 |
| 180 | 0.105 | 93 |
| 182 | 0.994 | 85 |
| 184 | 5.562 | 58 |
| 185 | 0.536 | 84 |
| 186 | 7.046 | 54 |

### Mouse Unilateral Ureteral Obstruction Kidney Fibrosis Model

All animal handling and experimental protocols were approved by AbbVie's Institutional Animal Care and Use Committee (IACUC) and were conducted in accordance with the ethical principles for pain-related animal research of the American Pain Society. Male CD-1 mice (28-30 g, Charles River) were group housed 10 per cage, housed in a temperature controlled room with a 12/12-hour day/night cycle, with food (2018 Tekland Global 18% protein rodent diet, Harlan®) and water available ad libitum. Mice underwent either complete unilateral ureter obstruction (UUO) or sham surgery to the left kidney. Briefly, animals were anesthetized with isoflurane, and the left ureter was visualized via a flank incision. Using 6-0 silk sutures, the ureter was tied off at two points near the renal pelvis and then completely cut between ligations (ligation step omitted for sham operated animals). The abdominal muscle was sutured and the skin closed using standard wound clips. Test compound or vehicle was delivered twice daily (30 mg/kg, po) via oral gavage, starting on the day before surgery (D-1) and continuing through day 6 after surgery (D6), at which time animals were sacrificed and kidney tissues collected. The renal capsule was removed from the kidney, and the tissue was cut into 2 sagittal sections. One section of the kidney was fixed in 10% neutral buffered formalin for histological assessment of tubulointerstitial collagen deposition using picrosirius red (PSR) staining. A biopsy punch (3 mm diameter) was taken from the remaining kidney segment and transferred to RNA-later for expression analysis (PCR) of the collagen, type I, alpha 1(Col1a1) mRNA. Statistical significance was determined by using a one-way analysis of variance (ANOVA) followed by a Bonferroni post-hoc test and was conducted using GraphPad prism.

Results:

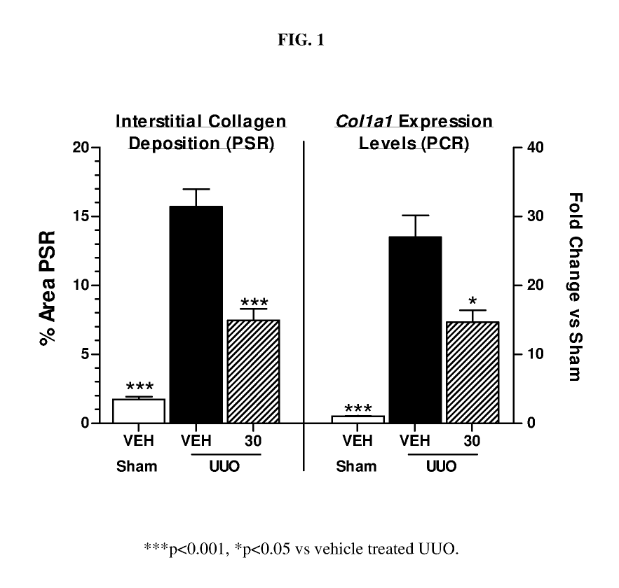

As shown in FIG. 1, there was a significant increase in interstitial collagen deposition (PSR) and Col1a1 expression in vehicle-treated UUO mice (black bars) as compared to sham controls (white bars) on day 6 post surgery. UUO animals treated with Example 7 (striped bars) showed a significant reduction in both tubulointerstitial fibrosis as measured by PSR (−53%) and Col1a1 expression (−46%) compared to the vehicle treated UUO group (black bars).

It is understood that the foregoing detailed description and accompanying examples are merely illustrative and are not to be taken as limitations upon the scope of the present disclosure, which is defined by the appended claims and their equivalents. Various changes and modifications to the described embodiments will be apparent to those skilled in the art. Such changes and modifications, including without limitation those relating to the chemical structures, substituents, derivatives, intermediates, syntheses, formulations, or methods, or any combination of such changes and modifications of use of the present disclosure, may be made without departing from the spirit and scope thereof.

We claim:

1. A compound having formula (I) or a pharmaceutically acceptable salt thereof, 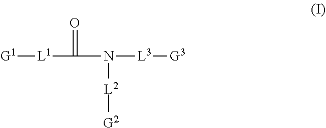
wherein:
G¹ is selected from 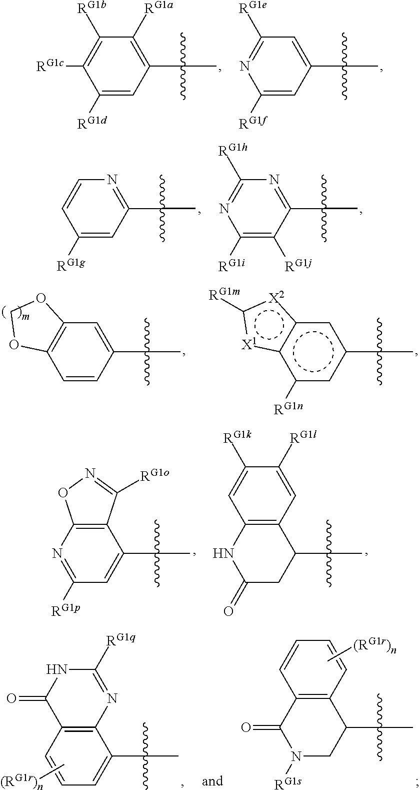
wherein
R^{G1a} is selected from the group consisting of hydrogen and fluorine;
R^{G1b} and R^{G1d} are independently selected from the group consisting of hydrogen, C₁-C₃alkoxy, and halogen; wherein C₁-C₃alkoxy is unsubstituted or optionally substituted with one, two, or three fluorines;
R^{G1c} is selected from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;
wherein at least one of R^{G1b}, R^{G1c}, and R^{G1d} is other than hydrogen;
R^{G1e} and R^{G1f} are independently selected from the group consisting of hydrogen, C₁-C₃alkoxy, and —NHC(O)R^{x}; wherein the C₁-C₃alkoxy is unsubstituted or optionally substituted with one, two, or three fluorines;
R^{x} is C₁-C₃alkyl; wherein the C₁-C₃alkyl is unsubstituted or optionally substituted with one, two or three fluorines;
R^{G1g} is selected from the group consisting of halogen and C₁-C₃alkoxy; wherein the C₁-C₃alkoxy is unsubstituted or optionally substituted with one, two, or three fluorines;
R^{G1h} and R^{G1i} are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines;
R^{G1j} is selected from the group consisting of hydrogen or halogen;
R^{G1k} and R^{G1l} are independently selected from the group consisting of hydrogen and fluorine;
R^{G1m}, R^{G1o}, R^{G1p} R^{G1q}, and R^{G1s} are independently selected at each occurrence from the group consisting of hydrogen and C₁-C₃alkyl; wherein the C₁-C₃alkyl is unsubstituted or optionally substituted with one, two, or three fluorines;
R^{G1n} is selected from the group consisting of halogen and C₁-C₃alkoxy; wherein the C₁-C₃alkoxy is unsubstituted or optionally substituted with one, two, or three fluorines;
R^{G1r} is independently selected at each occurrence from the group consisting of hydrogen, C₁-C₃alkoxy, C₁-C₃alkyl, C₃-C₅cycloalkyl, halogen, and —NO₂; wherein the C₁-C₃alkyl and C₁-C₃alkoxy are unsubstituted or optionally substituted with one hydroxy or one, two, or three fluorines;
one of X¹ and X² is O and the other is CH;
m is one, two or three;
n is one, two or three;
L¹ is a bond or C(R¹R²);
R¹ and R² are independently selected from the group consisting of hydrogen, C₁-C₃alkoxy, and C₁-C₃alkyl; wherein the C₁-C₃alkyl and C₁-C₃alkoxy are unsubstituted or optionally substituted with one, two, or three fluorines; or
R¹ and R² and the carbon atom to which they are attached form a C₃-C₆cycloalkylene or oxetane; wherein the C₃-C₆cycloalkylene is unsubstituted or optionally substituted with one, two, or three substituents selected from C₁-C₃alkoxy, C₁-C₃alkyl and oxo; wherein the C₁-C₃alkyl and C₁-C₃alkoxy are unsubstituted or optionally substituted with one, two, or three fluorines;
G² is selected from the group consisting of phenyl, 2-furanyl and 2-thiophenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines;
L² is —CH₂CH₂CH₂—, wherein the —CH₂CH₂CH₂— is unsubstituted or optionally substituted with C₁-C₃alkyl, wherein the —CH₂CH₂CH₂— or C₁-C₃alkyl substituent are each independently optionally substituted with one, two, or three fluorines, and wherein the central carbon of —CH₂CH₂CH₂— can optionally be attached with a methylene bridge to G²;
G³ is selected from the group consisting of —CO₂H, —P(O)(OH)₂, —P(O)(OH)(OC₁-C₆alkyl), —P(O)(CH₃)(OH), —B(OH)₂, —SO₃H, —CH(OH)CF₃, —C(O)NH(OH), —C(O)NH(CN), —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, —C(O)NHS(O)(R^{G3a})═NR^{G3b}, 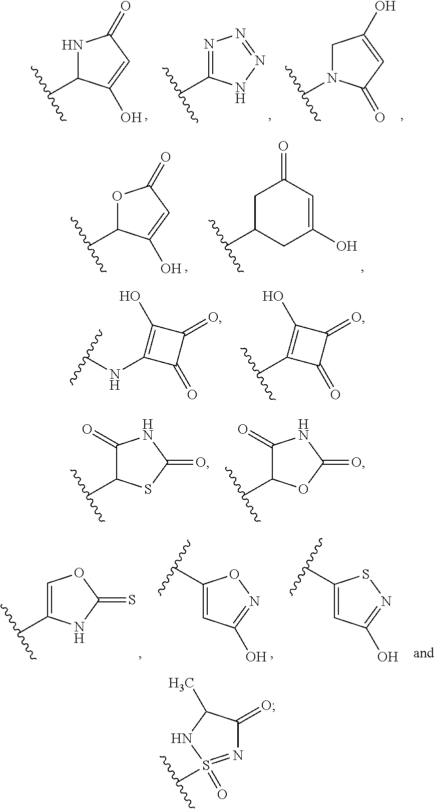
wherein,
R^{G3a} is C₁-C₆alkyl, C₁-C₆haloalkyl, or G^{A};
R^{G3b} is hydrogen, C₁-C₆alkyl, C₁-C₆haloalkyl, or G^{A};
G^{A} is cycloalkyl, cycloalkenyl, aryl, or heteroaryl, each of which is independently unsubstituted or substituted with 1, 2, or 3 independently selected R^{u} groups; wherein
R^{u}, at each occurrence, is independently C₁-C₆alkyl, C₂-C₆alkenyl, C₂-C₆alkynyl, halogen, C₁-C₆haloalkyl, —CN, oxo, —NO₂, —OR^{j}, —OC(O)R^{k}, —OC(O)N(R^{j})₂, —S(O)₂R^{j}, —S(O)₂N(R^{j})₂, —C(O)R^{k}, —C(O)OR^{j}, —C(O)N(R^{j})₂, —N(R^{j})C(O)R^{k}, —N(R^{j})S(O)₂R^{k}, —N(R^{j})C(O)O(R^{k}), or —N(R^{j})C(O)N(R)₂;
R^{j}, at each occurrence, is independently selected from the group consisting of hydrogen, C₁-C₆alkyl, or C₁-C₆haloalkyl;
R^{k}, at each occurrence, is independently selected from the group consisting of C₁-C₆alkyl or C₁-C₆haloalkyl;
L³ is selected from the group consisting of —(CH₂)₂₋₅—, —(CH₂)₁₋₄—(CR³R⁴)—, —(CH₂)—(CR⁵R⁶)₁₋₃—(CH₂)—, —(CR⁷R⁸)₁₋₄—(CH₂)—, —CH₂CH₂—X³—(CR⁹R¹⁰)₁₋₂—, —(CH₂)₁₋₂CH═CH—(CH₂)₁₋₂— and —CH₂C(O)NH(CR¹¹R¹²)—;
R³ and R⁴ are selected from the group consisting of hydrogen, C₁-C₆alkyl, —(C₁-C₆alkylenyl)-G^{B}, and hydroxy, wherein one of R³ and R⁴ is other than hydrogen; or
R³ and R⁴ and the carbon to which they are attached form a C₃-C₆cycloalkylene;
G^{B} is aryl or heteroaryl, each of which is independently unsubstituted or substituted with 1, 2, or 3 independently selected R^{u} groups;
R⁵ is independently selected, at each occurrence, from the group consisting of hydrogen and C₁-C₆alkyl;
one R⁶ is hydroxy, and any additional R⁶ is independently selected, at each occurrence, from the group consisting of hydrogen and C₁-C₆alkyl;
R⁷ and R⁸ are independently selected, at each occurrence, from the group consisting of hydrogen and C₁-C₆alkyl;
R⁹ and R¹⁰ are independently selected, at each occurrence, from the group consisting of hydrogen and C₁-C₆alkyl; or
one R⁹ and R¹⁰ and the carbon to which they are attached form a C₃-C₆cycloalkylene and any additional R⁹ and R¹⁰ are independently selected, at each occurrence, from the group consisting of hydrogen and C₁-C₆alkyl;
X³ is O, S, or S(O)₁₋₂; and
R¹¹ and R¹² are independently selected from the group consisting of hydrogen, C₁-C₆alkyl, and —(C₁-C₆alkylenyl)-G^{B}; or
R¹¹ and R¹² and the carbon to which they are attached form a C₃-C₆cycloalkylene, wherein the C₃-C₆cycloalkylene is unsubstituted or optionally substituted with one, two or three C₁-C₆alkyl or the C₃-C₆cycloalkylene is optionally fused to a phenyl ring.

2. The compound of claim 1 or a pharmaceutically acceptable salt thereof, wherein
G¹ is

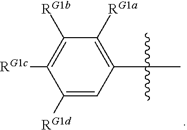

3. The compound of claim 1 or a pharmaceutically acceptable salt thereof, wherein
G¹ is selected from

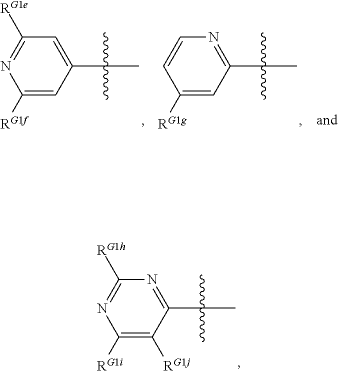

4. The compound of claim 1 or a pharmaceutically acceptable salt thereof, wherein
G¹ is selected from

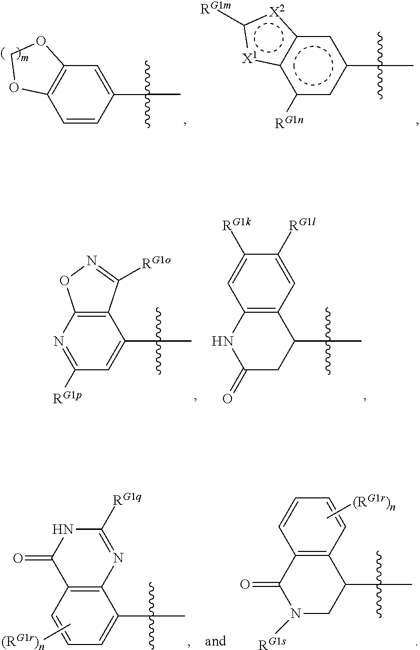

5. The compound of claim 1 or a pharmaceutically acceptable salt thereof, wherein
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines.

6. The compound of claim 1 or a pharmaceutically acceptable salt thereof, wherein
G² is selected from the group consisting of 2-furanyl and 2-thiophenyl; wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines.

7. The compound of claim 1 or a pharmaceutically acceptable salt thereof, wherein
G³ is —CO₂H.

8. The compound of claim 1 or a pharmaceutically acceptable salt thereof, wherein
G³ is selected from the group consisting of —P(O)(OH)₂, —P(O)(OH)(OC₁-C₆alkyl), and —P(O)(CH₃)(OH).

9. The compound of claim 1 or a pharmaceutically acceptable salt thereof, wherein
G³ is selected from the group consisting of —B(OH)₂, —SO₃H, —CH(OH)CF₃, —C(O)NH(OH), and —C(O)NH(CN).

10. The compound of claim 1 or a pharmaceutically acceptable salt thereof, wherein
G³ is selected from the group consisting of —C(O)NHSO₂R^{G3a}, —SO₂NHC(O)R^{G3a}, —C(O)NHSO₂NHR^{G3a}, —NHSO₂NHC(O)R^{G3a}, —OC(O)NHSO₂R^{G3a}, —SO₂NH₂, —SO₂NHR^{G3a}, —C(O)NHS(O)(R^{G3a})═NC(O)R^{G3a}, and —C(O)NHS(O)(R^{G3a})═NR^{G3b},

11. The compound of claim 1 or a pharmaceutically acceptable salt thereof, wherein
G³ is selected from the group consisting of

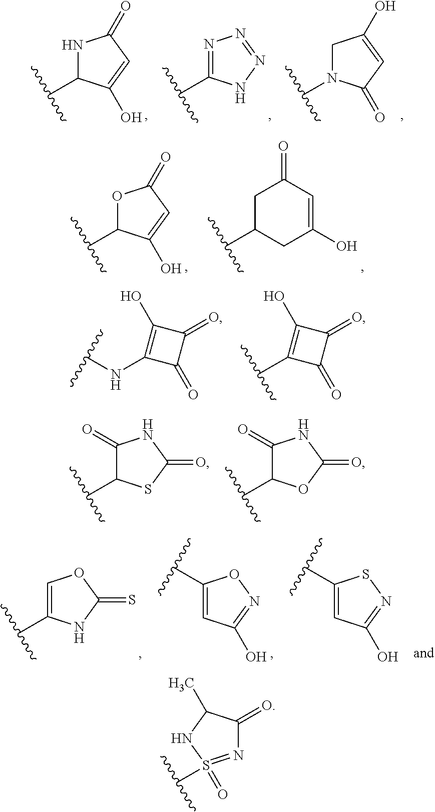

12. The compound of claim 1 or a pharmaceutically acceptable salt thereof, wherein
G¹ is 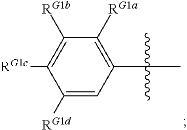
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and
G³ is —CO₂H.

13. The compound of claim 12 or a pharmaceutically acceptable salt thereof, wherein
G¹ is selected from 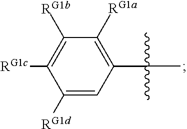
R^{G1a} is selected from the group consisting of hydrogen and fluorine;
R^{G1b} and R^{G1d} are independently selected from the group consisting of hydrogen, and C₁-C₃alkoxy;
R^{G1c} is selected from the group consisting of hydrogen, C₁-C₃alkoxy, C₁-C₃alkyl, C₃-C₅cycloalkyl, and halogen; wherein the C₁-C₃alkyl and C₁-C₃alkoxy are unsubstituted or optionally substituted with one hydroxy or one, two, or three fluorines;
wherein at least one of R^{G1b}, R^{G1c}, and R^{G1d} is other than hydrogen;
L¹ is a bond or C(R¹R²);
R¹ and R² and the carbon atom to which they are attached form a C₃-C₅cycloalkylene;
G² is phenyl; wherein the phenyl is unsubstituted or optionally substituted with 1, 2 or 3 substituents independently selected from C₁-C₃alkoxy, C₁-C₃alkyl, or halogen;
L² is —CH₂CH₂CH₂—;
G³ is —CO₂H;
L³ is selected from the group consisting of —(CH₂)₄₋₅—, —(CH₂)₃₋₄—(CR³R⁴)—, —CH₂CH₂—X³—(CR⁹R¹⁰)₂—, and —CH₂C(O)NH(CR¹¹R¹²)—;
R³ and R⁴ are selected from the group consisting of hydrogen, C₁-C₃alkyl, and hydroxy, wherein one of R³ and R⁴ is other than hydrogen; or
R⁹ and R¹⁰ are independently selected, at each occurrence, from the group consisting of hydrogen and C₁-C₆alkyl;
X³ is O; and
R¹¹ and R¹² are independently selected from the group consisting of hydrogen and C₁-C₃alkyl; or
R¹¹ and R¹² and the carbon to which they are attached form a C₃-C₆cycloalkylene, wherein the C₃-C₆cycloalkylene is optionally fused to a phenyl ring.

14. The compound of claim 1 or a pharmaceutically acceptable salt thereof, wherein
G¹ is 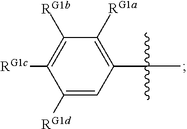
G² is selected from the group consisting of 2-furanyl and 2-thiophenyl; wherein the 2-furanyl and 2-thiophenyl are unsubstituted or optionally substituted with 1 or 2 substituents independently selected from halogen or C₁-C₃alkyl, wherein the C₁-C₃alkyl is unsubstituted or optionally substituted with one, two, or three fluorines; and
G³ is —CO₂H.

15. The compound of claim 14 or a pharmaceutically acceptable salt thereof, wherein
G¹ is selected from 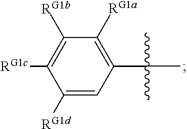
wherein
R^{G1a} is selected from the group consisting of hydrogen and fluorine;
R^{G1b} and R^{G1d} are C₁-C₃alkoxy;
R^{G1c} is selected from the group consisting of C₁-C₃alkoxy and halogen;
wherein at least one of R^{G1b}, R^{G1c}, and R^{G1d} is other than hydrogen;
L¹ is a bond;
G² is selected from the group consisting of 2-furanyl and 2-thiophenyl; wherein the 2-furanyl and 2-thiophenyl are unsubstituted or optionally substituted with 1 C₁-C₃alkyl;
L² is —CH₂CH₂CH₂—;
G³ is —CO₂H; and
L³ is —(CH₂)₄ 5-.

16. The compound of claim 1 or a pharmaceutically acceptable salt thereof, wherein
G is 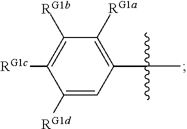
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and
G³ is selected from the group consisting of —P(O)(OH)₂, —P(O)(OH)(OC₁-C₆alkyl), and —P(O)(CH₃)(OH).

17. The compound of claim 16 or a pharmaceutically acceptable salt thereof, wherein
G¹ is selected from 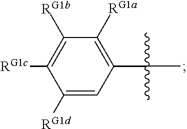
wherein
R^{G1a} is hydrogen;
R^{G1b} and R^{G1d} are each C₁-C₃alkoxy;
R^{G1c} is selected from the group consisting of hydrogen and C₁-C₃alkyl;
wherein at least one of R^{G1b}, R^{G1c}, and R^{G1d} is other than hydrogen;
L¹ is a bond;
G² is phenyl;
L² is —CH₂CH₂CH₂—;
G³ is —P(O)(OH)₂;
L³ is selected from the group consisting of —(CH₂)₄₋₅—, —(CH₂)₃₋₄—(CR³R⁴)—, and —CH₂CH₂—X³—(CR⁹R¹⁰)₁₋₂—;
R³ and R⁴ are selected from the group consisting of hydrogen and C₁-C₃alkyl, wherein one of R³ and R⁴ is other than hydrogen;
R⁹ and R¹⁰ are each hydrogen; and
X³ is O.

18. The compound of claim 1 or a pharmaceutically acceptable salt thereof, wherein
G¹ is selected from 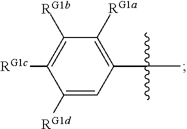
wherein
R^{G1a} is hydrogen;
R^{G1b} and R^{G1d} are each C₁-C₃alkoxy;
R^{G1c} is selected from the group consisting of hydrogen and C₁-C₃alkyl;
wherein at least one of R^{G1b}, R^{G1c}, and R^{G1d} is other than hydrogen;
L¹ is a bond;
G² is phenyl;
L² is —CH₂CH₂CH₂—;
G³ is —CO₂H or —P(O)(OH)₂;
L³ is selected from the group consisting of —(CH₂)₄₋₅ and —CH₂CH₂—X³—(CR⁹R¹⁰)₁₋₂—;
R⁹ and R¹⁰ are each hydrogen; and
X³ is O.

19. The compound of claim 1 or a pharmaceutically acceptable salt thereof, wherein
G¹ is 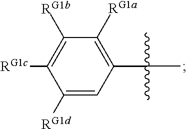
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and
G³ is selected from the group consisting of —C(O)NHSO₂R^{G3a}, —C(O)NHSO₂NHR^{G3a}, and —OC(O)NHSO₂R^{G3a}.

20. The compound of claim 19 or a pharmaceutically acceptable salt thereof, wherein
G¹ is selected from 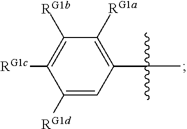
wherein
R^{G1a} is hydrogen;
R^{G1b} and R^{G1d} are each C₁-C₃alkoxy;
R^{G}c is C1-C₃alkyl;
L¹ is a bond;
G² is phenyl;
L² is —CH₂CH₂CH₂—;
G³ is —OC(O)NHSO₂R^{G3a};
R^{G3a} is C₁-C₆alkyl; and
L³ is —(CH₂)₂ 3-.

21. The compound of claim 1 or a pharmaceutically acceptable salt thereof, wherein
G¹ is 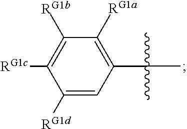
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and
G³ is selected from the group consisting of

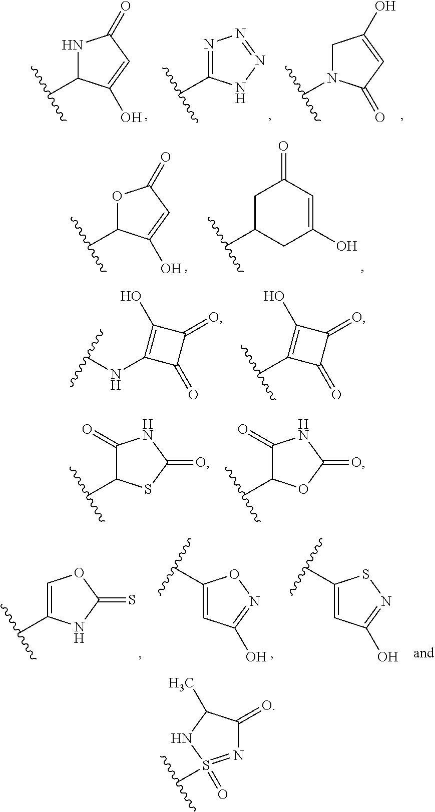

22. The compound of claim 21 or a pharmaceutically acceptable salt thereof, wherein
G¹ is 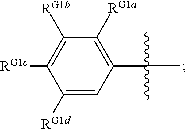
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and
G³ is

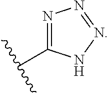 [SMILES: *c1nnn[nH]1]

23. The compound of claim 1 or a pharmaceutically acceptable salt thereof, wherein
G¹ is selected from the group consisting of 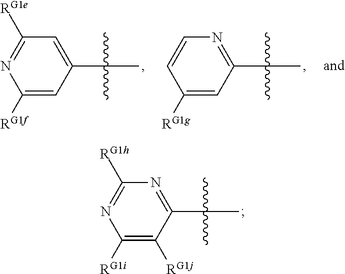
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and
G³ is selected from the group consisting of —CO₂H, —P(O)(OH)₂, —P(O)(OH)(OC₁-C₆alkyl), —P(O)(CH₃)(OH), —C(O)NHSO₂R^{G3a}, —C(O)NHSO₂NHR^{G3a}, —NHSO₂NHC(O)R^{G3a}, and —OC(O)NHSO₂R^{G3a}

24. The compound of claim 23 or a pharmaceutically acceptable salt thereof, wherein
G¹ is selected from the group consisting of 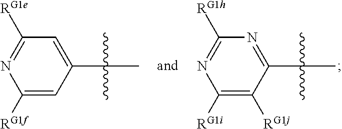
wherein
R^{G1e} and R^{G1f} are each C₁-C₃alkoxy;
R^{G1h} and R^{G1i} are each C₁-C₃alkoxy;
R^{G1j} is hydrogen;
L¹ is a bond;
G² is phenyl;
L² is —CH₂CH₂CH₂—;
G³ is —CO₂H; and
L³ is —(CH₂)₄₋₅—.

25. The compound of claim 1 or a pharmaceutically acceptable salt thereof, wherein
G¹ is selected from the group consisting of 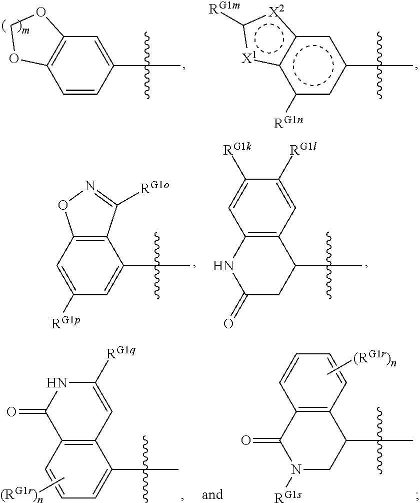
wherein
G² is phenyl; wherein the phenyl is unsubstituted or optionally substituted with 1, 2 or 3 substituents independently selected from C₁-C₃alkoxy, C₁-C₃alkyl, or halogen, wherein the C₁-C₃alkyl and C₁-C₃alkoxy are unsubstituted or optionally substituted with one, two, or three fluorines; and
G³ is selected from the group consisting of —CO₂H, —P(O)(OH)₂, —P(O)(OH)(OC₁-C₆alkyl), —P(O)(CH₃)(OH), —C(O)NHSO₂R^{G3a}, —C(O)NHSO₂NHR^{G3a}, —NHSO₂NHC(O)R^{G3a}, and —OC(O)NHSO₂R^{G3a}.

26. The compound of claim 25 or a pharmaceutically acceptable salt thereof, wherein
G¹ is selected from the group consisting of 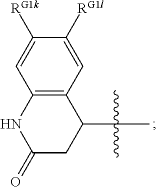
wherein
R^{G1k} and R^{G1l} are independently selected from the group consisting of hydrogen and fluorine;
L¹ is a bond;
G² is phenyl;
L² is —CH₂CH₂CH₂—;
G³ is —CO₂H; and
L³ is —(CH₂)₄₋₅—.

27. The compound or pharmaceutically acceptable salt of claim 1, selected from:
2-{[N-(3,5-dimethoxy-4-methylbenzoyl)-N-(3-phenylpropyl)glycyl]amino}-2,3-dihydro-1H-indene-2-carboxylic acid;
1-{[N-(3,5-dimethoxy-4-methylbenzoyl)-N-(3-phenylpropyl)glycyl]amino}cyclohexane-1-carboxylic acid;
1-{[N-(3,5-dimethoxy-4-methylbenzoyl)-N-(3-phenylpropyl)glycyl]amino}cyclopropane-1-carboxylic acid;
5-[(3,5-dimethoxy-4-methylbenzoyl)(3-phenylpropyl)amino]pentanoic acid;
6-[(3,5-dimethoxy-4-methylbenzoyl)(3-phenylpropyl)amino]hexanoic acid;
{2-[(3,5-dimethoxy-4-methylbenzoyl)(3-phenylpropyl)amino]ethoxy}acetic acid;
5-[(3,5-dimethoxybenzoyl)(3-phenylpropyl)amino]pentanoic acid;
5-{[1-(4-methoxyphenyl)cyclopropane-1-carbonyl](3-phenylpropyl)amino}pentanoic acid;
5-{[(3,5-dimethoxyphenyl)acetyl](3-phenylpropyl)amino}pentanoic acid;
5-[(3-phenylpropyl)(3,4,5-trimethoxybenzoyl)amino]pentanoic acid;
5-{(3,5-dimethoxy-4-methylbenzoyl)[3-(3-methylphenyl)propyl]amino}pentanoic acid;
5-[(3,5-dichlorobenzoyl)(3-phenylpropyl)amino]pentanoic acid;
5-[(3,5-difluoro-4-methoxybenzoyl)(3-phenylpropyl)amino]pentanoic acid;
5-[(2-ethoxypyridine-4-carbonyl)(3-phenylpropyl)amino]pentanoic acid;
{2-[(3,5-dimethoxybenzoyl)(3-phenylpropyl)amino]ethoxy}acetic acid;
5-[(3,5-dichloro-4-methylbenzoyl)(3-phenylpropyl)amino]pentanoic acid;
5-[(4-chloro-3-methoxybenzoyl)(3-phenylpropyl)amino]pentanoic acid;
5-[(3,5-diethoxybenzoyl)(3-phenylpropyl)amino]pentanoic acid;
5-[(3,5-dimethoxy-2-nitrobenzoyl)(3-phenylpropyl)amino]pentanoic acid;
5-[(4-bromo-3,5-dimethoxybenzoyl)(3-phenylpropyl)amino]pentanoic acid;
5-[(3-phenylpropyl)(3,4,5-triethoxybenzoyl)amino]pentanoic acid;
5-[(3-methoxy-4-nitrobenzoyl)(3-phenylpropyl)amino]pentanoic acid;
5-[(3,4-dihydro-2H-1,5-benzodioxepine-7-carbonyl)(3-phenylpropyl)amino]pentanoic acid;
5-[(7-methoxy-1-benzofuran-5-carbonyl)(3-phenylpropyl)amino]pentanoic acid;
5-[(3-phenylpropyl){1-[4-(trifluoromethoxy)phenyl]cyclopropane-1-carbonyl}amino]pentanoic acid;
5-{(3-phenylpropyl)[3-(trifluoromethoxy)benzoyl]amino}pentanoic acid;
5-{[1-(2H-1,3-benzodioxol-5-yl)cyclopropane-1-carbonyl](3-phenylpropyl)amino}pentanoic acid;
5-[(3-methoxy-4-methylbenzoyl)(3-phenylpropyl)amino]pentanoic acid;
5-{[3-methoxy-5-(trifluoromethoxy)benzoyl](3-phenylpropyl)amino}pentanoic acid;
5-[(2,4-difluoro-3,5-dimethoxybenzoyl)(3-phenylpropyl)amino]pentanoic acid;
5-[(4-methoxy-2-methyl-1-benzofuran-6-carbonyl)(3-phenylpropyl)amino]pentanoic acid;
5-{(3,5-dimethoxy-4-methylbenzoyl)[3-(2-fluorophenyl)propyl]amino}pentanoic acid;
5-{(3,5-dimethoxy-4-methylbenzoyl)[3-(3-fluorophenyl)propyl]amino}pentanoic acid;
5-{(3,5-dimethoxy-4-methylbenzoyl)[3-(4-fluorophenyl)propyl]amino}pentanoic acid;
5-[(4-cyclopropyl-3,5-dimethoxybenzoyl)(3-phenylpropyl)amino]pentanoic acid;
5-{[3-(4-chlorophenyl)propyl](3,5-dimethoxy-4-methylbenzoyl)amino}pentanoic acid;
5-{[3,5-dimethoxy-4-(trifluoromethyl)benzoyl](3-phenylpropyl)amino}pentanoic acid;
5-{[3-(3-chlorophenyl)propyl](3,5-dimethoxy-4-methylbenzoyl)amino}pentanoic acid;
5-[(2-chloro-3,5-dimethoxybenzoyl)(3-phenylpropyl)amino]pentanoic acid;
5-[(3,5-dimethoxy-4-methylbenzoyl){3-[3-(trifluoromethyl)phenyl]propyl}amino]pentanoic acid;
5-{(3,5-dimethoxy-4-methylbenzoyl)[3-(5-methylthiophen-2-yl)propyl]amino}pentanoic acid;
5-{(3,5-dimethoxy-4-methylbenzoyl)[3-(4-methylphenyl)propyl]amino}pentanoic acid;
5-{(3,5-dimethoxy-4-methylbenzoyl)[3-(4-methoxyphenyl)propyl]amino}pentanoic acid;
({2-[(3,5-dimethoxy-4-methylbenzoyl)(3-phenylpropyl)amino]ethyl}sulfanyl)acetic acid;
N-(3,5-dimethoxy-4-methylbenzoyl)-N-(3-phenylpropyl)glycyl-2-methylalanine;
N-(3,5-dimethoxy-4-methylbenzoyl)-N-(3-phenylpropyl)glycylphenylalanine;
N-(3,5-dimethoxy-4-methylbenzoyl)-N-(3-phenylpropyl)glycyl-3-thiophen-2-ylalanine;
5-[(2,6-dimethoxypyridine-4-carbonyl)(3-phenylpropyl)amino]pentanoic acid;
5-{[3-(2,4-dichlorophenyl)propyl](3,5-dimethoxy-4-methylbenzoyl)amino}pentanoic acid;
({2-[(3,5-dimethoxy-4-methylbenzoyl)(3-phenylpropyl)amino]ethoxy}methyl)phosphonic acid;
N-(3,5-dimethoxy-4-methylbenzoyl)-N-(3-phenylpropyl)glycylglycine;
2-benzyl-5-[(3,5-dimethoxy-4-methylbenzoyl)(3-phenylpropyl)amino]pentanoic acid;
{2-[(3,5-dimethoxy-4-methylbenzoyl)(3-phenylpropyl)amino]ethanesulfinyl}acetic acid;
5-[(3,5-dimethoxy-4-methylbenzoyl)(3-phenylpropyl)amino]-2-methylpentanoic acid;
2-{2-[(3,5-dimethoxy-4-methylbenzoyl)(3-phenylpropyl)amino]ethoxy}-2-methylpropanoic acid;
3-{2-[(3,5-dimethoxy-4-methylbenzoyl)(3-phenylpropyl)amino]ethoxy}propanoic acid;
5-[(3,5-dimethoxy-4-methylbenzoyl)(3-phenylpropyl)amino]-2,2-dimethylpentanoic acid;
5-[(2-fluoro-3,5-dimethoxybenzoyl)(3-phenylpropyl)amino]pentanoic acid;
1-({2-[(3,5-dimethoxy-4-methylbenzoyl)(3-phenylpropyl)amino]ethoxy}methyl)cyclopropane-1-carboxylic acid;
3-({2-[(3,5-dimethoxy-4-methylbenzoyl)(3-phenylpropyl)amino]ethyl}sulfanyl)propanoic acid;
1-[({2-[(3,5-dimethoxy-4-methylbenzoyl)(3-phenylpropyl)amino]ethyl}sulfanyl)methyl]cyclopropane-1-carboxylic acid;
3-({2-[(3,5-dimethoxy-4-methylbenzoyl)(3-phenylpropyl)amino]ethyl}sulfanyl)butanoic acid;
5-{[1-(5-methoxypyridin-2-yl)cyclopropane-1-carbonyl](3-phenylpropyl)amino}pentanoic acid;
5-{(3-phenylpropyl)[1-(pyridin-4-yl)cyclopropane-1-carbonyl]amino}pentanoic acid;
5-[(6-methoxy-1H-indole-3-carbonyl)(3-phenylpropyl)amino]pentanoic acid;
5-{[(2R)-2-methoxy-2-(4-methoxyphenyl)acetyl](3-phenylpropyl)amino}pentanoic acid;
N-(3,5-dimethoxy-4-methylbenzoyl)-N-(3-phenylpropyl)-beta-alanine;
3,5-dimethoxy-4-methyl-N-{3-[(methylsulfamoyl)amino]-3-oxopropyl}-N-(3-phenylpropyl)benzamide;
4-[(3,5-dimethoxy-4-methylbenzoyl)(3-phenylpropyl)amino]butanoic acid;
3,5-dimethoxy-4-methyl-N-{5-[(methylsulfamoyl)amino]-5-oxopentyl}-N-(3-phenylpropyl)benzamide;
{4-[(3,5-dimethoxy-4-methylbenzoyl)(3-phenylpropyl)amino]butyl}phosphonic acid;
{5-[(3,5-dimethoxy-4-methylbenzoyl)(3-phenylpropyl)amino]pentan-2-yl}phosphonic acid;
1-(4-methoxyphenyl)-N-{5-[(methylsulfamoyl)amino]-5-oxopentyl}-N-(3-phenylpropyl)cyclopropane-1-carboxamide;
3,5-dimethoxy-N-{5-[(methylsulfamoyl)amino]-5-oxopentyl}-N-(3-phenylpropyl)benzamide;
{4-[(3,5-dimethoxybenzoyl)(3-phenylpropyl)amino]butyl}phosphonic acid;
ethyl hydrogen {4-[(3,5-dimethoxybenzoyl)(3-phenylpropyl)amino]butyl}phosphonate;
(−)-(2R)-5-[(3,5-dimethoxy-4-methylbenzoyl)(3-phenylpropyl)amino]-2-methylpentanoic acid;
(+)-(2S)-5-[(3,5-dimethoxy-4-methylbenzoyl)(3-phenylpropyl)amino]-2-methylpentanoic acid;
5-[(3-fluoro-4-methoxybenzoyl)(3-phenylpropyl)amino]pentanoic acid;
5-[(2H-1,3-benzodioxole-5-carbonyl)(3-phenylpropyl)amino]pentanoic acid;
5-[(4-fluoro-3-methoxybenzoyl)(3-phenylpropyl)amino]pentanoic acid;
5-{[1-(3-methoxyphenyl)cyclopropane-1-carbonyl](3-phenylpropyl)amino}pentanoic acid;
5-[(3,4-dimethoxybenzoyl)(3-phenylpropyl)amino]pentanoic acid;
5-[(4-methoxybenzoyl)(3-phenylpropyl)amino]pentanoic acid;
5-{[2-(4-methoxyphenyl)-2-methylpropanoyl](3-phenylpropyl)amino}pentanoic acid;
5-{[1-(4-methoxyphenyl)cyclobutane-1-carbonyl](3-phenylpropyl)amino}pentanoic acid;
(2-{[1-(4-methoxyphenyl)cyclopropane-1-carbonyl](3-phenylpropyl)amino}ethoxy)acetic acid;
5-{[4-(2-hydroxyethoxy)-3,5-dimethoxybenzoyl](3-phenylpropyl)amino}pentanoic acid;
5-{[3-(4-methoxyphenyl)oxetane-3-carbonyl](3-phenylpropyl)amino}pentanoic acid;
5-{(3,5-dimethoxybenzoyl)[3-(3-fluorophenyl)propyl]amino}pentanoic acid;
5-{[3-(3-chlorophenyl)propyl](3,5-dimethoxybenzoyl)amino}pentanoic acid;
5-{[3-(3-fluorophenyl)propyl][1-(4-methoxyphenyl)cyclopropane-1-carbonyl]amino}pentanoic acid;
2-{3-[(3,5-dimethoxy-4-methylbenzoyl)(3-phenylpropyl)amino]propyl}hexanoic acid;
2-{3-[(3,5-dimethoxy-4-methylbenzoyl)(3-phenylpropyl)amino]propyl}-2-methylhexanoic acid;
5-[(3,5-dimethoxybenzoyl)(3-phenylpropyl)amino]-2-methylpentanoic acid;
5-[(4-fluoro-3,5-dimethoxybenzoyl)(3-phenylpropyl)amino]pentanoic acid;
({2-[(4-fluoro-3,5-dimethoxybenzoyl)(3-phenylpropyl)amino]ethyl}sulfanyl)acetic acid;
({2-[(3,5-dimethoxybenzoyl)(3-phenylpropyl)amino]ethyl}sulfanyl)acetic acid;
5-{[3-(3-chlorophenyl)propyl][1-(4-methoxyphenyl)cyclopropane-1-carbonyl]amino}pentanoic acid;
5-{[1-(2-fluoro-4-methoxyphenyl)cyclopropane-1-carbonyl](3-phenylpropyl)amino}pentanoic acid;
1-{[N-(3,5-dimethoxy-4-methylbenzoyl)-N-(3-phenylpropyl)glycyl]amino}-4-ethylcyclohexane-1-carboxylic acid;
5-{[1-(3-fluoro-4-methoxyphenyl)cyclopropane-1-carbonyl](3-phenylpropyl)amino}pentanoic acid;
5-{[1-(4-methoxyphenyl)cyclopentane-1-carbonyl](3-phenylpropyl)amino}pentanoic acid;
5-{[1-(3-chloro-4-methoxyphenyl)cyclopropane-1-carbonyl](3-phenylpropyl)amino}pentanoic acid;
5-[(3,5-dimethoxybenzoyl)(3-phenylpropyl)amino]-2,2-dimethylpentanoic acid;
(2-{[3-(3-chlorophenyl)propyl](3,5-dimethoxy-4-methylbenzoyl)amino}ethoxy)acetic acid;
(2-{(3,5-dimethoxy-4-methylbenzoyl)[3-(2-fluorophenyl)propyl]amino}ethoxy)acetic acid;
N-{5-[(methanesulfonyl)amino]-5-oxopentyl}-3,5-dimethoxy-4-methyl-N-(3-phenylpropyl)benzamide;
[(2-{[3-(2-chlorophenyl)propyl](3,5-dimethoxy-4-methylbenzoyl)amino}ethyl)sulfanyl]acetic acid;
[(2-{[3-(2,6-difluorophenyl)propyl](3,5-dimethoxy-4-methylbenzoyl)amino}ethyl)sulfanyl]acetic acid;
[(2-{[3-(2,4-difluorophenyl)propyl](3,5-dimethoxy-4-methylbenzoyl)amino}ethyl)sulfanyl]acetic acid;
[(2-{[1-(4-methoxyphenyl)cyclopropane-1-carbonyl](3-phenylpropyl)amino}ethyl)sulfanyl]acetic acid;
[(2-{(3,5-dimethoxy-4-methylbenzoyl)[3-(2-fluorophenyl)propyl]amino}ethyl)sulfanyl]acetic acid;
[(2-{(3,5-dimethoxy-4-methylbenzoyl)[3-(3-fluorophenyl)propyl]amino}ethyl)sulfanyl]acetic acid;
[(2-{(3,5-dimethoxy-4-methylbenzoyl)[3-(4-fluorophenyl)propyl]amino}ethyl)sulfanyl]acetic acid;
N-{5-[(cyclopropanesulfonyl)amino]-5-oxopentyl}-3,5-dimethoxy-4-methyl-N-(3-phenylpropyl)benzamide;
(4R)-5-[(3,5-dimethoxy-4-methylbenzoyl)(3-phenylpropyl)amino]-4-hydroxypentanoic acid;
(3E)-5-[(3,5-dimethoxy-4-methylbenzoyl)(3-phenylpropyl)amino]pent-3-enoic acid;
[(2-{[3-(3-chlorophenyl)propyl](3,5-dimethoxy-4-methylbenzoyl)amino}ethyl)sulfanyl]acetic acid;
N-{5-[(ethanesulfonyl)amino]-5-oxopentyl}-3,5-dimethoxy-4-methyl-N-(3-phenylpropyl)benzamide;
3,5-dimethoxy-4-methyl-N-{5-oxo-5-[(propane-2-sulfonyl)amino]pentyl}-N-(3-phenylpropyl)benzamide;
[(2-{[3-(3,5-difluorophenyl)propyl](3,5-dimethoxy-4-methylbenzoyl)amino}ethyl)sulfanyl]acetic acid;
5-{[3-(3,5-difluorophenyl)propyl](3,5-dimethoxy-4-methylbenzoyl)amino}pentanoic acid;
(2-{[3-(3,5-dichlorophenyl)propyl](3,5-dimethoxy-4-methylbenzoyl)amino}ethoxy)acetic acid;
(2-{[3-(2,6-difluorophenyl)propyl](3,5-dimethoxy-4-methylbenzoyl)amino}ethoxy)acetic acid;
(2-{(3,5-dimethoxy-4-methylbenzoyl)[3-(3-fluorophenyl)propyl]amino}ethoxy)acetic acid;
(2-{[3-(2,4-difluorophenyl)propyl](3,5-dimethoxy-4-methylbenzoyl)amino}ethoxy)acetic acid;
(2-{[3-(2-chlorophenyl)propyl](3,5-dimethoxy-4-methylbenzoyl)amino}ethoxy)acetic acid;
[(2-{(3,5-dimethoxy-4-methylbenzoyl)[3-(5-methylfuran-2-yl)propyl]amino}ethyl)sulfanyl]acetic acid;
5-{(3,5-dimethoxybenzoyl)[3-(5-methylfuran-2-yl)propyl]amino}pentanoic acid;
5-{(3,5-dimethoxybenzoyl)[3-(2-fluorophenyl)propyl]amino}pentanoic acid;
5-{[3-(2-chlorophenyl)propyl](3,5-dimethoxybenzoyl)amino}pentanoic acid;
5-{[3-(3,5-difluorophenyl)propyl](3,5-dimethoxybenzoyl)amino}pentanoic acid;
5-{[3-(2,6-difluorophenyl)propyl](3,5-dimethoxybenzoyl)amino}pentanoic acid;
5-{(3,5-dimethoxy-4-methylbenzoyl)[3-(5-methylfuran-2-yl)propyl]amino}pentanoic acid;
(2-{(3,5-dimethoxy-4-methylbenzoyl)[3-(4-fluorophenyl)propyl]amino}ethoxy)acetic acid;
N-{5-[(4-fluorobenzene-1-sulfonyl)amino]-5-oxopentyl}-3,5-dimethoxy-4-methyl-N-(3-phenylpropyl)benzamide;
3,5-dimethoxy-4-methyl-N-{5-oxo-5-[(pyridine-2-sulfonyl)amino]pentyl}-N-(3-phenylpropyl)benzamide;
3,5-dimethoxy-4-methyl-N-{5-oxo-5-[(pyridine-3-sulfonyl)amino]pentyl}-N-(3-phenylpropyl)benzamide;
3,5-dimethoxy-4-methyl-N-{5-oxo-5-[(pyridine-4-sulfonyl)amino]pentyl}-N-(3-phenylpropyl)benzamide;
N-{5-[(benzenesulfonyl)amino]-5-oxopentyl}-3,5-dimethoxy-4-methyl-N-(3-phenylpropyl)benzamide;
3-[(3,5-dimethoxy-4-methylbenzoyl)(3-phenylpropyl)amino]propyl (4-fluorobenzene-1-sulfonyl)carbamate;
5-{[3-(2,4-difluorophenyl)propyl](3,5-dimethoxy-4-methylbenzoyl)amino}pentanoic acid;
5-{[3-(2,6-difluorophenyl)propyl](3,5-dimethoxy-4-methylbenzoyl)amino}pentanoic acid;
5-{(3,5-dimethoxybenzoyl)[3-(4-fluorophenyl)propyl]amino}pentanoic acid;
5-{[2-ethoxy-1-(4-methoxyphenyl)cyclopropane-1-carbonyl](3-phenylpropyl)amino}pentanoic acid;
3,5-dimethoxy-4-methyl-N-{5-oxo-5-[(trifluoromethanesulfonyl)amino]pentyl}-N-(3-phenylpropyl)benzamide;
5-[(3,5-dimethoxy-4-methylbenzoyl)(3-phenylpropyl)amino]-2-hydroxypentanoic acid;
5-{[1-(4-methoxyphenyl)-3-oxocyclobutane-1-carbonyl](3-phenylpropyl)amino}pentanoic acid;
5-{(3,5-dimethoxy-4-methylbenzoyl) [(2R)-4-phenylbutan-2-yl]amino}pentanoic acid;
5-{(3,5-dimethoxy-4-methylbenzoyl) [(2S)-4-phenylbutan-2-yl]amino}pentanoic acid;
5-[(3,5-dimethoxy-4-methylbenzoyl)(3-phenylpropyl)amino]-2-hydroxy-2-methylpentanoic acid;
N-[2-({1-[(methanesulfonyl)amino]-2-methyl-1-oxopropan-2-yl}oxy)ethyl]-3,5-dimethoxy-4-methyl-N-(3-phenylpropyl)benzamide;
N-(2-{2-[(methanesulfonyl)amino]-2-oxoethoxy}ethyl)-3,5-dimethoxy-4-methyl-N-(3-phenylpropyl)benzamide;
5-{[cis-3-methoxy-1-(4-methoxyphenyl)cyclobutane-1-carbonyl](3-phenylpropyl)amino}pentanoic acid;
5-{[trans-3-methoxy-1-(4-methoxyphenyl)cyclobutane-1-carbonyl](3-phenylpropyl)amino}pentanoic acid;
5-[(2-methyl-4-oxo-3,4-dihydroquinazoline-8-carbonyl)(3-phenylpropyl)amino]pentanoic acid;
N-{5-[(methanesulfonyl)amino]-4-methyl-5-oxopentyl}-3,5-dimethoxy-4-methyl-N-(3-phenylpropyl)benzamide;
N-{5-[(methanesulfonyl)amino]-4,4-dimethyl-5-oxopentyl}-3,5-dimethoxy-4-methyl-N-(3-phenylpropyl)benzamide;
5-[(2,6-dimethoxypyrimidine-4-carbonyl)(3-phenylpropyl)amino]pentanoic acid;
5-[(3,5-dimethoxy-4-methylbenzoyl)(3-phenylpropyl)amino]hexanoic acid;
5-[(4-cyclopropyl-3,5-dimethoxybenzoyl)(3-phenylpropyl)amino]-2-hydroxypentanoic acid;
3,5-dimethoxy-4-methyl-N-(3-phenylpropyl)-N-[4-(1H-tetrazol-5-yl)butyl]benzamide;
5-[(5-chloro-2-methylpyrimidine-4-carbonyl)(3-phenylpropyl)amino]pentanoic acid;
5-[(6-fluoro-2-oxo-1,2,3,4-tetrahydroquinoline-4-carbonyl)(3-phenylpropyl)amino]pentanoic acid;
5-[(2-methyl-1-oxo-1,2-dihydroisoquinoline-4-carbonyl)(3-phenylpropyl)amino]pentanoic acid;
5-[(7-fluoro-2-oxo-1,2,3,4-tetrahydroquinoline-4-carbonyl)(3-phenylpropyl)amino]pentanoic acid;
5-[(2-acetamidopyridine-4-carbonyl)(3-phenylpropyl)amino]pentanoic acid;
5-[(3,6-dimethyl[,2]oxazolo[5,4-b]pyridine-4-carbonyl)(3-phenylpropyl)amino]pentanoic acid;
5-{(3,5-dimethoxy-4-methylbenzoyl)[3-(furan-2-yl)propyl]amino}pentanoic acid;
5-{(3,5-dimethoxy-4-methylbenzoyl)[3-(5-methylfuran-2-yl)propyl]amino}-2-hydroxy-2-methylpentanoic acid;
5-{(2,4-difluoro-3,5-dimethoxybenzoyl)[3-(5-methylfuran-2-yl)propyl]amino}pentanoic acid;
5-{(3,5-diethoxybenzoyl)[3-(5-methylfuran-2-yl)propyl]amino}pentanoic acid;
5-{[3,5-dimethoxy-4-(trifluoromethyl)benzoyl][3-(5-methylfuran-2-yl)propyl]amino}pentanoic acid;
5-{(4-cyclopropyl-3,5-dimethoxybenzoyl)[3-(5-methylfuran-2-yl)propyl]amino}pentanoic acid;
5-{(3,5-dimethoxy-4-methylbenzoyl)[3-(5-methylfuran-2-yl)propyl]amino}-2-methylpentanoic acid;
5-{(3,5-dimethoxy-4-methylbenzoyl)[3-(5-methylfuran-2-yl)propyl]amino}-2,2-dimethylpentanoic acid;
3,5-dimethoxy-4-methyl-N-{[(2R)-5-oxooxolan-2-yl]methyl}-N-(3-phenylpropyl)benzamide;
5-[(3,5-dimethoxy-4-methylbenzoyl)(3-phenylbutyl)amino]pentanoic acid;
2-[(3,5-dimethoxy-4-methylbenzoyl)(3-phenylpropyl)amino]ethyl (4-fluorobenzene-1-sulfonyl)carbamate;
2-[(3,5-dimethoxy-4-methylbenzoyl) (3-phenylpropyl)amino]ethyl (methanesulfonyl)carbamate;
5-{[3-(5-chlorofuran-2-yl)propyl](3,5-dimethoxy-4-methylbenzoyl)amino}pentanoic acid; and
3-[(3,5-dimethoxy-4-methylbenzoyl)(3-phenylpropyl)amino]propyl (methanesulfonyl)carbamate.

28. The compound or pharmaceutically acceptable salt of claim 1, wherein the compound is 5-[(3,5-dimethoxy-4-methylbenzoyl)(3-phenylpropyl)amino]pentanoic acid.

29. The compound or pharmaceutically acceptable salt of claim 1, wherein the compound is 5-[(3,5-dimethoxy-4-methylbenzoyl)(3-phenylpropyl)amino]-2-methylpentanoic acid.

30. The compound or pharmaceutically acceptable salt of claim 1, wherein the compound is (−)-(2R)-5-[(3,5-dimethoxy-4-methylbenzoyl)(3-phenylpropyl)amino]-2-methylpentanoic acid.

31. The compound or pharmaceutically acceptable salt of claim 1, wherein the compound is (+)-(2S)-5-[(3,5-dimethoxy-4-methylbenzoyl)(3-phenylpropyl)amino]-2-methylpentanoic acid.

32. The compound or pharmaceutically acceptable salt of claim 1, wherein the compound is ({2-[(3,5-dimethoxy-4-methylbenzoyl)(3-phenylpropyl)amino]ethoxy}methyl)phosphonic acid.

33. A pharmaceutical composition comprising a therapeutically effective amount of a compound of formula (I) according to claim 1, or a pharmaceutically acceptable salt thereof, in combination with a pharmaceutically acceptable carrier.

34. A method for treating liver, kidney, skin, heart and lung disease, cancer-associated disease, proliferative disease, inflammation/immune system disease, disease of secretory dysfunction, and fibrosis, in a subject comprising administering a therapeutically effective amount of a compound of formula (I) according to claim 1 or a pharmaceutically acceptable salt thereof, to a subject in need thereof.
